(19) United States
(12) Patent Application Publication
(10) Pub. No.: US 2025/0092415 A1
(43) Pub. Date: Mar. 20, 2025

(54) GLYCOALKALOID METABOLISM ENYZYMES (GAMES) AND USES THEREOF

(71) Applicants: Yeda Research and Development Co. Ltd., Rehovot (IL); Yissum Research Development Company of The Hebrew University of Jerusalem Ltd., Jerusalem (IL)

(72) Inventors: Asaph AHARONI, Tel-Aviv (IL); Prashant SONAWANE, Rehovot (IL); Pablo D. CARDENAS, Rehovot (IL); Saul BURDMAN, Rehovot (IL)

(73) Assignees: Yeda Research and Development Co. Ltd., Rehovot (IL); Yissum Research Development Company of The Hebrew University of Jerusalem Ltd., Jerusalem (IL)

(21) Appl. No.: 18/910,862

(22) Filed: Oct. 9, 2024

Related U.S. Application Data
(62) Division of application No. 16/484,200, filed on Aug. 7, 2019, now Pat. No. 12,129,475.

(30) Foreign Application Priority Data
Feb. 9, 2017 (IL) 250538

(51) Int. Cl. C12N 15/82; C12N 9/02; C12N 9/04

(52) U.S. Cl. CPC C12N 15/8279; C12N 9/0006; C12N 9/0071; C12N 15/8218; C12N 15/8243

(57) ABSTRACT

Disclosed herein are genetically modified plants having altered biological activity of 3-β-hydroxysteroid dehydrogenase/isomerase (GAME25), or 2-oxoglutarate-dependent dioxygenase (GAME31), or a combination thereof, wherein the genetically modified plants have an altered content of at least one cholesterol derived compound selected from the group including a steroidal alkaloid or a glycosylated derivative thereof and an unsaturated or saturated steroidal saponin or a glycoside derivative thereof. Further disclosed herein are genetically modified plants having altered expression of a gene encoding a 3-β-hydroxysteroid dehydrogenase/isomerase (GAME25), or a 2-oxoglutarate-dependent dioxygenase (GAME31), or a combination thereof, wherein the genetically modified plant has an altered content of at least one cholesterol derived compound selected from the group including a steroidal alkaloid or a glycosylated derivative thereof and an unsaturated or saturated steroidal saponin or a glycoside derivative thereof. Methods of producing these genetically modified plants are also disclosed.

### CROSS-REFERENCE TO RELATED APPLICATIONS

This application is Divisional Application of U.S. application Ser. No. 16/484,200 filed on Aug. 7, 2019, which is a National Phase Application of PCT International Application Number PCT/IL2018/050142, International filing date Feb. 8, 2018, which claims the benefit of Israeli Patent Application Number 250538, filed Feb. 9, 2017, which are all hereby incorporated by reference in their entirety herein.

### SEQUENCE LISTING STATEMENT

The instant application contains a Sequence Listing which has been submitted electronically in XML format and is hereby incorporated by reference in its entirety. Said XML copy, created on Dec. 1, 2024, is named P-80579-US1_SL and is 63,800 bytes in size.

### FIELD OF THE DISCLOSURE

Identification and use of key genes in the biosynthesis of steroidal alkaloids to genetically modified plants, wherein the genetically modified plants have an altered content of steroidal alkaloids and glycosylated derivatives thereof. Genetically modified plants described comprise Solanaceae crop plants, including those with reduced content of anti-nutritional steroidal glycoalkaloids.

### BACKGROUND

The plant kingdom produces hundreds of thousands of different small compounds that are often genus or family specific. These molecules, referred to as secondary metabolites, are not vital to cells that produce them, but contribute to the overall fitness of the organisms. Steroidal alkaloids are one example of secondary metabolites. They are low molecular weight nitrogen-containing organic compounds, typically with a heterocyclic structure. Steroidal alkaloid biosynthesis in plants is tightly controlled during development and in response to stress and pathogens.

Consisting of a C-27 cholestane skeleton and a heterocyclic nitrogen component, steroidal alkaloids (SAs) were suggested to be synthesized in the cytosol from cholesterol. Conversion of cholesterol to the alkamine SA should require several hydroxylation, oxidation and transamination reactions (Eich E. 2008. Solanaceae and Convolvulaceae—secondary metabolites: biosynthesis, chemotaxonomy, biological and economic significance: a handbook. Berlin: Springer), and in most cases further glycosylation to form steroidal glycoalkaloids (SGAs) (Amqvist L. et al. 2003. Plant Physiol 131:1792-1799). The oligosaccharide moiety components of SGAs directly conjugate to the hydroxyl group at C-3β of the alkamine steroidal skeleton (aglycone). The oligosaccharide moiety includes D-glucose, D-galactose, L-rhamnose, D-xylose, and L-arabinose, the first two monosaccharides being the predominant units.

SGA biosynthesis depends on genes encoding UDP-glycosyltransferases (UGTs) that add to the aglycone various sugar moieties (McCue K F et al., 2005. Plant Sci. 168:267-273; Itkin M et al., 2011. Plant Cell 23:4507-4525). The tomato GLYCOALKALOID METABOLISM 1 (GAME1) glycosyltransferase, a homolog of the potato SGT1 (McCue et al., 2005, supra), catalyzes galactosylation of the alkamine tomatidine (Itkin et al., 2011, supra).

SAs play a role in protecting plants against a broad range of pathogens and are thus referred to as phytoanticipins (antimicrobial compounds). SGAs are also known to contribute to plant resistance towards a wide range of pathogens, pests, and predators, including bacteria, fungi, oomycetes, viruses, insects and animals. Many SGAs are harmful to a variety of organisms including mammals and humans. When present in edible plant parts, these harmful SGAs are referred to as anti-nutritional substances. These SGAs cause gastrointestinal and neurological disorders and, at high concentrations, may be lethal to humans. For this reason, total SGA levels exceeding 200 mg per kilogram fresh weight of edible tuber are deemed unsafe for human consumption.

Thus, SGA are well known anti-nutritional secondary metabolites produced by numerous members of Solanaceae family (e.g. potato, tomato, eggplant). Well-known examples of SGA anti-nutritional secondary metabolite compounds include α-tomatine and dehydrotomatine in tomato (Solanum lycopersicum), α-chaconine and α-solanine in potato (Solanum tuberosum), and α-solamargine and α-solasonine in aubergine (Solanum melongena).

In tomato, α-tomatine and dehydrotomatine represent the major SGAs accumulating predominantly in green tissues; young and mature leaves, flower buds, skin and seeds of immature and mature green fruit. Dehydrotomatidine (i.e. tomatidenol) is the first SA aglycone formed in SGA biosynthesis which could further be hydrogenated at the C-5 position to form tomatidine (FIGS. 1A-1C). Both aglycones are further glycosylated (tetra-saccharide moiety i.e. lycotetrose) to produce dehydrotomatine and α-tomatine respectively (FIGS. 1A-1C). Thus, the SGA pathway branches at dehydrotomatidine for either formation of tomatidine derived SGAs or glycosylated dehydrotomatine derivatives (FIGS. 1A-1C). Notably, dehydrotomatidine and tomatidine are only different in their structures by the presence or absence of the double bond at the C-5 position. The conversion of dehydrotomatidine to tomatidine was hypothesized in the past as a single reaction catalyzed by a hypothetical hydrogenase. In most tomato plant tissues, the relative portion of dehydrotomatine as compared to α-tomatine ranges from ˜2.5-˜10%. As tomato fruit matures and reaches to the red stage, the entire pool of α-tomatine and dehydrotomatine is largely being converted to esculeosides (major SGAs) and dehydroesculeosides (minor SGAs), respectively (FIGS. 1A-1C).

In cultivated potato, α-chaconine and α-solanine are the major SGAs sharing the same aglycone, solanidine (in which a C-5,6 double bond is present) and possess chacotriose and solatriose moieties, respectively. As there is no demissidine or demissine detected in cultivated potatoes, it was suggested that a hydrogenase enzyme able to convert solanidine to demissidine is lacking in these species. Several wild potato species (e.g. S. demissum, S. chacoense, S. commersonii) and their somatic hybrids (S. brevidens X S. tuberosum), predicted to contain an active hydrogenase, do produce demissidine or its glycosylated form, demissine being one of their major SGAs (FIGS. 2A-2B). In eggplant, α-solamargine and α-solasonine are the most abundant SGAs derived from the solasodine aglycone (in which a C-5,6 double bond is present); while some wild solanum species, e.g. S. dulcamara produce soladulcidine or its glycosylated forms, soladulcine A and β-soladulcine (C-5,6 double bond is absent), as major SGAs from the solasodine aglycone (FIGS. 3A-3C).

In addition to SGAs, many Solanum species also produce cholesterol-derived unsaturated or saturated steroidal saponins. Unsaturated and saturated steroidal saponins are widespread in the plant kingdom, especially among monocots, e.g. the Agavaceae, Asparagaceae, Dioscoreaceae and Liliaceae families. Similar to SGAs, steroidal saponins are highly diverse in structures and could be either saturated (e.g. sarasapogenin) or unsaturated (e.g. diosgenin) in the C-5,6 position.

Cholesterol, the main sterol produced by all animals, serves as a key building block in the biosynthesis of SGAs. An array of tomato and potato GLYCOALKALOIDMETABOLISM (GAME) genes participating in core SGA biosynthesis starting from cholesterol were reported in recent years. The tomato SGAs biosynthetic pathway can be divided into two main parts. In the first, the SA aglycone is formed from cholesterol by the likely action of the GAME6, GAME8, GAME11, GAME4 and GAME12 enzymes. The second part results in the generation of SGA through the action of UDP-glycosyltransferases (UGTs): GAME1, GAME2, GAME17 and GAME18 in tomato, and STEROL ALKALOID GLYCOSYL TRANSFERASE1 (SGT1), SGT2 and SGT3 in potato.

The formation of unsaturated steroidal saponin aglycone is also a main step in steroidal saponin biosynthesis pathway (FIG. 3A). The aglycone of steroidal saponin is either a spirostanol (closed ring) or a furostanol (open ring). Both these saponin aglycones (e.g. diosgenin) undergoes either glycosylation to form unsaturated saponin glycosides (e.g. dioscin) or hydrogenation at C-5,6 position to form saturated saponin aglycones (e.g. sarasapogenin). These saturated saponin aglycones are further glycosylated to produce downstream saturated saponin glycosides (e.g. parillin) (FIG. 3A). Therefore, like SGAs, unsaturated and saturated aglycone forms of steroidal saponin metabolites also primarily contribute for their structural diversity. Notably, dehydrotomatidine/tomatidine, solanidine/demissidine, solasodine/soladulcidine SA aglycones and unsaturated/saturated steroidal saponin aglycones are only different in their structures by the presence or absence of the double bond at the C-5,6 position. However, the biosynthetic basis of the formation of saturated steroidal alkaloid and steroidal saponin aglycones from their unsaturated forms in Solanaceae or in any other plant families remains unclear till date. In fact, the conversion of dehydrotomatidine to tomatidine in tomato, and solanidine to demissidine in wild potato species by elimination of the C-5,6 double bond was hypothesized for decades to be carried out in a single reaction catalyzed by a hypothetical hydrogenase enzyme.

There is an ongoing attempt to elucidate the biosynthesis pathway of steroidal alkaloids and to control their production. It would be advantageous to both the farmer and the consumer to have a Solanaceae plant wherein the levels of SGA present would provide the necessary plant resistance to pathogens and predators, while the fruits, tubers and vegetables had reduced anti-nutritional secondary metabolites. The ability to manipulate the synthesis of the SGAs may provide the means to develop, through classical breeding or genetic engineering, crops with modified levels and composition of SGAs, conferring the plant with a preexisting chemical barrier against a broad range of pathogens and insects. At the same time, anti-nutritional compounds (e.g., chaconine and solanine from potato) would be removed.

Disclosed herein are newly identified genes present in Solanaceae family members encoding enzymes active in the steroidal glycoalkaloids metabolic pathway, whose manipulation may provide just such a balance between plant resistance and decreased anti-nutritional secondary metabolites. For example, in tomato plants genes encoding enzymes active in the conversion of dehydrotomatidine to tomatidine and from α-tomatine to hydroxytomatine have been identified, whose manipulation within tomato plants may provide just such a balance between tomato plant resistance during growth and fruit development, and decreased anti-nutritional secondary metabolites present in resultant tomatoes.

### SUMMARY

In one aspect, provided herein is a genetically modified plant comprising an altered content of at least one cholesterol derived compound selected from the group comprising a steroidal alkaloid or a glycosylated derivative thereof, and an unsaturated or saturated steroidal saponin or a glycoside derivative thereof, said plant comprising at least one cell having an altered biological activity of at least one enzyme selected from the group comprising a 3-β-hydroxysteroid dehydrogenase/isomerase (GAME25) and a 2-oxoglutarate-dependent dioxygenase (GAME31), or a combination thereof, or an altered expression of at least one gene selected from the group comprising a gene encoding a 3-β-hydroxysteroid dehydrogenase/isomerase (GAME25) and a gene encoding a 2-oxoglutarate-dependent dioxygenase (GAME31), or a combination thereof, or a combination thereof of an altered biological activity of at least one enzyme selected from the group comprising a 3-p-hydroxysteroid dehydrogenase/isomerase (GAME25) and a 2-oxoglutarate-dependent dioxygenase (GAME31), or a combination thereof, and an altered expression of at least one gene selected from the group comprising a gene encoding a 3-β-hydroxysteroid dehydrogenase/isomerase (GAME25) and a gene encoding a 2-oxoglutarate-dependent dioxygenase (GAME31), or a combination thereof, wherein the at least one cell of said genetically modified plant has an altered content of at least one cholesterol derived compound selected from the group comprising a steroidal alkaloid or a glycosylated derivative thereof and an unsaturated or saturated steroidal saponin or a glycoside derivative thereof compared to a corresponding unmodified plant.

In a related aspect, the amino acid sequence of 3-β-hydroxysteroid dehydrogenase/isomerase (GAME25) comprises the amino acid sequence set forth in any one of SEQ ID NO: 3, SEQ ID NO: 12, and SEQ ID NO: 15, or a protein homologue thereof, wherein said protein homologue is at least 80% homologous to any of SEQ ID NO: 3, SEQ ID NO: 12, and SEQ ID NO: 15.

In a related aspect, 3-β-hydroxysteroid dehydrogenase/isomerase (GAME25) is encoded by a gene comprising the polynucleotide sequence set forth in any one of SEQ ID NO: 1, SEQ ID NO: 2, SEQ ID NO: 11, SEQ ID NO: 13, and SEQ ID NO: 14, or a gene homologue thereof, wherein said gene homologue is at least 80% homologous to any of SEQ ID NO: 1, SEQ ID NO: 2, SEQ ID NO: 11, SEQ ID NO: 13, and SEQ ID NO: 14.

In a related aspect, the amino acid sequence of 2-oxoglutarate-dependent dioxygenase (GAME31) comprises the amino acid sequence set forth in any one of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53, or a protein homologue thereof, wherein said protein homologue is at least 80% homologous to any of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53.

In a related aspect, 2-oxoglutarate-dependent dioxygenase (GAME31) is encoded by a gene comprising the polynucleotide sequence set forth in any one of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52, or a nucleic acid sequence having at least 80% identity to any of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52.

In a related aspect, altered expression comprises a reduced or inhibited expression of said GAME25 gene, or GAME31 gene, or a combination thereof compared to its expression in a corresponding unmodified plant; or an increased expression of said GAME25 gene, or GAME31 gene, or a combination thereof compared to its expression in a corresponding unmodified plant; or a combination of reduced or inhibited expression of one of said GAME25 gene or said GAME31 gene, and increased expression the other of said GAME25 gene or said GAME31 gene of compared to their expression in a corresponding unmodified plant and increased expression of.

In a related aspect, altered biological activity comprises increased enzyme activity of said 3-β-hydroxysteroid dehydrogenase/isomerase (GAME25) or said 2-oxoglutarate-dependent dioxygenase (GAME31), or the combination thereof, or increased stability of said 3-β-hydroxysteroid dehydrogenase/isomerase (GAME25) or said 2-oxoglutarate-dependent dioxygenase (GAME31), or the combination thereof, or decreased enzyme activity of said 3-β-hydroxysteroid dehydrogenase/isomerase (GAME25) or said 2-oxoglutarate-dependent dioxygenase (GAME31), or the combination thereof, or decreased stability of said 3-β-hydroxysteroid dehydrogenase/isomerase (GAME25) or said 2-oxoglutarate-dependent dioxygenase (GAME31), or the combination thereof, compared to the biological activity in an unmodified plant.

In a related aspect, the expression of the at least one gene or combination thereof in said genetically modified plant is altered, said altering comprises mutagenizing the at least one gene, said mutation present within a coding region of said at least one gene, or a regulatory sequence of said at least one gene, or a combination thereof.

In a related aspect, mutagenesis comprises one or more point mutations, a site-directed point mutagenesis, random point mutagenesis, genome editing, mutagenesis using uracil-containing templates, oligonucleotide-directed mutagenesis, phosphorothioate-modified DNA mutagenesis, mutagenesis using gapped duplex DNA, point mismatch repair mutagenesis, mutagenesis using a repair-deficient host strains, restriction-selection and restriction-purification, deletion mutagenesis, mutagenesis by total gene synthesis, mutagenesis during double-strand break repair, mutagenesis by chimeric constructs, mutagenesis by a CRISPR/Cas system, mutagenesis by a zinc-finger nucleases (ZFN) system, mutagenesis by a transcription activator-like effector nucleases (TALEN) system, or any combination thereof.

In a related aspect, the plant is a Solanaceae crop plant. In a related aspect, a Solanaceae crop plant is selected from the group comprising a cultivated tomato plant, a wild-tomato plant, a cultivated potato plant, a wild-potato plant, an aubergine plant, a chili pepper plant, a bell pepper plant, and a bittersweet plant.

In a related aspect, altered content of at least one cholesterol derived compound selected from the group comprising a steroidal alkaloid or a glycosylated derivative thereof and an unsaturated or saturated steroidal saponin or a glycoside derivative thereof comprises a reduced content of at least one steroidal alkaloid or a glycosylated derivative thereof compared to said corresponding unmodified plant, or an increased content of at least one steroidal alkaloid or a glycosylated derivative thereof compared to said corresponding unmodified plant, or a reduced content of at least one unsaturated or saturated steroidal saponin or a glycosylated derivative thereof compared to said corresponding unmodified plant, or an increased content of at least one unsaturated or saturated steroidal saponin or a glycosylated derivative thereof compared to said corresponding unmodified plant, or a combination of a reduced content of at least one steroidal alkaloid or a glycosylated derivative thereof, and an increased content of at least one steroidal alkaloid or a glycosylated derivative thereof, or an appearance of at least one steroidal alkaloid or a glycosylated derivative thereof compared to said corresponding unmodified plant that does not contain said at least one steroidal alkaloid or a glycosylated derivative thereof, or an appearance of at least one unsaturated or saturated steroidal saponin or a glycosylated derivative thereof compared to said corresponding unmodified plant that does not contain said at least one unsaturated or saturated steroidal saponin or a glycosylated derivative thereof, or any combination thereof, compared to said corresponding unmodified plant.

In a related aspect, the reduced content of at least one steroidal alkaloid or a glycosylated derivative thereof comprises reduced content of at least one anti-nutritional steroidal alkaloid or a glycosylated derivative thereof, or reduced content of at least one toxic steroidal alkaloid or a glycosylated derivative thereof, or a combination thereof. In a related aspect, the increased content of at least one steroidal alkaloid or a glycosylated derivative thereof results in increased plant resistance to at least one plant pathogen, pest, or predator, or any combination thereof, and optionally generates precursor molecules for steroidal alkaloid molecules that provide resistance to at least one plant pathogen, pest, or predator, or any combination thereof.

In a related aspect, the at least one steroidal alkaloid or glycosylated derivative thereof is selected from the group comprising tomatidine, α-tomatine, α-tomatine isomer (1 and 2), α-tomatine isomer 1, α-tomatine isomer 2, hydroxytomatine, acetoxytomatine, dehydrotomatidine, dehydrotomatine, dehydrotomatine isomer 1, dehydrotomatine+4-hexose, acetoxy-hydroxytomatine, acetoxy-hydroxy-dehydrotomatine, tomatidine+4 hexose, esculeosides, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydroesculeosides, dehydroesculeoside A, dehydroesculeoside A+hexose, leptinine I, leptinine II, leptine I, leptine II, lycoperosides, soladulcidine, β-soladulcine, soladulcine A, solanidine, α-solanine, α-chaconine, solasodine, α-solasonine, α-solamargine, hydroxysolasonine, and hydroxysolamargine, or any derivatives thereof, or any combination thereof.

In a related aspect, the at least one unsaturated or saturated steroidal saponin or glycosylated derivative thereof is selected from the group comprising dioscin, diosgenin, parillin, and sarasapogenin. In a related aspect, the plant is a potato plant and said at least one steroidal alkaloid or glycosylated derivative thereof is selected from the group comprising α-solanine, α-chaconine, leptinine I, leptinine II, leptine I, and leptine II. In a related aspect, the plant is a tomato plant and said at least one steroidal alkaloid or glycosylated derivative thereof is selected from the group comprising α-tomatine, α-tomatine isomer (1 and 2), α-tomatine isomer 2, hydroxytomatine, acetoxytomatine, dehydrotomatidine, dehydrotomatine, dehydrotomatine isomer 1, dehydrotomatine+4-hexose, esculeosides, lycoperoside, or any derivatives thereof, or any combination thereof.

In a related aspect, the plant is an eggplant plant and said at least one steroidal alkaloid or glycosylated derivative thereof is selected from the group comprising soladulcidine, β-soladulcine, soladulcine A, or any derivatives thereof, and said unsaturated or saturated steroidal saponin or glycosylated derivative thereof is selected from the group comprising dioscin, diosgenin, parillin, and sarasapogenin, or any derivatives thereof, or any combination thereof. In a related aspect, the genetically modified plant is a transgenic plant comprising said at least one cell comprising at least one silencing molecule targeted to a gene selected from the group comprising GAME25 and GAME31, or a combination thereof.

In a related aspect, the silencing molecule is selected from the group comprising an RNA interference molecule and an antisense molecule. In a related aspect, the silencing molecule comprises a polynucleotide having a nucleic acid sequence substantially complementary to a region of the GAME25 gene or a complementary sequence thereof. In a related aspect, the nucleic acid sequence comprises the nucleic acid sequence set forth in SEQ ID NO: 8, or a fragment thereof. In a related aspect, the silencing molecule comprises a polynucleotide having a nucleic acid sequence substantially complementary to a region of the GAME31 gene or a complementary sequence thereof. In a related aspect, the nucleic acid sequence is set forth in any one of SEQ ID NO: 58 or SEQ ID NO: 59.

In a related aspect, the at least one cell having altered biological activity, or altered expression, or a combination thereof, is selected from the group consisting of leaf cell, a young leaf cell, a mature leaf cell, a bud cell, a petal cell, a flower cell, a stem cell, a shoot cell, a peel cell, a root cell, a fruit cell, a tuber cell, and a vegetable cell. In a related aspect, the fruit is a green fruit, a breaker fruit, or a red ripe fruit.

In one aspect, provided herein is a method of reducing the content of at least one cholesterol derived compound selected from the group comprising a steroidal alkaloid or a glycosylated derivative thereof and an unsaturated or saturated steroidal saponin or a glycoside derivative thereof in a plant, said method comprising transforming at least one plant cell within said plant with at least one silencing molecule targeted to a nucleic acid sequence encoding at least one protein selected from the group comprising 3-β-hydroxysteroid dehydrogenase/isomerase (GAME25), 2-oxoglutarate-dependent dioxygenase (GAME31), or any combination thereof, or transforming at least one plant cell within said plant with at least one polynucleotide sequence encoding at least one protein selected from the group comprising 3-β-hydroxysteroid dehydrogenase/isomerase (GAME25), or a 2-oxoglutarate-dependent dioxygenase (GAME31), or any combination thereof, wherein said at least one polynucleotide sequence comprises a mutation in a coding region or a regulatory region; or a combination thereof; thereby producing a plant with a reduced content of said at least one cholesterol derived compound selected from the group comprising a steroidal alkaloid or a glycosylated derivative thereof and an unsaturated or saturated steroidal saponin or a glycoside derivative thereof compared to a corresponding non-transformed plant.

In a related aspect, the at least one steroidal alkaloid or a glycosylated derivative thereof comprises α-tomatine, α-tomatine isomer (1 and 2), hydroxytomatine, or acetoxytomatine, and said at least one unsaturated or saturated steroidal saponin or glycosylated derivative thereof comprises a sarasapogenin or any combination thereof. In a related aspect, the further at least one steroidal alkaloid or a glycosylated derivative thereof is increased, said at least one steroidal alkaloid or a glycosylated derivative thereof comprising a dehydrotomatine, a dehydrotomatine isomer 1, or a dehydrotomatidine+4-hexose, and said at least one unsaturated or saturated steroidal saponin or glycosylated derivative thereof comprises a diosgenin, or any combination thereof.

In one aspect, provided herein is a method of enhancing the content of at least one cholesterol derived compound selected from the group comprising a steroidal alkaloid or a glycosylated derivative thereof and an unsaturated or saturated steroidal saponin or a glycoside derivative thereof in a plant, comprising transforming at least one plant cell within said plant with a nucleic acid sequence encoding 3-β-hydroxysteroid dehydrogenase/isomerase (GAME25), 2-oxoglutarate-dependent dioxygenase (GAME31), or a combination thereof, wherein said transforming results in overexpression of said GAME25, GAME31, or a combination thereof; or transforming at least one plant cell with at least one polynucleotide sequence encoding at least one protein selected from the group comprising 3-β-hydroxysteroid dehydrogenase/isomerase (GAME25), or a 2-oxoglutarate-dependent dioxygenase (GAME31), or any combination thereof, wherein said at least one polynucleotide sequence comprises a mutation in a coding region or a regulatory region; thereby producing a plant with an enhanced content of said at least cholesterol derived compound selected from the group comprising a steroidal alkaloid or a glycosylated derivative thereof and an unsaturated or saturated steroidal saponin or a glycoside derivative thereof compared to a corresponding non-transformed plant.

In a related aspect, the at least one steroidal alkaloid or a glycosylated derivative thereof comprises a α-tomatine, α-tomatine isomer (1 and 2), hydroxytomatine, acetoxytomatine, soladulcidine, β-soladulcine, soladulcine A, an unsaturated or saturated steroidal saponin, a leptin, or a leptinine, and said unsaturated or saturated steroidal saponin or glycosylated derivative thereof comprises a sarasapogenin, or any combination thereof.

In one aspect, provided herein is a method of producing beneficial steroidal derivatives, said method comprising the steps of incubating a recombinant plant 3-β-hydroxysteroid dehydrogenase/isomerase (GAME25), or 2-oxoglutarate-dependent dioxygenase (GAME31), or a combination thereof, with selected precursor molecules under biosynthetic conditions; and collecting and isolating the steroidal derivatives from the biosynthetic medium.

In one aspect, provided herein is a recombinant protein having the amino acid sequence set forth in SEQ ID NO: 3, SEQ ID NO: 12, or SEQ ID NO: 15, or protein homologue thereof, wherein said protein homolog is at least 50% homologous to any of SEQ ID NO: 3, SEQ ID NO: 12, or SEQ ID NO: 15 and has the same catalytic function as the protein encoded by SEQ ID NO: 3, SEQ ID NO: 12, or SEQ ID NO: 15, for the production of a steroidal derivative comprising a steroidal alkaloid or a glycosylated derivative thereof, an unsaturated or saturated steroidal saponin or a glycoside derivative thereof, or a biosynthetic product thereof.

In one aspect, provided herein is the use of a recombinant protein having the amino acid sequence set forth in SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 or SEQ ID NO: 53, or protein homologue thereof, wherein said protein homolog is at least 50% homologous to any of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 or SEQ ID NO: 53 and has the same catalytic function as the protein encoded by SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 or SEQ ID NO: 53, for the production of a steroidal derivative comprising a steroidal alkaloid or a glycosylated derivative thereof, an unsaturated or saturated steroidal saponin or a glycoside derivative thereof, or a biosynthetic product thereof.

In one aspect, provided herein is the use of a plant nucleic acid sequence encoding a 3-β-hydroxysteroid dehydrogenase/isomerase (GAME25) enzyme, said nucleic acid comprising the sequence set forth in SEQ ID NO: 1, SEQ ID NO: 2, SEQ ID NO: 11, SEQ ID NO: 13, or SEQ ID NO: 14 or a nucleic acid sequence having a sequence which is at least 50% identical to SEQ ID NO: 1, SEQ ID NO: 2, SEQ ID NO: 11, SEQ ID NO: 13, or SEQ ID NO: 14, wherein said encoded enzyme has the same catalytic function as the protein encoded by SEQ ID NO: 1, SEQ ID NO: 2, SEQ ID NO: 11, SEQ ID NO: 13, or SEQ ID NO: 14, or a plant 2-oxoglutarate-dependent dioxygenase (GAME31) enzyme, said nucleic acid comprising the sequence set forth in SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52 or a nucleic acid sequence having a sequence which is at least 50% identical to SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52, wherein said encoded enzyme has the same catalytic function as the protein encoded by SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52, or a combination of a plant nucleic acid encoding 3-β-hydroxysteroid dehydrogenase/isomerase (GAME25) and a plant nucleic acid encoding 2-oxoglutarate-dependent dioxygenase (GAME31), for the production of a recombinant cell capable of biosynthesis of a steroidal alkaloid or a glycosylated derivative thereof or an unsaturated or saturated steroidal saponin or a glycosylated derivative thereof, wherein said cell comprises a non-plant cell.

In one aspect, provided herein is a method for breeding a plant having altered content of at least one cholesterol derived compound selected from the group comprising a steroidal alkaloid or a glycosylated derivative thereof and an unsaturated or saturated steroidal saponin or a glycoside derivative thereof, said method comprising: providing a first plant, wherein the expression level of a polynucleotide encoding a 3-β-hydroxysteroid dehydrogenase/isomerase (GAME25), a 2-oxoglutarate-dependent dioxygenase (GAME31), or a combination thereof is in a pre-determined range of values, or a biological activity of a 3-β-hydroxysteroid dehydrogenase/isomerase (GAME25), a 2-oxoglutarate-dependent dioxygenase (GAME31), or a combination thereof, is in a pre-determined range of values; providing a second plant; crossing said first and second plants to generate an offspring plant; and selecting an offspring plant that has a significantly different content of at least one cholesterol derived compound selected from the group comprising a steroidal alkaloid or a glycosylated derivative thereof and an unsaturated or saturated steroidal saponin or a glycoside derivative thereof, compared to said second plant.

In a related aspect, the pre-determined value of expression comprises under-expression or over-expression or said pre-determine value of biological activity comprises increases enzyme activity, or decreased enzyme activity, or increased stability, or decreased stability of said 3-β-hydroxysteroid dehydrogenase/isomerase (GAME25) or 2-oxoglutarate-dependent dioxygenase (GAME31), or the combination thereof.

In one aspect, provided herein is a method for breeding a plant having an altered expression of at least one gene selected from the group comprising a gene encoding 3-β-hydroxysteroid dehydrogenase/isomerase (GAME25), a gene encoding a 2-oxoglutarate-dependent dioxygenase (GAME31), or a combination thereof, said method comprising: providing a first transformed plant, wherein said first transformed plant is transformed with an expression vector comprising a polynucleotide comprising at least one silencing molecule targeted to a nucleic acid sequence encoding at least one protein selected from the group comprising a3-β-hydroxysteroid dehydrogenase/isomerase (GAME25) and a 2-oxoglutarate-dependent dioxygenase (GAME31), or a combination thereof, wherein said at least one silencing molecule is operably linked to a promoter; or providing a first transformed plant, wherein said first transformed plant is transformed with an expression vector comprising at least one polynucleotide which overexpresses at least one protein selected from the group comprising a 3-β-hydroxysteroid dehydrogenase/isomerase (GAME25), a 2-oxoglutarate-dependent dioxygenase (GAME31), or a combination thereof, or providing a first transformed plant, wherein said first transformed plant is transformed with an expression vector comprising at least one polynucleotide which comprises a mutation in a gene encoding at least one protein selected from the group comprising a 3-β-hydroxysteroid dehydrogenase/isomerase (GAME25), a 2-oxoglutarate-dependent dioxygenase (GAME31), or a combination thereof; providing a second non-transformed plant; crossing said first transformed plant with a second plant to generate a hybrid plant, wherein the hybrid plant comprises the expression vector; and selecting a hybrid plant that has an altered expression of said at least one gene selected from the group comprising a gene encoding 3-β-hydroxysteroid dehydrogenase/isomerase (GAME25), a gene encoding a 2-oxoglutarate-dependent dioxygenase (GAME31), or a combination thereof compared to a corresponding unmodified plant; and wherein optionally, said plant comprises an altered content of at least one cholesterol derived compound selected from the group comprising a steroidal alkaloid or a glycosylated derivative thereof and an unsaturated or saturated steroidal saponin or a glycoside derivative thereof, compared to a corresponding unmodified plant.

In a related aspect, the at least one silencing molecule or said overexpressing polynucleotide is operably linked to a constitutive promoter, an inducible promoter, a tissue-specific promoter, or a developmental-stage specific promoter. In a related aspect, the at least one polynucleotide comprising a mutation is operably linked to a constitutive promoter, an inducible promoter, a tissue-specific promoter, or a developmental-stage specific promoter. In a related aspect, the expression level and/or biological activity of the 3-β-hydroxysteroid dehydrogenase/isomerase (GAME25), or the 2-oxoglutarate-dependent dioxygenase (GAME31), or a combination thereof, provide a biological marker for a plant comprising altered content of at least one cholesterol derived compound selected from the group comprising a steroidal alkaloid or a glycosylated derivative thereof and an unsaturated or saturated steroidal saponin or a glycoside derivative thereof.

In a related aspect, the altered content comprises reduced content of an anti-nutritional or toxic steroidal alkaloid or a glycosylated derivative thereof. In a related aspect, the altered content comprises increased content of a steroidal alkaloid or a glycosylated derivative thereof that provides resistance to a plant pathogen, pest, or predator.

In some aspects, provided herein is a method for selecting plant progenitors, said method comprising a step of determining the expression level of a gene encoding a 3-β3-hydroxysteroid dehydrogenase/isomerase (GAME25), or a 2-oxoglutarate-dependent dioxygenase (GAME31), or a combination thereof, wherein expression levels of said GAME25 gene, or said GAME31 gene, or the combination thereof, is predictive of altered content of at least one cholesterol derived compound selected from the group comprising a steroidal alkaloid or a glycosylated derivative thereof and an unsaturated or saturated steroidal saponin or a glycoside derivative thereof in an offspring plant; or determining the biological activity of a 3-β-hydroxysteroid dehydrogenase/isomerase (GAME25), or a 2-oxoglutarate-dependent dioxygenase (GAME31), or a combination thereof, wherein biological activity of said GAME25 enzyme, or said GAME31 enzyme, or the combination thereof, is predictive of altered content of at least one cholesterol derived compound selected from the group comprising a steroidal alkaloid or a glycosylated derivative thereof and an unsaturated or saturated steroidal saponin or a glycoside derivative thereof in an offspring plant.

In some aspects, provided herein is a method for determining the capacity of a plant to produce at least one cholesterol derived compound selected from the group comprising a steroidal alkaloid or a glycosylated derivative thereof and an unsaturated or saturated steroidal saponin or a glycoside derivative thereof in at least apart of said plant, said method comprising a step of measuring the expression level of GAME25, or GAME31, or a combination thereof, in at least a part of said plant; or measuring the biological activity of a 3-β-hydroxysteroid dehydrogenase/isomerase (GAME25), or a 2-oxoglutarate-dependent dioxygenase (GAME31), or a combination thereof, in at least a part of said plant; or a combination thereof.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The following drawings form part of the present specification and are included to further demonstrate certain embodiments of the present disclosure, the compositions and formulations described herein may be better understood by reference to one or more of these drawings in combination with the detailed description of specific embodiments presented herein.

FIGS. 1A-1C together provide an overview of Steroidal Glycoalkaloid (SGA) biosynthesis in tomato. SGA biosynthesis starts with the conversion of cholesterol to unsaturated dehyrotomatidine (tomatidenol), the first SA aglycone in the green stage of fruit development. This occurs through the action of GAME enzymes (GAME-6, 8, 11, 4, 12). Dehydrotomatidine is further converted to saturated tomatidine, another steroidal alkaloid aglycone. Both dehydrotomatidine and tomatidine are glycosylated by various UGTs (GAME-1, 17, 18, 2) to form dehydrotomatine and α-tomatine respectively. The presence or absence of C-5,6 double bond is the only difference between dehydrotomatidine and tomatidine steroidal alkaloid aglycones structure. This difference in the C-5,6 double bond at steroidal aglycones level further creates a vast structural diversity of SGAs (either unsaturated or saturated derivatives) during tomato fruit development and ripening stages (see all structures). α-tomatine and dehydrotomatine are major SGAs in green tomato fruit. Subsequently, hydroxy- and/or acetoxy-derivatives of α-tomatine and dehydrotomatine accumulate in the breaker tomato fruit. In red fruit, esculeosides and lycoperosides are the most abundant SGAs. α-tomatine derived saturated SGAs are highly abundant compared to dehydrotomatine-derived unsaturated SGAs throughout tomato fruit development and ripening. The conversion of dehydrotomatidine to tomatidine was previously predicted as a single step reaction by the action of a hypothetical hydrogenase. FIG. 1A shows a schematic representation of the main steps of SGA biosynthesis in tomato. FIG. 1B shows a schematic representation of SGA biosynthesis in tomato illustrating also the molecular structure of the compounds generated. FIG. 1C shows a further schematic representation of SGA biosynthesis in tomato illustrating also the molecular structure of the compounds generated. α-tomatine derived saturated SGAs are shown in black color and dehydrotomatine-derived unsaturated SGAs are shown in red color.

FIGS. 2A-2B provide an overview of SGA biosynthesis in potato. Cholesterol is first converted to the aglycone solanidine in potato. Solanidine is further glycosylated by SGT enzymes to produce α-solanine and α-chaconine in cultivated potato. Some wild potato species (e.g. S. chacoense and S. demissum etc.) produce demissidine or its glycosylated form demissine from the solanidine aglycone. The enzymatic conversion of solanidine to demissidine in wild potato plants was hypothesized previously to be carried out in a single reaction catalyzed by a hypothetical hydrogenase GAME. GTs—GAME Glycosyltransferase. SGT—Sterol alkaloid Glycosyltransferase. Dotted arrows represent multiple glycosyltransferase enzymatic steps between the compounds shown. Solid arrows represent a single enzymatic step between compounds. FIG. 2A shows a schematic representation of the main steps of SGA biosynthesis in potato. FIG. 2B shows a schematic representation of SGA biosynthesis in potato illustrating also the molecular structure of the compounds generated.

FIGS. 3A-3C shows the proposed biosynthetic pathway for steroidal saponin glycosides in steroidal saponin producing plant species and steroidal glycoalkaloids in eggplant. FIG. 3A shows biosynthetic pathway of steroidal saponins and their glycosides. First, cholesterol is converted to either unsaturated furostanol type saponin aglycone or spirostanol type saponin aglycone. Both these unsaturated aglycones are further glycosylated by GTs (glycosyltransferases) to form their unsaturated saponin glycosides respectively. Alternatively, both unsaturated saponin aglycones are further hydrogenated at C-5,6 position to form their saturated saponin aglycone forms. Similar to unsaturated ones, these saturated aglycone forms also undergoes glycosylation to form diverse saturated saponin glycosides. The enzymatic conversion responsible for formation of saturated saponin aglycone from unsaturated ones is unknown till date in any well-known steroidal saponin producing plant species. FIG. 3B shows a schematic representation of the main steps of SGA biosynthesis in eggplant. FIG. 3C shows a schematic representation of SGA biosynthesis in eggplant illustrating also the molecular structure of the compounds generated. In eggplant SGA biosynthesis, cholesterol is first converted to solasodine, unsaturated SA aglycone. Solasodine is further glycosylated by SGT (STEROL ALKALOID GLYCOSYL TRANSFERASE) enzymes to produce unsaturated α-solasonine and α-solamargine SGAs in cultivated eggplant. Cultivated eggplant is deficient in GAME25 gene and therefore lacks saturated SGAs. In contrary, some Solanum species, e.g. S. dulcamara produce saturated soladulcidine alkaloid aglycone and further its glycosylated derivatives soladulcine A and β-soladulcine from the solasodine aglycone. This suggests the presence of GAME25 homolog in those Solanum species. The formation of saturated alkaloid aglycone in any known Solanum species remains unclear till date.

FIGS. 4A-4B show levels of major SGAs and expression profile of GAME25 in various tomato wild accessions. FIG. 4A GAME25 normalized expression level among various tomato tissue types and developmental stages (from RNA-Seq data). Briefly, RNA-Seq transcriptome data was obtained from different tomato tissues and organs (flesh, peel, roots, young leaf, flower buds and young flower petals) and five developmental stages for peel and flesh tissues (19 experiments in total).

FIG. 4B shows expression of GAME25 in four stages of fruit development of different wild tomato species (normalized RNA-seq data). IG: immature green, MG: mature green, BR: breaker, R: ripe. FPKM: Fragments Per Kilobase of transcript per Million mapped reads.

FIG. 5 shows the alignment of tomato, S. pennellii and potato GAME25 proteins investigated in this study. GAME25 is a classical SDR family member protein possessing TGxxxGxG cofactor binding and the YxxxK catalytic motifs (marked in boxes). The Asp (D) residue at 40th position indicating preference for NAD+ over NADP+ was shown in asterisk.

FIG. 6 shows that GAME25 proteins form a separate clade in the large short-chain dehydrogenases/reductases family. Tomato, potato and S. pennellii (wild tomato species) GAME25 proteins characterized in this disclosure are marked in red, blue and green triangles, respectively. Five large short-chain dehydrogenases/reductases families (SDR65C, SDR108E, SDR110C, SDR114C and SDR119C) were included in the phylogenetic analysis that comprises enzymes involved predominantly in secondary metabolism. TR-like, tropane alkaloids; ACR, anthocyanidins; 4-DFR, 4-dihydroflavonols; LCR, leucoanthocyanidins; NOS, narcotinehemeacetal precursors; ABA2, abscisic acid; MS and MIS, diterpenoid; SR, quinoline alkaloids; 11/17 β-hydroxysteroid dehydrogenase, steroid metabolism. Another separate clade, ADH (‘alcohol dehydrogenase’) comprises SDRs from various plant species whose functions are still unknown. Details of the amino acid sequences used are provided in below. The evolutionary history was inferred using the maximum-likelihood method in MEGA6.0. Numbers on branches indicate bootstrap values in percentage of 1,000 replicates.

FIG. 7 shows the GAME25 expression levels in leaves and different fruit tissues (developmental stages) of GAME25-RNAi (#2, #3 and #4) transgenic tomato lines (qRT PCR assay). Briefly, RNA-Seq transcriptome data was obtained from different tomato tissues and organs (flesh, peel, roots, young leaf, flower buds and young flower petals) and five developmental stages for peel and flesh tissues (19 experiments in total). Values represent mean±standard error (n=3). Asterisks indicate significant changes from control samples (wild-type) as calculated by a Student's t-test (*P-value<0.05; **P-value<0.01; ***P-value<0.001).

FIG. 8 shows the SGAs levels in leaves of wild-type (non-transformed) and GAME25-RNAi tomato lines determined by Liquid Chromatography-Mass Spectrometry (LC-MS). Samples #2, #3 and #4 are three independent GAME25-RNAi transgenic tomato lines. Values indicate means of three biological replicates±standard error. Asterisks indicate significant changes from wild-type samples as calculated by a Student's t-test (*P-value<0.05; **P-value<0.01; ***P-value<0.001).

FIG. 9 shows levels of less abundant SGAs in GAME25 silenced tomato leaf tissues. Line #2, #3 and #4 are three independent GAME25-RNAi transgenic tomato lines. Values indicate means of three biological replicates±standard error. Asterisks indicate significant changes from wild-type samples as calculated by a Student's t-test (*P-value<0.05; **P-value<0.01; ***P-value<0.001).

FIG. 10 shows the silencing of GAME25 in S. pennellii. Expression of GAME25 in leaves of GAME25i S. pennellii transgenic lines (qRT PCR assay). Line #21 and #23 are two independent GAME25-RNAi transgenic lines.

FIGS. 11A-11I show substantially altered SGA metabolism in fruit of GAME25-silenced tomato lines (#2, #3 and #4 are three independent lines). Values represent mean±standard error (n=3). FIG. 11A shows reduced levels of saturated α-tomatine and its downstream derivatives in green fruit of GAME25-silenced tomato compared to wild-type. FIG. 11B shows levels of less abundant SGAs in GAME25 silenced tomato green fruits. FIG. 11C shows increased levels of unsaturated dehydrotomatine and its downstream SGAs in green fruit of GAME25-silenced tomato lines compared to wild-type. FIG. 11D shows increased dehydrotomatine derived SGA levels in breaker fruits of GAME25i lines compared to wild-type breaker fruits. FIG. 11E shows levels of less abundant SGAs in GAME25 silenced tomato breaker fruits. FIG. 11F shows increased α-tomatine derived SGA levels in breaker fruits of GAME25i lines compared to wild-type breaker fruits. FIG. 11G shows substantial reduction in levels of esculeoside A, major saturated SGA in red stage fruit, in GAME25-silenced red ripe fruit. α-tomatine and its derived saturated SGAs were not detected in GAME25-silenced red ripe fruit. FIG. 11H shows a massive increase in levels of unsaturated dehydroesculeoside A in GAME25-silenced red fruit compared to wild-type red fruits. FIG. 11I shows levels of less abundant SGAs in GAME25 silenced tomato red fruits. Asterisks indicate significant changes from wild-type samples as calculated by a Student's t-test (*P-value<0.05; **P-value<0.01; ***P-value<0.001).

FIG. 12 shows that altering GAME25 expression does not affect other SGA biosynthetic genes in tomato. The figure shows the expression of selected genes involved in SGA biosynthesis from leaves of GAME25i tomato transgenic lines (qRT PCR assay). Line #2, #3 and #4 are three independent GAME25-RNAi transgenic tomato lines. Values represent mean±standard error (n=3). Asterisks indicate significant changes from control samples (wild-type) as calculated by a Student's t-test (*P-value<0.05; **P-value<0.01; ***P-value<0.001).

FIGS. 13A-13E show that overexpression of GAME25 in tomato enhances the levels of the α-tomatine branch in the SGA pathway. FIG. 13A shows GAME25 expression in leaves, green and red fruit of transgenic tomato lines overexpressing the tomato GAME25 gene (GAME25-Ox) as determined by quantitative Real Time-PCR (qRT-PCR). FIG. 13B shows levels of α-tomatine, and FIG. 13C shows levels of additional SGAs in leaves of GAME25-Ox tomato lines as compared to wild-type ones. FIGS. 13D-13E show levels of α-tomatine, dehydrotomatine and additional SGAs in breaker (FIG. 13D) and red (FIG. 13E) fruit of GAME25-Ox lines as compared to wild-type fruit. Overexpression of GAME25 resulted in accumulation of either α-tomatine or its derived SGAs (e.g. acetoxy- or acetoxyhydroxytomatine) in leaves and fruit tissues. The values indicate means of three biological replicates±standard error. Student's t-test was used to assess the significance of the difference between transgenic and wild-type tissues (*P-value<0.05; **P-value<0.01; ***P-value<0.001). #91, #92 and #93 represent three independent GAME25-Ox tomato transgenes. LC-MS was used for metabolite analysis.

FIG. 14 shows that overexpression of tomato GAME25 results in accumulation of new saturated SGAs and steroidal saponins in eggplant. Comparison of SGA (upper panel) and steroidal saponin (lower panel) profile of wild-type (WT) and GAME25 overexpression transgenic eggplant lines; the naturally occurring unsaturated steroidal alkaloids (Solmargine, m/z 868.5106; Solasonine, m/z 884.5076 and malonyl-solamargine, m/z 954.5066) are reduced in the overexpression lines compared to the WT, while the saturated derivatives (lacking the 5-6 double bond) accumulate soladulcine A (m/z 870.5265), β-soladulcine (m/z 886.5131) & saturated malonyl-solamargine (m/z 956.5238); similarly the steroidal saponins (m/z 1031.5396 & m/z 1117.5479) are reduced, while their saturated forms (m/z 1033.5608 & 1119.5586) accumulate in GAME25 overexpression transgenic eggplants.

FIG. 15 shows the relative expression level of tomato GAME25 gene in leaves of GAME25-Ox eggplant (cv. Tudela) as compared to wild-type plants determined by quantitative real-time PCR (qRT-PCR). Tomato GAME25 gene was overexpressed in eggplant. #E1 and #E2 are two independent GAME25-Ox eggplant transgenic lines. Values indicate means of three biological replicates±standard error. Asterisks indicate significant changes from wild-type samples as calculated by a Student's t-test (*P-value<0.05; **P-value<0.01; ***P-value<0.001).

FIGS. 16A-16F show the structures of detected steroidal alkaloids and saponins in leaves of GAME25-Ox eggplant (cv. Tudela). Chemical structures were putatively assigned by calculating elemental compositions from the accurate mass and interpretation of mass fragmentation patterns, loss of water in positive ionization mode from steroidal saponins is typical for furostanol-type compounds (Heinig & Aharoni, 2014). FIG. 16A Solasonine (m/z 884.5076) and β-soladulcine (m/z 886.5131). FIG. 16B Solmargine (m/z 868.5106) and soladulcine A (m/z 870.5265). FIG. 16C malonyl-solamargine (m/z 954.5066) and saturated malonyl-solamargine (m/z 956.5238). FIG. 16D steroidal saponin (m/z 1031.5396) and saturated steroidal saponin (m/z 1033.5608). FIG. 16E steroidal saponin (m/z 1117.5479) and saturated steroidal saponin (m/z 1119.5586). FIG. 16F shows a comparison of mass fragmentation of steroidal alkaloid and steroidal saponin aglycones; Upper panel: Overlays of mass spectra of saturated SA aglycones (red) and unsaturated SA aglycones (black), lower panel: Overlays of mass spectra of saturated steroidal saponin aglycones (red) and unsaturated steroidal saponin aglycones (black). Characteristic fragment structures are shown, fragments after loss of the side chain of steroidal alkaloids or saponins are identical, m/z 253.19 & 271.21 (blue) for unsaturated compounds and m/z 255.21 & 273.22 (red) for saturated compounds.

FIGS. 17A-17C show overexpression of GAME25 in potato. FIG. 17A shows GAME25 gene expression (qRT-PCR) in GAME25-Ox lines in potato. Line #11, #12 and #13 are independent GAME25-Ox transgenic lines. FIGS. 17B-17C show levels of α-solanine and α-chaconine in leaves (FIG. 17B) and tuber peel (FIG. 17C) of GAME25-Ox lines as determined by LC-MS. Values represent mean±standard error (n=3). Student's t-test was used to assess whether the transgenic lines significantly differ from wild-type plants (*P-value<0.05; **P-value<0.01; ***P-value<0.001). #13 transgenic plants did not produce tubers.

FIGS. 18A-18B show expression of tomato and potato GAME25 protein in the microsomal fraction of Sf9 cells. Recombinant GAME25 proteins were expressed in Sf9 cells and analyzed by immunoblot with anti-myc antibodies. FIG. 18A shows tomato GAME25 protein. FIG. 18B shows potato GAME25 protein. M: Molecular weight protein marker.

FIGS. 19A-19L show the activity of the recombinant tomato and potato GAME25 produced in Sf9 insect cells with dehydrotomatidine and solanidine as substrates. FIGS. 19A-19B show an overlay of extracted ion chromatograms of m/z 412.32 Da, [M+H⁺]⁺ (mass of the GAME25 reaction product and the control reaction (sf9 cells microsomes) obtained with dehydrotomatidine as a substrate and tomato GAME25. FIGS. 19C-19D show the mass spectra and structures of the detected product (upper panel) and substrate (lower panel) of potato GAME25 enzymatic reaction with dehydrotomatidine as a substrate. FIG. 19E shows the mass fragmentation spectrum of the GAME25 enzymatic reaction product (with dehydrotomatidine as substrate) including the interpretation of the detected mass fragments. The fragmentation pattern corresponds to the proposed structure of the GAME25 product obtained with tomato GAME25. FIGS. 19F-19G shows an overlay of extracted ion chromatograms of m/z 396.32 Da, [M+H⁺]⁺ (mass of the GAME25 reaction product) and the control reaction (sf9 cells microsomes) with solanidine as a substrate and tomato GAME25. FIGS. 19H-19I shows the mass spectra and structures of the detected product (upper panel) and substrate (lower panel) of the potato GAME25 enzymatic reaction with solanidine as a substrate. FIG. 19J shows chromatograms of the potato GAME25 enzymatic reaction (upper panel), control reaction (middle panel), both with solanidine as substrate, and the solanid-4-en-3-one authentic standard injection (lower panel). The newly formed product (at RT 23.2 min.) co-elutes with solanid-4-en-3one. FIG. 19K shows an overlay of extracted ion chromatograms of potato GAME25 enzyme reaction product [m/z 412.32 Da, [M+H+]+] and the control reaction (sf9 cells microsomes) with solasodine as a substrate. FIG. 19L shows the mass spectra and structures of the detected product (upper panel) and substrate (lower panel) of potato GAME25 enzymatic reaction with solasodine as a substrate.

FIGS. 20A-20D show a characterization of GAME25 (tomato) by in vitro enzyme activity assay. GAME25 (tomato) was expressed in Sf9 insect cells. FIG. 20A shows an overlay of extracted ion chromatograms of m/z 412.32 Da, [M+H+]+, mass of product of GAME25 of enzyme reaction and control (reaction mixture without GAME25) using Solasodine as a substrate. FIG. 20B shows the mass spectra and structures of detected product (upper panel) and substrate (lower panel) of GAME25 enzymatic reaction with Solasodine as substrate. FIG. 20C shows the structures of fragments detected in MS-MS analysis of product of GAME25 (tomato) using Solasodine as a substrate. FIG. 20D shows MSMS spectrum of product of GAME25 (tomato) with Solasodine as a substrate.

FIGS. 21A-21C show GAME25 and GAME35 protein expression in BL21(DE3). FIG. 21A shows the expression of recombinant GAME25 and GAME35 proteins in E. coli BL21 (DE3) cells analyzed on SDS-PAGE. FIG. 21B and FIG. 21C show Western blot analysis of His-tagged recombinant GAME25 and GAME35 proteins. M: Molecular weight protein marker, W: Whole cell extract, El: Imidazole eluted fractions from Ni-NTA column, pET28: Empty pET28 vector transformed into BL21(DE3) cells was used as negative control. Recombinant proteins are marked with red arrows.

FIGS. 22A-22B show enzyme assays of the purified recombinant tomato GAME25 and GAME 25 produced in E. coli BL21 (DE3) cells. FIG. 22A shows chromatograms of the GAME25 enzymatic reaction using solanidine as substrate, control reaction (empty pET28 vector transformed into BL21(DE3) cells) and the solanid-4-en-3-one authentic standard injection (lower panel). GAME25 efficiently converted solanidine to the solanid-4-en-3-one product. FIG. 22B shows GAME35 enzyme reaction using solanid-4-en-3-one as substrate. GAME35 did not show any enzyme activity with solanid-4-en-3-one as a substrate.

FIGS. 23A-23J show inhibition of growth and spore germination of Colletotrichum gloeosporioides and Botrytis cinereal fungi on medium containing leaf extracts derived from GAME25 silenced tomato plants. Methanol extracts of tomato leaves from wild-type (WT) and GAME25 silenced lines (i.e. GAME25i, #2, #3 and #4 are three independent GAME25i transgenic lines) were used for fungal inhibition assays. FIG. 23A shows the inhibition area of C. gloeosporioides mycelial growth resulting from addition of leaf extracts of GAME25i plants compared to wild-type leaf extracts. FIG. 23B shows a representative image of the C. gloeosporioides mycelial growth inhibition after application of leaf extracts from GAME25i plants. FIG. 23C shows the inhibition area of Botrytis cinereal mycelial growth resulting from addition of leaf extracts of GAME25i plants compared to wild-type leaf extracts. FIG. 23D shows a representative image of the Botrytis cinereal mycelial growth inhibition after application of leaf extracts from GAME25i plants. FIG. 23E shows the percentage of C. gloeosporioides conidia germination resulting from addition of leaf extracts of GAME25i plants compared to wild-type leaf extracts. FIGS. 23F-G show representative images of C. gloeosporioides conidia germination in the presence of a GAME25i leaf extract (FIG. 23F) and wild-type leaf extract (FIG. 23G). FIG. 23H shows the percentage of Botrytis cinereal conidia germination resulting from addition of leaf extracts of GAME25i plants compared to wild-type leaf extracts. FIGS. 23I-J show representative images of Botrytis cinerea conidia germination in the presence of a GAME25i leaf extract (FIG. 23I) and wild-type leaf extract (FIG. 23J). FIGS. 23A, 23C, 23E and 23H indicate show means±standard error (n=15). This 15 process replicates were obtained from three separate experiment repetitions. Asterisks indicate significant changes from wild-type samples as calculated by a Student's t-test (*P-value<0.05; **P-value<0.01; ***P-value<0.001).

FIGS. 24A-24B show the conversion of Diosgenin, a steroidal saponin to Diosgen-4-en-3-one by tomato GAME25 enzyme. FIG. 24A shows an overlay of extracted ion chromatograms of m/z 415.32 (M+H⁺ of Diosgenin substrate) and 413.31 (M+H⁺ of detected reaction product), red: reaction with recombinant GAME25 protein from tomato, produced in E. coli, black: control reaction with E. coli protein extract (empty vector). GAME25 converts Diosgenin quantitatively to the putative product Diosgen-4-en-3-one. FIG. 24B shows mass fragmentation spectra of Diosgenin substrate (lower panel) and reaction product (upper panel) with explanation of characteristic fragments for Diosgenin and Diosgen-4-en-3-one, loss of the side chain leads to fragments m/z 271.21 and 269.19 respectively, followed by a neutral loss of water resulting in fragments m/z 253.19 and 251.18. Abbreviations: E. coli: Escherichia coli, EIC: extracted ion chromatogram, m/z mass to charge, M: molecular mass.

FIG. 25 shows that GAME25 enzymes plays a key role in the formation of saturated (elimination of the C-5,6 double bond) SA and steroidal saponin aglycones produced in different plant species in a sequence of three reactions. A three step reaction sequence for the conversion of dehydrotomatidine to tomatidine in tomato, solanidine to demissidine in wild potatoes (S. chacoense) and solasodine to soladulcidine in certain solanum species (e.g. S. dulcamara) is proposed. Additionally, it suggests three step reactions for the conversion of unsaturated steroidal saponin aglycone to saturated steroidal saponin aglycone in steroidal saponin producing plant species. GAME25, a novel 3β-hydroxysteroid dehydrogenase/isomerase perform the first step in this reaction sequence and produce 3-oxo-Δ^{5,4} steroidal alkaloid/saponin aglycone derivatives from the respective unsaturated steroidal alkaloid/saponin aglycone substrates (e.g. dehydrotomatidine or diosgenin) which are further converted to saturated (C₅-C₆ deficient) products by successive actions of putative 5-reductases and aldo-keto reductases respectively. This three step conversion partly resembles to steroid metabolism (e.g. progesterone formation and further catabolism) in plant species such as Digitalis spp. that produce cardiac glycoside secondary metabolites. Dashed arrows indicate multi-step reactions.

FIGS. 26A-26G show variation in SGA levels in the BIL/IL populations. The SGA content in leaf tissues was determined by leaf-dipping method and further analysis by UPLC-qTOF-MS. In all figures, the x-axis represents all 671 lines from the populations and the Y axis represents relative peak abundance. FIG. 26A shows levels of α-tomatine isomer 1. FIG. 26B shows levels of α-tomatine isomer 2. FIG. 26C shows levels of dehydrotomatine isomer 1. FIG. 26D shows levels of dehydrotomatine isomer 2. FIG. 26E shows levels of hydroxytomatine. FIG. 26F shows levels of acetoxytomatine. FIG. 26G shows levels of di-dehydrotomatine. While α-tomatine (isomer 1 and 2) and dehydrotomatine (isomer 1) were present in most of the lines, other SGAs were either absent of present predominantly in few tomato lines.

FIGS. 27A-27G show the SGA levels in the IL populations. The SGA content was determined from ground-tissue extracts by UPLC-qTOF-MS. The peak areas were determined by target analysis using the TargetLynx software Values represent mean±s.d. (n=3). FIG. 27A shows levels of α-tomatine isomer 1. FIG. 27B shows levels of α-tomatine isomer 2. FIG. 27C shows levels of dehydrotomatine isomer 1. FIG. 27D shows levels of dehydrotomatine isomer 2. FIG. 27E shows levels of hydroxytomatine. FIG. 27F shows levels of acetoxytomatine. FIG. 27G shows levels of di-dehydrotomatine.

FIGS. 28A and 28B present a region in tomato chromosome 1 linked to the accumulation of dehydrotomatine isomer 2. FIG. 28A presents the schematic representation of chromosome 1 showing the region in IL 1-1-1 controlling the content of dehydrotomatine isomer 2. FIG. 28B presents the suggested structures of the two isomers of dehydrotomatine (Schilmiler et al., (2010) Mass spectrometry screening reveals widespread diversity in trichome specialized metabolites of tomato chromosomal substitution lines. Plant J. 62: 391-403.).

FIGS. 29A and 29B present in tabular form a detailed list of genes present in the regions linked to SGA content in the BIL/IL populations. The table presented in FIG. 29A lists the genes in chromosome 1 region linked to content of dehydrotomatine isomer 2. The Table presented in FIG. 29B lists the genes in chromosome 2 region linked to content of hydroxytomatine and acetoxytomatine.

FIGS. 30A-30C present figures supporting the finding that a region in tomato chromosome 2 is linked to hydroxytomatine and acetoxytomatine content. FIG. 30A presents a schematic representation of chromosome 2 showing the region controlling the content of hydroxytomatine and acetoxytomatine in the BIL/IL populations. FIG. 30B schematically shows that out of the 17 genes in the region, four correspond to 2-oxoglutarate-dependent dioxygenases, named in this study SlGAME31 and SlGAME31-like genes. SlGAME31 and SlGAME31-like3 are full length coding sequences, while SlGAME31-like1 and SlGAME31-like2 are partial. FIG. 30C presents the predicted reaction catalyzed by GAME31, from α-tomatine to hydroxytomatine (position pointed by red arrow) and downstream pathway to esculeosides.

FIGS. 31A and 31B present graphs of SlGAME31 and SlGAME31-like gene expression. FIG. 31A presents a normalized expression profile of SlGAME31 and SlGAME31-like genes in RNA-Seq data from vegetative apex of the ILs (Chitwood et al., (2013) A quantitative genetic basis for leaf morphology in a set of precisely defined tomato introgression lines. Plant Cell 25: 2465-2481). SlGAME31 showed about 4.6-fold increased expression in IL2-1 (marked by *) in comparison with the other ILs, while SlGAME31-like genes showed very low expression levels across the population (<15 normalized Reads Per Million, RPM). FIG. 31B presents a normalized expression profile (from RNA-Seq dataset previously reported (Cárdenas et al., (2016) Nat. Commun. 7, 10654) of SlGAME31 in different tomato (cv. Microtom) tissue types and developmental stages. SlGAME31 showed an expression pattern correlating with fruit ripening, being lower in immature stages of fruit and other tissues (leaf, buds, petals and root). SlGAME31-like3 was slightly expressed while SlGAME31-like1 and SlGAME31-like2 genes were not detected in this dataset. IG: immature green; MG: mature green; Br: breaker; Or: or ange; RR: red ripe. FPKM: Fragments Per Kilobase of transcript per Million mapped reads.

FIGS. 32A and 32B present the results of sequence searches for SlGAME31 homologs in eggplant and potato. FIG. 32A shows that the sequence homology searches identified SmGAME31, encoding a 2-oxoglutarate-dependent dioxygenase in chromosome 1 of eggplant, as the closest homolog of the tomato GAME31. SmGAME31 is predicted to catalyze the hydroxylation of α-solamargine to hydrosolamargine. FIG. 32B shows that in potato, a tandem gene cluster of 8 homologs of SlGAME31 was identified, and which spanned ˜205 Kbp. At the protein level, StGAME31 displayed 54% identity to StGAME31-like1, 94% to StGAME31-like2, 67% to StGAME31-like3, 51% StGAME31-like4 and StGAME31-like5, 58% to StGAME31-like6 and 54% identity to StGAME31-like7. GAME31 in potato is predicted to hydroxylate α-chaconine and α-solanine, precursors of leptinine I and II, respectively. These later compounds can be further acetylated to produce leptine I and II, respectively.

FIGS. 33A-33H shows the hydroxylation of SA/SGAs by recombinant SIGAME31. Enzymatic reactions were performed with SlGAME31 using multiple SA/SGA substrates: α-tomatine (FIG. 33A), tomatidine (FIG. 33B), dehydrotomatine (FIG. 33C), α-solamargine (FIG. 33D), solasodine (FIG. 33E), α-chaconine (FIG. 33F), α-solanine (FIG. 33G) and solanidine (FIG. 33H). The enzymatic reaction was carried out in multiple conditions (FIGS. 33A-33G-see box) and formation of hydroxy-derivatives was assessed by UPLC-qTOF-MS. For each compound its m/z and the m/z of its hydroxylated derivatives are shown. The x-axes show the retention time (RT).

FIGS. 34A-34B show SlGAME31 in vitro enzyme activity. SlGAME31 expressed and purified from E. coli produced the same isomer (FIG. 34A) found in plants (FIG. 34B) (e.g. tomato line BIL 2037). The chromatogram shows the specific m/z=1050.5 for hydroxytomatine.

FIGS. 35A-35D show the hydroxylation of SA/SGAs by SmGAME31. Enzymatic reactions were performed with the same set of SA/SGAs as for SIGAME31. Hydroxylated derivatives were obtained for: α-solamargine (FIG. 35A), solasodine (FIG. 35B), α-tomatine (FIG. 35C), and dehydrotomatine (FIG. 35D). The enzymatic reaction was carried out in multiple conditions (A-G-see box) and formation of hydroxy-derivatives were assessed by UPLC-qTOF-MS. For each compound, its m/z and the m/z of its hydroxylated derivatives are shown. The x-axes show the retention time (RT).

FIGS. 36A-36D present data showing SlGAME31 downregulation alters SGA profile in red ripe tomato fruit. The figures show SIGAME31 gene expression (qRT-PCR) in SlGAME31-RNAi (silencing) (FIG. 36A) and SlGAME31-Cosup (co-suppression) (FIG. 36B) tomato lines. WT: wild-type. Tomato independent T₀ primary transgenic plants, SlGAME31-RNAi (#17, #18, #19) and SlGAME31-Cosup (#5, #6, #7). FIG. 36C presents a schematic representation of the SGA biosynthetic pathway. FIG. 36D shows changes in SGAs when SIGAME31 is downregulated.

### DETAILED DESCRIPTION

In the following detailed description, numerous specific details are set forth in order to provide a thorough understanding of the genetically modified plants and methods presented herein. However, it will be understood by those skilled in the art that these genetically modified plants and methods may be practiced without these specific details. In other instances, well-known methods, procedures, and components have not been described in detail so as not to obscure the formulations and compositions disclosed herein.

### Genetically Modified Plants

Disclosed herein are genetically modified plants, wherein expression of key genes in the steroidal glycoalkaloids metabolic pathway (biosynthesis pathway of steroidal alkaloids and glycosylated derivatives thereof) have been altered. Altering the expression of these genes results in concomitant alteration in the steroidal alkaloid profile. Changing the production level of steroidal alkaloid can result in improved plants comprising elevated content of steroidal alkaloids having increased resistance to pathogens, or plants having a reduced content of these secondary compounds in the plant edible parts and thus producing improved crops, wherein the improved crop has reduced or eliminated anti-nutritional content. Alternatively, or additionally, controlling the expression of genes disclosed herein may be used for the production of desired steroidal alkaloids for further use, for example in the pharmaceutical industry. In particular, disclosed herein are the means and methods for producing crop plants of the Solanaceae family that are devoid of toxic amounts of deleterious steroidal alkaloids typically present in edible parts of these plants. The plants disclosed herein are thus of significant nutritional and commercial value.

The Examples presented below demonstrate that unexpectedly by modifying the expression of a gene within the steroidal glycoalkaloid metabolic pathway, the level of at least one steroidal alkaloid, and/or at least one steroidal glycoalkaloid may be altered. For example, silencing of a single gene within the steroidal glycoalkaloid metabolic pathway, for example GAME25 in a tomato plant, resulted in significant reduction in the amount of α-tomatine concurrent with a significant accumulation of dehydrotomatine and its isomers.

In one embodiment, disclosed herein is a genetically modified plant comprising at least one cell having an altered expression of at least one gene selected from the group comprising a gene encoding a 3-β-hydroxysteroid dehydrogenase/isomerase (GAME25) and a gene encoding a 2-oxoglutarate-dependent dioxygenase (GAME31), or a combination thereof, wherein the genetically modified plant has an altered content of at least one cholesterol derived compound selected from the group comprising a steroidal alkaloid or a glycosylated derivative thereof and an unsaturated or saturated steroidal saponin or a glycoside derivative thereof compared to a corresponding unmodified plant.

In some embodiments, disclosed herein is a genetically modified plant comprising an altered content of at least one cholesterol derived compound selected from the group comprising a steroidal alkaloid or a glycosylated derivative thereof and an unsaturated or saturated steroidal saponin or a glycoside derivative thereof, said plant comprising at least one cell having
an altered biological activity of at least one enzyme selected from the group comprising a 3-β-hydroxysteroid dehydrogenase/isomerase (GAME25) and a 2-oxoglutarate-dependent dioxygenase (GAME31), or a combination thereof, or
an altered expression of at least one gene selected from the group comprising a gene encoding a 3-β-hydroxysteroid dehydrogenase/isomerase (GAME25) and a gene encoding a 2-oxoglutarate-dependent dioxygenase (GAME31), or a combination thereof, or
a combination thereof of an altered biological activity of at least one enzyme selected from the group comprising a 3-β-hydroxysteroid dehydrogenase/isomerase (GAME25) and a 2-oxoglutarate-dependent dioxygenase (GAME31), or a combination thereof, and an altered expression of at least one gene selected from the group comprising a gene encoding a 3-β-hydroxysteroid dehydrogenase/isomerase (GAME25) and a gene encoding a 2-oxoglutarate-dependent dioxygenase (GAME31), or a combination thereof;
   wherein the at least one cell of said genetically modified plant has an altered content of at least one cholesterol derived compound selected from the group comprising a steroidal alkaloid or a glycosylated derivative thereof and an unsaturated or saturated steroidal saponin or a glycoside derivative thereof compared to a corresponding unmodified plant.

One of ordinary skill in the art would appreciate that the term “gene” may encompass a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The skilled artisan would appreciate that the term “gene” optionally also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

In one embodiment, a gene comprises DNA sequence comprising upstream and downstream regions, as well as the coding region, which comprises exons and any intervening introns of the gene. In some embodiments, upstream and downstream regions comprise non-coding regulatory regions. In some embodiments, upstream and downstream regions comprise regulatory sequences, for example but not limited to promoters, enhancers, and silencers. Non-limiting examples of regulatory sequences include, but are not limited to, AGGA box, TATA box, Inr, DPE, ZmUbi1, PvUbi1, PvUbi2, CaMV, 35S, OsAct1, zE19, E8, TA29, A9, pDJ3S, B33, PAT1, alcA, G-box, ABRE, DRE, and PCNA. Regulatory regions, may in some embodiments, increase or decrease the expression of specific genes within a plant described herein.

In another embodiment, a gene comprises the coding regions of the gene, which comprises exons and any intervening introns of the gene. In another embodiment, a gene comprises its regulatory sequences. In another embodiment, a gene comprises the gene promoter. In another embodiment, a gene comprises its enhancer regions. In another embodiment, a gene comprises 5′ non-coding sequences. In another embodiment, a gene comprises 3′ non-coding sequences.

In one embodiment, the skilled artisan would appreciate that DNA comprises a gene, which may include upstream and downstream sequences, as well as the coding region of the gene. In another embodiment, DNA comprises a cDNA (complementary DNA). One of ordinary skill in the art would appreciate that cDNA may encompass synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence. Further, cDNA may include upstream and downstream regulatory sequences. In still another embodiment, DNA comprises CDS (complete coding sequence). One of ordinary skill in the art would appreciate that CDS may encompass a DNA sequence, which encodes a full-length protein or polypeptide. A CDS typically begins with a start codon (“ATG”) and ends at (or one before) the first in-frame stop codon (“TAA”, “TAG”, or “TGA”). The skilled artisan would recognize that a cDNA, in one embodiment, comprises a CDS.

As used herein, the terms “polynucleotide”, “polynucleotide sequence”, “nucleic acid sequence”, and “isolated polynucleotide” may be used interchangeably herein, having all the same qualities and meanings. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

One of ordinary skill in the art would appreciate that a genetically modified plant may encompass a plant comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a “transgenic plant”.

A skilled artisan would appreciate that a comparison of a “genetically modified plant” to a “corresponding unmodified plant” as used herein encompasses comparing a plant comprising at least one genetically modified cell and to a plant of the same type lacking the modification.

The skilled artisan would appreciate that the term “transgenic” when used in reference to a plant as disclosed herein encompasses a plant that contains at least one heterologous transcribable polynucleotide in one or more of its cells. The term “transgenic material” encompasses broadly a plant or a part thereof, including at least one cell, multiple cells or tissues that contain at least one heterologous polynucleotide in at least one of cell. Thus, comparison of a “transgenic plant” and a “corresponding non transgenic plant”, or of a “genetically modified plant comprising at least one cell having altered expression, wherein said plant comprising at least one cell comprising a heterologous transcribable polynucleotide” and a “corresponding un modified plant” encompasses comparison of the “transgenic plant” or “genetically modified plant” to a plant of the same type lacking said heterologous transcribable polynucleotide. A skilled artisan would appreciate that, in some embodiments, a “transcribable polynucleotide” comprises a polynucleotide that can be transcribed into an RNA molecule by an RNA polymerase.

One of ordinary skill in the art would appreciate that the terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The skilled artisan would appreciate that the term “transient transformation” or “transiently transformed” may encompass the introduction of one or more exogenous polynucleotides into a cell in the absence of integration of the exogenous polynucleotide into the host cell's genome. In contrast, the term “stable transformation” or “stably transformed” may encompass the introduction and integration of one or more exogenous polynucleotides into the genome of a cell. The skilled artisan would therefore appreciate that the term “stable transformant” may encompass a cell which has stably integrated one or more exogenous polynucleotides into the genomic or organellar DNA. It is to be understood that an organism or its cell transformed with the nucleic acids, constructs and/or vectors as disclosed herein can be transiently as well as stably transformed.

The skilled artisan would appreciate that the term “construct” may encompass an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The skilled artisan would appreciate that the term “expression” may encompass the production of a functional end-product e.g., an mRNA or a protein.

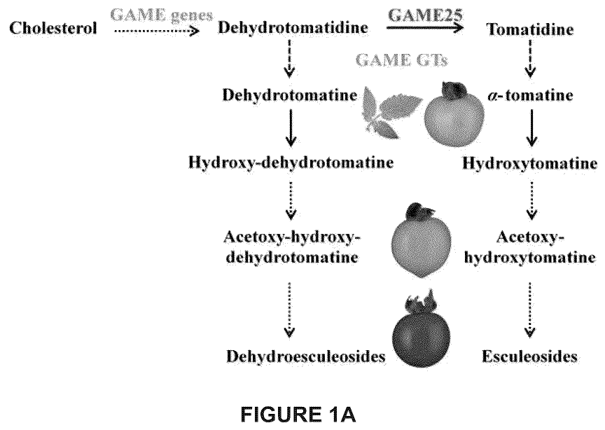

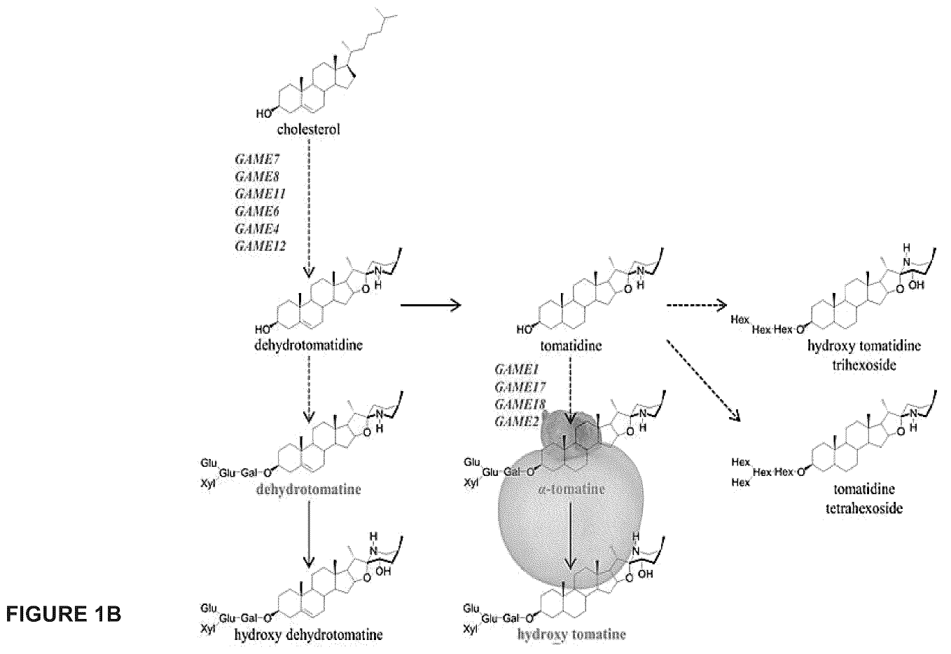

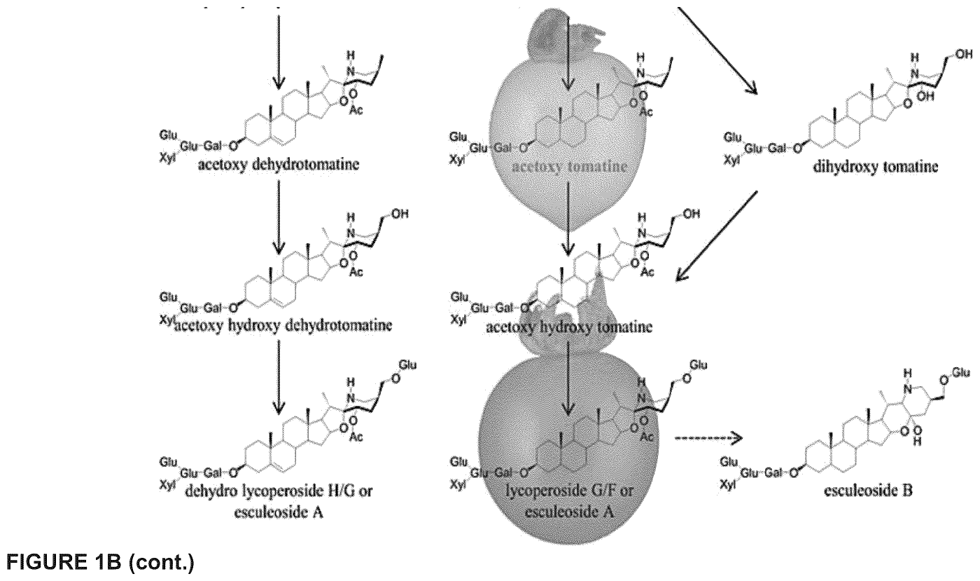

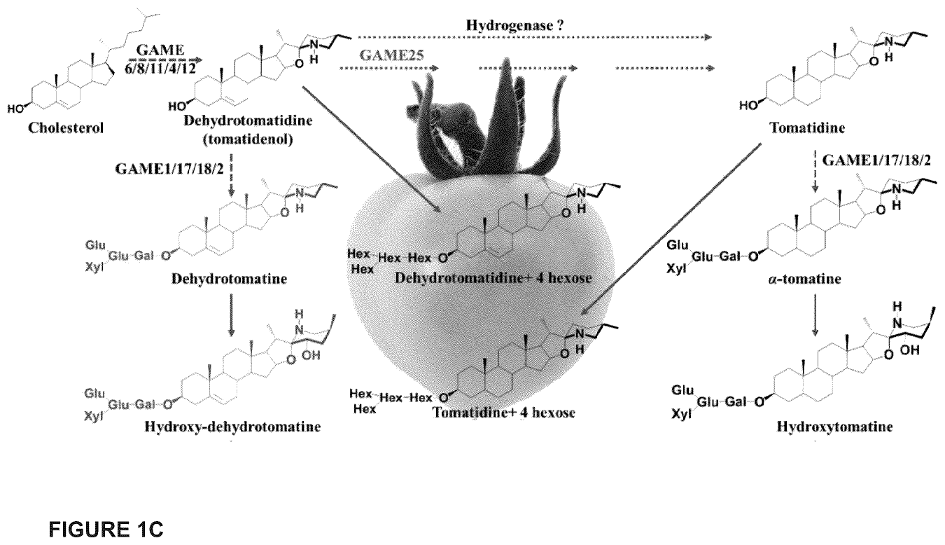

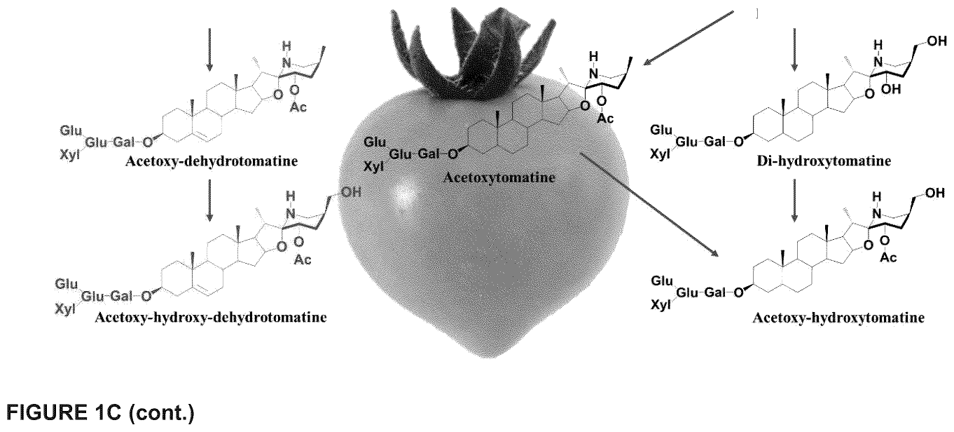

The enzymes encoded by GAME25 and GAME31 are involved in particular steps of cholesterol metabolism (FIGS. 1A-1C, FIGS. 2A-2B, FIGS. 3A-3C). For example, a tomato GAME25 gene (SEQ ID NO: 1) encodes a side chain reductase enzyme, 3-β-hydroxysteroid dehydrogenase/isomerase, (GAME25; SEQ ID NO: 3), which in tomato catalyzes the first step in the multi-step conversion of dehydrotomatidine to tomatidine (FIGS. 1A-1C).

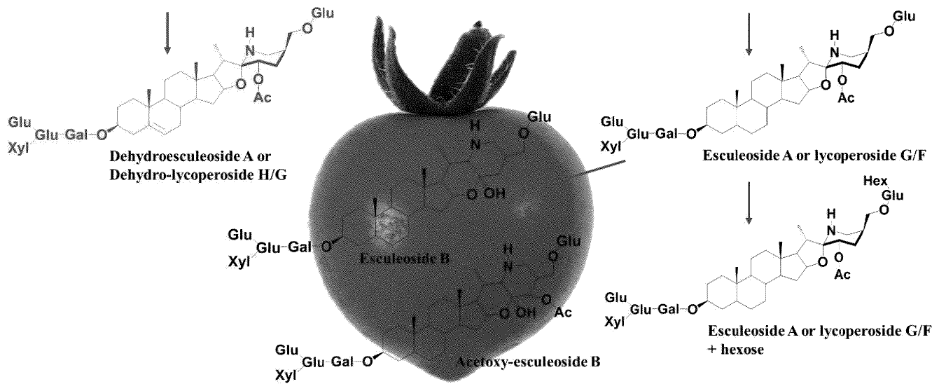

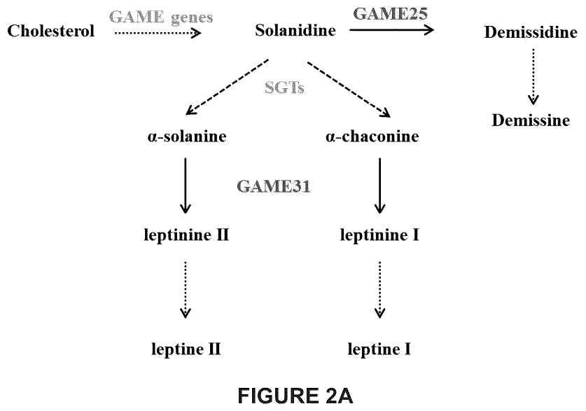

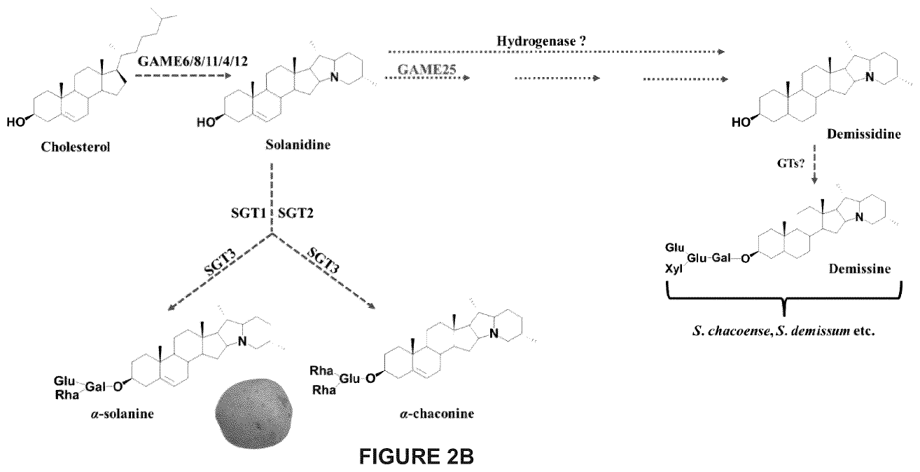

A homologue of the GAME25 gene was identified in potato (SEQ ID NO: 13), wherein the encoded enzyme (SEQ ID NO: 15) may catalyze the first step in the conversion of solanidine to demissidine (FIGS. 2A-2B).

In some embodiments, a tomato plant comprises a cultivated tomato plant. In some embodiments, a cultivated tomato plant comprises a Solanum lycopersicum (S. lycopersicum) species of tomato plant. In some embodiment, a tomato plant comprises a wild tomato plant. In some embodiments, a wild tomato plant comprises a Solanum pennellii (S. pennellii) species.

In one embodiment, a tomato GAME25 gene comprises the nucleic acid sequence of SEQ ID NO: 1. In another embodiment, a tomato GAME25 gene consists of the nucleic acid sequence of SEQ ID NO: 1. In another embodiment, SEQ ID NO: 1 comprises or consists of the following nucleic acid sequence:

(SEQ ID NO: 1)
GTGATATATTTCAAAAAATAATTAAAATACATATATATTGCATACATAAT
TCACTTTTAATACATATTGCAGATTTAACTTAAACATTGTTATAAATGGT
GATAAATAAAAAAATCGTTAAAATTAGTAATTATTCATTAAACTGCATCT
ATTTATGTAATTTTTCCAATTAAAAATCTATTATTTTTTTTCAATCCAAT
CCAAACAGGCTCTAAAGCATCAATGTTTTTGAAATTACCAAAATAGCCTC
GGTTGTTAAGCGCTTCCTTCTATATATTAGTGAATTCAAACTACAGTCGG
TACAAAGGAAGTTATTTACTCTTATAATGGCAAATAAGCTCAGGTATAGC
ATAGTTAGTATTTGTTTAAATTAATGGTGCTAATCAGTACATTAATTTAT
TTTCTCAAAATTGTGTAATTACATATAATTAAATGTGTTTAATCAAATGT
TTTTCTTTTTTATATGCATCGATCCTGTAGGTTGGAGGGCAAAGTAGCTA
TAATTACCGGTGCTGCTAGTGGCATTGGAGAAGCAAGTGCTAGATTGTTC
GTTGAACATGGTGCTCGTGTCGTCGTCGCCGATATTCAAGATGAACTTGG
TCAAAAAGTAGTTGATTCTATCGGATCTGACAAAGCCAGCTACCGGCACT
GCGACGTTACAGACGAGAAGCAAGTTGAGGAAACCGTAGCTTACGCGGTA
GAGAAATACGGTACTCTTGACATTATGTTTAGTAATGTCGGGACGCTGAA
TTTCTGCAGCGTCCTCGACATGGACGTGCTGGCCTTCGATGAGACCATGG
CCATCAACGTACGCGGATCCGCGTTAGCGGTTAAGCACGCGGCTAAAGTT
ATGGTTGATAAGAAAATTCGGGGATCTATTATATGTAACGCGAGTTTAGA
AGGGATTTTAGCTGGGGCCGCTTCGCTCGCCTACATTGCGTCAAAGCACG
CAGTGGTAGGCATTATAAAAGCGGCCGCACGTGAACTGGGTCCACATGGG
ATAAGGGTGAATGGGGTGTCGCCCTATGGAATAGCGACGCCCCTTGTGAC
TAAGGCGTATGGACTGGATGCGGCTCTATTGGAAGAAGCAATTTACGGTA
ATGGACACTTGAAAGGAGTTAAGTTGAGCACGATGCATGTAGCACAATCA
GCACTTTTTTTGGCGTCTGATGAATCTGCTTATACAAGTGGTCAAAATTT
AGCTGTTGATGGTGGACTAAGTTCTATTTTGAAGCTACAATAAATTGTCA
CGCTATTTGTGTTGGCGTGCTGTGGCGTGGGCCTTAATCCTCACTCTCTT
GTGTCTGTACTTCTGTTTCATCTCGTTTCGTTTCAAATTTTCAACTTAAT
AATACTCTCATATTTTATGCGATATTTTTCAGATTTATACTAAGTTTTTT
ATAGATATTTTAAACGTTGTGACTTAAAAAGATATAAATTTCATTTTTTT
AAAATTAAAAATTTTATG.

In one embodiment, a potato comprises a cultivated potato plant or a wild potato plant.

In some embodiments, a potato plant comprises a cultivated potato plant. In some embodiments, a cultivated potato plant comprises a Solanum tuberosum (S. tuberosum) species of potato plant. In some embodiment, a potato plant comprises a wild potato plant. In some embodiments, a wild potato plant comprises a Solanum pennellii (S. pennellii) species.

In another embodiment, a potato GAME25 gene comprises the nucleic acid sequence of SEQ ID NO: 13. In another embodiment, a potato GAME25 gene consists of the nucleic acid sequence of SEQ ID NO: 13. In another embodiment, SEQ ID NO: 13 comprises or consists of the following nucleic acid sequence:

(SEQ ID NO: 13)
AAAAAATTTAACATACAGTTGCTGCAAAGGAAGCTACCTACTCGTATAAT
GGCAAATAAGCTCAGGTACTTAATTAGTACATTAATTTCTTTCTTTCTTT
TCTCAAATTGTATATGAGAATTAAATGTGTATTTTTAGCTTTAATCAAAT
GTTTTTGTGGTATATTATATGCATCGTGTAGGTTGGAGGGCAAAGTGGCT
ATAATTACAGGTGCTGCAAGTGGCATTGGAGAAGCAAGTGCTAGATTGTT
CGCCGAACATGGTGCTCGTATTGTCGTAGCCGATATTCAAGATGAACTTG
GTCTGAAAGTAGTTGAATCTATCGGAGCTGACAAAGCCAGCTACCGACAC
TGCGACGTTACAGACGAGAAGCAAGTTGAGGATACCGTAGCTTACACGGT
AGAGAAATACGGTACTCTTGACATCATGTTTAGTAATGTTGGGACGCTGA
ATTTTTGCAGCGTCCTGGACATGGACGTGATGGTCTTCGATAAGACGATG
GCCATCAACGCACGAGGATCCGCGTTAGCGGTCAAGCACGCGGCTAGATT
TATGGTTGATAAGAAAATTCGGGGATCCATTATATGCAACGCGAGTTTAG
ATGGTATTGTAGCTGGGGCCACTTCGCTTGCCTACATTGCGTCAAAGCAC
GCAGTTGTAGGCATTGTGAAAGCGGCCGCACGTGACCTAGGTCCATACGG
GATAAGGGTGAATGGGGTGTCGCCATATGGAATAGCGACGCCCCTGGTGT
GCAAAGCGTATGGGTTGGATGCGGGTCCATTGGAAGCAGCAATATATGGA
AATGGAAACTTGAAAGGTGTTAGGTTGAGCACGATGCATGTAGCACAATC
AGCACTTTTCTTGGCGTCTGATGAATCTGCTTACACAAGTGGTCAAAATT
TAGCTGTTGATGGTGGACTTAGTTCTATTTTGAAGGTACAATAGATTGTC
ACTCTATTGTGCTGGTGTGCTGTGATGTGTGCATTAGTTCTATTTTGAAG
CTACAATAATTCCTTTGTCATGTAGTACTGTTTATCTTGTTTCATTTCGA
ATTTTCAACTTAAATAATATTCTCTCACAG.

In one embodiment, a tomato GAME25 cDNA comprises the nucleic acid sequence of SEQ ID NO: 2. In another embodiment, a tomato GAME25 cDNA consists of the nucleic acid sequence of SEQ ID NO: 2. In another embodiment, SEQ ID NO: 2 comprises or consists of the following nucleic acid sequence:

(SEQ ID NO: 2)
ATGGCAAATAAGCTCAGGTTGGAGGGCAAAGTAGCTATAATTACCGGTGC
TGCTAGTGGCATTGGAGAAGCAAGTGCTAGATTGTTCGTTGAACATGGTG
CTCGTGTCGTCGTCGCCGATATTCAAGATGAACTTGGTCAAAAAGTAGTT
GATTCTATCGGATCTGACAAAGCCAGCTACCGGCACTGCGACGTTACAGA
CGAGAAGCAAGTTGAGGAAACCGTAGCTTACGCGGTAGAGAAATACGGTA
CTCTTGACATTATGTTTAGTAATGTCGGGACGCTGAATTTCTGCAGCGTC
CTCGACATGGACGTGCTGGCCTTCGATGAGACCATGGCCATCAACGTACG
CGGATCCGCGTTAGCGGTTAAGCACGCGGCTAAAGTTATGGTTGATAAGA
AAATTCGGGGATCTATTATATGTAACGCGAGTTTAGAAGGGATTTTAGCT
GGGGCCGCTTCGCTCGCCTACATTGCGTCAAAGCACGCAGTGGTAGGCAT
TATAAAAGCGGCCGCACGTGAACTGGGTCCACATGGGATAAGGGTGAATG
GGGTGTCGCCCTATGGAATAGCGACGCCCCTTGTGACTAAGGCGTATGGA
CTGGATGCGGCTCTATTGGAAGAAGCAATTTACGGTAATGGACACTTGAA
AGGAGTTAAGTTGAGCACGATGCATGTAGCACAATCAGCACTTTTTTTGG
CGTCTGATGAATCTGCTTATACAAGTGGTCAAAATTTAGCTGTTGATGGT
GGACTAAGTTCTATTTTGAAGCTACAATAA.

In another embodiment, a tomato GAME25 cDNA comprises the nucleic acid sequence of SEQ ID NO: 11. In another embodiment, a tomato GAME25 cDNA consists of the nucleic acid sequence of SEQ ID NO: 11. In another embodiment, SEQ ID NO: 11 comprises or consists of the following nucleic acid sequence:

(SEQ ID NO: 11)
ATGGCAAATAAGCTCAGGTTGGAGGGCAAAGTAGCTATAATTACTGGTGC
TGCTAGTGGCATTGGAGAGGCAAGTGCTAGATTGTTCGTTGAACATGGTG
CTCGTGTCGTCGTCGCCGATATTCAAGATGAACTTGGTCAAAAAGTAGTT
GATTCTATCGGAGCTGACAAAGCCAGCTACCGGCACTGCGACGTTACAGA
CGAGAAGCAAGTTGAGGAAACCGTAGCCTACGCGGTAGAGAAATACGGTA
CTCTTGACATTATGTTTAGTAATGTCGGGACGCTGAATTTCTGCAGCGTC
CTCGACATGGACGTGATGGCCTTCGATGAGACGATGGCCATCAACGTACG
TGGATCCGCGCTAGCGGTTAAGCACGCGGCTAAAGTTATGGTTGATAAGA
AAATTCGGGGATCTATTATATGTAACGCGAGTTTAGAGGGGATTTTAGCT
GGGGCCGCTTCGCTTGCCTACATTGCGTCAAAGCACGCAGTCGTAGGCAT
AATAAAAGCGGCCGCACGTGAACTGGGTCCACATGGGATAAGGGTGAATG
GGGTGTCGCCATATGGAATAGCGACGCCCCTGGTGTGTAAGGCGTATGGA
CTGGATGCGGCTCTATTGGAAGAAGCAATTTATGGTAATGGACACTTGAA
AGGTGTTAAGTTGAGCACGATGCATGTAGCACAATCAGCACTTTTTTTGG
CGTCTGATGAATCTGCTTACACAAGTGGTCAAAATTTAGCTGTTGATGGT
GGACTAAGTTCTATTTTGAAGCTACAATAA.

In another embodiment, a potato GAME25 cDNA comprises the nucleic acid sequence of SEQ ID NO: 14. In another embodiment, a potato GAME25 cDNA consists of the nucleic acid sequence of SEQ ID NO: 14. In another embodiment, SEQ ID NO: 14 comprises or consists of the following nucleic acid sequence:

(SEQ ID NO: 14)
ATGGCAAATAAGCTCAGGTTGGAGGGCAAAGTGGCTATAATTACAGGTGC
TGCAAGTGGCATTGGAGAAGCAAGTGCTAGATTGTTCGCCGAACATGGTG
CTCGTATTGTCGTAGCCGATATTCAAGATGAACTTGGTCTGAAAGTAGTT
GAATCTATCGGAGCTGACAAAGCCAGCTACCGACACTGCGACGTTACAGA
CGAGAAGCAAGTTGAGGATACCGTAGCTTACACGGTAGAGAAATACGGTA
CTCTTGACATCATGTTTAGTAATGTTGGGACGCTGAATTTTTGCAGCGTC
CTGGACATGGACGTGATGGTCTTCGATAAGACGATGGCCATCAACGCACG
AGGATCCGCGTTAGCGGTCAAGCACGCGGCTAGATTTATGGTTGATAAGA
AAATTCGGGGATCCATTATATGCAACGCGAGTTTAGATGGTATTGTAGCT
GGGGCCACTTCGCTTGCCTACATTGCGTCAAAGCACGCAGTTGTAGGCAT
TGTGAAAGCGGCCGCACGTGACCTAGGTCCATACGGGATAAGGGTGAATG
GGGTGTCGCCATATGGAATAGCGACGCCCCTGGTGTGCAAAGCGTATGGG
TTGGATGCGGGTCCATTGGAAGCAGCAATATATGGAAATGGAAACTTGAA
AGGTGTTAGGTTGAGCACGATGCATGTAGCACAATCAGCACTTTTCTTGG
CGTCTGATGAATCTGCTTACACAAGTGGTCAAAATTTAGCTGTTGATGGT
GGACTTAGTTCTATTTTGAAGGTACAATAG.

In one embodiment, a tomato GAME25 polypeptide comprises the amino acid sequence set forth in SEQ ID NO: 3. In another embodiment, a tomato GAME25 polypeptide consists of the amino acid sequence set forth in SEQ ID NO: 3. In another embodiment, SEQ ID NO: 3 comprises or consists of the following amino acid sequence:

(SEQ ID NO: 3)
MANKLRLEGKVAIITGAASGIGEASARLFVEHGARVVVADIQDELGQKVV
DSIGSDKASYRHCDVTDEKQVEETVAYAVEKYGTLDIMFSNVGTLNFCSV
LDMDVLAFDETMAINVRGSALAVKHAAKVMVDKKIRGSIICNASLEGILA
GAASLAYIASKHAVVGIIKAAARELGPHGIRVNGVSPYGIATPLVTKAYG
LDAALLEEAIYGNGHLKGVKLSTMHVAQSALFLASDESAYTSGQNLAVDG
GLSSILKLQ.

In another embodiment, a tomato GAME25 polypeptide comprises the amino acid sequence set forth in SEQ ID NO: 12. In another embodiment, a tomato GAME25 polypeptide consists of the amino acid sequence set forth in SEQ ID NO: 12. In another embodiment, SEQ ID NO: 12 comprises or consists of the following amino acid sequence:

(SEQ ID NO: 12)
MANKLRLEGKVAIITGAASGIGEASARLFVEHGARVVVADIQDELGQKVV
DSIGADKASYRHCDVTDEKQVEETVAYAVEKYGTLDIMFSNVGTLNFCSV
LDMDVMAFDETMAINVRGSALAVKHAAKVMVDKKIRGSIICNASLEGILA
GAASLAYIASKHAVVGIIKAAARELGPHGIRVNGVSPYGIATPLVCKAYG
LDAALLEEAIYGNGHLKGVKLSTMHVAQSALFLASDESAYTSGQNLAVDG
GLSSILKLQ.

In another embodiment, a potato GAME25 polypeptide comprises the amino acid sequence set forth in SEQ ID NO: 15. In another embodiment, a potato GAME25 polypeptide consists of the amino acid sequence set forth in SEQ ID NO: 15. In another embodiment, SEQ ID NO: 15 comprises or consists of the following amino acid sequence:

(SEQ ID NO: 15)
MANKLRLEGKVAIITGAASGIGEASARLFAEHGARIVVADIQDELGLKVV
ESIGADKASYRHCDVTDEKQVEDTVAYTVEKYGTLDIMFSNVGTLNFCSV
LDMDVMVFDKTMAINARGSALAVKHAARFMVDKKIRGSIICNASLDGIVA
GATSLAYIASKHAVVGIVKAAARDLGPYGIRVNGVSPYGIATPLVCKAYG
LDAGPLEAAIYGNGNLKGVRLSTMHVAQSALFLASDESAYTSGQNLAVDG
GLSSILKVQ.

In one embodiment, a GLYCOALKALOID METABOLISM 25 (GAME25) polypeptide comprises a 3-β-hydroxysteroid dehydrogenase/isomerase enzyme activity. In another embodiment, a3-β-hydroxysteroid dehydrogenase/isomerase (GAME25) enzyme comprises the amino acid sequence set forth in any one of SEQ ID NO: 3, SEQ ID NO: 12, and SEQ ID NO: 15, or a protein homologue thereof, wherein said protein homologue is at least 80% homologous to any of SEQ ID NO: 3, SEQ ID NO: 12, and SEQ ID NO: 15.

One of ordinary skill in the art would recognize that a 3-β-hydroxysteroid dehydrogenase/isomerase is a side chain reductase enzyme. In one embodiment, homologues of the GAME25 enzyme comprise a 3-β-hydroxysteroid dehydrogenase/isomerase enzyme activity. In some embodiments, a homologue also encompasses deletion, insertion, or substitution variants, including an amino acid substitution, thereof and biologically active polypeptide fragments thereof. In one embodiment, the variant comprises conservative substitutions, or deletions, insertions, or substitutions that do not significantly alter the three dimensional structure of the GAME25 enzyme. In another embodiment, the deletion, insertion, or substitution does not alter the function of interest of the GAME25 enzyme, which in one embodiment wherein said plant is a tomato plant is a first step in the multi-step conversion of dehydrotomatidine to tomatidine. In another embodiment, the deletion, insertion, or substitution does not alter the function of interest of the GAME25 enzyme, which in another embodiment wherein said plant is a potato plant is a first step in the multi-step conversion of solanidine to demissidine.

In some embodiments, when a genetically modified plant is a tomato plant, the altered at least one steroidal alkaloid or a glycosylated derivative thereof is selected from the group comprising α-tomatine, α-tomatine isomer (1 and 2), α-tomatine isomer 2, hydroxytomatine, acetoxytomatine, dehydrotomatidine, dehydrotomatine, dehydrotomatine isomer 1, dehydrotomatine+4-hexose, or any derivatives thereof, or any combination thereof. In some embodiments, when a genetically modified plant is an eggplant plant, the altered at least one steroidal alkaloid or a glycosylated derivative thereof is selected from the group comprising β-soldulcine, soladulcine A, and the unsaturated or saturated steroidal saponin or glycosylated derivative thereof is selected from dioscin, diosgenin, parillin, sarasapogenin, or any derivatives thereof, or any combination thereof.

In another embodiment, the disclosure includes a homologue of a GAME25 enzyme. In another embodiment, the disclosure includes a homologue of a GAME25 enzyme having a 3-β-hydroxysteroid dehydrogenase/isomerase enzyme activity. In another embodiment, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a GAME25 enzyme as determined using BlastP software of the National Center of Biotechnology Information (NCBI) using default parameters.

In another embodiment, the amino acid sequence of a GAME25 enzyme homologue is at least 70% homologous to a GAME25 amino acid sequence or a peptide thereof, described herein. In another embodiment, the amino acid sequence of a GAME25 enzyme homologue is at least 80% homologous a GAME25 amino acid sequence or a peptide thereof, described herein. In another embodiment, the amino acid sequence of a GAME25 enzyme homologue is at least 90% homologous a GAME25 amino acid sequence or a peptide thereof, described herein. In another embodiment, the amino acid sequence of a GAME25 enzyme homologue is at least 95% homologous a GAME25 amino acid sequence or a peptide thereof, described herein. In another embodiment, the amino acid sequence of a GAME25 enzyme homologue is at least 98% homologous a GAME25 amino acid sequence or a peptide thereof, described herein.

A skilled artisan would appreciate that the terms “polypeptides”, “polypeptide”, “enzyme” or grammatical equivalents thereof, may be used interchangeably to encompass amino acid sequences or proteins and may encompass polymers of amino acids of any length. These polypeptides may in some embodiments include proteins which have been modified post-translationally by reactions such as glycosylation, phosphorylation, acetylation or protein processing. The structure of the polypeptide may be modified, for example, by substitutions, deletions or insertions of amino acids and fusion with other proteins while retaining its biological activity.

In some embodiment, homologues of GAME25 enzyme are produced in plants, wherein said plants comprise a cultivated tomato plant, a wild tomato plant, a cultivated potato plant, a wild potato plant, or a bittersweet plant.

In another embodiment, a3-β-hydroxysteroid dehydrogenase/isomerase comprises the amino acid sequence set forth in any one of SEQ ID NO: 3, SEQ ID NO: 12, or SEQ ID NO: 15.

In one embodiment, the 3-β-hydroxysteroid dehydrogenase/isomerase (GAME25) polypeptide is encoded by a gene comprising the nucleotide sequence set forth in any one of SEQ ID NO: 1, SEQ ID NO. 13, or a gene homologue thereof, wherein said gene homologue is at least 80% homologous to any of SEQ ID NO: 1, SEQ ID NO. 13. In another embodiment, the 3-β-hydroxysteroid dehydrogenase/isomerase (GAME25) polypeptide is encoded by a cDNA comprising the nucleotide sequence set forth in any one of SEQ ID NO: 2, SEQ ID NO: 11, or SEQ ID NO. 14, or a gene homologue thereof, wherein said gene homologue is at least 80% homologous to any of SEQ ID NO: 2, SEQ ID NO: 11, or SEQ ID NO. 14.

In another embodiment, the 3-β-hydroxysteroid dehydrogenase/isomerase is encoded by a gene comprising the nucleotide sequence set forth in SEQ ID NO: 1, SEQ ID NO. 13. In another embodiment, a 3-β-hydroxysteroid dehydrogenase/isomerase (GAME25) is encoded by the nucleic acid sequence set forth in any one of SEQ ID NO: 2, SEQ ID NO: 11, or SEQ ID NO. 14, or a polynucleotide homologue thereof, wherein said polynucleotide homologue is at least 80% homologous to any of SEQ ID NO: 2, SEQ ID NO: 11, or SEQ ID NO. 14. In another embodiment, a 3-β-hydroxysteroid dehydrogenase/isomerase (GAME25) is encoded by the nucleic acid sequence set forth in any one of SEQ ID NO: 1, SEQ ID NO: 2, SEQ ID NO: 11, SEQ ID NO: 13, or SEQ ID NO. 14, or a polynucleotide homologue thereof, wherein said polynucleotide homologue is at least 80% homologous to any of SEQ ID NO: 1, SEQ ID NO: 2, SEQ ID NO: 11, SEQ ID NO: 13, or SEQ ID NO. 14.

In some embodiments, said 3-β-hydroxysteroid dehydrogenase/isomerase (GAME25) is encoded by a gene comprising the polynucleotide sequence set forth in any one of SEQ ID NO: 1, SEQ ID NO: 2, SEQ ID NO: 11, SEQ ID NO: 13, and SEQ ID NO: 14, or a gene homologue thereof, wherein said gene homologue is at least 80% homologous to any of SEQ ID NO: 1, SEQ ID NO: 2, SEQ ID NO: 11, SEQ ID NO: 13, and SEQ ID NO: 14.

A skilled artisan would appreciate that a “homologue” of a nucleic acid sequence as disclosed herein, for example a homologue of a gene sequence or a homologue of a cDNA sequence, comprises a percent homology with a corresponding nucleic acid sequence disclosed herein.

Homology is, in some embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In one embodiment, homologues of a GAME25 gene or a GAME25 cDNA encode a polypeptide comprising a 3-β-hydroxysteroid dehydrogenase/isomerase enzyme activity. In some embodiments, a homologue also encompasses deletion, insertion, or substitution variants, thereof, and biologically active polynucleotide fragments thereof. In one embodiment, the variant comprises conservative substitutions, or deletions, insertions, or substitutions that do not significantly alter the three dimensional structure of the encoded GAME25 enzyme. In another embodiment, the deletion, insertion, or substitution does not alter the function of interest of the encoded GAME25 enzyme, which in one embodiment wherein said plant is a tomato plant, is a first step in the multi-step conversion of dehydrotomatidine to tomatidine. In another embodiment, the deletion, insertion, or substitution does not alter the function of interest of the encoded GAME25 enzyme, which in another embodiment wherein said plant is a potato plant, is a first step in the multi-step conversion of solanidine to demissidine.

In another embodiment, the disclosure includes a homologue of a GAME25 gene. In another embodiment, the disclosure includes a homologue of a GAME25 cDNA. In another embodiment, the disclosure includes a homologue of a GAME25 gene encoding an enzyme having a 3-β-hydroxysteroid dehydrogenase/isomerase enzyme activity. In another embodiment, the disclosure includes a homologue of a GAME25 cDNA encoding an enzyme having a 3-β-hydroxysteroid dehydrogenase/isomerase enzyme activity. In another embodiment, homologues comprise a polynucleotide sequence which is at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a GAME25 gene nucleic acid sequence.

In one embodiment, the phrase “a polynucleotide sequence” encompasses a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In one embodiment, a “complementary polynucleotide sequence” encompasses a sequence, which results from reverse transcription of messenger RNA using a reverse transcriptase or any other RNA-dependent DNA polymerase. In one embodiment, the sequence can be subsequently amplified in vivo or in vitro using a DNA polymerase.

In one embodiment, a “genomic polynucleotide sequence” encompasses a sequence derived (isolated) from a chromosome and thus it represents a contiguous portion of a chromosome.

In one embodiment, a “composite polynucleotide sequence” encompasses a sequence, which is at least partially complementary and at least partially genomic. In one embodiment, a composite sequence can include some exonal sequences required to encode the polypeptide disclosed herein, as well as some intronic sequences interposing therebetween. In one embodiment, the intronic sequences can be of any source, including of other genes, and typically will include conserved splicing signal sequences. In one embodiment, intronic sequences include cis-acting expression regulatory elements.

In another embodiment, the nucleic sequence of a GAME25 gene homologue is at least 70% homologous to a GAME25 nucleic acid sequence or a fragment thereof, described herein. In another embodiment, the nucleic sequence of a GAME25 gene homologue is at least 80% homologous to a GAME25 nucleic acid sequence or a fragment thereof, described herein. In another embodiment, the nucleic sequence of a GAME25 gene homologue is at least 90% homologous to a GAME25 nucleic acid sequence or a fragment thereof, described herein. In another embodiment, the nucleic sequence of a GAME25 gene homologue is at least 95% homologous to a GAME25 nucleic acid sequence or a fragment thereof, described herein. In another embodiment, the nucleic sequence of a GAME25 gene homologue is at least 98% homologous to a GAME25 nucleic acid sequence or a fragment thereof, described herein.

In another embodiment, the nucleic sequence of a GAME25 cDNA homologue is at least 70% homologous to a GAME25 nucleic acid sequence or a fragment thereof, described herein. In another embodiment, the nucleic sequence of a GAME25 cDNA homologue is at least 80% homologous to a GAME25 nucleic acid sequence or a fragment thereof, described herein. In another embodiment, the nucleic sequence of a GAME25 cDNA homologue is at least 90% homologous to a GAME25 nucleic acid sequence or a fragment thereof, described herein. In another embodiment, the nucleic sequence of a GAME25 cDNA homologue is at least 95% homologous to a GAME25 nucleic acid sequence or a fragment thereof, described herein. In another embodiment, the nucleic sequence of a GAME25 cDNA homologue is at least 98% homologous to a GAME25 nucleic acid sequence or a fragment thereof, described herein.

In one embodiment, homologues of a GAME25 gene are expressed in plants, wherein said plants comprise cultivated tomato, wild tomato, cultivated potato, wild potato, aubergine, sweet or chili pepper plants, or bittersweet plants. In one embodiment, homologues of a GAME25 cDNA are expressed in plants, wherein said plants comprise cultivated tomato, wild tomato, cultivated potato, wild potato, aubergine, sweet or chili pepper plants, or bittersweet plants.

In one embodiment, a GLYCOALKALOID METABOLISM 31 (GAME31) polypeptide comprises a 2-oxoglutarate-dependent dioxygenase enzyme activity. In one embodiment, a GAME31 polypeptide is encoded by a GAME31 gene. In another embodiment, a GAME31 polypeptide is encoded by a GAME31 cDNA.

In one embodiment, a tomato GAME31 gene comprises the nucleic acid sequence of SEQ ID NO: 16. In another embodiment, a tomato GAME31 gene consists of the nucleic acid sequence of SEQ ID NO: 16. In another embodiment, SEQ ID NO: 16 comprises or consists of the following nucleic acid sequence:

(SEQ ID NO: 16)
TGACTATAATTGATACTCAATCTGTTGAAATATAATGAACCAATTCTTAT
CAAAACACAGTGGAGTACAAGTAATCACGTTGGTTCCTATGAAATGGTTC
ATCTATTTCCCATTATATATAGGCTACTTATTTCCTCACCTATAAAGTAA
AAAACTTTCTAGTGTTTTCTTCTTTCTTTTGTTTTTTTCTCTTTGCTCAT
ATTCTAAAAATATTTCATCAATGGCATCTATCAAATCAGTTAAAGTTCCT
ACTATAGATTTTTCCAATTATCAAGAGCTAAAACCAAACACTCCACTATG
GGAATCCACAAAAATTCAAGTTTTTGAAGCTTTACAAGAATATGGTTGTT
TTGAAGCAATATATGATAAAGTTTCAAAGGAAATTAGAGAGGAAACATTT
GATATGTCAAAAGAAATATTTGAATTTCCTTTAGAGACTAAAGTGAAAAA
TATCTCAGAAAAACCAATGCATGGCTATATGGGGATGATTCCACAATTGC
CATTGTATGAGAGTTTGTGTATTCCTGATTTGCTTAATCCTCAAAGTCTT
GAAAAATTTTCTAATATCTTTTGGCCTCAGGGTAATCAACATTTCTGGTA
TGTTTACTTTTATTTCTTTTTCATTTTTGTTTTCTTATTATCTTTAAATT
TTGTTCTAGTGGAACTGTTCAAAAGCTACTATCTTTAGAAATAATAATTT
TTATTAGCTTAGTTGATTGATTATGCGATATTATTAATAGCTTAAAAAAA
TAATTTTTATTAGCTTAGAAATAATAATTTTTATTAACTTAGTTGATTGA
CTATGCGATATTATTAATAGCTTAAAAGAGCTTAGTTGATCAGACTACGA
AAGTAAAATAAAAAGAGACGGAAGTCTGTGTCTCGCATCTATTTTTTATT
GCACCGTTTAAACTAAATAAAATATAGACAACAACATCAAAATATTTGGT
AGGAAGACACGATTTATTCAACAGAAATATAGACAACAACATTAAAGTAT
TTGGTACATGAAATCACTATCCAATAAGTGACAGTTCGTTGGCCTTCTCA
TTTTTTATAAAATAAATAGAAACACAAGAGTTGTCTCAAGTGAAAAAATT
GAATTATGTTCAACCTTCTTCATATGTTTATACTAATATTACATGAGCGT
TAATTTTTGCAGCAATTTGATAAAATCTTATTCTAATCCACTTGTGGAAT
TGGATGGGATGTTGAAAAGGATGATTTCGGAGAATTTGGGATTGAAAAAT
CACATTGATGAATTATTGAATGCCAATTACTTCCTATTTAGATTTACACA
TTATAAGGGATCATCAATTGCTAGTGGAGATGAAAATAATAAAGCTGCTG
GATTGGGTGGCCACACGGATGGTAACTTCTTGACTTTTATATCGCAAAAT
CAAGTTAATGGATTGCAAATCAACAAAAATGGAGAATGGATTGATGTGAT
TATTTCACCAAATTCTTACGTTGTTTTGGCCGGTGATTCCTTCAAAGTAA
GTATTTTAAGTTTTGAACTAGTGTTACTTATCTTGTTGGGAACTGTTTTG
TTTGATTTTTAAAAGAAAAAATATTAAATGATCAAAAAAATTATAATATC
TTTTTTGTTTTAAGGTTAAATAAATTGATTTAAAAATTTCATTTTTAATT
AAAAGAGGGTAGTAAAATGCTTAAAAAGCTAAAATAATTTAGTGTGAAAT
ATATTATTTTATTATCATTCTAATCAAAATTTCTGGTCACACCTTATAGC
ATAGGGGTTTCAGAGGGCCCCGAGATATTTTGTTTTGATCTTATATTTCT
CGAATCTATGAAAATGTTATTCCACTAGTGTTTATATTATTTTCTGAAAT
GCATATTTTTGAATGATTTGATATATGCTCAATATTTTCATGCAAAACTG
AAAATGAATTTTGGTATTATTGACCGTATTTGTATTGTTTTACTCTCCAA
AAATATTATCGATCGCATCTATCTTTGTATTTATACAGGCTTGGACAAAT
GGTCGATTGCATTCACCTCTCCACAGAGTAACAATGTCCGGACAAAATGA
TAGACTCTCCATTCAATTGTTTTCATTATCAAAGCCAGGTCACTTCATCC
AGGCACCAAAAGAACTAGTAGATGAAGAACACCCATTACTCTTCAAGCCA
TTTGAAATTCTTGAATTATTCAAGTATGGTACCACAGAAGCTGGCTATAC
AGCTCCTCCAAGTGATCTTTTCAAGATTTATTGTGGTGTTTGATATGCTA
ATTGTTGAATTTCCGCTTCAACAAGCAACTTTTCTAATGAGTTTCATCTT
GTTTTTTTAAGTAGTATGCATTTTATGTTTGAATTGTTGCAGTTGGCAAT
TCATGTTTAATTTGTTTTTGTTTTTTTGAGAAAATATTTCCAATGGGTTT
CGTTGGAAATTCGTCTTGTTTTTTTTTTTCAAGTAGTGTACATCTTATTT
TTGGATTGTTGATGTTGAGCGCTAATGTTTAATTTGTTTGTGTTTTGAAG
AGGATGATTATACTCTTTAAGAGGATTCACCGTAATCTTTTAGTATTATT
TG.

The start codon (ATG) for SEQ ID NO: 16 starts at nucleotide 221 of SEQ ID NO: 16. The coding sequence present within SEQ ID NO: 16 ends at nucleotide 2243. Therefore, the skilled artisan would recognize that the coding sequence comprised in SEQ ID NO: 16 is found between nucleotides 221-2243.

In one embodiment, a tomato GAME31-like gene comprises the nucleic acid sequence of SEQ ID NO: 19. In another embodiment, a tomato GAME31-like gene consists of the nucleic acid sequence of SEQ ID NO: 19. In another embodiment, SEQ ID NO: 19 comprises or consists of the following nucleic acid sequence:

ATGGCATCTACCAAATTAGTTAAAGTTCCCACAATAGATTTTTCAAATCA
TCAAGATCTAAAACCAAACACTCCACTATGGGAATCCAAAAAAATTCAAG
TTTTTGAAGCTTTGCAAGAATATGGTTGTTTTGAAGCAATTTATGATAAA
GTTCCAAAAGATATTAGAGAGGAAACATTTAGTATTTCAAAAGAAATATT
TGAATTTCCTTTAGAGACTAAATTGAAAAATATTTCAGAAAAACCAACGC
ATGGATATATGGGAATGATTCCACAATTGCCATTGTATGAGAGTTTGTGT
ATTCCTGATTTGCTTAATCCTAAAAGTCTTCAAAGTTTTGCTAATATCTT
TTGGCCTCAGGGTAACCAACATTTCTGGTATGTTTACTTATGTTTTTATT
TCGCCCTAGCAGAAGTGTTCAAAAGTACGACATTAGAAATTCTTAGTGAC
TTAATTGATTGA (SEQ ID NO: 19; SlGAME31-like1)

In one embodiment, a tomato GAME31-like gene comprises the nucleic acid sequence of SEQ ID NO: 22. In another embodiment, a tomato GAME31-like gene consists of the nucleic acid sequence of SEQ ID NO: 22. In another embodiment, SEQ ID NO: 22 comprises or consists of the following nucleic acid sequence:

TGAGATGTTGAAAAGGATGATTTCGGAGAATTTGGGATTAAAAAATCACA
TTGATGAATTATTGAATGCCAATTACATCCTATTTAGATTTACACAGTAT
AAGGGATCATCAATTGCTAGTGGAGATGAAAATAATAAAGCAGCTGGATT
GGGTGGCCACACAGATGGTAACTTCTTGTCTATTATATCACAAAATGAAG
TTAATGGATTGCAAATCAACAAAAATGGAGAGTGGATTGATGTCAACATT
TCGCCAAATTCTTATGTTGTTTTATCCGGTGATTCCTTCACAGTAAGTGT
TAAGTTTTGAGCTAGTGTTATTATCTTGTTGGGAACTGTGTTGTTTGATT
TTCTAAAGGGATAATGCTAAATGACAAGAAACTCAAAAAATCAATAAGAT
ATTTGTTGAATCTTACGTCTCTAAATATATTATCATGCTAGTGTTAATTA
TTTCCCGAAATGCATATTTTTGAAGAATCTGACATACTGAGTGATATTCT
GGAAGAGTCCAACCAAGACACTTTGTTGAAACTACATGCTCAATATTTTC
ATGCAAAACTGAAAATGAATCTTGATATTTGTTGACCCTATGTTGCTCTA
TTCTCCAAAAATACTACTGCGACTATCTTTGTATTTATGCAGGCATGGAC
AAATGGCCGATTGCATTCTCCTGTTCATAGAGTTGAAATGCCCAGAGGAA
GTGATAGATATTCCATTCAATTATTTTCATTATCAAAACCAGGTCACTTC
ATCGAGGCACCAAAAGAAATGGTGGATGAAGAACACCCTTTGCTTTTCAA
GCCATTTGAAATTCTTGGATTACTTGGGTATGGTGCCACAGAAGCTGGCT
ATACAACTCCTCCCAGTGATCTTTTCAAGGCATATTGCGGTGTCTGATAT
GCTAATTGCGAATTTCCATTTCTATTAGAATAAAGTTAGTATTTATGAGA
TTTTTGTTGGTAATTCATGTTTAATTGGTTTGTGTTTTTTTGGAAAATAT
TTCTAATGTGTTCCGTTGGAAATTCGTGTGCATCTTATGTTTGGATTGTT
GGTATTGGGAATTCATGTTTAATTTGTTTGTGTTCTTGGGCAAATAATAA
ATTTGAAGCGGAT (SEQ ID NO: 22; SlGAME31-like2).

In one embodiment, a tomato GAME31-like gene comprises the nucleic acid sequence of SEQ ID NO: 25. In another embodiment, a tomato GAME31-like gene consists of the nucleic acid sequence of SEQ ID NO: 25. In another embodiment, SEQ ID NO: 25 comprises or consists of the following nucleic acid sequence:

TGACTAGAAATAGACATAAAACCCTGTACTTTTTGACACACATGTAATAG
CACTTTCTCTATCTAATACGCAACTCTTTATTAATTTTGCGTAAATTTTG
AGCTATTTCCCATTATATATAGGCTACTTATTTCCTCACCTCTTAAGTAA
AAAACTTTCAAGTGTTTCTTCTTTCTTTTATTTCTCTTTGTTCACATATT
CTAAAAATATTTCATCAATGGCATCTATCAAATCAGTTAAAGTTCCTACT
ATAGATTTTTCCAATTATCAAGAGCTAAAACCAAACACTCCACTATGGGA
ATCCACAAAAATTCAAGTTTTTGAAGCTTTTCAAGAATATGGTTGTTTTG
AAGCAATATATGATAAAGTTCCAAATGAAATTAGAGAGGAAACATTTGAT
ATGTCAAAAGAAATATTTGAATTTCCTTTAGATACTAAAGTGAAAAATAT
TTCAGAAAAACCAATGCATGGATATATGGGAATGATTCCACAATTGCCAT
TGTATGAGAGTTTGTGTATTCCTGATTTGCTTAATCCTCAAAGTCTTCAA
AATTTTGCTAATATCTTTTGGCCTCAGGGTAATCAACATTTCTGGTATGT
CTATTTCACTGTTTTCATCTTTTTTATTTTCTTACTATCATTATCTTTAA
ATTTAAGAAAAAACGATAAATATATCCTTAAATATAAATGGTATGCAGAT
ATTCTCCATCATATTTTTGGGACATATATTTTTACCGTTCAAAAATTAAA
GCATATATACCATTTTATACTAATGGATATAGACGTGTCATAATCTTATC
TACCGCCCCAACATTGGATCGATGGATAAGATTGTGCCAAGTTTCCTAAT
TTAACCATTCGTTAGAGTGAAGGGCAGAAATTTTCGACTTTTTAAATGTC
AGGGACATCAATGTCCCAAAAGTATGACGGAGGAAAAATATAACGAAAAA
TATGTGCATACTATTTACGATCGTTTGAAAAAATATTTGTCTTTTTTCCT
TTTAAATTTTGGCCTAATGAAAGTGTTCAAAAAGTATAACATTAGAGATT
CCCAGTAAATTGATTAACTATTTGACCATTCAATAGTTTTATGGAATAAG
TTAATTTTTTCTTTGAGAAAAACAAAATAGGAGATTATAAATGGAAGTTT
CCTTCTCGAATCTATTCATTAGCACACCCCTAAACAAACAAAAGTGCCGA
TAACGTTAAAATATTTGGTATGAAGTCTCGATCTACTCAATAAAAACAAA
TAAATTAATAGGAGACAATAGACAGTATCTTTCTCGAATCAATTTCGAAT
TTATTTCTTTTATCACATTGTTAATAAAATGAAAATTATCAACAACATCA
GAATATTTGGTATGTGTCACGATCTAATAATTGTAGAAATCGAGATATAA
ATACGATTTGAGAAATCAAAGATTGTATTGATGAAAAATAATGTTAAGTT
ACAAGGTTTTTATATGGAGAGAATTGTAGAGTTCTAAGTTAACTATAATA
AAATACTATTACAATACATATTACTATTATAATAATAATAATAATGCAAA
TCCTAGTCGTAATATAATTCTAATCGACTTCAACTAGTACAATAAGTAAA
TAAGTAGCACTGCGTCTAATCCTAGTATGAATCTAACTCGTCAGTCAGTT
CCGCTTTCCCATTTTTGTTTTCACTATCCTAGTTTTAAAAATAAAATAAA
ATATAAGACTTAGTTAACGTAAATATGGCCTCATTTGTTTGTATTTAATT
GGGGGTCTAAATCTTAATCAATCAGATTCGCCTCATTCAGTATGTTTGTT
TTTTTATGACTGAATCTTAATTATTTAGATTTAATTCATTAAGTTTGTTT
GTTTTATTTTCTTAGAAGTCTCTTAACGAGTCTGAATACATCTGAGTTAA
TCAGATCTGTAATACACTCTTAAGACCATTCAGACTCAAAAGTAATTCCT
ATCTTAATTCAACTACATCACAAAAACTCATAAAAGTTTTTTTCTTATTA
AATTAATGTTAATTACATGCTTACCCGTTATAATTTCCTTTATTTTACTA
ACTTAATATACGTCCTTTACTTTCATAAATTAATCAATATATTTGAATTG
ATAAACACTTCATGATATAATATTTAGCACGATTCTAGAAAACAAGAAAG
TATTGATTAGTTGATCGATAACAATAACAAATCTGCATTATAAAATAAAA
TGCTTTCATAAACATTATATTACTACTCTATATAAACTATTTTACATTGC
ATTATATTAGATTATCAAAGTTTTTGAGTGCAAAAAAGAATAGTCATATA
TTAGTGATGTAACTTAATGTTAAATTCTTAATAGATAAATCATATGACCT
ATTCATGATAAGAATGTCCAAAAATTTATTTTCCATATAAAAAATTATTT
TACTAAAATGAGGTTTTTATAATTTTTTGTTGATACATCGTTTAATTTCA
TATGTACATTCAAATATTAAAAACGAATTATCTCAATAATCCAGTTTTCA
TATTCAGAGAAAATACCTTAATATTAAAATGTTTATTCAGATTTACATAT
CTAGATCTTAATGCATATTTTAATATTTAGATGTATATTCAGATTCAGAC
GTTTTGATCTTAATAGAAACAAATAAGGCCTAAGTGAAAGAATGGTATCA
ACTTGAAATGTTTCTAAATCTGTTCAACCTTCTTTATATGTTTATAAACA
TTATATGTGTATTTTTTTTTTGCAGCAATTTGGTAAAGTCTTATTCTAAT
CCACTTGTGGAATTGGATGAGATTTTGAAAAGGATGATTTCGGAGAATTT
GAGATTAAAAATTCACATTGATGAATTGTTGAATGCCAATTATTTCCTAT
TTAGATTTACACATTACAAGGGATCATCAATTACTGGTGGAGATGAGAAT
AACAAAGTTGCTGGATTGGGTGGCCACACAGATGGTAACTTCTTGACTTT
TATATCGCAAAATCAAGTCAATGGATTGCAAATCAACAAAAATGGAGAAT
GGATTGATGTGAATATTTCACCAAATTCTTATGTTGTTTTGGCTGGTGAT
TCCTTCAAAGTAAGTGTTAAGTTTTGAATTATTGTTATTATCTTGTTGGG
AACTGTTTTGTTTGATTTTTAAAAGAAAAATGCTAAATGGTCACAAATTT
TTAAAGTCAATAATATTTTTTTTGTTTTAAGGTAAATAAATTGATAAAAA
AAGAATTCATTTTTAATTAAAAGATATTGAAATTAAAAGGGTAAAAATAC
TTTAAACATAGTGTGAATTATGTTATTTTATCATTCTAATCAAAATTTGT
GGCCAATATTGTTACACCTTATAGGATTTATCAAAAAAACATAGTTTTCA
GAGGCTCAAGATATTTGTTGGATCTTATGTTTCTCGAATCTCTGAAAATG
TTGTTCCGCTTGTGTTGAATGTATTATTTTCTGAAATGTATATTTTTGAA
GAATTTGATATATTAATGATATGCTCAATATTTTCATGCAAAACGGAAAA
TGAATTTTGGTATTATTGACCCTATTTGTATTGTTCTACTCTCCAAAAAT
ATTATCGATCACGTCTATCTTTGTATTTATACAGGCTTGGACAAATGGTC
GATTGCATTCTCCTCTTCACAGAGTAACAATGTCCGGAGAAAATGATAGA
CTCTCCATTCAATTATTTTCATTATCAAAACCAGGTCACTTCATCGAGGC
ACCAAAAGAACTAGTGGATGAAGAACACCCTTTACTCTTCAAGCCATTTG
AAATTATTGGATTATTTGAGTATGGTACCACAGAAGCTGGCTATACAGCT
CCTCCAAGTGATCTTCTCAAGAGTTATTGCGGTGTTTGATATGCTAATTG
CGAATTTCCGCTTCAGCAACCAACTTTTCTAATAAGTTTCGTCTGAAATT
CGTGTTGTTTTAATTATTATGCATTTTATGTTTGAATTGTTGTAGTTGGC
AATTCATGTTTAATTTGTTTGTGTTTTTTTTTTTGAGAAAATATTGCATT
GGGTTTCATTGGAAATTTGTGTTTTTTAAAAAGTAGTGTGCATCTTATGT
TTGGATTGTTGGTGTTGAGAATTCATTTTTAATTTGTTTTTTTTTTTGGG
CAAATAATGAATTTAAAATTGTTGATTTTACTCTTTAGTGGAAATGAT
(SEQ ID NO: 25; SlGAME31-like3).

The start codon (ATG) for SEQ ID NO: 25 starts at nucleotide 218. The coding sequence present within SEQ ID NO: 25 ends at nucleotide 1182. Therefore, the skilled artisan would recognize that the CDS comprised in SEQ ID NO: 25 comprises nucleotides 21-1182.

In another embodiment, a potato GAME31 gene comprises the nucleic acid sequence of SEQ ID NO: 30. In another embodiment, a potato GAME31 gene consists of the nucleic acid sequence of SEQ ID NO: 30. In another embodiment, SEQ ID NO: 30 comprises or consists of the following nucleic acid sequence:

(SEQ ID NO: 30)
GAAACTTTGAAGTCTTTCTTGTTTCCTAAATATTCCTCAAATGGCATC
TACCAAAGTTACGATTCCCACCATAGATTTTTGCGATTCTGAGCTTAAAC
CAAACACTCCACAATGGGAATCAACAAAAGTTCAAGTTTTTGAAGCCTTA
CAAGAATTTGGTTGTTTTGAAGCAATATATAACAAAGTTCCAAATGAAAT
TAGAGAGGGCATGTTTGATACTTTAAAAGAAGTATTTGATTTTCCACTGC
CCAAATTGATAGAATATAGAGAGAAACCCTTTCATATATATGATGGGCAA
ATTCCAAGTGTACCACTCTTTGGTAGTGTGTACTCTGCTGATTTGGTCCT
CCCAAATAGTGTTGAAACATTTGCCAATACCTTTTGGTCTCATGGAAACC
CTAATTTTAGGTATGCATTACTTCTTTTTCATTAATTATAGGGAGTCACG
ATAGTGTAAGATGCATTAAAAGGAGAAACGTTTCCTAGTAGAATTGTTTC
TATTCCTAGGGCTAGAATCAGGAACCTTTAGTTAAATTAATAGAGATAAT
ATTCATTCCATCACTAAAGGTGAAAATTAAGTATCTTATATTGTCCATAA
ATTTTATATAGAAGAGATGTGAGAATTAATAAAATAAAAATTAAAAACTC
ACGAGTAAACAAAATAATTATATCTAATTTATATTAATAAAGAAGAGTAT
TTGATTATTATATTAAGTCAAATGATAAGCTAATAAATCAATATTAACAA
TCTAATCACATGATTTATATAAAATTGGTTATGGGTATGGGAAGGGAGGG
AGGGAAGTACATTTCATTGAGGAACAATGCAATAGTTAGACAGGATTTAA
CATACTTGAACAAGATATCATAATCTAAAATGATTAAAAATAATTTTTTA
ATATTATCTACACATCGCGCGAATATATATATATATATATATTAAGTGTA
TTTCTTAAATAATATTGTATTACTATTTATATAAATTTTGTATGTTTTAA
TTTTGCAGCAATGTGGCAAAGTCCTACTTCAAGCAACTTATGGAATTAAA
TGACATGGTTAAAAAGATGGTTTTGGAGAGTCTTGGGCTAAAAAATTACA
TTGATGAATTCTTGAATTCCAATGTTTATATGTCAAGATTTACTAATTAC
AAGGTAATTAAAGGTGAAAATGAGAATAAATCAGGATTACCTTCCCACAC
AGATAGTTCCTACTTGACCATAATTAAACAAAATCAAAATGGATTGCAAG
TTCTCTACAAAAATGGAGAGTGGATTGAGCTCAATCGTCAAAATGGACTG
CAAGTTCTCTACAAAAATGGAGAGTGGATTGAGCTCAATCATACTTCACC
AAATTCCTATATTGTTTTATCAGAAGATGTTTTTATGGTAAGTTATTATT
TATTTTTTATTACAGAAGTCAAAAATACACCTAAACTTTTTATTTATATG
TATTTTTGACGCTTAACTCTTTATTTTTTTGTGTGTAGGGGTGGTTTGTT
GCTATAGTAGAGGAGAATAAAAGAAATAGATTTTTTTTGTATGATTGATT
ATTCAAGCCCAACTAGAAGCTAAGATTAGAGGAGTTTTGAAGCAACGAAA
AAAAATGTTGTGTGTGATTTATAGATATTGATGCAGGCTCGATCCGTGAA
AGAAATCACTAATATTTATATTAGATTAGATCGTTTACCTAACTAAACAT
CCCTTGAAGTACTGCCCTTTCTCCAAACCATATGTGAACGTCAAATATTT
TATGCATCAACCTGTCTTTTTTTATTTGGCCCCAACTAACTTCAATCCAC
ATAAATTATTAAATCTTGATATTAGTTGGAATAACATATCTCTTTTCTGA
GAAATTGAAAATAATGCCAGAACTATCATAATCTTTTTTTAAAAAAATTG
TCTTGTTATTATCTTATTAATTTAAAATTTTCTTTCTTCAGAGGAAATTT
AAGTCAATCTTTTTGTTCCTTAATTATTAATTAAACAAATAAATTCTTAT
ACATACTTTTTATGTGTTGATGCTATGAATTAATTATACAGGCATGGACA
AATGATAGATTGACATCTGCTCAACACAGGGTTGTAACAACAGGAGACAA
AGAAAGATTCTCTATTCAAGTTTTTTCCTTTCCAAATCCAGATTACACTG
TGAAGGTCCCACAAGAATTAGTGGATGAAGAACACCCTTTAATGTACAAG
CCTTTTAAGATGTCTGAATATAATAAATATATTATGTTAGGTGCTAAAAA
TGGATTGGGTGTCAAGAATTATTGTGGTCTTTAAAAATTTAGTAGCTATG
AAAATTTATTTATGTATTGTTTTGATGAATAAAATGTATCAGATGGC.

In one embodiment, a potato GAME31-like gene comprises the nucleic acid sequence of SEQ ID NO: 33. In another embodiment, a potato GAME31-like gene consists of the nucleic acid sequence of SEQ ID NO: 33. In another embodiment, SEQ ID NO: 33 comprises or consists of the following nucleic acid sequence:

″GAAACTTTGAAGTCTTTCTTGTTTCCTAAATATTCCTCAAATGGCATCT
ACCAAAGTTACGATTCCCACCATAGATTTTTGCGATTCTGAGCTTAAACC
AAACACTCCACAATGGGAATCAACAAAAGTTCAAGTTTTTGAAGCCTTAC
AAGAATTTGGTTGTTTTGAAGCAATATATAACAAAGTTCCAAATGAAATT
AGAGAGGGCATGTTTGATACTTTAAAAGAAGTATTTGATTTTCCACTGCC
CAAATTGATAGAATATAGAGAGAAACCCTTTCATATATATGATGGGCAAA
TTCCAAGTGTACCACTCTTTGGTAGTGTGTACTCTGCTGATTTGGTCCTC
CCAAATAGTGTTGAAACATTTGCCAATACCTTTTGGTCTCATGGAAACCC
TAATTTTAGGTATGCATTACTTCTTTTTCATTAATTATAGGGAGTCACGA
TAGTGTAAGATGCATTAAAAGGAGAAACGTTTCCTAGTAGAATTGTTTCT
ATTCCTAGGGCTAGAATCAGGAACCTTTAGTTAAATTAATAGAGATAATA
TTCATTCCATCACTAAAGGTGAAAATTAAGTATCTTATATTGTCCATAAA
TTTTATATAGAAGAGATGTGAGAATTAATAAAATAAAAATTAAAAACTCA
CGAGTAAACAAAATAATTATATCTAATTTATATTAATAAAGAAGAGTATT
TGATTATTATATTAAGTCAAATGATAAGCTAATAAATCAATATTAACAAT
CTAATCACATGATTTATATAAAATTGGTTATGGGTATGGGAAGGGAGGGA
GGGAAGTACATTTCATTGAGGAACAATGCAATAGTTAGACAGGATTTAAC
ATACTTGAACAAGATATCATAATCTAAAATGATTAAAAATAATTTTTTAA
TATTATCTACACATCGCGCGAATATATATATATATATATATTAAGTGTAT
TTCTTAAATAATATTGTATTACTATTTATATAAATTTTGTATGTTTTAAT
TTTGCAGCAATGTGGCAAAGTCCTACTTCAAGCAACTTATGGAATTAAAT
GACATGGTTAAAAAGATGGTTTTGGAGAGTCTTGGGCTAAAAAATTACAT
TGATGAATTCTTGAATTCCAATGTTTATATGTCAAGATTTACTAATTACA
AGGTAATTAAAGGTGAAAATGAGAATAAATCAGGATTACCTTCCCACACA
GATAGTTCCTACTTGACCATAATTAAACAAAATCAAAATGGATTGCAAGT
TCTCTACAAAAATGGAGAGTGGATTGAGCTCAATCGTCAAAATGGACTGC
AAGTTCTCTACAAAAATGGAGAGTGGATTGAGCTCAATCATACTTCACCA
AATTCCTATATTGTTTTATCAGAAGATGTTTTTATGGTAAGTTATTATTT
ATTTTTTATTACAGAAGTCAAAAATACACCTAAACTTTTTATTTATATGT
ATTTTTGACGCTTAACTCTTTATTTTTTTGTGTGTAGGCGTGGTTTGTTG
CTATAGTAGAGGAGAATAAAAGAAATAGATTTTTTTTGTATGATTGATTA
TTCAAGCCCAACTAGAAGCTAAGATTAGAGGAGTTTTGAAGCAACGAAAA
AAAATGTTGTGTGTGATTTATAGATATTGATGCAGGCTCGATCCGTGAAA
GAAATCACTAATATTTATATTAGATTAGATCGTTTACCTAACTAAACATC
CCTTGAAGTACTGCCCTTTCTCCAAACCATATGTGAACGTCAAATATTTT
ATGCATCAACCTGTCTTTTTTTATTTGGCCCCAACTAACTTCAATCCACA
TAAATTATTAAATCTTGATATTAGTTGGAATAACATATCTCTTTTCTGAG
AAATTGAAAATAATGCCAGAACTATCATAATCTTTTTTTAAAAAAATTGT
CTTGTTATTATCTTATTAATTTAAAATTTTCTTTCTTCAGAGGAAATTTA
AGTCAATCTTTTTGTTCCTTAATTATTAATTAAACAAATAAATTCTTATA
CATACTTTTTATGTGTTGATGCTATGAATTAATTATACAGGCATGGACAA
ATGATAGATTGACATCTGCTCAACACAGGGTTGTAACAACAGGAGACAAA
GAAAGATTCTCTATTCAAGTTTTTTCCTTTCCAAATCCAGATTACACTGT
GAAGGTCCCACAAGAATTAGTGGATGAAGAACACCCTTTAATGTACAAGC
CTTTTAAGATGTCTGAATATAATAAATATATTATGTTAGGTGCTAAAAAT
GGATTGGGTGTCAAGAATTATTGTGGTCTTTAAAAATTTAGTAGCTATGA
AAATTTATTTATGTATTGTTTTGATGAATAAAATGTATCAGATGGC
(SEQ ID NO: 33; StGAME31-like1.

In one embodiment, a potato GAME31-like gene comprises the nucleic acid sequence of SEQ ID NO: 36. In another embodiment, a potato GAME31-like gene consists of the nucleic acid sequence of SEQ ID NO: 36. In another embodiment, SEQ ID NO: 36 comprises or consists of the following nucleic acid sequence:

TTATTATTTGCACAAAAAATACAAACAAAGCATGAATTCCCATCGAA
ATTACTCACTGTTACAACAATTCAAACATAAGATACACACTACTAAAGAA
ACACGAATTTCGACAAGCATTCTGTTAGAAATCCCATATATACTTACAGT
ATTCTAACAAGAACGTTCATAAAAAAATCACGTTTTTTTATTCTAGCATA
TATAAAACAAAGAACTCTAAAGCTAAAGATACACACTATTATAAAAACAC
GAATTCCAAATGAAATCCATTGGAAATGATGAAGACATTTTCGATGAATA
TTCTGTTAGAAATCCCAAATATACTAACAGTACTCTGACAAAAATGTTCA
TTAGAAATTCACGTTTATCATATCAAAGACCACAATAAGCCTTGAAAGCA
TCACTGGGAGCTGCATAGCCAGCTTGGGAAGTAACATACTCAGATAATCC
AACCATTTCAAATGGCTTGAAGAGTAAAGGGTGTTCTTCATCCACAAGTT
CTTTTGGGGCCTTTATAAAGTGACCTGGTTTTGAGAATGAAAATAATTGA
ATTGAGAATCTATCACTTTCACCAAATATTTTTACCTTGTGGATGGGAGA
ATGCAATCGACCATTTGTCCATGCCTGTATAAATTCACGTCGTCAGTGCA
TAAAACTCAAACACATTTATTTGAAGGATCCAACATTTTTAGAGATTCAA
AAAGCATAGACTCCAACAAATATCAAAATTCATTTTTCAGTTTTGCATGA
AAATATTAAATATGTAGGTAGTTCCATTTAATATTCGAGAAACATAGGTT
AATTCCAACAAATATGAAGATTCATTTTCAATTTTGCATCAAGATATTAA
ACCTAAATTTTGTTTGGTATATTCCAACCTTGGAAATATTCTTCACAAAT
ATCGTTAGGTATGCGTCAGATCCTCTAAAATCTATATTTTGTTTTTGAAG
GTGCAATAATAATATTTTTGAAGAGTTCGAGTAACATGGATTTCAACAAA
GTAATATGACTAGCTAAAAAAATAAAATGAGTAACACTTACTTTGAATGA
ATCACCAGACAAAACAACATAAGAATTTGGTGAAATATTGACATCAATCC
ACTCTCCATTTTTGTTGATTTGCAAACCATTGACTTGATTTTGTGATATA
AAAGTCAAGAAGTTACCATCTGTGTGGCCATTCAATCCATCTTGTTTAAT
ATTATTTTCATGATCTCCACTAATAATTGATGATCCCTTGTAATGTGTAA
ATCTAAATCTCATATAATTAATATCCAGCAATTCATCAATATGATTTTCT
AATCCCAAATTCTCCAAAATCATCTTTTTCAACATTTCATCCAATTCCAT
AAGTGGATTTGAGTAAGCTTTTACCAAATTGCTGCAAAAATTTATCACTA
TCTCAAAAATAACTTTCTCGCTACTCCACGACTCTAATCAATGCACAAAA
TAATTTTATTTTAAAAAATAAAATAAAGTAGACATACCAGAAATCAGGAT
TACCATGAGGCCAAAAGATATTAGCAAAAGTTTCAACATTTTGAGGATTA
AGCAAATCAGGAATACACAAACTCTCATAAAATGGCAAGTGTGGAATCAT
TCCTACATAGCCATGTAATGTTATATCTGAATAATTTTTCACTTTGGTCT
CTAAAGGAAATTGAAATATTTCTTTTGTAATACCAAAAATACCCTCTCTA
ATTTCATTTGGAATTTTATCATATGTTGCTTCAAAACAACCATATTCTTT
TAAAGCTTCAAAAACTTGAACTTTTGTGGATTCCCATAGTGGAGTGTTTG
GTTTTAGTTCTAGATTAGAAAAATCTATGGTGGGAATCTTAACTTTGGTA
GATGCCATTTGAAAGAAACAAAGAAGGAATTAAAGACTTCACAATGTGAA
(SEQ ID NO: 36; StGAME31-like2).

In one embodiment, a potato GAME31-like gene comprises the nucleic acid sequence of SEQ ID NO: 39. In another embodiment, a potato GAME31-like gene consists of the nucleic acid sequence of SEQ ID NO: 39. In another embodiment, SEQ ID NO: 39 comprises or consists of the following nucleic acid sequence:

ATTTTTTAATAAAAATAATACAGTACAATACATTACAATACAACGCA
ACACAATACAATACGTTATGTAATCATATGCAATAACCATTCAAATATAA
ATTTTCAACCACCCATACATACGATACATTTTATTCATAAAGAAAATACA
CATATATAAATTTTCATACCCTACAAAAATTTTAAAGACCACAATAATTC
TTGAGATTAATTCCATTTTTATCACCTGACATAGTTTATTTATGAAATTC
AAGCAAGTTAAAAGGCTTGAAGAGTAAAGGGTGGTCTTCATCCACTAATT
CTTTTGGGGTCCTTCACAGTATAATCTGGATGTGGTATGGAAAATAATTG
AATAGATAATCTATCTTTGTCTCCTGTTGGTACTACTCTGTGTTCAGCAG
ATGTCAAACTATTATTTGTCCATGCCTGTATAAATCCACAACATCGACAC
ATAAAAAGCATTAGTGATTTCTTTCACAGAGAAAGGGCAGCACCTCAAGG
GGATGTTAGGAAAACAATCTAATCTAATAGAAAATATTAGTGATTTTTTC
TCGATCAAGCCCGCATCGTAATCTATATATCACATACAAACAATCTTATC
GTTACTTCAAAACTGCTCTGATCTTAGCTTCTAGTTGGGCTTAAATAATC
AACCATACAAAAAAAATCTCTATTTCTTTNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNGGTGTGTTTGGTA
CGAAGGAAAATATTTTCTGGAAAATGTTTTTTAATTTCCCATGTTTGGTT
GACTTTAATTATTTGGAAAATGTTTTCCAAATCAACTTATTTTCCTCAAA
TTTAAGGAAAATGATTTCCCTTAAAAATTAAGGAAAACATTTTCGAAACT
TCTACTTCATCCTTAAATTATAATATTTTTTTACCCCTACTACCAAACCA
GCCCGCCACCCCTGTCAAATTTTATTTTATTTAAAAAATTACTTTTGAAA
AATATTTCAGGGTCGGAAGTTTGGCTGGGGTCCTAGGTCAGATCTCTAGG
TCGGATTTTGAACGGTCATCAAGGTCATGTCCTAGTTCGGGTGTTAGGGT
CGGGTCCTAGATTGATTATTGGGATTGATTTTCGAGTTAAAAGTTATTTT
CCTAAAGAGTATTTTCTAGTCTTAAGCGAAAAATAAAAGATATTTTCTGG
AAAAAAAATTCATTCACCAACCAAACATTAAAAAATAGTTTCTACTCATC
AACTAAATATGAGAAAATAAGTTAGAAATCCACTTGTTTTCCAAGAAAAC
ATTTTCCTTCATACCAAACACACCCTTAATATATATNNNNNNNNNNNNNN
NNNNTTATATATGTATAGATAGCTAGTATACATACTCGAGCGATGTGCGG
AAAATATTAATATGTTATTTTTAATCTTACCCCTACCCATGCCCCTACCC
GACCTTCCCCATTCAAAAAAATAAATTAAAATTTTTAAAATTTCAAATAT
ATTTTTATCACTACCACCAAACTAGCTCCCCCGCCCCCCTCCCCTCAAAC
ATAAAAAAAAAAAATTAAAATTATTTTTGAAAAATTTTTAAATCTTAAAT
TTTTTTTACCTCACCAACCCCTACCACCCCTACTCCCTTCCCCCTCATTT
TTAATTTCCCATGTTTGGTTGACTTTAATGATTTGGAAAATGTTTTCCAA
ATCAACTTATTTTCCTCAAATTTAAGGAAAATGATTATCCTAAAAGTTAT
TTTCCTAAAGAGTATTTTCTAGTCTCAAGAGAAAATAAAAGATATTTTTT
CAGAAAATAATTTTCATTCACCAACCAAACATGAGAAAATAAGTTAGAAA
TCCACTTGTTTTCCAAGAAAACATTTTCCTTCATACCAAACACACCCTTA
ATATATAATGTATATATACCTAGTATACATACTCGCGTGATGTGTGAAAA
ATATTTAAATGTTATTTTAAATCATTTTAGATTGTGATATCTTGTTCGAG
CATGTTAAATCACGTCCAACTATTGCATTGTTACTCAATGAAATGTACTC
CCCTCCATCCCTTCACATACCCGTAACCAATTCTATATGAATAGCGACAA
TGAATCATGTGATTAGATTGTTAGTATTGATTTTATTAGCTTATCACTTG
GATATAATTATTAAATACTCTTCTAATTAATATAAATTAGATATATTTAT
TTTGTTTACTCGTGAGTTCTTAATTTTTATTTTATTAATGATCACATCTC
TTTTATATAAAATGTATGGACAATAAGATACTTAATTTTCACCTTTAGTG
ATGGAATGAATATTATCTCTATAATTTAACTAAAGATTCATGATTCTAGC
CTTAGGAATAAAAACAATTCTAGTAGGAAACGTTTCTCCTTTTAATGTGT
CTTACACTATCGTGACTCCATATTATTAATCTGAATCCCAAAATAAATAC
CGAATACATAATGAAAAAAAAAAAATCTCCTACAAATATATGAGCAAGAA
TAATTGGTGTACTAACATGACTAAAACTAAATGATATCACCTACATAAAT
CTTATTCTTTCAAATATCATTAATGAAAAATGACTTTCATAATACCGAGC
ACATTATATGTAATTAAAAGAAGTAATACATACCTAAAGTTAGGGTTTCC
ATCAGACCAAAAGGTATTGGCAAATGTTTCAATACTATTTGGGAGGACCA
AATCAGCAGAGCTCACACTACCATAGAGTGGTATACTTGGAATTTTCCCA
TCATATATATGAAAGGGTTTTTCTCTATATTCTATCAATTTGGACAATGG
AAAATCAAATACTTCTTTTAAATTATCAAACATGCCCTCTCTAATTTCAT
TTGGAACTTTGTCATATATTGCTTCAAAACAACCAAATTCTTTTAAGGCT
TCAAAAACTTGAACTTTTGTTGATTCCCATTGTGGAGTGTTTGGTTTTAG
CTCAAGATTGCAAAAATCTATGGTGGGAATCTTACCTTTAGTAGATGCCA
TTGATGAATAAATTAAAAAGAGAGGAAATAGAAAGAAATAAAGAAGGAGA
AATACTTTACAATATGAAAATTAAAG (SEQ ID NO: 39; St.
GAME31-like3; N represents unknown nucleotide
sequence).

In one embodiment, a potato GAME31-like gene comprises the nucleic acid sequence of SEQ ID NO: 42. In another embodiment, a potato GAME31-like gene consists of the nucleic acid sequence of SEQ ID NO: 42. In another embodiment, SEQ ID NO: 42 comprises or consists of the following nucleic acid sequence:

GAAACTTTGAAGTCTTTCTTGTTTCCTAAATATTCCTCAAATGGCATC
TACCAAAGTTACGATTCCCACCATAGATTTTTGCGATTCTGAGCTTAAAC
CAAACACTCCACAATGGGAATCAACAAAAGTTCAAGTTTTTGAAGCCTTA
CAAGAATTTGGTTGTTTTGAAGCAATATATAACAAAGTTCCAAATGAAAT
TAGAGAGGGCATGTTTGATACTTTAAAAGAAGTATTTGATTTTCCACTGC
CCAAATTGATAGAATATAGAGAGAAACCCTTTCATATATATGATGGGCAA
ATTCCAAGTGTACCACTCTTTGGTAGTGTGTACTCTGCTGATTTGGTCCT
CCCAAATAGTGTTGAAACATTTGCCAATACCTTTTGGTCTCATGGAAACC
CTAATTTTAGGTATGCATTACTTCTTTTTCATTAATTATAGGGAGTCACG
ATAGTGTAAGATGCATTAAAAGGAGAAACGTTTCCTAGTAGAATTGTTTC
TATTCCTAGGGCTAGAATCAGGAACCTTTAGTTAAATTAATAGAGATAAT
ATTCATTCCATCACTAAAGGTGAAAATTAAGTATCTTATATTGTCCATAA
ATTTTATATAGAAGAGATGTGAGAATTAATAAAATAAAAATTAAAAACTC
ACGAGTAAACAAAATAATTATATCTAATTTATATTAATAAAGAAGAGTAT
TTGATTATTATATTAAGTCAAATGATAAGCTAATAAATCAATATTAACAA
TCTAATCACATGATTTATATAAAATTGGTTATGGGTATGGGAAGGGAGGG
AGGGAAGTACATTTCATTGAGGAACAATGCAATAGTTAGACAGGATTTAA
CATACTTGAACAAGATATCATAATCTAAAATGATTAAAAATAATTTTTTA
ATATTATCTACACATCGCGCGAATATATATATATATATATATTAAGTGTA
TTTCTTAAATAATATTGTATTACTATTTATATAAATTTTGTATGTTTTAA
TTTTGCAGCAATGTGGCAAAGTCCTACTTCAAGCAACTTATGGAATTAAA
TGACATGGTTAAAAAGATGGTTTTGGAGAGTCTTGGGCTAAAAAATTACA
TTGATGAATTCTTGAATTCCAATGTTTATATGTCAAGATTTACTAATTAC
AAGGTAATTAAAGGTGAAAATGAGAATAAATCAGGATTACCTTCCCACAC
AGATAGTTCCTACTTGACCATAATTAAACAAAATCAAAATGGATTGCAAG
TTCTCTACAAAAATGGAGAGTGGATTGAGCTCAATCGTCAAAATGGACTG
CAAGTTCTCTACAAAAATGGAGAGTGGATTGAGCTCAATCATACTTCACC
AAATTCCTATATTGTTTTATCAGAAGATGTTTTTATGGTAAGTTATTATT
TATTTTTTATTACAGAAGTCAAAAATACACCTAAACTTTTTATTTATATG
TATTTTTGACGCTTAACTCTTTATTTTTTTGTGTGTAGGGGTGGTTTGTT
GCTATAGTAGAGGAGAATAAAAGAAATAGATTTTTTTTGTATGATTGATT
ATTCAAGCCCAACTAGAAGCTAAGATTAGAGGAGTTTTGAAGCAACGAAA
AAAAATGTTGTGTGTGATTTATAGATATTGATGCAGGCTCGATCCGTGAA
AGAAATCACTAATATTTATATTAGATTAGATCGTTTACCTAACTAAACAT
CCCTTGAAGTACTGCCCTTTCTCCAAACCATATGTGAACGTCAAATATTT
TATGCATCAACCTGTCTTTTTTTATTTGGCCCCAACTAACTTCAATCCAC
ATAAATTATTAAATCTTGATATTAGTTGGAATAACATATCTCTTTTCTGA
GAAATTGAAAATAATGCCAGAACTATCATAATCTTTTTTTAAAAAAATTG
TCTTGTTATTATCTTATTAATTTAAAATTTTCTTTCTTCAGAGGAAATTT
AAGTCAATCTTTTTGTTCCTTAATTATTAATTAAACAAATAAATTCTTAT
ACATACTTTTTATGTGTTGATGCTATGAATTAATTATACAGGCATGGACA
AATGATAGATTGACATCTGCTCAACACAGGGTTGTAACAACAGGAGACAA
AGAAAGATTCTCTATTCAAGTTTTTTCCTTTCCAAATCCAGATTACACTG
TGAAGGTCCCACAAGAATTAGTGGATGAAGAACACCCTTTAATGTACAAG
CCTTTTAAGATGTCTGAATATAATAAATATATTATGTTAGGTGCTAAAAA
TGGATTGGGTGTCAAGAATTATTGTGGTCTTTAAAAATTTAGTAGCTATG
AAAATTTATTTATGTATTGTTTTGATGAATAAAATGTATCAGATGGC
(SEQ ID NO: 42; StGAME31-like4).

In one embodiment, a potato GAME31-like gene comprises the nucleic acid sequence of SEQ ID NO: 45. In another embodiment, a potato GAME31-like gene consists of the nucleic acid sequence of SEQ ID NO: 45. In another embodiment, SEQ ID NO: 45 comprises or consists of the following nucleic acid sequence:

GAAACTTTGAAGTCTTTCTTGTTTCCTAAATATTCCTCAAATGGCATCTA
CCAAAGTTACGATTCCCACCATAGATTTTTGCGATTCTGAGCTTAAACCA
AACACTCCACAATGGGAATCAACAAAAGTTCAAGTTTTTGAAGCCTTACA
AGAATTTGGTTGTTTTGAAGCAATATATAACAAAGTTCCAAATGAAATTA
GAGAGGGCATGTTTGATACTTTAAAAGAAGTATTTGATTTTCCACTGCCC
AAATTGATAGAATATAGAGAGAAACCCTTTCATATATATGATGGGCAAAT
TCCAAGTGTACCACTCTTTGGTAGTGTGTACTCTGCTGATTTGGTCCTCC
CAAATAGTGTTGAAACATTTGCCAATACCTTTTGGTCTCATGGAAACCCT
AATTTTAGGTATGCATTACTTCTTTTTCATTAATTATAGGGAGTCACGAT
AGTGTAAGATGCATTAAAAGGAGAAACGTTTCCTAGTAGAATTGTTTCTA
TTCCTAGGGCTAGAATCAGGAACCTTTAGTTAAATTAATAGAGATAATAT
TCATTCCATCACTAAAGGTGAAAATTAAGTATCTTATATTGTCCATAAAT
TTTATATAGAAGAGATGTGAGAATTAATAAAATAAAAATTAAAAACTCAC
GAGTAAACAAAATAATTATATCTAATTTATATTAATAAAGAAGAGTATTT
GATTATTATATTAAGTCAAATGATAAGCTAATAAATCAATATTAACAATC
TAATCACATGATTTATATAAAATTGGTTATGGGTATGGGAAGGGAGGGAG
GGAAGTACATTTCATTGAGGAACAATGCAATAGTTAGACAGGATTTAACA
TACTTGAACAAGATATCATAATCTAAAATGATTAAAAATAATTTTTTAAT
ATTATCTACACATCGCGCGAATATATATATATATATATATTAAGTGTATT
TCTTAAATAATATTGTATTACTATTTATATAAATTTTGTATGTTTTAATT
TTGCAGCAATGTGGCAAAGTCCTACTTCAAGCAACTTATGGAATTAAATG
ACATGGTTAAAAAGATGGTTTTGGAGAGTCTTGGGCTAAAAAATTACATT
GATGAATTCTTGAATTCCAATGTTTATATGTCAAGATTTACTAATTACAA
GGTAATTAAAGGTGAAAATGAGAATAAATCAGGATTACCTTCCCACACAG
ATAGTTCCTACTTGACCATAATTAAACAAAATCAAAATGGATTGCAAGTT
CTCTACAAAAATGGAGAGTGGATTGAGCTCAATCGTCAAAATGGACTGCA
AGTTCTCTACAAAAATGGAGAGTGGATTGAGCTCAATCATACTTCACCAA
ATTCCTATATTGTTTTATCAGAAGATGTTTTTATGGTAAGTTATTATTTA
TTTTTTATTACAGAAGTCAAAAATACACCTAAACTTTTTATTTATATGTA
TTTTTGACGCTTAACTCTTTATTTTTTTGTGTGTAGGGGTGGTTTGTTGC
TATAGTAGAGGAGAATAAAAGAAATAGATTTTTTTTGTATGATTGATTAT
TCAAGCCCAACTAGAAGCTAAGATTAGAGGAGTTTTGAAGCAACGAAAAA
AAATGTTGTGTGTGATTTATAGATATTGATGCAGGCTCGATCCGTGAAAG
AAATCACTAATATTTATATTAGATTAGATCGTTTACCTAACTAAACATCC
CTTGAAGTACTGCCCTTTCTCCAAACCATATGTGAACGTCAAATATTTTA
TGCATCAACCTGTCTTTTTTTATTTGGCCCCAACTAACTTCAATCCACAT
AAATTATTAAATCTTGATATTAGTTGGAATAACATATCTCTTTTCTGAGA
AATTGAAAATAATGCCAGAACTATCATAATCTTTTTTTAAAAAAATTGTC
TTGTTATTATCTTATTAATTTAAAATTTTCTTTCTTCAGAGGAAATTTAA
GTCAATCTTTTTGTTCCTTAATTATTAATTAAACAAATAAATTCTTATAC
ATACTTTTTATGTGTTGATGCTATGAATTAATTATACAGGCATGGACAAA
TGATAGATTGACATCTGCTCAACACAGGGTTGTAACAACAGGAGACAAAG
AAAGATTCTCTATTCAAGTTTTTTCCTTTCCAAATCCAGATTACACTGTG
AAGGTCCCACAAGAATTAGTGGATGAAGAACACCCTTTAATGTACAAGCC
TTTTAAGATGTCTGAATATAATAAATATATTATGTTAGGTGCTAAAAATG
GATTGGGTGTCAAGAATTATTGTGGTCTTTAAAAATTTAGTAGCTATGAA
AATTTATTTATGTATTGTTTTGATGAATAAAATGTATCAGATGGC (SEQ
ID NO: 45; St.GAME31-like5).

In one embodiment, a potato GAME31-like gene comprises the nucleic acid sequence of SEQ ID NO: 48. In another embodiment, a potato GAME31-like gene consists of the nucleic acid sequence of SEQ ID NO: 48. In another embodiment, SEQ ID NO: 48 comprises or consists of the following nucleic acid sequence:

CACTAATATATTTTTAATAAAAACAATACAGTACAATACATTACAATACA
ACGCAACACAACACAATACAATACGTTATGAAATCATATGCAATAACCAC
ACAAATATAAATTTTCAACCACCCATGCATACGATACATTGTATTCATAA
AGAAAATACACATAAATAAATACCCTACTAAATTTTTAAAGACCACAATA
ATTCTTGAGATTAATTCAATTTTTATTACCTGACATAGTTTATTTATGAA
ATTCAAGCAAGTTAAAAGGCTTGAAGAGTAAGGGTGGTCTTCATCCACTA
ATTATTTTGGGGCCTTCACAGCAAAATCTTGATTTGGGAAAGGAAAATAA
TTGAATAGATAGTCTATCTTTGTCTCCTGTTGTTACTACTCTGTGTTCAG
CAGATGTCAAACTATCATTTGTCCATGCCTGTATAAATCCACAACATCGA
CACATAAAAAGTATTAGTGATTTCTTTTCACAGAGAAAGGGCAACACCTC
AAGGGATGTTAGGAAAACAATCTAATCTAATACAAAATATTAGTGATTTT
TTCTCGATCAAGCCCGCATCGTAATCTATAAATCACATACAAACAATCTT
ATCGTTACTTCAAAACTGCTCTGATCTTAGCTTCTAGTTGGGCTTGAATA
ATCAACCATACAAAAAAATCTCTATTTCTTTTATTCTCCTCTTCTATAGC
ATCAACCCACCCCCTCACACACAAAATAATAAAGAGTTAAGCGTCAAAAA
TACACATAAATAAATAATTTAGGTGTATTTTTTACCTTGATACTCAAAAA
TAAATAATAACTTACCCTGAAAGCATCTGCTGATAAAACAATATAGGAAT
TTGGTGTTGTATTATTGAGCTCTATCCACTCTCCATTTTTGTAGAGAACT
TGCAATCCATTTTGATTTTGTTTAATTATAGTCAAGTAGCCACTATCTGT
GTGGGGAGGTAATTCTGATCTATTCTCATCTTCACCTTTAATTACCTTGT
AATTAGTAAATCTTGACACAAAAACATTGGAATTCAAGATTTCATCAATG
TAATTTGTTTTCCCAAGACTCTCCAAAACCATTTTTTCCACCATGTCATT
CAATTCCATAAGTTGCTTGAAGTAGGACTTTGCCACATTGCTGCCAAATT
AAAACATACAAAACTTATATAATGGTAATGCAATATTGTGATATTACTCA
AAATTATGTAAGAAATACACTTAGGGTGTGTTTGGTACGAAGGAAAATAT
TTTCTAGAAAATGTTTTTTAATTTCCCATGTTTGGTTGACTTTAATGATT
TGGAAAATGTTTTCCAAATCAACTTATTTTCCTCAAATTTAAGGAAAATG
ATTATCCTAAAAGTTATTTTCCTAAAGAGTATTTTCTAGTCTTAAGTGAA
AAATAAGTTAGAAATCCACTTGTTTTCCAAGAAAACATTTTCCTTCATAC
CAAACACACCCTTAATATATAATGTATATATACCTAGTATACATACTCGC
GCGATGTGTGAAAAATATTTAAATGTTATTTTAATCATTTTAGATTGTGA
TATCTTGTTCGAGCATGTTAAATCATGTCCAACTATTGCATTGTTACTCA
ATGAAATGTACTCCCCTCCATCCCTTCACATACCCGTAACCAATTCTATA
TGAATAGCGATGTGTGAAAAATATTTAAATGTTATTTTTAATCATTTTAG
ATTGTGATATCTTGTTCGAGCATGTTAAATCATGTCCAACTATTGCATTG
TTACTCAATGAAATGTACTCCCCTCCATCCCTTCACATACCCGTAACCAA
TTCTATATGAATAGCGACAATGAATCATGTGATTAGATTGTTAATATTGA
TTTTATTAGCTTATCACTTGGATATAATTATTAAATACTCTTCTAATTAA
TATAAATTAGATATATTTATTTTGTTTACTCGTGAGTTCTTAATTTTTAT
TTTATTAATGATCACATCTCTTTTATATAAAATGTATGGACAATAAGATA
CTTAATTTTCACCTTTAGTGATGGAATGAATATTATCTCTATAATTTAAC
TAAAGATTCCTGATTCTAGCCTTAGGAATAAAAACAATTCTAGTAGGAAA
CGTTTCTCCTTTTAATGTGTCTTACACTATCGTGACTCCATATTATTAAA
TCTGAAATCCCAAAAATAAATACCGAATACATAATGAAAAAAAAAAAATC
TCCTACAAAATATATGAGCAAGAATAATTGGTGTACTAACATGACTAAAC
TAAATGATATCACCTACATAAATCTTATTCTTTCAAATATCATTAATGAA
AAATAACTTTCATAATACCGAACACATTATATATAATTAAAAGAAGTAAT
ACATACCTAAAGTTAGGGTTTCCATCAGACCAAAAGGTATTGGCAAATGT
TTCAACACTATTTGGGAGGACCAAATCAGCAGAGCTCACACTACCATAGA
GTGGTATACTTGGAATTTGCCCATCATATAAATGGGGTTTTTCTCTATAT
TCTATCAATTTGGACACTGGAAAATCAAATACTTCTTTTAAAGTATCAAA
CATGCCCTCTCTAATTTCATTTGGAACTTTGTCATATATTGCTTCAAAAC
AACCAAATTCTTTTAAGGCTTCAAAAACTTGAACTTTTGTTGATTCCCAT
TGTGGAGTGTTTGGTTTTAGCTCAAGATTGCAAAAATCTATGGTGGGAAT
CTTAACTTTGGTAGATGCCATTGATGAATAAATTAAATAGAGAGGAAATA
GAAAGAAATAAA (SEQ ID NO: 48; StGAME31-like6).

In one embodiment, a potato GAME31-like gene comprises the nucleic acid sequence of SEQ ID NO: 51. In another embodiment, a potato GAME31-like gene consists of the nucleic acid sequence of SEQ ID NO: 51. In another embodiment, SEQ ID NO: 51 comprises or consists of the following nucleic acid sequence:

GCAGCAATGTGGCAAAGTCCTACTTCAAGCAACTTATGGAATTGAATGGC
ATGGTGGAAAAGATGGTTTTGGAGAGTCTTGGGCTAAAAAATTACATTGA
TGAATTCTTGAATTCCAATGTTTATATGTCAAGATTTACTAATTACAAGG
TAATTAAAGGTGAAAATGAGAATAAATCAGGATTACCTTCCCACACAGAT
AGTTCCTACTTGACCATAATTAAACAAAATCAAAATGGATTGCAAGTTCT
TCTACAAAAATGGAGAGTGGATTGAGCTCAATCATACCTCACCAAATTCC
TATATTGTTTTATCAGCAGATGCTCTTATGGTAAGTTATTATTTATTTTT
GATTACAGCAGTCAAAAATACGCCTAAACTTCTAATTTATATGTATTTTT
GACGCTTTAACTCATTATTATTTTTTGTGTGTGGGGTGGTTTGTTGCTAT
AGTAGAGGAGAATAAAAGAAATAGAGATTTTTTTTGTATGATTGATTATT
CAAGCCCAACTAGAAGCTAAGATTAGAGGAGTTTTCAACCAACGAAAAAA
ATGTTTGTGTGTGATTTATATATCATGATGCAGGCTCAATCCGTAAAAGA
AATCACTAATATTTGTATTAGATTAGATTGTTTACCTAACTAACATCCCT
TGAAGTGTTGCCCTTTCTCCAAACCCTATGTGAACGTCAAATATTTTATG
CATCAACCTGTCTTATTTTATTTGACCTCAACTAACTTCAATCCACAAAA
AATATTAAATCTTGATATTATTTGGAATAACGTATCTCTTTTTCTGGAAA
ATTGAAAATGATACCAGAACTATCATAATAATTTTTTAAATTGTCTTGTT
ATTATCTTATTAATTTAAAATTTTCATTCTCCATAGGAAATTTAAGTCAA
TCTTTTTGTTCCTTAATTATTAATTAAACAAATAAATTCTTATACATACT
TTTTATGTGTTGATGATATGAATTAATTATACAGGCATGGACAAATGATA
GATTGACATCTGCTCAACATAGGGTTGTAACAACAGGAGACAAAGATAGA
TTCTCTGTTCAATTATTTTCCCTCGTAAATCCAGATTATACTTTGAAGGT
CCCAAAAGAATTAGTGGATGAAGAACACCCTTTAATGTACAAGCCTTTTA
AGATGCCTGAATATAATAAATATCTTATGTTAGGTGCTAAAAATGGATTG
GGTGTCAAGAATTATTGTGGTCTTTAAAAATTTAGTAGCTATGAAAATTT
ATTTATGTATTGTTTTGATGAATAAAATGTATCAGATGGCTGGTTGAATA
CTTTGAATTTATATTTGGATGGTTATTACGTATGATTGCGTAAAGTATTG
TATTGTATTTTATTTTGTTGTGTTGTTTTGTATTGTGTTGCGTTGTATAT
ATTGTTTTGATGAATAAAATATATGAGTG (SEQ ID NO: 51;
StGAME31-like7).

In one embodiment, a tomato GAME31 cDNA comprises the nucleic acid sequence of SEQ ID NO: 17. In another embodiment, a tomato GAME31 cDNA consists of the nucleic acid sequence of SEQ ID NO: 17. In another embodiment, SEQ ID NO: 17 comprises or consists of the following nucleic acid sequence:

(SEQ ID NO: 17)
TGACTATAATTGATACTCAATCTGTTGAAATATAATGAACCAATTCTTAT
CAAAACACAGTGGAGTACAAGTAATCACGTTGGTTCCTATGAAATGGTTC
ATCTATTTCCCATTATATATAGGCTACTTATTTCCTCACCTATAAAGTAA
AAAACTTTCTAGTGTTTTCTTCTTTCTTTTGTTTTTTTCTCTTTGCTCAT
ATTCTAAAAATATTTCATCAATGGCATCTATCAAATCAGTTAAAGTTCCT
ACTATAGATTTTTCCAATTATCAAGAGCTAAAACCAAACACTCCACTATG
GGAATCCACAAAAATTCAAGTTTTTGAAGCTTTACAAGAATATGGTTGTT
TTGAAGCAATATATGATAAAGTTTCAAAGGAAATTAGAGAGGAAACATTT
GATATGTCAAAAGAAATATTTGAATTTCCTTTAGAGACTAAAGTGAAAAA
TATCTCAGAAAAACCAATGCATGGCTATATGGGGATGATTCCACAATTGC
CATTGTATGAGAGTTTGTGTATTCCTGATTTGCTTAATCCTCAAAGTCTT
GAAAAATTTTCTAATATCTTTTGGCCTCAGGGTAATCAACATTTCTGCAA
TTTGATAAAATCTTATTCTAATCCACTTGTGGAATTGGATGGGATGTTGA
AAAGGATGATTTCGGAGAATTTGGGATTGAAAAATCACATTGATGAATTA
TTGAATGCCAATTACTTCCTATTTAGATTTACACATTATAAGGGATCATC
AATTGCTAGTGGAGATGAAAATAATAAAGCTGCTGGATTGGGTGGCCACA
CGGATGGTAACTTCTTGACTTTTATATCGCAAAATCAAGTTAATGGATTG
CAAATCAACAAAAATGGAGAATGGATTGATGTGATTATTTCACCAAATTC
TTACGTTGTTTTGGCCGGTGATTCCTTCAAAGCTTGGACAAATGGTCGAT
TGCATTCACCTCTCCACAGAGTAACAATGTCCGGACAAAATGATAGACTC
TCCATTCAATTGTTTTCATTATCAAAGCCAGGTCACTTCATCCAGGCACC
AAAAGAACTAGTAGATGAAGAACACCCATTACTCTTCAAGCCATTTGAAA
TTCTTGAATTATTCAAGTATGGTACCACAGAAGCTGGCTATACAGCTCCT
CCAAGTGATCTTTTCAAGATTTATTGTGGTGTTTGATATGCTAATTGTTG
AATTTCCGCTTCAACAAGCAACTTTTCTAATGAGTTTCATCTTGTTTTTT
TAAGTAGTATGCATTTTATGTTTGAATTGTTGCAGTTGGCAATTCATGTT
TAATTTGTTTTTGTTTTTTTGAGAAAATATTTCCAATGGGTTTCGTTGGA
AATTCGTCTTGTTTTTTTTTTTCAAGTAGTGTACATCTTATTTTTGGATT
GTTGATGTTGAGCGCTAATGTTTAATTTGTTTGTGTTTTGAAGAGGATGA
TTATACTCTTTAAGAGGATTCACCGTAATCTTTTAGTATTATTTG.

The start codon (ATG) for SEQ ID NO: 17 starts at nucleotide 221 of SEQ ID NO: 17. The coding sequence present within SEQ ID NO: 17 ends at nucleotide 1186. Therefore, the skilled artisan would recognize that the CDS comprised in SEQ ID NO: 17 comprises nucleotides 221-1186. In one embodiment, the tomato GAME31 CDS comprises the nucleic acid sequence of SEQ ID NO: 59. In another embodiment, the tomato GAME31 CDS consists of the nucleic acid sequence of SEQ ID NO: 59. In another embodiment, SEQ ID NO: 59 comprises or consists of the following nucleic acid sequence:

(SEQ ID NO: 59)
ATGGCATCTATCAAATCAGTTAAAGTTCCTACTATAGATTTTTCCAATTA
TCAAGAGCTAAAACCAAACACTCCACTATGGGAATCCACAAAAATTCAAG
TTTTTGAAGCTTTACAAGAATATGGTTGTTTTGAAGCAATATATGATAAA
GTTTCAAAGGAAATTAGAGAGGAAACATTTGATATGTCAAAAGAAATATT
TGAATTTCCTTTAGAGACTAAAGTGAAAAATATCTCAGAAAAACCAATGC
ATGGCTATATGGGGATGATTCCACAATTGCCATTGTATGAGAGTTTGTGT
ATTCCTGATTTGCTTAATCCTCAAAGTCTTGAAAAATTTTCTAATATCTT
TTGGCCTCAGGGTAATCAACATTTCTGCAATTTGATAAAATCTTATTCTA
ATCCACTTGTGGAATTGGATGGGATGTTGAAAAGGATGATTTCGGAGAAT
TTGGGATTGAAAAATCACATTGATGAATTATTGAATGCCAATTACTTCCT
ATTTAGATTTACACATTATAAGGGATCATCAATTGCTAGTGGAGATGAAA
ATAATAAAGCTGCTGGATTGGGTGGCCACACGGATGGTAACTTCTTGACT
TTTATATCGCAAAATCAAGTTAATGGATTGCAAATCAACAAAAATGGAGA
ATGGATTGATGTGATTATTTCACCAAATTCTTACGTTGTTTTGGCCGGTG
ATTCCTTCAAAGCTTGGACAAATGGTCGATTGCATTCACCTCTCCACAGA
GTAACAATGTCCGGACAAAATGATAGACTCTCCATTCAATTGTTTTCATT
ATCAAAGCCAGGTCACTTCATCCAGGCACCAAAAGAACTAGTAGATGAAG
AACACCCATTACTCTTCAAGCCATTTGAAATTCTTGAATTATTCAAGTAT
GGTACCACAGAAGCTGGCTATACAGCTCCTCCAAGTGATCTTTTCAAGAT
TTATTGTGGTGTTTGA

In one embodiment, a tomato GAME31 cDNA comprises the nucleic acid sequence of SEQ ID NO: 20. In another embodiment, a tomato GAME31 cDNA consists of the nucleic acid sequence of SEQ ID NO: 20. In another embodiment, SEQ ID NO: 20 comprises or consists of the following nucleic acid sequence:

ATGGCATCTACCAAATTAGTTAAAGTTCCCACAATAGATTTTTCAAATCA
TCAAGATCTAAAACCAAACACTCCACTATGGGAATCCAAAAAAATTCAAG
TTTTTGAAGCTTTGCAAGAATATGGTTGTTTTGAAGCAATTTATGATAAA
GTTCCAAAAGATATTAGAGAGGAAACATTTAGTATTTCAAAAGAAATATT
TGAATTTCCTTTAGAGACTAAATTGAAAAATATTTCAGAAAAACCAACGC
ATGGATATATGGGAATGATTCCACAATTGCCATTGTATGAGAGTTTGTGT
ATTCCTGATTTGCTTAATCCTAAAAGTCTTCAAAGTTTTGCTAATATCTT
TTGGCCTCAGGGTAACCAACATTTCTGGTATGTTTACTTATGTTTTTATT
TCGCCCTAGCAGAAGTGTTCAAAAGTACGACATTAGAAATTCTTAGTGAC
TTAATTGATTGA (SEQ ID NO: 20; SlGAME31-like1).

In one embodiment, a tomato GAME31 cDNA comprises the nucleic acid sequence of SEQ ID NO: 23. In another embodiment, a tomato GAME31 cDNA consists of the nucleic acid sequence of SEQ ID NO: 23. In another embodiment, SEQ ID NO: 23 comprises or consists of the following nucleic acid sequence:

TGAGATGTTGAAAAGGATGATTTCGGAGAATTTGGGATTAAAAAATCACA
TTGATGAATTATTGAATGCCAATTACATCCTATTTAGATTTACACAGTAT
AAGGGATCATCAATTGCTAGTGGAGATGAAAATAATAAAGCAGCTGGATT
GGGTGGCCACACAGATGGTAACTTCTTGTCTATTATATCACAAAATGAAG
TTAATGGATTGCAAATCAACAAAAATGGAGAGTGGATTGATGTCAACATT
TCGCCAAATTCTTATGTTGTTTTATCCGGTGATTCCTTCACAGCATGGAC
AAATGGCCGATTGCATTCTCCTGTTCATAGAGTTGAAATGCCCAGAGGAA
GTGATAGATATTCCATTCAATTATTTTCATTATCAAAACCAGGTCACTTC
ATCGAGGCACCAAAAGAAATGGTGGATGAAGAACACCCTTTGCTTTTCAA
GCCATTTGAAATTCTTGGATTACTTGGGTATGGTGCCACAGAAGCTGGCT
ATACAACTCCTCCCAGTGATCTTTTCAAGGCATATTGCGGTGTCTGA
(SEQ ID NO: 23; SlGAME31-like2).

In one embodiment, a tomato GAME31 cDNA comprises the nucleic acid sequence of SEQ ID NO: 26. In another embodiment, a tomato GAME31 cDNA consists of the nucleic acid sequence of SEQ ID NO: 26. In another embodiment, SEQ ID NO: 26 comprises or consists of the following nucleic acid sequence:

TGACTAGAAATAGACATAAAACCCTGTACTTTTTGACACACATGTAATA
GCACTTTCTCTATCTAATACGCAACTCTTTATTAATTTTGCGTAAATTT
TGAGCTATTTCCCATTATATATAGGCTACTTATTTCCTCACCTCTTAAG
TAAAAAACTTTCAAGTGTTTCTTCTTTCTTTTATTTCTCTTTGTTCACA
TATTCTAAAAATATTTCATCAATGGCATCTATCAAATCAGTTAAAGTTC
CTACTATAGATTTTTCCAATTATCAAGAGCTAAAACCAAACACTCCACT
ATGGGAATCCACAAAAATTCAAGTTTTTGAAGCTTTTCAAGAATATGGT
TGTTTTGAAGCAATATATGATAAAGTTCCAAATGAAATTAGAGAGGAAA
CATTTGATATGTCAAAAGAAATATTTGAATTTCCTTTAGATACTAAAGT
GAAAAATATTTCAGAAAAACCAATGCATGGATATATGGGAATGATTCCA
CAATTGCCATTGTATGAGAGTTTGTGTATTCCTGATTTGCTTAATCCTC
AAAGTCTTCAAAATTTTGCTAATATCTTTTGGCCTCAGGGTAATCAACA
TTTCTGCAATTTGGTAAAGTCTTATTCTAATCCACTTGTGGAATTGGAT
GAGATTTTGAAAAGGATGATTTCGGAGAATTTGAGATTAAAAATTCACA
TTGATGAATTGTTGAATGCCAATTATTTCCTATTTAGATTTACACATTA
CAAGGGATCATCAATTACTGGTGGAGATGAGAATAACAAAGTTGCTGGA
TTGGGTGGCCACACAGATGGTAACTTCTTGACTTTTATATCGCAAAATC
AAGTCAATGGATTGCAAATCAACAAAAATGGAGAATGGATTGATGTGAA
TATTTCACCAAATTCTTATGTTGTTTTGGCTGGTGATTCCTTCAAAGCT
TGGACAAATGGTCGATTGCATTCTCCTCTTCACAGAGTAACAATGTCCG
GAGAAAATGATAGACTCTCCATTCAATTATTTTCATTATCAAAACCAGG
TCACTTCATCGAGGCACCAAAAGAACTAGTGGATGAAGAACACCCTTTA
CTCTTCAAGCCATTTGAAATTATTGGATTATTTGAGTATGGTACCACAG
AAGCTGGCTATACAGCTCCTCCAAGTGATCTTCTCAAGAGTTATTGCGG
TGTTTGATATGCTAATTGCGAATTTCCGCTTCAGCAACCAACTTTTCTA
ATAAGTTTCGTCTGAAATTCGTGTTGTTTTAATTATTATGCATTTTATG
TTTGAATTGTTGTAGTTGGCAATTCATGTTTAATTTGTTTGTGTTTTTT
TTTTTTCTTTAGTGGAAATGAT (SEQ ID NO: 26; SIGAME31-
like3).

The start codon (ATG) for SEQ ID NO: 26 starts at nucleotide 217. The coding sequence present within SEQ ID NO: 26 ends at nucleotide 1182. Therefore, the skilled artisan would recognize that the CDS comprised in SEQ ID NO: 26 comprises nucleotides 21-1182.

In another embodiment, an aubergineGAME31 cDNA comprises the nucleic acid sequence of SEQ ID NO: 28. In another embodiment, an aubergineGAME31 cDNA consists of the nucleic acid sequence of SEQ ID NO: 28. In another embodiment, SEQ ID NO: 28 comprises or consists of the following nucleic acid sequence:

(SEQ ID NO: 28)
ATGGGATCTACCAAATCAATTAAAGTTCCCACTATCGATTTTTCCAA
CCATCAAGATCTAAAACCAAACACTCCACAATGGGAATCCACAAAAGATC
AAGTTTTTGAAGCTTTTCAAGAATTTGGTTGTTTTGAAGCAATATATGAT
AAAGTGCCAAATGAAATTAGAAAGGGCATGTTTGATGTTTCAAAAGAAAT
ATTTGAATTTCCCCTAGAGACCAAATTGAAAAACTTATCAGACAAACCAT
TACATGGCTACATGGGGATGATTCCAAACTTGCCTTTGTATGAGAGTTTG
TGTATTCCTGATTTGCTTAATCCTCAAAGTCTTCAAAATTTTGAAAATAT
CTTTTGGCCACATGGAAATCCTGATTTTTGCAATTTGGTAAAATGTTACT
CAAATCCACTTGTGGAATTGGATGAAATGTTGAAGAGGATGATTTTGGAG
AAATTGGGAGTAGAAAATCAGATTGATGAGTTATTGGATCCCAAATATGT
CCTATTTAGATTTACACACTACAAGGGGTCATCACCAACTAATGGAGATA
AAAATACTAAAAGTGAGGGACTAGGTGGCCACACTGATGGTAACTTCTTG
ACTTTTATAGCACAAAATCAAGTAAGTGGATTGCAAATTAATAAAAATGG
AGAGTGGATTGATGTCAACATCTCACCAAATTCTTTTGCTGTTTTGTCTG
CTGATTCCTTCAAAGCATGGACAAATGGTCGATTGCATTCTCCAATTCAC
AGAGTAACAATGGCTGGAGAAAATGATAGATTCTCCATTCAATTATTTTC
ACTATCCAAACCAGGTCACTTCATAGAGGCCCCAAAAGAACTTGTGGATG
AACAACACCCTTTACTCTTCAAACCATATGAAATGCTTGGATTATTTAAG
TATGTTACTTCACAAAGTGGATATGGAGCTCCTGGTGATGCTTTCAAGGC
TTATTGTGGTGTTTGA.

In another embodiment, a potato GAME31 cDNA comprises the nucleic acid sequence of SEQ ID NO: 31. In another embodiment, a potato GAME31 cDNA consists of the nucleic acid sequence of SEQ ID NO: 31. In another embodiment, SEQ ID NO: 31 comprises or consists of the following nucleic acid sequence:

(SEQ ID NO: 31)
ATGGCATCTACCAAAGTTAAGATTCCCACCATAGATTTTTCTAATCTAGA
ACTAAAACCAAACACTCCACTATGGGAATCCACAAAAGTTCAAGTTTTTG
AAGCTTTAAAAGAATATGGTTGTTTTGAAGCAACATATGATAAAATTCCA
AATGAAATTAGAGAGGGTATTTTTGGTATTACAAAAGAAATATTTCAATT
TCCTTTAGAGACCAAAGTGAAAAATTATTCAGATATAACATTACATGGCT
ATGTAGGAATGATTCCACACTTGCCATTTTATGAGAGTTTGTGTATTCCT
GATTTGCTTAATCCTCAAAATGTTGAAACTTTTGCTAATATCTTTTGGCC
TCATGGTAATCCTGATTTCTGCAATTTGGTAAAAGCTTACTCAAATCCAC
TTATGGAATTGGATGAAATGTTGAAAAAGATGATTTTGGAGAATTTGGGA
TTAGAAAATCATATTGATGAATTGCTGGATATTAATTATATGAGATTTAG
ATTTACACATTACAAGGGATCATCAATTATTAGTGGAGATCATGAAAATA
ATATTAAACAAGATGGATTGAATGGCCACACAGATGGTAACTTCTTGACT
TTTATATCACAAAATCAAGTCAATGGTTTGCAAATCAACAAAAATGGAGA
GTGGATTGATGTCAATATTTCACCAAATTCTTATGTTGTTTTGTCTGGTG
ATTCATTCAAAGCATGGACAAATGGTCGATTGCATTCTCCCATCCACAAG
GTAAAAATATTTGGTGAAAGTGATAGATTCTCAATTCAATTATTTTCATT
CTCAAAACCAGGTCACTTTATAAAGGCCCCAAAAGAACTTGTGGATGAAG
AACACCCTTTACTCTTCAAGCCATTTGAAATGGTTGGATTATCTGAGTAT
GTTACTTCCCAAGCTGGCTATGCAGCTCCCAGTGATGCTTTCAAGGCTTA
TTGTGGTCTTTGA.

In another embodiment, a potato GAME31 cDNA comprises the nucleic acid sequence of SEQ ID NO: 34. In another embodiment, a potato GAME31 cDNA consists of the nucleic acid sequence of SEQ ID NO: 34. In another embodiment, SEQ ID NO: 34 comprises or consists of the following nucleic acid sequence:

ATGGCATCTACCAAAGTTACGATTCCCACCATAGATTTTTGCGATTCTGA
GCTTAAACCAAACACTCCACAATGGGAATCAACAAAAGTTCAAGTTTTTG
AAGCCTTACAAGAATTTGGTTGTTTTGAAGCAATATATAACAAAGTTCCA
AATGAAATTAGAGAGGGCATGTTTGATACTTTAAAAGAAGTATTTGATTT
TCCACTGCCCAAATTGATAGAATATAGAGAGAAACCCTTTCATATATATG
ATGGGCAAATTCCAAGTGTACCACTCTTTGGTAGTGTGTACTCTGCTGAT
TTGGTCCTCCCAAATAGTGTTGAAACATTTGCCAATACCTTTTGGTCTCA
TGGAAACCCTAATTTTAGCAATGTGGCAAAGTCCTACTTCAAGCAACTTA
TGGAATTAAATGACATGGTTAAAAAGATGGTTTTGGAGAGTCTTGGGCTA
AAAAATTACATTGATGAATTCTTGAATTCCAATGTTTATATGTCAAGATT
TACTAATTACAAGGTAATTAAAGGTGAAAATGAGAATAAATCAGGATTAC
CTTCCCACACAGATAGTTCCTACTTGACCATAATTAAACAAAATCAAAAT
GGATTGCAAGTTCTCTACAAAAATGGAGAGTGGATTGAGCTCAATCGTCA
AAATGGACTGCAAGTTCTCTACAAAAATGGAGAGTGGATTGAGCTCAATC
ATACTTCACCAAATTCCTATATTGTTTTATCAGAAGATGTTTTTATGGCA
TGGACAAATGATAGATTGACATCTGCTCAACACAGGGTTGTAACAACAGG
AGACAAAGAAAGATTCTCTATTCAAGTTTTTTCCTTTCCAAATCCAGATT
ACACTGTGAAGGTCCCACAAGAATTAGTGGATGAAGAACACCCTTTAATG
TACAAGCCTTTTAAGATGTCTGAATATAATAAATATATTATGTTAGGTGC
TAAAAATGGATTGGGTGTCAAGAATTATTGTGGTCTTTAA (SEQ ID
NO: 34; StGAME31-like1).

In another embodiment, a potato GAME31 cDNA comprises the nucleic acid sequence of SEQ ID NO: 37. In another embodiment, a potato GAME31 cDNA consists of the nucleic acid sequence of SEQ ID NO: 37. In another embodiment, SEQ ID NO: 37 comprises or consists of the following nucleic acid sequence:

ATGATTCCACACTTGCCATTTTATGGGAGTTTGTGTATTCCTGATTTGCT
TAATCCTCAAAATGTTGAAACTTTTGCTAATATCTTTTGGCCTCATGGTA
ATCCTGATTTCTGCAATTTGGTAAAAGCTTACTCAAATCCACTTATGGAA
TTGGATGAATTGTTGAAAAGGATGATTTTGGAGAATTTGGGATTAGAAAA
TCATATTGATGAATTGTTGGATCCTAATTATATGAGATTTAGATTTACAC
ATTACAAGGGATCATCAATTATTAGTGGAGATCATGAAAATAATATTAAA
CATGATGGATTGAATGCCACACAGATGGTAGCTTCTTGA (SEQ ID
NO: 37; StGAME31-like2).

In another embodiment, a potato GAME31 cDNA comprises the nucleic acid sequence of SEQ ID NO: 40. In another embodiment, a potato GAME31 cDNA consists of the nucleic acid sequence of SEQ ID NO: 40. In another embodiment, SEQ ID NO: 40 comprises or consists of the following nucleic acid sequence:

ATGGCATCTACTAAAGGTAAGATTCCCACCATAGATTTTTGCAATCTTGAG
CTAAAACCAAACACTCCACAATGGGAATCAACAAAAGTTCAAGTTTTTGAA
GCCTTAAAAGAATTTGGTTGTTTTGAAGCAATATATGACAAAGTTCCAAAT
GAAATTAGAGAGGGCATGTTTGATAATTTAAAAGAAGTATTTGATTTTCCA
TTGTCCAAATTGATAGAATATAGAGAAAAACCCTTTCATATATATGATGGG
AAAATTCCAAGTATACCACTCTATGGTAGTGTGAGCTCTGCTGATTTGGTC
CTCCCAAATAGTGTTGAAACATTTGCCAATACCTTTTGGTCTGATGGAAAC
CCTAACTTTAGGTATGTATTACTTCTTTTAATTATATATAATGTGCTTGGT
ATTATGAAAGTCATTTTTCATTAA (SEQ ID NO: 40; StGAME31-
like3).

In another embodiment, a potato GAME31 cDNA comprises the nucleic acid sequence of SEQ ID NO: 43. In another embodiment, a potato GAME31 cDNA consists of the nucleic acid sequence of SEQ ID NO: 43. In another embodiment, SEQ ID NO: 43 comprises or consists of the following nucleic acid sequence:

ATGGCATCTACCAAAGTTACGATTCCCACCATAGATTTTTGCGATTCTGAG
CTTAAACCAAACACTCCACAATGGGAATCAACAAAAGTTCAAGTTTTTGAA
GCCTTACAAGAATTTGGTTGTTTTGAAGCAATATATAACAAAGTTCCAAAT
GAAATTAGAGAGGGCATGTTTGATACTTTAAAAGAAGTATTTGATTTTCCA
CTGCCCAAATTGATAGAATATAGAGAGAAACCCTTTCATATATATGATGGG
CAAATTCCAAGTGTACCACTCTTTGGTAGTGTGTACTCTGCTGATTTGGTC
CTCCCAAATAGTGTTGAAACATTTGCCAATACCTTTTGGTCTCATGGAAAC
CCTAATTTTAGCAATGTGGCAAAGTCCTACTTCAAGCAACTTATGGAATTG
AATGACATGGTGGAAAAGATGGTTTTGGAGAGTCTTGGGCTAAAAAATTAC
ACTGATGAATTCTTGAATTCCAATGTTTATATGTCAAGATTTACTAATTAC
AAGGTAATTAAAGGTGAAAATGAGAATAAATCAGCATTACCTTCACACACA
GATAGTTCCTACTTGACCATAATTAAACAAAATCAAAATGGATTGCAAGCA
TGGACAAATGATAGATTGACATCTGCTCAACACAGGGTTGTAACAACAGGA
GACAAAGATAGATTCTCTGTTCAATTATTTTCCCTCCTAAATCCAGATTAT
ACTGTGAAGGTCCCAAAAGAATTAGTGGATGAAGAACACCCTTTAATGTAC
AAGCCTTTTAAGATGCCTGAATATAATAAATATCTTATGTTAGGTGCTAAA
AATGGATTGGGTGTCAAGAATTATTGTGGTCTTTAA (SEQ ID
NO: 43; StGAME31-like4).

In another embodiment, a potato GAME31 cDNA comprises the nucleic acid sequence of SEQ ID NO: 46. In another embodiment, a potato GAME31 cDNA consists of the nucleic acid sequence of SEQ ID NO: 46. In another embodiment, SEQ ID NO: 46 comprises or consists of the following nucleic acid sequence:

ATGATATCTTGTTCTGGGCTAAAAAATTACATTGATGAATTCTTGAATTC
CAATGTTTTTATGTCAAGATTTACTAATTACAGGGTAATTAAAGGTGAAA
ATGAGAATAAATCAGCACTACCTTCCCACACAGATAGTTCCTACTTGACC
ATAATTAAACAAAATCAAAATGGATTGCAAGTTCTCTACAAAAATGGAGA
GTGGATTGAGCTCAATCATACTTCACCAAATTCCTATATTGTTTTATCAG
AAGATGTTTTTATGGCATGGACAAATGATAGATTGACATCTGCTCAACAC
AGGGTTGTAACAACAGGAGACAAAGATAGATTCTCTATTCAAGTTTTTTC
CTTTCCAAATCCAGATTACACTGTGAAGGTCCCACAAGAATTAGTGGATG
AAGAACACCCTTTAATGTTCAAGCCTTTTAAGTTGCCTGAATTTAATAAA
TATATTAAGTTAGGTGCTAAAAATGGACCGGGTCTCAAGAATTATTGTGG
TTTTTAA (SEQ ID NO: 46; StGAME31-like5).

In another embodiment, a potato GAME31 cDNA comprises the nucleic acid sequence of SEQ ID NO: 49. In another embodiment, a potato GAME31 cDNA consists of the nucleic acid sequence of SEQ ID NO: 49. In another embodiment, SEQ ID NO: 49 comprises or consists of the following nucleic acid sequence:

ATGGCATCTACCAAAGTTAAGATTCCCACCATAGATTTTTGCAATCTTGA
GCTAAAACCAAACACTCCACAATGGGAATCAACAAAAGTTCAAGTTTTTG
AAGCCTTAAAAGAATTTGGTTGTTTTGAAGCAATATATGACAAAGTTCCA
AATGAAATTAGAGAGGGCATGTTTGATACTTTAAAAGAAGTATTTGATTT
TCCAGTGTCCAAATTGATAGAATATAGAGAAAAACCCCATTTATATGATG
GGCAAATTCCAAGTATACCACTCTATGGTAGTGTGAGCTCTGCTGATTTG
GTCCTCCCAAATAGTGTTGAAACATTTGCCAATACCTTTTGGTCTGATGG
AAACCCTAACTTTAGCAATGTGGCAAAGTCCTACTTCAAGCAACTTATGG
AATTGAATGACATGGTGGAAAAAATGGTTTTGGAGAGTCTTGGGAAAACA
AATTACATTGATGAAATCTTGAATTCCAATGTTTTTGTGTCAAGATTTAC
TAATTACAAGGTAATTAAAGGTGAAGATGAGAATAGATCAGAATTACCTC
CCCACACAGATAGTGGCTACTTGACTATAATTAAACAAAATCAAAATGGA
TTGCAAGTTCTCTACAAAAATGGAGAGTGGATAGAGCTCAATAATACAAC
ACCAAATTCCTATATTGTTTTATCAGCAGATGCTTTCAGGGCATGGACAA
ATGATAGTTTGACATCTGCTGAACACAGAGTAGTAACAACAGGAGACAAA
GATAGACTATCTATTCAATTATTTTCCTTTCCCAAATCAAGATTTTGCTG
TGAAGGCCCCAAAATAATTAGTGGATGA (SEQ ID NO: 49;
StGAME31-like6).

In another embodiment, a potato GAME31 cDNA comprises the nucleic acid sequence of SEQ ID NO: 52. In another embodiment, a potato GAME31 cDNA consists of the nucleic acid sequence of SEQ ID NO: 52. In another embodiment, SEQ ID NO: 52 comprises or consists of the following nucleic acid sequence:

ATGGATTGCAAGTTCTTCTACAAAAATGGAGAGTGGATTGAGCTCAATCA
TACCTCACCAAATTCCTATATTGTTTTATCAGCAGATGCTCTTATGGCAT
GGACAAATGATAGATTGACATCTGCTCAACATAGGGTTGTAACAACAGGA
GACAAAGATAGATTCTCTGTTCAATTATTTTCCCTCGTAAATCCAGATTA
TACTTTGAAGGTCCCAAAAGAATTAGTGGATGAAGAACACCCTTTAATGT
ACAAGCCTTTTAAGATGCCTGAATATAATAAATATCTTATGTTAGGTGCT
AAAAATGGATTGGGTGTCAAGAATTATTGTGGTCTTTAA (SEQ ID
NO: 52; StGAME31-like7).

In one embodiment, a tomato GAME31 polypeptide comprises the amino acid sequence set forth in SEQ ID NO: 18. In another embodiment, a tomato GAME31 polypeptide consists of the amino acid sequence set forth in SEQ ID NO: 18. In another embodiment, SEQ ID NO: 18 comprises or consists of the following amino acid sequence:

(SEQ ID NO: 18)
MASIKSVKVPTIDFSNYQELKPNTPLWESTKIQVFEALQEYGCFEAIYDK
VSKEIREETFDMSKEIFEFPLETKVKNISEKPMHGYMGMIPQLPLYESLC
IPDLLNPQSLEKFSNIFWPQGNQHFCNLIKSYSNPLVELDGMLKRMISEN
LGLKNHIDELLNANYFLFRFTHYKGSSIASGDENNKAAGLGGHTDGNFLT
FISQNQVNGLQINKNGEWIDVIISPNSYVVLAGDSFKAWTNGRLHSPLHR
VTMSGQNDRLSIQLFSLSKPGHFIQAPKELVDEEHPLLFKPFEILELFKY
GTTEAGYTAPPSDLFKIYCGV.

In one embodiment, a tomato GAME31 polypeptide comprises the amino acid sequence set forth in SEQ ID NO: 21. In another embodiment, a tomato GAME31 polypeptide consists of the amino acid sequence set forth in SEQ ID NO: 21. In another embodiment, SEQ ID NO: 21 comprises or consists of the following amino acid sequence:

MASTKLVKVPTIDFSNHQDLKPNTPLWESKKIQVFEALQEYGCFEAIYDK
VPKDIREETFSISKEIFEFPLETKLKNISEKPTHGYMGMIPQLPLYESLC
IPDLLNPKSLQSFANIFWPQGNQHFWYVYLCFYFALAEVFKSTTLEILSD
LID (SEQ ID NO: 21; SlGAME31-like1).

In one embodiment, a tomato GAME31 polypeptide comprises the amino acid sequence set forth in SEQ ID NO: 24. In another embodiment, a tomato GAME31 polypeptide consists of the amino acid sequence set forth in SEQ ID NO: 24. In another embodiment, SEQ ID NO: 24 comprises or consists of the following amino acid sequence:

EMLKRMISENLGLKNHIDELLNANYILFRFTQYKGSSIASGDENNKAAG
LGGHTDGNFLSIISQNEVNGLQINKNGEWIDVNISPNSYVVLSGDSFTA
WTNGRLHSPVHRVEMPRGSDRYSIQLFSLSKPGHFIEAPKEMVDEEHPL
LFKPFEILGLLGYGATEAGYTTPPSDLFKAYCGV (SEQ ID NO:
24; SlGAME31-like2).

In one embodiment, a tomato GAME31 polypeptide comprises the amino acid sequence set forth in SEQ ID NO: 27. In another embodiment, a tomato GAME31 polypeptide consists of the amino acid sequence set forth in SEQ ID NO: 27. In another embodiment, SEQ ID NO: 27 comprises or consists of the following amino acid sequence:

MASIKSVKVPTIDFSNYQELKPNTPLWESTKIQVFEAFQEYGCFEAIYD
KVPNEIREETFDMSKEIFEFPLDTKVIKNISEKPMHGYMGMIPQLPLYE
SLCIPDLLNPQSLQNFANIFWPQGNQHFCNLVKSYSNPLVELDEILKRM
ISENLRLKIHIDELLNANYFLFRFTHYKGSSITGGDENNKVAGLGGHTD
GNFLTFISQNQVNGLQINKNGEWIDVNISPNSYVVLAGDSFKAWTNGRL
HSPLHRVTMSGENDRLSIQLFSLSKPGHFIEAPKELVDEEHPLLFKPFE
IIGLFEYGTTEAGYTAPPSDLLKSYCGV (SEQ ID NO: 27;
SlGAME31-like3).

In another embodiment, an aubergine GAME31 polypeptide comprises the amino acid sequence set forth in SEQ ID NO: 29. In another embodiment, an aubergine GAME31 polypeptide consists of the amino acid sequence set forth in SEQ ID NO: 29. In another embodiment, SEQ ID 29 comprises or consists of the following amino acid sequence:

(SEQ ID NO: 29)
MGSTKSIKVPTIDFSNHQDLKPNTPQWESTKDQVFEAFQEFGCFEAWDK
VPNEIRKGMFDVSKEIFEFPLETKLKNLSDKPLHGYMGMIPNLPLYESL
CIPDLLNPQSLQNFENIFWPHGNPDFCNLVKCYSNPLVELDEMLKRMIL
EKLGVENQIDELLDPKYVLFRFTHYKGSSPTNGDKNTKSEGLGGHTDGN
FLTFIAQNQVSGLQINKNGEWIDVNISPNSFAVLSADSFKAWTNGRLHS
PIHRVTMAGENDRFSIQLFSLSKPGHFIEAPKELVDEQHPLLFKPYEML
GLFKYVTSQSGYGAPGDAFKAYCGV.

In another embodiment, a potato GAME31 polypeptide comprises the amino acid sequence set forth in SEQ ID NO: 32. In another embodiment, a potato GAME31 polypeptide consists of the amino acid sequence set forth in SEQ ID NO: 32. In another embodiment, SEQ ID NO: 32 comprises or consists of the following amino acid sequence:

(SEQ ID NO: 32)
MASTKVKIPTIDFSNLELKPNTPLWESTKVQVFEALKEYGCFEATYDKI
PNEIREGIFGITKEIFQFPLETKVKNYSDITLHGYVGMIPHLPFYESLC
IPDLLNPQNVETFANIFWPHGNPDFCNLVKAYSNPLMELDEMLKKMILE
NLGLENHIDELLDINYMRFRFTHYKGSSIISGDHENNIKQDGLNGHTDG
NFLTFISQNQVNGLQINKNGEWIDVNISPNSYVVLSGDSFKAWTNGRLH
SPIHKVKIFGESDRFSIQLFSFSKPGHFIKAPKELVDEEHPLLFKPFEM
VGLSEYVTSQAGYAAPSDAFKAYCGL.

In another embodiment, a potato GAME31 polypeptide comprises the amino acid sequence set forth in SEQ ID NO: 35 In another embodiment, a potato GAME31 polypeptide consists of the amino acid sequence set forth in SEQ ID NO: 35. In another embodiment, SEQ ID NO: 35 comprises or consists of the following amino acid sequence:

MASTKVTIPTIDFCDSELKPNTPQWESTKVQVFEALQEFGCFEAIYNKV
PNEIREGMFDTLKEVFDFPLPKLIEYREKPFHIYDGQIPSVPLFGSVYS
ADLVLPNSVETFANTFWSHGNPNFSNVAKSYFKQLMELNDMVKKMVLES
LGLKNYIDEFLNSNVYMSRFTNYKVIKGENENKSGLPSHTDSSYLTIIK
QNQNGLQVLYKNGEWIELNRQNGLQVLYKNGEWIELNHTSPNSYIVLSE
DVFMAWTNDRLTSAQHRVVTTGDKERFSIQVFSFPNPDYTVKVPQELVD
EEHPLMYKPFKMSEYNKYIMLGAKNGLGVKNYCGL (SEQ ID NO:
35; StGAME31-like1).

In another embodiment, a potato GAME31 polypeptide comprises the amino acid sequence set forth in SEQ ID NO: 38 In another embodiment, a potato GAME31 polypeptide consists of the amino acid sequence set forth in SEQ ID NO: 38. In another embodiment, SEQ ID NO: 38 comprises or consists of the following amino acid sequence:

MIPHLPFYGSLCIPDLLNPQNVETFANIFVVPHGNPDFCNLVKAYSNPL
MELDELLKRMILENLGLENHIDELLDPNYMRFRFTHYKGSSIISGDHEN
NIKHDGLNATQMVAS (SEQ ID NO: 38; StGAME31-like2).

In another embodiment, a potato GAME31 polypeptide comprises the amino acid sequence set forth in SEQ ID NO: 41. In another embodiment, a potato GAME31 polypeptide consists of the amino acid sequence set forth in SEQ ID NO: 41. In another embodiment, SEQ ID NO: 41 comprises or consists of the following amino acid sequence:

MASTKGKIPTIDFCNLELKPNTPQWESTKVQVFEALKEFGCFEAIYDKV
PNEIREGMFDNLKEVFDFPLSKLIEYREKPFHIYDGKIPSIPLYGSVSS
ADLVLPNSVETFANTFWSDGNPNFRYVLLLLITYNVLGINIKVIFH
(SEQ ID NO: 41; StGAME31-like3).

In another embodiment, a potato GAME31 polypeptide comprises the amino acid sequence set forth in SEQ ID NO: 44. In another embodiment, a potato GAME31 polypeptide consists of the amino acid sequence set forth in SEQ ID NO: 44. In another embodiment, SEQ ID NO: 44 comprises or consists of the following amino acid sequence:

MISCSGLKNYIDEFLNSNVFMSRFTNYRVIKGENENKSALPSHTDSSYL
TIIKQNQNGLQVLYKNGEWIELNHTSPNSYIVLSEDVFMAWTNDRLTSA
QHRVVTTGDKDRFSIQVFSFPNPDYTVKVPQELVDEEHPLMFKPFKLPE
FNKYIKLGAKNGPGLKNYCGF (SEQ ID NO: 44; StGAME31-
like4).

In another embodiment, a potato GAME31 polypeptide comprises the amino acid sequence set forth in SEQ ID NO: 47. In another embodiment, a potato GAME31 polypeptide consists of the amino acid sequence set forth in SEQ ID NO: 47. In another embodiment, SEQ ID NO: 47 comprises or consists of the following amino acid sequence:

MISCSGLKNYIDEFLNSNVFMSRFTNYRVIKGENENKSALPSHTDSSYL
TIIKQNQNGLQVLYKNGEWIELNHTSPNSYIVLSEDVFMAWTNDRLTSA
QHRVVTTGDKDRFSIQVFSFPNPDYTVKVPQELVDEEHPLMFKPFKLPE
FNKYIKLGAKNGPGLKNYCGF (SEQ ID NO: 47; StGAME31-
like5).

In another embodiment, a potato GAME31 polypeptide comprises the amino acid sequence set forth in SEQ ID NO: 50 In another embodiment, a potato GAME31 polypeptide consists of the amino acid sequence set forth in SEQ ID NO: 50. In another embodiment, SEQ ID NO: 50 comprises or consists of the following amino acid sequence:

MASTKVKIPTIDFCNLELKPNTPQWESTKVQVFEALKEFGCFEAIYDKV
PNEIREGMFDTLKEVFDFPVSKLIEYREKPHLYDGQIPSIPLYGSVSSA
DLVLPNSVETFANTFWSDGNPNFSNVAKSYFKQLMELNDMVEKMVLESL
GKTNYIDEILNSNVFVSRFTNYKVIKGEDENRSELPPHTDSGYLTIIKQ
NQNGLQVLYKNGEWIELNNTTPNSYIVLSADAFRAWTNDSLTSAEHRVV
TTGDKDRLSIQLFSFPKSRFCCEGPKIISG (SEQ ID NO: 50;
StGAME31-like6).

In another embodiment, a potato GAME31 polypeptide comprises the amino acid sequence set forth in SEQ ID NO: 53. In another embodiment, a potato GAME31 polypeptide consists of the amino acid sequence set forth in SEQ ID NO: 53. In another embodiment, SEQ ID NO: 53 comprises or consists of the following amino acid sequence:

MDCKFFYKNGEWIELNHTSPNSYIVLSADALMAWTNDRLTSAQHRVVTT
GDKDRFSVQLFSLVNPDYTLKVPKELVDEEHPLMYKPFKMPEYNKYLML
GAKNGLGVKNYCGL (SEQ ID NO: 53; StGAME31-like7).

In another embodiment, a 2-oxoglutarate-dependent dioxygenase enzyme (GAME31) comprises the amino acid sequence set forth in any one of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53, or a protein homologue thereof, wherein said protein homologue is at least 80% homologous to any of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53.

In one embodiment, homologues of the GAME31 enzyme comprise a 2-oxoglutarate-dependent dioxygenase enzyme activity. In some embodiments, a homologue also encompasses deletion, insertion, or substitution variants thereof and biologically active polypeptide fragments thereof. In one embodiment, the variant comprises conservative substitutions, or deletions, insertions, or substitutions that do not significantly alter the three dimensional structure of the GAME31 enzyme. In another embodiment, the deletion, insertion, or substitution does not alter the function of interest of the GAME31 enzyme, which in one embodiment wherein said plant is a tomato plant, is a step in the conversion of dehydrotomatine to hydroxy-dehydrotomatine, and a step in the conversion of α-tomatine to hydroxytomatine. In another embodiment, the deletion, insertion, or substitution does not alter the function of interest of the GAME31 enzyme, which in one embodiment wherein said plant is a potato plant, is a step in the conversion of α-solanine to leptinine II and a step in the conversion of α-chaconine to leptinine I. In another embodiment, the deletion, insertion, or substitution does not alter the function of interest of the GAME31 enzyme, which in one embodiment wherein said plant is an aubergine plant, is a step in the conversion of solasonine to hydroxysolasonine and a step in the conversion of α-solamargine to hydroxysolamargine.

In another embodiment, the disclosure includes a homologue of a GAME31 enzyme. In another embodiment, the disclosure includes a homologue of a GAME31 enzyme having a 2-oxoglutarate-dependent dioxygenase enzyme activity. In another embodiment, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a GAME31 enzyme as determined using BlastP software of the National Center of Biotechnology Information (NCBI) using default parameters.

In another embodiment, the amino acid sequence of a GAME31 enzyme homologue is at least 70% homologous to a GAME31 amino acid sequence or a peptide thereof, described herein. In another embodiment, the amino acid sequence of a GAME31 enzyme homologue is at least 80% homologous a GAME31 amino acid sequence or a peptide thereof, described herein. In another embodiment, the amino acid sequence of a GAME31 enzyme homologue is at least 90% homologous a GAME31 amino acid sequence or a peptide thereof, described herein. In another embodiment, the amino acid sequence of a GAME31 enzyme homologue is at least 95% homologous a GAME31 amino acid sequence or a peptide thereof, described herein. In another embodiment, the amino acid sequence of a GAME31 enzyme homologue is at least 98% homologous a GAME31 amino acid sequence or a peptide thereof, described herein.

In one embodiment, homologues of GAME31 enzyme are expressed in a plant, wherein said plant comprises a cultivated tomato, a wild tomato, a cultivated potato, a wild potato, or an aubergine plant.

In another embodiment, a 2-oxoglutarate-dependent dioxygenase comprises the amino acid sequence set forth in any one of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 53.

In one embodiment, the 2-oxoglutarate-dependent dioxygenase (GAME31) polypeptide is encoded by a gene comprising the nucleotide sequence set forth in any one of SEQ ID NO: 16, SEQ ID NO: 19, SEQ ID NO: 22, SEQ ID NO: 25, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 51, or a gene homologue thereof, wherein said gene homologue is at least 80% homologous to any of SEQ ID NO: 16, SEQ ID NO: 19, SEQ ID NO: 22, SEQ ID NO: 25, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 51, In another embodiment, the 2-oxoglutarate-dependent dioxygenase (GAME31) polypeptide is encoded by a cDNA comprising the nucleotide sequence set forth in any one of SEQ ID NO: 17, SEQ ID NO: 20, SEQ ID NO: 23, SEQ ID NO: 26, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 52, or a cDNA homologue thereof, wherein said cDNA homologue is at least 80% homologous to any of SEQ ID NO: 17, SEQ ID NO: 20, SEQ ID NO: 23, SEQ ID NO: 26, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 52.

In one embodiment, the 2-oxoglutarate-dependent dioxygenase (GAME31) polypeptide is encoded by a gene comprising the nucleotide sequence set forth in any one of SEQ ID NO: 16, SEQ ID NO: 19, SEQ ID NO: 22, SEQ ID NO: 25, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 51. In another embodiment, the 2-oxoglutarate-dependent dioxygenase (GAME31) polypeptide is encoded by a cDNA comprising the nucleotide sequence set forth in any one of SEQ ID NO: 17, SEQ ID NO: 20, SEQ ID NO: 23, SEQ ID NO: 26, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 52.

In one embodiment, the 2-oxoglutarate-dependent dioxygenase (GAME31) polypeptide is encoded by a nucleic acid sequence set forth in any one of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51 and SEQ ID NO: 52, or a nucleic acid homologue thereof, wherein said homologue is at least 80% homologous to any of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51 and SEQ ID NO: 52.

In one embodiment, the 2-oxoglutarate-dependent dioxygenase (GAME31) polypeptide is encoded by a nucleic acid sequence set forth in any one of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51 and SEQ ID NO: 52.

In some embodiment, the 2-oxoglutarate-dependent dioxygenase (GAME31) is encoded by a gene comprising the polynucleotide sequence set forth in any one of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52, or a nucleic acid sequence having at least 80% identity to any of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, or SEQ ID NO: 52.

In one embodiment, homologues of a GAME31 gene or a GAME31 cDNA encode a polypeptide comprising a 2-oxoglutarate-dependent dioxygenase enzyme activity. In some embodiments, a homologue also encompasses deletion, insertion, or substitution variants, thereof, and biologically active polynucleotide fragments thereof. In one embodiment, the variant comprises conservative substitutions, or deletions, insertions, or substitutions that do not significantly alter the three dimensional structure of the encoded GAME31 enzyme. In another embodiment, the deletion, insertion, or substitution does not alter the function of interest of the encoded GAME31 enzyme, which in one embodiment wherein said plant is a tomato plant, is a step in the conversion of dehydrotomatine to hydroxy-dehydrotomatine and/or a step in the conversion of α-tomatine to hydroxy tomatine. In another embodiment, the deletion, insertion, or substitution does not alter the function of interest of the encoded GAME31 enzyme, which in another embodiment wherein said plant is a potato plant, is a step in the conversion of α-solanine to leptinine II and/or conversion of α-chaconine to leptinine I. In another embodiment, the deletion, insertion, or substitution does not alter the function of interest of the encoded GAME31 enzyme, which in another embodiment wherein said plant is an aubergine plant, is a step in the conversion of solasonine to hydroxysolasonine and/or the conversion of solamargine to hydroxysolamargine.

In another embodiment, disclosed herein are GAME31 gene homologues. In another embodiment, disclosed herein are GAME31 cDNA homologues. In another embodiment, disclosed herein are GAME31 gene homologues encoding an enzyme having a 2-oxoglutarate-dependent dioxygenase enzyme activity. In another embodiment, the disclosure includes a homologue of a GAME31 cDNA encoding an enzyme having a 2-oxoglutarate-dependent dioxygenase enzyme activity. In another embodiment, homologues comprise a polynucleotide sequence which is at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a GAME31 gene nucleic acid sequence.

In another embodiment, the nucleic sequence of a GAME31 gene homologue is at least 70% homologous to a GAME31 nucleic acid sequence or a fragment thereof, described herein. In another embodiment, the nucleic sequence of a GAME31 gene homologue is at least 80% homologous to a GAME31 nucleic acid sequence or a fragment thereof, described herein. In another embodiment, the nucleic sequence of a GAME31 gene homologue is at least 90% homologous to a GAME31 nucleic acid sequence or a fragment thereof, described herein. In another embodiment, the nucleic sequence of a GAME31 gene homologue is at least 95% homologous to a GAME31 nucleic acid sequence or a fragment thereof, described herein. In another embodiment, the nucleic sequence of a GAME31 gene homologue is at least 98% homologous to a GAME31 nucleic acid sequence or a fragment thereof, described herein.

In another embodiment, the nucleic sequence of a GAME31 cDNA homologue is at least 70% homologous to a GAME31 nucleic acid sequence or a fragment thereof, described herein. In another embodiment, the nucleic sequence of a GAME31 cDNA homologue is at least 80% homologous to a GAME31 nucleic acid sequence or a fragment thereof, described herein. In another embodiment, the nucleic sequence of a GAME31 cDNA homologue is at least 90% homologous to a GAME31 nucleic acid sequence or a fragment thereof, described herein. In another embodiment, the nucleic sequence of a GAME31 cDNA homologue is at least 95% homologous to a GAME31 nucleic acid sequence or a fragment thereof, described herein. In another embodiment, the nucleic sequence of a GAME31 cDNA homologue is at least 98% homologous to a GAME31 nucleic acid sequence or a fragment thereof, described herein.

In one embodiment, homologues of a GAME31 gene are expressed in a plant, wherein said plant includes a cultivated tomato, a wild tomato, a cultivated potato, a wild potato, a sweet or chili pepper, an aubergine plant, and a bittersweet plant. In one embodiment, homologues of a GAME31 cDNA are expressed in a plant, wherein said plant comprises a cultivated tomato, a wild tomato, a cultivated potato, a wild potato, a sweet or chili pepper, an aubergine plant, and a bittersweet plant.

In one embodiment, a genetically modified plant as disclosed herein comprises a Solanaceae crop plant. A skilled artisan would appreciate that the term “Solanaceae” refers to a family of plants that includes the genus Solanum. In some embodiments, a Solanaceae crop plant is selected from the group comprising Solanum lycopersicum, Solanum pennellii, Solanum tuberosum, Solanum chacoense, Capiscum annuum, Solanum melongena, and Solanum dulcamara. In some embodiments, a Solanaceae crop plant comprises any Solanaceae crop plant that produces a steroidal alkaloid or a glycosylated derivative thereof, or an unsaturated or saturated steroidal saponin or a glycoside derivative thereof, or any combination thereof.

Steroidal alkaloids (SAs), also known as “Solanum alkaloids” and the glycosylated derivatives thereof, are common constituents of numerous plants belonging to the Solanaceae family, particularly of the genus Solanum. Estimated in the order of 1350 species, Solanum is one of the largest genera of flowering plants, representing about a half of the species in the Solanaceae. SAs the glycosylated derivatives thereof have diverse structural composition and biological activity, and they occur in food plants included in the Solanum genus, including tomato (Solanum lycopersicum), wild-tomato (Solanum pennellii), potato (Solanum tuberosum), eggplant (Solanum melongena), and bittersweet plant (Solanum dulcamara). In one embodiment, a plant disclosed herein is selected from the Solanum genus of plants comprising cultivated tomato (Solanum lycopersicum), wild-tomato (Solanum pennellii), cultivated potato (Solanum tuberosum), wild potato Solanum chacoense, eggplant (Solanum melongena), and bittersweet plant (Solanum dulcamara). In another embodiment, a plant disclosed herein is selected from the family of Solanaceae crop plants comprising a cultivated tomato plant (Solanum lycopersicum), a wild-tomato plant (Solanum pennellii), a cultivated potato plant (Solanum tuberosum), a wild potato plant Solanum chacoense, a sweet bell pepper plant (Capsicum annuum), a sweet or chili pepper plant (Capsicum annuum), an eggplant plant (Solanum melongena), and a bittersweet plant (Solanum dulcamara).

In one embodiment, an altered expression of said at least one gene comprising GAME25, or GAME31, or a combination thereof, comprises a reduced or inhibited expression of said at least one gene compared to its expression in a corresponding unmodified plant, and wherein said altered content comprises a reduced content of at least one cholesterol derived compound selected from the group comprising a steroidal alkaloid or a glycosylated derivative thereof and an unsaturated or saturated steroidal saponin or a glycoside derivative thereof compared to said corresponding unmodified plant.

In another embodiment, altered expression of said at least one gene is comprises reduced or inhibited expression of said gene. In another embodiment, altered expression of said at least one gene comprises reduced expression of said gene. In another embodiment, altered expression of said at least one gene comprises inhibited expression of said gene.

In another embodiment, altered expression of a GAME25 gene comprises reduced or inhibited expression of the GAME25 gene. In another embodiment, altered expression of said GAME25 gene comprises reduced expression of the GAME25 gene. In another embodiment, altered expression of said GAME25 gene comprises inhibited expression of the GAME25 gene.

In another embodiment, altered expression of a GAME31 gene comprises reduced or inhibited expression of the GAME31 gene. In another embodiment, altered expression of said GAME31 gene comprises reduced expression of the GAME31 gene. In another embodiment, altered expression of said GAME31 gene comprises inhibited expression of the GAME31 gene.

In another embodiment, altered expression of a GAME31 gene and a GAME25 gene comprises reduced or inhibited expression of the GAME31 gene and the GAME25 gene. In another embodiment, altered expression of said GAME31 gene and said GAME25 gene comprises reduced expression of both genes. In another embodiment, altered expression of said GAME31 gene and said GAME25 gene comprises inhibited expression of both genes. In another embodiment, altered expression of said GAME31 gene and said GAME25 gene comprises reduced expression of said GAME31 gene and inhibited expression of said GAME25 gene. Alternatively, in another embodiment, altered expression of said GAME31 gene and said GAME25 gene comprises reduced expression of said I gene and inhibited expression of said GAME31 gene.

In some embodiments, altered expression comprises a reduced or inhibited expression of said GAME25 gene, or GAME31 gene, or a combination thereof compared to its expression in a corresponding unmodified plant; or an increased expression of said GAME25 gene, or GAME31 gene, or a combination thereof compared to its expression in a corresponding unmodified plant; or a combination of reduced or inhibited expression of one of said GAME25 gene or said GAME31 gene, and increased expression the other of said GAME25 gene or said GAME31 gene of compared to their expression in a corresponding unmodified plant and increased expression of.

In one embodiment, gene expression of at least one gene is reduced by at least 5% compared to a corresponding unmodified plant. In another embodiment, gene expression of at least one gene is reduced by at least 10% compared to a corresponding unmodified plant. In another embodiment, gene expression of at least one gene is reduced by at least 15% compared to a corresponding unmodified plant. In another embodiment, gene expression of at least one gene is reduced by at least 20% compared to a corresponding unmodified plant. In another embodiment, gene expression of at least one gene is reduced by at least 25% compared to a corresponding unmodified plant. In another embodiment, gene expression of at least one gene is reduced by at least 30% compared to a corresponding unmodified plant. In another embodiment, gene expression of at least one gene is reduced by at least 35% compared to a corresponding unmodified plant. In another embodiment, gene expression of at least one gene is reduced by at least 40% compared to a corresponding unmodified plant. In another embodiment, gene expression of at least one gene is reduced by at least 45% compared to a corresponding unmodified plant. In another embodiment, gene expression of at least one gene is reduced by at least 50% compared to a corresponding unmodified plant. In another embodiment, gene expression of at least one gene is reduced by at least 55% compared to a corresponding unmodified plant. In another embodiment, gene expression of at least one gene is reduced by at least 60% compared to a corresponding unmodified plant. In another embodiment, gene expression of at least one gene is reduced by at least 65% compared to a corresponding unmodified plant. In another embodiment, gene expression of at least one gene is reduced by at least 70% compared to a corresponding unmodified plant. In another embodiment, gene expression of at least one gene is reduced by at least 75% compared to a corresponding unmodified plant. In another embodiment, gene expression of at least one gene is reduced by at least 80% compared to a corresponding unmodified plant. In another embodiment, gene expression of at least one gene is reduced by at least 85% compared to a corresponding unmodified plant. In another embodiment, gene expression of at least one gene is reduced by at least 90% compared to a corresponding unmodified plant. In another embodiment, gene expression of at least one gene is reduced by at least 95% compared to a corresponding unmodified plant. In another embodiment, gene expression of at least one gene is reduced by at least 98% compared to a corresponding unmodified plant. In another embodiment, gene expression of at least one gene is reduced by 100% compared to a corresponding unmodified plant.

In another embodiment, gene expression of at least two genes is reduced by at least 5% compared to a corresponding unmodified plant. In another embodiment, gene expression of at least two genes is reduced by at least 10% compared to a corresponding unmodified plant. In another embodiment, gene expression of at least two genes is reduced by at least 15% compared to a corresponding unmodified plant. In another embodiment, gene expression of at least two genes is reduced by at least 20% compared to a corresponding unmodified plant. In another embodiment, gene expression of at least two genes is reduced by at least 25% compared to a corresponding unmodified plant. In another embodiment, gene expression of at least two genes is reduced by at least 30% compared to a corresponding unmodified plant. In another embodiment, gene expression of at least two genes is reduced by at least 35% compared to a corresponding unmodified plant. In another embodiment, gene expression of at least two genes is reduced by at least 40% compared to a corresponding unmodified plant. In another embodiment, gene expression of at least two genes is reduced by at least 45% compared to a corresponding unmodified plant. In another embodiment, gene expression of at least two genes is reduced by at least 50% compared to a corresponding unmodified plant. In another embodiment, gene expression of at least two genes is reduced by at least 55% compared to a corresponding unmodified plant. In another embodiment, gene expression of at least two genes is reduced by at least 60% compared to a corresponding unmodified plant. In another embodiment, gene expression of at least two genes is reduced by at least 65% compared to a corresponding unmodified plant. In another embodiment, gene expression of at least two genes is reduced by at least 70% compared to a corresponding unmodified plant. In another embodiment, gene expression of at least two genes is reduced by at least 75% compared to a corresponding unmodified plant. In another embodiment, gene expression of at least two genes is reduced by at least 80% compared to a corresponding unmodified plant. In another embodiment, gene expression of at least two genes is reduced by at least 85% compared to a corresponding unmodified plant. In another embodiment, gene expression of at least two genes is reduced by at least 90% compared to a corresponding unmodified plant. In another embodiment, gene expression of at least two genes is reduced by at least 95% compared to a corresponding unmodified plant. In another embodiment, gene expression of at least two genes is reduced by at least 98% compared to a corresponding unmodified plant. In another embodiment, gene expression of at least two genes is reduced by 100% compared to a corresponding unmodified plant.

It would be appreciated by the skilled artisan that while the gene expression of more than one gene may be altered, the percent change of expression, for example reduction, may not be the same for both genes. That is one gene may have reduced expression of a percent greater than or less than the percent change in a second gene.

Altered gene expression in at least one cell of a genetically modified plant described herein, results in the genetically modified plant having an altered content of at least one steroidal alkaloids or a glycosylated derivative thereof, compared to a corresponding unmodified plant. In one embodiment, an at least one steroidal alkaloid or glycosylated derivative thereof a genetically modified plant described herein comprises a steroidal alkaloid or glycosylated derivative thereof selected from the group comprising solasodine, hydroxyl solasodine, α-solanine, α-chaconine, tomatidine, α-tomatine, hydroxytomatine, acetoxytomatine, acetoxy-hydroxytomatine, tomatidine+4 hexose, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydrotomatidine, dehydroesculeosides, leptinine I, leptinine II, leptine I, leptine II, and hydroxysolamargine, or any derivatives thereof, or any combination thereof. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a part of the plant selected from the group comprising a peel, a leaf, a young leaf, a mature leaf, a bud, a petal, a root, and an edible part of the plant. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in an edible plant part. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a non-edible plant part. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a leaf. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a young leaf. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a mature leaf. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a bud. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a petal. In another embodiment, the altered content of at least one cholesterol derived compound selected from the group comprising a steroidal alkaloid or a glycosylated derivative thereof and an unsaturated or saturated steroidal saponin or a glycoside derivative thereof is altered in a root. In another embodiment, the genetically modified plant has an altered content of a at least one cholesterol derived compound selected from the group comprising a steroidal alkaloid or a glycosylated derivative thereof and an unsaturated or saturated steroidal saponin or a glycoside derivative thereof is selected from the family of Solanaceae crop plants.

In some embodiments, altered content comprises an appearance of at least one steroidal alkaloid or a glycosylated derivative thereof compared to said corresponding unmodified plant that does not contain said at least one steroidal alkaloid or a glycosylated derivative thereof. In some embodiments, the appearance of at least one steroidal alkaloid or a glycosylated derivative thereof comprises a new appearance of said steroidal alkaloid or a glycosylated derivative thereof, wherein the plant does not naturally produce said steroidal alkaloid or a glycosylated derivative thereof.

In some embodiments, altered content comprises an appearance of at least one unsaturated or saturated steroidal saponin or a glycosylated derivative thereof compared to said corresponding unmodified plant that does not contain said at least one unsaturated or saturated steroidal saponin or a glycosylated derivative thereof. In some embodiments, the altered content comprises an appearance of said at least one unsaturated or saturated steroidal saponin or a glycosylated derivative thereof, wherein the plant does not naturally produce said unsaturated or saturated steroidal saponin or a glycosylated derivative thereof.

In another embodiment, the altered content of at least one steroidal alkaloid or a glycosylated derivative thereof comprises altering the content of an anti-nutritional steroidal alkaloid or glycosylated derivative thereof. In yet another embodiment, the altered content of at least one steroidal alkaloid or a glycosylated derivative thereof comprises altering the content of a steroidal alkaloid or glycosylated derivative thereof that provides plant resistance to pests or pathogens. In still a further embodiment, the altered content of at least one steroidal alkaloid or a glycosylated derivative thereof comprises altering the content of both an anti-nutritional steroidal alkaloid or glycosylated derivative thereof, and a steroidal alkaloid or glycosylated derivative thereof that provides plant resistance to pests or pathogens.

In another embodiment, the altered content of at least one steroidal alkaloid (SA) or a glycosylated derivative thereof (SGA) compared to a corresponding unmodified plant comprises reduction of said SA or SGA content by at least 5% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 10% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 15% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 20% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 25% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 30% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 35% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 40% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 45% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 50% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 55% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 60% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 65% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 70% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 75% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 80% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 85% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 90% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by at least 95% compared to the corresponding unmodified plant. In another embodiment, the altered content of at least one steroidal alkaloid (SA) or a glycosylated derivative thereof (SGA) compared to a corresponding unmodified plant comprises reduction of said SA or SGA content by 100% compared to the corresponding unmodified plant.

In some embodiments, the altered content of at least one cholesterol derived compound selected from the group comprising a steroidal alkaloid or a glycosylated derivative thereof and an unsaturated or saturated steroidal saponin or a glycoside derivative thereof comprises
a reduced content of at least one steroidal alkaloid or a glycosylated derivative thereof compared to said corresponding unmodified plant, or
an increased content of at least one steroidal alkaloid or a glycosylated derivative thereof compared to said corresponding unmodified plant, or
a reduced content of at least one unsaturated or saturated steroidal saponin or a glycosylated derivative thereof compared to said corresponding unmodified plant, or
an increased content of at least one unsaturated or saturated steroidal saponin or a glycosylated derivative thereof compared to said corresponding unmodified plant, or
a combination of a reduced content of at least one steroidal alkaloid or a glycosylated derivative thereof, and an increased content of at least one steroidal alkaloid or a glycosylated derivative thereof,
or any combination thereof, compared to said corresponding unmodified plant.

In some embodiments, the reduced content of at least one steroidal alkaloid or a glycosylated derivative thereof comprises reduced content of at least one anti-nutritional steroidal alkaloid or a glycosylated derivative thereof, or reduced content of at least one toxic steroidal alkaloid or a glycosylated derivative thereof, or a combination thereof. In some embodiments, the increased content results in increased plant resistance to at least one plant pathogen, pest, or predator, or any combination thereof, and optionally generates precursor molecules for steroidal alkaloid molecules that provide resistance to at least one plant pathogen, pest, or predator, or any combination thereof.

In some embodiments, an altered steroidal alkaloid or a glycosylated derivative thereof is selected from the group comprising tomatidine, α-tomatine, α-tomatine isomer (1 and 2), α-tomatine isomer 1, α-tomatine isomer 2, hydroxytomatine, acetoxytomatine, dehydrotomatidine, dehydrotomatine, dehydrotomatine isomer 1, dehydrotomatine+4-hexose, acetoxy-hydroxytomatine, acetoxy-hydroxy-dehydrotomatine, tomatidine+4 hexose, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydroesculeosides, dehydroesculeoside A, dehydroesculeoside A+hexose, leptinine I, leptinine II, leptine I, leptine II, soladulcidine, β-soladulcine, soladulcine A, solanidine, α-solanine, α-chaconine, solasoidine, α-solasonine, α-solamargine, hydroxysolasonine, and hydroxysolamargine, or any derivatives thereof, or any combination thereof.

In some embodiments, an altered unsaturated or saturated steroidal saponin or a glycosylated derivative thereof is selected from the group comprising dioscin, diosgenin, parillin, and sarasapogenin.

In some embodiments, an altered unsaturated or saturated steroidal saponin or a glycosylated derivative thereof is selected from the group comprising aescin, araloside A, astragaloside, bacopaside, bacoside, bacoside A, chaconine, charantin, daucosterol, esculeoside A, ginsenoside, glycyrrhizin, α-hederin, holothurin, momordicine, momordin, osladin, protodioscin, pseudoginsenoside F11, QS21, solanine, triterpenoid saponin, and ziziphin.

In some embodiments, the reduced content of at least one steroidal alkaloid or a glycosylated derivative thereof comprises reduced content of at least one anti-nutritional steroidal alkaloid or a glycosylated derivative thereof. In some embodiments, an anti-nutritional steroidal alkaloid or a glycosylated derivative thereof comprises a steroidal alkaloid or glycosylated derivative endogenous to the plant.

In some embodiments, an anti-nutritional steroidal alkaloid or a glycosylated derivative thereof comprises anti-nutritional properties towards a mammal. In some embodiments, an anti-nutritional steroidal alkaloid or a glycosylated derivative thereof comprises anti-nutritional properties towards a human subject. In some embodiments, an anti-nutritional steroidal alkaloid or a glycosylated derivative thereof comprises anti-nutritional properties towards a farm animal. In some embodiments, an anti-nutritional steroidal alkaloid or a glycosylated derivative thereof comprises anti-nutritional properties towards a domesticated animal. A skilled artisan would appreciate that an anti-nutritional steroidal alkaloid, or a glycosylated derivative may encompass lethal toxins or compounds that disrupt digestion and nutrient absorption, or any combination thereof. In another embodiment, an anti-nutritional steroidal alkaloid or a glycosylated derivative comprises a toxin. In some embodiments, the reduced content of at least one steroidal alkaloid or a glycosylated derivative thereof comprises reduced content of at least one toxic steroidal alkaloid or a glycosylated derivative thereof.

In another embodiment, the altered content of at least one steroidal alkaloid (SA) or a glycosylated derivative thereof (SGA) compared to a corresponding unmodified plant comprises increase of said SA or SGA content by at least 5% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 10% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 15% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 20% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 25% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 30% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 35% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 40% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 45% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 50% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 55% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 60% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 65% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 70% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 75% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 80% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 85% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 90% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by at least 95% compared to the corresponding unmodified plant. In another embodiment, the altered content of at least one steroidal alkaloid (SA) or a glycosylated derivative thereof (SGA) compared to a corresponding unmodified plant comprises increase of said SA or SGA content by 100% compared to the corresponding unmodified plant.

In another embodiment, the altered content of at least one steroidal alkaloid (SA) or a glycosylated derivative thereof (SGA) comprises increased content of at least one steroidal alkaloid or glycosylated derivative thereof that provides resistance to a plant pathogen, pest, or predator compared to a corresponding unmodified plant.

In another embodiment, the altered content of at least one steroidal alkaloid (SA) or a glycosylated derivative thereof (SGA) comprises increased content of at least one precursor molecule for at least steroidal alkaloid or glycosylated derivative thereof that provides resistance to a plant pathogen, pest, or predator compared to a corresponding unmodified plant. In another embodiment the content of said at least one precursor is increased by at least 5% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 10% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 15% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 20% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 25% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 30% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 35% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 40% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 45% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 50% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 55% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 60% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 65% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 70% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 75% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 80% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 85% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 90% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by at least 95% compared to the corresponding unmodified plant. In another embodiment, the altered content of at least one precursor steroidal alkaloid (SA) or a glycosylated derivative thereof (SGA) compared to a corresponding unmodified plant comprises increase of said precursor SA or SGA content by 100% compared to the corresponding unmodified plant.

A skilled artisan would appreciate that the terms “plant pathogen” or “plant predator” or “plant pest” may encompass any organism that can infect and cause harm to a plant or any part thereof. In some embodiments, a plant can be harmed by an inhibition or slowing of the growth of a plant, by damage to a tissue or tissues of a plant, by a weakening of the defense mechanism of a plant, by a reduction in the resistance of a plant to abiotic stresses, by a premature death of the plant, and the like. Plant pathogens, pests, and or predators include, but are not limited to insects, fungi, oomycetes, viruses, and bacteria.

In another embodiment, a genetically modified potato plant comprises increased content of at least one steroidal alkaloid (SA) or a glycosylated derivative thereof (SGA) compared to a corresponding unmodified plant, wherein said SA or SGA is selected from the group comprising leptinine I, leptinine II, leptine I, or leptine II, or any combination thereof. In another embodiment, a genetically modified potato plant comprises increased content of at least one steroidal alkaloid (SA) or a glycosylated derivative thereof (SGA) compared to a corresponding unmodified plant, wherein said SA or SGA comprises a precursor of a compound selected from the group comprising leptinine I, leptinine II, leptine I, or leptine II, or any combination thereof. In some embodiments, a genetically modified potato plant comprises an altered content of at least one steroidal alkaloid (SA) or a glycosylated derivative thereof (SGA) compared to a corresponding unmodified plant, wherein said SA or SGA is selected from the group comprising α-solanine, α-chaconine, leptinine I, leptinine II, leptine I, and leptine II, or any combination thereof.

In one embodiment, disclosed herein are methods of enhancing the content of at least one cholesterol derived compound selected from the group comprising a steroidal alkaloid or a glycosylated derivative thereof and an unsaturated or saturated steroidal saponin or a glycoside derivative thereof in a plant, comprising transforming at least one plant cell within said plant with a nucleic acid sequence operably linked to a 2-oxoglutarate-dependent dioxygenase gene (GAME31), wherein said nucleic acid sequence operably linked to a GAME31 gene results in overexpression of said GAME31 gene, thereby producing a plant with an enhanced content of said at least one cholesterol derived compound selected from the group comprising a steroidal alkaloid or a glycosylated derivative thereof and an unsaturated or saturated steroidal saponin or a glycoside derivative thereof compared to a corresponding non-transformed plant. A non-limiting example of a nucleic acid sequence that could be operably linked to a GAME31 gene to achieve overexpression would be a 35S promotor. In another embodiment, an at least one steroidal alkaloid or a glycosylated derivative thereof comprises a leptin or a derivative thereof, or leptinine or a derivative thereof, or any combination thereof. In another embodiment, the increase of a leptin or a derivative thereof, or leptinine or a derivative thereof, or any combination thereof provides resistance to at least one plant pathogen, pest, or predator.

In some embodiments, a genetically modified plant comprising increased expression of GAME25 in at least one cell, comprises increased saturated steroidal alkaloids or steroidal glycoalkaloids, or derivatives thereof. In some embodiments, a genetically modified plant comprising increased expression of GAME25 in at least one cell, comprises decreased unsaturated steroidal alkaloids or steroidal glycoalkaloids, or derivatives thereof. In some embodiments, a genetically modified plant comprising increased expression of GAME25 in at least one cell, comprises increased saturated steroidal alkaloids or steroidal glycoalkaloids, or derivatives thereof, wherein said change in saturation is at a C5,C6 bond. In some embodiments, a genetically modified plant comprising increased expression of GAME25 in at least one cell, comprises decreased unsaturated steroidal alkaloids or steroidal glycoalkaloids, or derivatives thereof, wherein said change in saturation is at a C5,C6 bond.

In some embodiments, said increased SA, or SGA, or derivatives thereof occurs in a fruit. In some embodiments, the increased SA, or SGA, or derivatives thereof, occurs in a green fruit, a breaker fruit, a turning fruit, a pink fruit, a light red fruit, or a red fruit, or a combination thereof. In some embodiments, said decreased SA, or SGA, or derivatives thereof occurs in a fruit. In some embodiments, the decreased SA, or SGA, or derivatives thereof, occurs in a green fruit, a breaker fruit, a turning fruit, a pink fruit, a light red fruit, or a red fruit, or a combination thereof.

In some embodiments, a genetically modified plant comprising increased expression of GAME25 in at least one cell, comprises increased unsaturated or saturated steroidal saponins or a glycoside derivative thereof.

In some embodiments, a genetically modified tomato plant comprising increased expression of GAME25 in at least one cell, comprises increased saturated steroidal alkaloids or steroidal glycoalkaloids, or derivatives thereof in said at least one cell, said SA or SGA comprising increased α-tomatine, or hydroxytomatine, or acetoxytomatine, or α-tomatine isomers (2), or acetoxy-hydroxytomatine, or esculeosides, or lycoperosides, or derivatives thereof, or any combination thereof, compared to a non-modified tomato plant. In some embodiments, a genetically modified tomato plant comprising increased expression of GAME25 in at least one cell, comprises decreased unsaturated steroidal alkaloids or steroidal glycoalkaloids, or derivatives thereof, said SA or SGA comprising dehydrotomatine, or hydroxy-dehydrotomatine, or acetoxy-dehydrotomatine, or acetoxy-hydroxy-dehydrotomatine, or dehydroesculeosides, or dehydrolycoperosides, or any derivatives thereof, or any combination thereof, compared to a non-modified tomato plant.

A skilled artisan would appreciate that a genetically modified plant comprising a GAME25 enzyme having increased biological activity in at least one cell, would similarly comprises increased saturated steroidal alkaloids or steroidal glycoalkaloids, or derivatives thereof, compared to a non-modified plant. Similarly, a genetically modified plant comprising a GAME25 enzyme having increased biological activity in at least one cell, would similarly comprise decreased unsaturated steroidal alkaloids or steroidal glycoalkaloids, or derivatives thereof, compared to a non-modified plant.

In a further embodiment, the altered content of at least one steroidal alkaloid (SA) or a glycosylated derivative thereof (SGA) compared to a corresponding unmodified plant comprises an increase in at least one SA or SGA, and a reduction in at least one other SA or SGA, wherein the percent increase or reduction of said SAs or SGAs may independently be increased or reduced by different percentages. Alternatively, in another embodiment, the altered content of at least one steroidal alkaloid (SA) or a glycosylated derivative thereof (SGA) compared to a corresponding unmodified plant comprises an increase in at least one SA or SGA, and a reduction in at least one other SA or SGA, wherein the percent increase or reduction of said SAs or SGAs may be equal to equivalent percent increase and decrease. In another embodiment, the altered content of at least one SA or SGA comprises altered contented or multiple SA or SGA compounds, wherein the number of SA or SGA compounds increased or reduced is not equivalent, e.g., decrease of two SA or SGA compounds and increase of one SA or SGA compound. One of ordinary skill in the art would appreciate that there are many possible combinations of increased and/or decreased SA or SGA compounds, wherein each comprises an embodiment herein.

According to certain embodiments, a genetically modified plant described herein, comprises non-toxic amount of at least one steroidal alkaloid or a glycosylated derivative thereof. A skilled artisan would recognize that the term “non-toxic amount” encompasses less than 200 mg of anti-nutritional steroidal; alkaloids or glycoalkaloids per kilogram fresh weight of an edible plant part. According to certain embodiments, the genetically modified plant comprises non-detectable amount of anti-nutritional steroidal alkaloid or a glycosylated derivative thereof.

In another embodiment, a genetically modified plant comprises a Solanaceae crop plant and said altered content comprises reduction of at least one steroidal alkaloid or glycosylated derivative thereof selected from the group comprising solanidine, solasodine hydroxy solasodine, α-solanine, α-chaconine, tomatidine, α-tomatine, hydroxytomatine, acetoxytomatine, acetoxy-hydroxytomatine, tomatidine+4 hexose, esculeosides, esculeoside A, esculeoside A+hexose, esculeoside B, acetoxyesculeoside B, demissidine, demissine, dehydrosolasodine, dehydrotomatidine, hydroxy-dehydrotomatine, acetoxy-dehydrotomatine, acetoxy-hydroxy-dehydrotomatine, dehydroesculeosides, leptinine I, leptinine II, leptine I, leptine II, lycoperosides, and hydroxysolamargine, or any derivatives thereof, or any combination thereof.

In one embodiment, disclosed herein is a method of producing beneficial steroidal derivatives, said method comprising the steps of: incubating a recombinant GAME25 enzyme, as disclosed herein, or GAME31 enzyme, as disclosed herein, or a combination thereof with selected precursor molecules under biosynthetic conditions; and collecting and isolating the steroidal derivatives from the biosynthetic medium. In another embodiment, the recombinant GAME25 enzyme or GAME31 enzyme or the combination thereof are expressed in a microbial cell or an insect cell, and wherein said incubating comprises incubating said cell in media comprising necessary precursor molecules. In some embodiments, said beneficial steroidal derivatives comprise precursors of a steroidal alkaloid or glycosylated derivative thereof. In some embodiments, a beneficial steroidal derivative is selected from the group comprising demissidine, soladelucidine, leptin, leptinine 1, and leptinine 2.

In some embodiments, disclosed herein are uses of a recombinant protein disclosed herein having the amino acid sequence set forth in SEQ ID NO: 3, SEQ ID NO: 12, or SEQ ID NO: 15, or a protein homologue thereof, wherein said protein homolog is at least 50% homologous to any of SEQ ID NO: 3, SEQ ID NO: 12, or SEQ ID NO: 15 and has the same catalytic function as the protein set forth in SEQ ID NO: 3, SEQ ID NO: 12, or SEQ ID NO: 15, for the production of a cholesterol derived compound selected from the group comprising a steroidal alkaloid or a glycosylated derivative thereof and an unsaturated or saturated steroidal saponin or a glycoside derivative thereof or a biosynthetic product thereof. In some embodiment, disclosed herein are uses of a recombinant protein having the amino acid sequence set forth in SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 or SEQ ID NO: 53, or a protein homologue thereof, wherein said protein homolog is at least 50% homologous to any of SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 or SEQ ID NO: 53 and has the same catalytic function as the protein set forth in SEQ ID NO: 18, SEQ ID NO: 21, SEQ ID NO: 24, SEQ ID NO: 27, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 or SEQ ID NO: 53, for the production of a cholesterol derived compound selected from the group comprising a steroidal alkaloid or a glycosylated derivative thereof, an unsaturated or saturated steroidal saponin or a glycoside derivative thereof, or a hydroxylated derivative thereof, or a biosynthetic product thereof. In some embodiments, uses disclosed herein are in vitro uses.

In some embodiments, disclosed herein are uses of a nucleic acid sequence encoding a 3-β-hydroxysteroid dehydrogenase/isomerase (GAME25) enzyme, said nucleic acid comprising the sequence set forth in SEQ ID NO: 1, SEQ ID NO: 2, SEQ ID NO: 11, SEQ ID NO: 13, or SEQ ID NO: 14, or a nucleic acid sequence having a sequence which is at least 50% identical to SEQ ID NO: 1, SEQ ID NO: 2, SEQ ID NO: 11, SEQ ID NO: 13, or SEQ ID NO: 14, wherein said encoded enzyme has the same catalytic function as the protein of SEQ ID NO: 1, SEQ ID NO: 2, SEQ ID NO: 11, SEQ ID NO: 13, or SEQ ID NO: 14, for the production of a recombinant cholesterol derived compound selected from the group comprising a steroidal alkaloid or a glycosylated derivative thereof and an unsaturated or saturated steroidal saponin or a glycoside derivative thereof. In some embodiments, disclosed herein are uses of a nucleic acid sequence encoding a 3-β-hydroxysteroid dehydrogenase/isomerase (GAME25) enzyme, said nucleic acid comprising the sequence set forth in SEQ ID NO: 1, SEQ ID NO: 2, SEQ ID NO: 11, SEQ ID NO: 13, or SEQ ID NO: 14, or a nucleic acid sequence having a sequence which is at least 50% identical to SEQ ID NO: 1, SEQ ID NO: 2, SEQ ID NO: 11, SEQ ID NO: 13, or SEQ ID NO: 14, wherein said encoded enzyme has the same catalytic function as the protein of SEQ ID NO: 1, SEQ ID NO: 2, SEQ ID NO: 11, SEQ ID NO: 13, or SEQ ID NO: 14, for the production of a recombinant cell capable of biotransformation of a cholesterol derived compound selected from the group comprising a steroidal alkaloid or a glycosylated derivative thereof and an unsaturated or saturated steroidal saponin or a glycoside derivative thereof. In one embodiment, a use for production of a recombinant cholesterol derived compound selected from the group comprising a steroidal alkaloid or a glycosylated derivative thereof and an unsaturated or saturated steroidal saponin or a glycoside derivative thereof comprises an in vitro use. In one embodiment, a use for production of a recombinant cholesterol derived compound selected from the group comprising a steroidal alkaloid or a glycosylated derivative thereof and an unsaturated or saturated steroidal saponin or a glycoside derivative thereof comprises an in vivo use.

In some embodiments, uses disclosed herein comprise use of a nucleic acid sequence encoding a 2-oxoglutarate-dependent dioxygenase (GAME31) enzyme, said nucleic acid comprising the sequence set forth in SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52 or a nucleic acid sequence having a sequence which is at least 50% identical to SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52, wherein said encoded enzyme has the same catalytic function as the protein of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52, for the production of a recombinant cholesterol derived compound selected from the group comprising a steroidal alkaloid or a glycosylated derivative thereof and an unsaturated or saturated steroidal saponin or a glycoside derivative thereof. In some embodiments, uses disclosed herein comprise use of a nucleic acid sequence encoding a 2-oxoglutarate-dependent dioxygenase (GAME31) enzyme, said nucleic acid comprising the sequence set forth in SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52 or a nucleic acid sequence having a sequence which is at least 50% identical to SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52, wherein said encoded enzyme has the same catalytic function as the protein of SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 39, SEQ ID NO: 40, SEQ ID NO: 42, SEQ ID NO: 43, SEQ ID NO: 45, SEQ ID NO: 46, SEQ ID NO: 48, SEQ ID NO: 49, SEQ ID NO: 51, and SEQ ID NO: 52, for the production of a recombinant cell capable of biotransformation of a cholesterol derived compound selected from the group comprising a steroidal alkaloid or a glycosylated derivative thereof and an unsaturated or saturated steroidal saponin or a glycoside derivative thereof. In one embodiment, a use for production of a recombinant. In one embodiment, a use for production of a recombinant cholesterol derived compound selected from the group comprising a steroidal alkaloid or a glycosylated derivative thereof and an unsaturated or saturated steroidal saponin or a glycoside derivative thereof comprises an in vitro use. In one embodiment, a use for production of a recombinant cholesterol derived compound selected from the group comprising a steroidal alkaloid or a glycosylated derivative thereof and an unsaturated or saturated steroidal saponin or a glycoside derivative thereof comprises an in vivo use.

In another embodiment, a genetically modified plant comprises a Solanaceae crop plant and said altered content comprises increase of at least one steroidal alkaloid or glycosylated derivative thereof selected from the group comprising hydroxy solasodine, dehydrotomatine or an isomer thereof, hydroxy-dehydrotomatine, acetoxy-dehydrotomatine, acetoxy-hydroxy-dehydrotomatine, dehydrotomatidine+4 hexose, dehydroesculeoside A, dehydroesculeoside A+hexose, solanidine, α-solanine, α-chaconine, leptinine I, leptinine II, leptine I, leptine II, solasodine, α-solasonine, α-solamargine, hydroxysolasonine, and hydroxysolamargine, or any derivatives thereof, or any combination thereof.

In another embodiment, said Solanaceae crop plant comprises a tomato plant, said altered expression comprises altered expression of GAME25, and said altered content comprises reduction of at least tomatidine and α-tomatine, or derivatives thereof compared to a corresponding non-genetically modified plant. In another embodiment, said Solanaceae crop plant comprises a tomato plant, said altered expression comprises altered expression of GAME25, and said altered content comprises increase of at least dehydrotomatidine or derivatives thereof compared to a corresponding non-genetically modified plant. In another embodiment, said Solanaceae crop plant comprises a tomato plant, said altered expression comprises altered expression of GAME25, and said altered content comprises reduction of at least tomatidine and α-tomatine, or derivatives thereof compared to a corresponding non-genetically modified plant increase of at least dehydrotomatidine or derivatives thereof compared to a corresponding non-genetically modified plant.

In another embodiment, said Solanaceae crop plant comprises a potato plant, said altered expression comprises altered expression of GAME25, and said altered content comprises reduction of at least demissidine or derivatives thereof compared to a corresponding non-genetically modified plant. In another embodiment, said Solanaceae crop plant comprises a potato plant, said altered expression comprises altered expression of GAME25, and said altered content comprises increase of at least solanidine or derivatives thereof compared to a corresponding non-genetically modified plant. In another embodiment, said Solanaceae crop plant comprises a potato plant, said altered expression comprises altered expression of GAME25, and said altered content comprises reduction of at least demissidine or derivatives thereof compared to a corresponding non-genetically modified plant and increase of at least solanidine or derivatives thereof compared to a corresponding non-genetically modified plant.

In another embodiment, said Solanaceae crop plant comprises an aubergine plant, said altered expression comprises altered expression of GAME25, and said altered content comprises reduction of at least dihydrosolasodine or derivatives thereof compared to a corresponding non-genetically modified plant. In another embodiment, said Solanaceae crop plant comprises an aubergine plant, said altered expression comprises altered expression of GAME25, and said altered content comprises increase of at least solasoidine or derivatives thereof compared to a corresponding non-genetically modified plant. In another embodiment, said Solanaceae crop plant comprises an aubergine plant, said altered expression comprises altered expression of GAME25, and said altered content comprises reduction of at least dihydrosolasodine or derivatives thereof compared to a corresponding non-genetically modified plant and increase of at least solasoidine or derivatives thereof compared to a corresponding non-genetically modified plant.

In another embodiment, said Solanaceae crop plant comprises a tomato plant, said altered expression comprises altered expression of GAME31, and said altered content comprises reduction of at least hydroxy-dehydrotomatine, or hydroxytomatine, or any combination thereof, or derivatives thereof, compared to a corresponding non-genetically modified plant. In another embodiment, said Solanaceae crop plant comprises a tomato plant, said altered expression comprises altered expression of GAME31, and said altered content comprises increase of at least dehydrotomatine, or α-tomatine, or any combination thereof, or derivatives thereof compared to a corresponding non-genetically modified plant. In another embodiment, said Solanaceae crop plant comprises a tomato plant, said altered expression comprises altered expression of GAME31, and said altered content comprises reduction of at least hydroxy-dehydrotomatine, or hydroxytomatine, or any combination thereof, or derivatives thereof, compared to a corresponding non-genetically modified plant and increase of at least dehydrotomatine, or α-tomatine, or any combination thereof, or derivatives thereof compared to a corresponding non-genetically modified plant.

In another embodiment, said Solanaceae crop plant comprises a potato plant, said altered expression comprises altered expression of GAME31, and said altered content comprises reduction of at least leptinine II, leptinine I, or derivatives thereof or any combination thereof, compared to a corresponding non-genetically modified plant. In another embodiment, said Solanaceae crop plant comprises a potato plant, said altered expression comprises altered expression of GAME31, and said altered content comprises increase of at least α-solanine, α-chaconine, or derivatives thereof, or any combination thereof, compared to a corresponding non-genetically modified plant. In another embodiment, said Solanaceae crop plant comprises a potato plant, said altered expression comprises altered expression of GAME31, and said altered content comprises reduction of at least leptinine II, leptinine I, or derivatives thereof or any combination thereof, compared to a corresponding non-genetically modified plant and increase of at least α-solanine, α-chaconine, or derivatives thereof, or any combination thereof, compared to a corresponding non-genetically modified plant.

In another embodiment, said Solanaceae crop plant comprises an aubergine plant, said altered expression comprises altered expression of GAME31, and said altered content comprises reduction of at least hydroxysolasonine, hydroxysolamargine, or derivatives thereof, or any combination thereof compared to a corresponding non-genetically modified plant. In another embodiment, said Solanaceae crop plant comprises an aubergine plant, said altered expression comprises altered expression of GAME31, and said altered content comprises increase of at least solasonine, solamargine, or derivatives thereof, or any combination thereof compared to a corresponding non-genetically modified plant. In another embodiment, said Solanaceae crop plant comprises an aubergine plant, said altered expression comprises altered expression of GAME31, and said altered content comprises reduction of at least hydroxysolasonine, hydroxysolamargine, or derivatives thereof, or any combination thereof compared to a corresponding non-genetically modified plant and increase of at least solasonine, solamargine, or derivatives thereof, or any combination thereof compared to a corresponding non-genetically modified plant.

While being exemplified in a genetically modified plant, the disclosure herein may further enable manipulating the synthesis of steroidal alkaloids or glycosylated derivatives thereof in any organism naturally capable of steroidal alkaloid synthesis. Thus, according in another embodiment, a genetically modified organism comprising at least one cell having altered expression of at least one gene selected from the group comprising a gene encoding a 3-β-hydroxysteroid dehydrogenase/isomerase (GAME25) and a gene encoding 2-oxoglutarate-dependent dioxygenase (GAME31), or a combination thereof compared to an unmodified organism, wherein the genetically modified organism has an altered content of at least one compound selected from steroidal alkaloids and glycosylated derivatives thereof compared to a corresponding unmodified organism.

Down-regulation or inhibition of the gene expression can be affected on the genomic and/or the transcript level using a variety of molecules that interfere with transcription and/or translation (e.g., antisense, siRNA, Ribozyme, or DNAzyme), or on the protein level using, e.g., antagonists, enzymes that cleave the polypeptide, and the like. One of ordinary skill in the art would appreciate that molecules that interfere with transcription and/or translation may be termed “silencing molecules”.

In one embodiment, the genetically modified plant described herein comprising at least one cell having an altered gene expression comprises at least one silencing molecule targeted to a gene selected from the group comprising GAME25 and GAME31, or a combination thereof.

In another embodiment, the genetically modified plant is a transgenic plant comprising at least one cell comprising at least one silencing molecule targeted to a gene selected from the group comprising GAME25 and GAME31, and a combination thereof.

A silencing molecule targeted to at least one of GAME25 and/or GAME31 can be designed as is known to a person skilled in the art (See below and Methods for Examples 3-9).

In one embodiment, a silencing molecule is selected from the group comprising an RNA interference molecule, a co-suppression molecule, and an antisense molecule.

In one embodiment, a silencing molecule comprises a polynucleotide having a nucleic acid sequence substantially complementary to a region of the GAME25 gene or a complementary sequence thereof. In another embodiment, the silencing molecule comprises a polynucleotide having a nucleic acid sequence substantially complementary to a region of the GAME25 gene, the gene having the nucleic acids sequence set forth in any one of SEQ ID 1, and SEQ ID NO: 13, In yet another embodiment, the silencing molecule comprises a polynucleotide having a nucleic acid sequence substantially complementary to a region of the GAME25 cDNA, the cDNA having the nucleic acids sequence set forth in any one of SEQ ID 2, SEQ ID NO: 11, and SEQ ID NO: 14.

In one embodiment, the nucleic acid sequence of a silencing molecule comprises the nucleic acid sequence set forth in SEQ ID NO: 8, or a fragment thereof.

In one embodiment, a silencing molecule comprises a polynucleotide having a nucleic acid sequence substantially complementary to a region of the GAME31 gene or a complementary sequence thereof. In another embodiment, the silencing molecule comprises a polynucleotide having a nucleic acid sequence substantially complementary to a region of the GAME31 gene, the gene having the nucleic acids sequence set forth in any one of SEQ ID NO: 16, SEQ ID NO: 19, SEQ ID NO: 22, SEQ ID NO: 25, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 51. In yet another embodiment, the silencing molecule comprises a polynucleotide having a nucleic acid sequence substantially complementary to a region of the GAME31 cDNA, the cDNA having the nucleic acids sequence set forth in any one of SEQ ID NO: 17, SEQ ID NO: 20, SEQ ID NO: 23, SEQ ID NO: 26, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 52.

In one embodiment, the nucleic acid sequence of a silencing molecule comprises the nucleic acid sequence set forth in SEQ ID NO: 58, or a fragment thereof. In one embodiment, the nucleic acid sequence of a silencing molecule comprises the nucleic acid sequence set forth in SEQ ID NO: 59, or a fragment thereof.

In some embodiments, a genetically modified plant comprising reduced expression of GAME25 in at least one cell, comprises decreased saturated steroidal alkaloids or steroidal glycoalkaloids, or derivatives thereof. In some embodiments, a genetically modified plant comprising reduced expression of GAME25 in at least one cell, comprises increased unsaturated steroidal alkaloids or steroidal glycoalkaloids, or derivatives thereof. In some embodiments, a genetically modified plant comprising reduced expression of GAME25 in at least one cell, comprises decreased saturated steroidal alkaloids or steroidal glycoalkaloids, or derivatives thereof, wherein said change in saturation is at a C5,C6 bond. In some embodiments, a genetically modified plant comprising reduced expression of GAME25 in at least one cell, comprises increased unsaturated steroidal alkaloids or steroidal glycoalkaloids, or derivatives thereof, wherein said change in saturation is at a C5,C6 bond. In some embodiments, said decreased SA, or SGA, or derivatives thereof occurs in a fruit. In some embodiments, the decreased SA, or SGA, or derivatives thereof, occurs in a green fruit, a breaker fruit, a turning fruit, a pink fruit, a light red fruit, or a red fruit, or a combination thereof. In some embodiments, said increased SA, or SGA, or derivatives thereof occurs in a fruit. In some embodiments, the increased SA, or SGA, or derivatives thereof, occurs in a green fruit, a breaker fruit, a turning fruit, a pink fruit, a light red fruit, or a red fruit, or a combination thereof.

In some embodiments, a genetically modified tomato plant comprising reduced expression of GAME25 in at least one cell, comprises decreased saturated steroidal alkaloids or steroidal glycoalkaloids, or derivatives thereof in said at least one cell, said SA or SGA comprising decreased α-tomatine, or hydroxytomatine, or acetoxytomatine, or α-tomatine isomers (1 and 2), or acetoxy-hydroxytomatine, or tomatidine+4 hexoses, or esculeoside A, or esculeosides, or lycoperosides, or any derivatives thereof, or any combination thereof, compared to a non-modified tomato plant. In some embodiments, a genetically modified tomato plant comprising reduced expression of GAME25 in at least one cell, comprises increased unsaturated steroidal alkaloids or steroidal glycoalkaloids, or derivatives thereof, said SA or SGA comprising dehydrotomatine, or hydroxy-dehydrotomatine, or dehydrotomatine isomer (1 and 2), or acetoxy-dehydrotomatine, or acetoxy-hydroxy-dehydrotomatine, or dehydrotomatidine+4 hexose, or dehydroesculeoside A, or dehydroesculeosides, or dehydrolycoperosides, or any derivatives thereof, or any combination thereof.

A skilled artisan would appreciate that a genetically modified plant comprising a GAME25 enzyme having decreased biological activity in at least one cell, would similarly comprises decreased saturated steroidal alkaloids or steroidal glycoalkaloids, or derivatives thereof, compared to a non-modified plant. Similarly, a genetically modified plant comprising a GAME25 enzyme having decreased biological activity in at least one cell, would similarly comprise increased unsaturated steroidal alkaloids or steroidal glycoalkaloids, or derivatives thereof, compared to a non-modified plant.

### Antisense Molecules

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double stranded molecule, antisense technology can be applied to it, building a triplex formation.

One skilled in the art would appreciate that the terms “complementary” or “complement thereof” are used herein to encompass the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of expression levels of GAME25, GAME31, or any combination thereof, in cells and/or tissues of an organisms may be effected by transforming the organism cells or tissues with at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog. In some embodiment, an organism is a plant. In other embodiments, an organism is not a plant. In some embodiments, a cell is a plant cell. In other embodiments, a cell is not a plant cell.

### RNA Interference (RNAi) Molecules

RNAi refers to the introduction of homologous double stranded RNA (dsRNA) to target a specific gene product, resulting in post transcriptional silencing of that gene. This phenomenon was first reported in Caenorhabditis elegans by Guo and Kemphues (1995, Cell, 81(4):611-620) and subsequently Fire et al. (1998, Nature 391:806-811) discovered that it is the presence of dsRNA, formed from the annealing of sense and antisense strands present in the in vitro RNA preps, that is responsible for producing the interfering activity.

In certain embodiments, disclosed herein altered gene expression comprises the use of RNA interference (RNAi) to down regulate the expression of GAME25, GAME31, or combination thereof to attenuate the level of steroidal alkaloids/glycoalkaloids in plants.

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available from commercial sources.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

One skilled in the art would appreciate that the terms “promoter element,” “promoter,” or “promoter sequence” may encompass a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full length of the gene or more.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

One of ordinary skill in the art would appreciate that the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

According to some embodiments, the silencing molecule is RNAi targeted to the GAME25 gene, comprising the nucleic acid sequence set forth in SEQ ID NO:8 or a complementary sequence thereof.

According to some embodiments, the silencing molecule is RNAi targeted to the GAME31 gene, comprising the nucleic acid sequence set forth in SEQ ID NO: 58 or a complementary sequence thereof.

### Co-Suppression Molecules

Another agent capable of down-regulating the expression of GAME25, or GAME31, or a combination thereof is a Co-Suppression molecule. Co-suppression is a post-transcriptional mechanism where both the transgene and the endogenous gene are silenced.

Surprisingly, in some embodiments, overexpression of GAME25 results in suppression of the GAME25 gene. According to some embodiments, the co-suppression molecule is polynucleotide homologous to the GAME25 coding sequence. In some embodiments, the co-suppression molecule comprising a polynucleotide homologous to the GAME 25 coding sequence comprises a sequence selected from SEQ ID NO: 8, SEQ ID NO: 2, SEQ ID NO: 11, and SEQ ID NO: 14, or a fragment thereof, or a complementary sequence thereof.

Surprisingly, in some embodiments, overexpression of GAME31 results in suppression of the GAME31 gene. According to some embodiments, the co-suppression molecule is polynucleotide homologous to the GAME31 coding sequence. In some embodiments, the co-suppression molecule comprising a polynucleotide homologous to the GAME 31 coding sequence comprises a sequence selected from SEQ ID NO: 58, SEQ ID NO: 59, and SEQ ID NO: 30, or a fragment thereof, or a complementary sequence thereof. According to some embodiments, the co-suppression molecule is polynucleotide homologous to the GAME31 coding sequence, comprising the nucleic acid sequence set forth in SEQ ID NO: 59 or a complementary sequence thereof

### DNAzyme Molecules

Another agent capable of down-regulating the expression of GAME25, or GAME31, or a combination thereof is a DNAzyme molecule, which is capable of specifically cleaving an mRNA transcript or a DNA sequence of the GAME25, and/or GAME31. DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (for review of DNAzymes, see: Khachigian, L. M. (2002) Curr Opin Mol Ther 4, 119-121).

Examples of construction and amplification of synthetic, engineered DNAzymes recognizing single- and double-stranded target cleavage sites are disclosed in U.S. Pat. No. 6,326,174, the disclosure of which is incorporated herein in its entirety.

### Enzymatic Oligonucleotide

The terms “enzymatic nucleic acid molecule” or “enzymatic oligonucleotide” refers to a nucleic acid molecule which has complementarity in a substrate binding region to a specified gene target, and also has an enzymatic activity which is active to specifically cleave target RNA of GAME25, or GAME31, thereby silencing each of the genes. The complementary regions allow sufficient hybridization of the enzymatic nucleic acid molecule to the target RNA and subsequent cleavage. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, RNAenzyme. The specific enzymatic nucleic acid molecules described in the instant application are not limiting and an enzymatic nucleic acid molecule of this invention requires a specific substrate binding site which is complementary to one or more of the target nucleic acid regions, and that it have nucleotide sequences within or surrounding that substrate binding site which impart a nucleic acid cleaving and/or ligation activity to the molecule. U.S. Pat. No. 4,987,071 discloses examples of such molecules.

### Mutagenesis

Altering the expression of endogenous or exogenous GAME25, or GAME31 genes or a combination thereof, can be also achieved by the introduction of one or more point mutations into a nucleic acid molecule encoding the corresponding proteins. Mutations can be introduced using, for example, site-directed mutagenesis (see, e.g. Wu Ed., 1993 Meth. In Enzymol. Vol. 217, San Diego: Academic Press; Higuchi, “Recombinant PCR” in Innis et al. Eds., 1990 PCR Protocols, San Diego: Academic Press, Inc). Such mutagenesis can be used to introduce a specific, desired amino acid insertion, deletion or substitution. Several technologies for targeted mutagenesis are based on the targeted induction of double-strand breaks (DSBs) in the genome followed by error-prone DNA repair. Mostly commonly used for genome editing by these methods are custom designed nucleases, including zinc finger nucleases and Xanthomonas-derived transcription activator-like effector nuclease (TALEN) enzymes.

In some embodiments, when the expression of the at least one gene or combination thereof is altered, said altering comprises mutagenizing the at least one gene, said mutation present within a coding region of said at least one gene, or a regulatory sequence of said at least one gene, or a combination thereof.

Various types of mutagenesis can be used to modify GAME25 or GAME31 and their encoded polypeptides in order to produce conservative or non-conservative variants. Any available mutagenesis procedure can be used. In some embodiments, the mutagenesis procedure comprises site-directed point mutagenesis. In some embodiments, the mutagenesis procedure comprises random point mutagenesis. In some embodiments, the mutagenesis procedure comprises in vitro or in vivo homologous recombination (DNA shuffling). In some embodiments, the mutagenesis procedure comprises mutagenesis using uracil-containing templates. In some embodiments, the mutagenesis procedure comprises oligonucleotide-directed mutagenesis. In some embodiments, the mutagenesis procedure comprises phosphorothioate-modified DNA mutagenesis. In some embodiments, the mutagenesis procedure comprises mutagenesis using gapped duplex DNA. In some embodiments, the mutagenesis procedure comprises point mismatch repair. In some embodiments, the mutagenesis procedure comprises mutagenesis using repair-deficient host strains. In some embodiments, the mutagenesis procedure comprises restriction-selection and restriction-purification. In some embodiments, the mutagenesis procedure comprises deletion mutagenesis. In some embodiments, the mutagenesis procedure comprises mutagenesis by total gene synthesis. In some embodiments, the mutagenesis procedure comprises double-strand break repair. In some embodiments, the mutagenesis procedure comprises mutagenesis by chimeric constructs. In some embodiments, the mutagenesis procedure comprises mutagenesis by CRISPR/Cas. In some embodiments, the mutagenesis procedure comprises mutagenesis by zinc-finger nucleases (ZFN). In some embodiments, the mutagenesis procedure comprises mutagenesis by transcription activator-like effector nucleases (TALEN). In some embodiments, the mutagenesis procedure comprises any other mutagenesis procedure known to a person skilled in the art.

In some embodiments, mutagenesis can be guided by known information about the naturally occurring molecule and/or the mutated molecule. By way of example, this known information may include sequence, sequence comparisons, physical properties, crystal structure and the like. In some embodiments, the mutagenesis is essentially random. In some embodiments the mutagenesis procedure is DNA shuffling.

A skilled artisan would appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system.

In some embodiments, a CRISPR/Cas system comprises a single-guide RNA (sgRNA) and/or a Cas protein known in the art. In some embodiments, a CRISPR/Cas system comprises a single-guide RNA (sgRNA) and/or a Cas protein newly created to cleave at a preselected site. The skilled artisan would appreciate that the terms “single-guide RNA”, “sgRNA”, and “gRNA” are interchangeable having all the same qualities and meanings, wherein an sgRNA may encompass a chimeric RNA molecule which is composed of a CRISPR RNA (crRNA) and trans-encoded CRISPR RNA (tracrRNA). In some embodiments, a crRNA is complementary to a preselected region of GAME25 or GAME31 DNA, wherein the crRNA “targets” the CRISPR associated polypeptide (Cas) nuclease protein to the preselected target site.

In some embodiments, the length of crRNA sequence complementary is 19-22 nucleotides long e.g., 19-22 consecutive nucleotides complementary to the target site. In another embodiment, the length of crRNA sequence complementary to the region of DNA is about 15-30 nucleotides long. In another embodiment, the length of crRNA sequence complementary to the region of DNA is about 15, 16, 17, 18, 19, 20, 21, 22, 23, 24, 25, 26, 27, 28, 29, or 30 nucleotides long. In another embodiment, the length of crRNA sequence complementary to the region of DNA is 20 nucleotides long. In some embodiments, the crRNA is located at the 5′ end of the sgRNA molecule. In another embodiment, the crRNA comprises 100% complementation within the preselected target sequence. In another embodiment, the crRNA comprises at least 80% complementation within the preselected target sequence. In another embodiment, the crRNA comprises at least 85% complementation within the preselected target sequence. In another embodiment, the crRNA comprises at least 90% complementation within the preselected target sequence. In another embodiment, the crRNA comprises at least 95% complementation within the preselected target sequence. In another embodiment, the crRNA comprises at least 97% complementation within the preselected target sequence. In another embodiment, the crRNA comprises at least 99% complementation within the preselected target sequence. In another embodiment, a tracrRNA is 100-300 nucleotides long and provides a binding site for the Cas nuclease e.g., a Cas9 protein forming the CRISPR/Cas9 complex.

In one embodiment, a mutagenesis system comprises a CRISPR/Cas system. In another embodiment, a CRISPR/Cas system comprises a Cas nuclease and a gRNA molecule, wherein said gRNA molecule binds within said preselected endogenous target site thereby guiding said Cas nuclease to cleave the DNA within said preselected endogenous target site.

In some embodiments, a CRISPR/Cas system comprise an enzyme system including a guide RNA sequence (“gRNA” or “sgRNA”) that contains a nucleotide sequence complementary or substantially complementary to a region of a target polynucleotide, for example a preselected endogenous target site, and a protein with nuclease activity.

In another embodiment, a CRISPR/Cas system comprises a Type I CRISPR-Cas system, or a Type II CRISPR-Cas system, or a Type III CRISPR-Cas system, or derivatives thereof. In another embodiment, a CRISPR-Cas system comprises an engineered and/or programmed nuclease system derived from naturally accruing CRISPR-Cas systems. In another embodiment, a CRISPR-Cas system comprises engineered and/or mutated Cas proteins. In another embodiment, a CRISPR-Cas system comprises engineered and/or programmed guide RNA.

A skilled artisan would appreciate that a guide RNA may contain nucleotide sequences other than the region complementary or substantially complementary to a region of a target DNA sequence, for example a preselected endogenous target site. In another embodiment, a guide RNA comprises a crRNA or a derivative thereof. In another embodiment, a guide RNA comprises a crRNA: tracrRNA chimera.

In another embodiment, a gRNA molecule comprises a domain that is complementary to and binds to a preselected endogenous target site on at least one homologous chromosome. In another embodiment, a gRNA molecule comprises a domain that is complementary to and binds to a polymorphic allele on at least one homologous chromosome. In another embodiment, a gRNA molecule comprises a domain that is complementary to and binds to a preselected endogenous target site on both homologous chromosomes. In another embodiment, a gRNA molecule comprises a domain that is complementary to and binds to polymorphic alleles on both homologous chromosomes.

Cas enzymes comprise RNA-guided DNA endonuclease able to make double-stranded breaks (DSB) in DNA. The term “Cas enzyme” may be used interchangeably with the terms “CRISPR-associated endonucleases” or “CRISPR-associated polypeptides” having all the same qualities and meanings. In one embodiment, a Cas enzyme is selected from the group comprising Cas1, Cas1B, Cas2, Cas3, Cas4, Cas5, Cas6, Cas7, Cas8, Cas9, Cas10, C2cl, CasX, NgAgo, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, or homologs thereof, or modified versions thereof. In another embodiment, a Cas enzyme comprises Cas9. In another embodiment, a Cas enzyme comprises Cas1. In another embodiment, a Cas enzyme comprises Cas1B. In another embodiment, a Cas enzyme comprises Cas2. In another embodiment, a Cas enzyme comprises Cas3. In another embodiment, a Cas enzyme comprises Cas4. In another embodiment, a Cas enzyme comprises Cas5. In another embodiment, a Cas enzyme comprises Cas6. In another embodiment, a Cas enzyme comprises Cas7. In another embodiment, a Cas enzyme comprises Cas8. In another embodiment, a Cas enzyme comprises Cas10. In another embodiment, a Cas enzyme comprises Cpf1. In another embodiment, a Cas enzyme comprises Csy1. In another embodiment, a Cas enzyme comprises Csy2. In another embodiment, a Cas enzyme comprises Csy3. In another embodiment, a Cas enzyme comprises Cse1. In another embodiment, a Cas enzyme comprises Cse2. In another embodiment, a Cas enzyme comprises Csc1. In another embodiment, a Cas enzyme comprises Csc2. In another embodiment, a Cas enzyme comprises Csa5. In another embodiment, a Cas enzyme comprises Csn2. In another embodiment, a Cas enzyme comprises Csm2. In another embodiment, a Cas enzyme comprises Csm3. In another embodiment, a Cas enzyme comprises Csm4. In another embodiment, a Cas enzyme comprises Csm5. In another embodiment, a Cas enzyme comprises Csm6. In another embodiment, a Cas enzyme comprises Cmr1. In another embodiment, a Cas enzyme comprises Cmr3. In another embodiment, a Cas enzyme comprises Cmr4. In another embodiment, a Cas enzyme comprises Cmr5. In another embodiment, a Cas enzyme comprises Cmr6. In another embodiment, a Cas enzyme comprises Csb1. In another embodiment, a Cas enzyme comprises Csb2. In another embodiment, a Cas enzyme comprises Csb3. In another embodiment, a Cas enzyme comprises Csx17. In another embodiment, a Cas enzyme comprises Csx14. In another embodiment, a Cas enzyme comprises Csx10. In another embodiment, a Cas enzyme comprises Csx16, CsaX. In another embodiment, a Cas enzyme comprises Csx3. In another embodiment, a Cas enzyme comprises Csx1, Csx15, Csf1. In another embodiment, a Cas enzyme comprises Csf2. In another embodiment, a Cas enzyme comprises Csf3. In another embodiment, a Cas enzyme comprises Csf4. In another embodiment, a Cas enzyme comprises Cpf1. In another embodiment, a Cas enzyme comprises C2cl. In another embodiment, a Cas enzyme comprises CasX. In another embodiment, a Cas enzyme comprises NgAgo. In another embodiment, a Cas enzyme is Cas homologue. In another embodiment, a Cas enzyme is a Cas orthologue. In another embodiment, a Cas enzyme is a modified Cas enzyme. In another embodiment, a Cas enzyme is any CRISPR-associated endonucleases known in the art.

A skilled artisan would appreciate that the terms “zinc finger nuclease” or “ZFN” are interchangeable having all the same meanings and qualities, wherein a ZFN encompasses a chimeric protein molecule comprising at least one zinc finger DNA binding domain operatively linked to at least one nuclease capable of double-strand cleaving of DNA. In some embodiments, a ZFN system comprises a ZFN known in the art. In some embodiments, a ZFN system comprises a ZFN newly created to cleave a preselected site.

In some embodiments, a ZFN creates a double-stranded break at a preselected endogenous target site. In some embodiments, a ZFN comprises a DNA-binding domain and a DNA-cleavage domain, wherein the DNA binding domain is comprised of at least one zinc finger and is operatively linked to a DNA-cleavage domain. In another embodiment, a zinc finger DNA-binding domain is at the N-terminus of the chimeric protein molecule and the DNA-cleavage domain is located at the C-terminus of the molecule. In another embodiment, a zinc finger DNA-binding domain is at the C-terminus of the chimeric protein molecule and the DNA-cleavage domain is located at the N-terminus of the molecule. In another embodiment, a zinc finger binding domain encompasses the region in a zinc finger nuclease that is capable of binding to a target locus, for example a preselected endogenous target site as disclosed herein. In another embodiment, a zinc finger DNA-binding domain comprises a protein domain that binds to a preselected endogenous target site on at least one homologous chromosome. In another embodiment, a zinc finger DNA-binding domain comprises a protein domain that binds to a polymorphic allele on at least one homologous chromosome. In another embodiment, a zinc finger DNA-binding domain comprises a protein domain that binds to a preselected endogenous target site on both homologous chromosomes. In another embodiment, a zinc finger DNA-binding domain comprises a protein domain that binds to polymorphic alleles on both homologous chromosomes.

The skilled artisan would appreciate that the term “chimeric protein” is used to describe a protein that has been expressed from a DNA molecule that has been created by operatively joining two or more DNA fragments. The DNA fragments may be from the same species, or they may be from a different species. The DNA fragments may be from the same or a different gene. The skilled artisan would appreciate that the term “DNA cleavage domain” of a ZFN encompasses the region in the zinc finger nuclease that is capable of breaking down the chemical bonds between nucleic acids in a nucleotide chain. Examples of proteins containing cleavage domains include restriction enzymes, topoisomerases, recombinases, integrases and DNAses.

In some embodiments, a TALEN system comprises a TAL effector DNA binding domain and a DNA cleavage domain, wherein said TAL effector DNA binding domain binds within said preselected endogenous target site, thereby targeting the DNA cleavage domain to cleave the DNA within said preselected endogenous target site.

A skilled artisan would appreciate that the terms “transcription activator-like effector nuclease”, “TALEN”, and “TAL effector nuclease” may be used interchangeably having all the same meanings and qualities, wherein a TALEN encompasses a nuclease capable of recognizing and cleaving its target site, for example a preselected endogenous target site as disclosed herein. In another embodiment, a TALEN comprises a fusion protein comprising a TALE domain and a nucleotide cleavage domain. In another embodiment, a TALE domain comprises a protein domain that binds to a nucleotide in a sequence-specific manner through one or more TALE-repeat modules. A skilled artisan would recognize that TALE-repeat modules comprise a variable number of about 34 amino acid repeats that recognize plant DNA sequences. Further, repeat modules can be rearranged according to a simple cipher to target new DNA sequences. In another embodiment, a TALE domain comprises a protein domain that binds to a preselected endogenous target site on at least one homologous chromosome. In another embodiment, a TALE domain comprises a protein domain that binds to a polymorphic allele on at least one homologous chromosome. In another embodiment, a TALE domain comprises a protein domain that binds to a preselected endogenous target site on both homologous chromosomes. In another embodiment, a TALE domain comprises a protein domain that binds to polymorphic alleles on both homologous chromosomes.

In one embodiment, a TALE domain comprises at least one of the TALE-repeat modules. In another embodiment, a TALE domain comprises from one to thirty TALE-repeat modules. In another embodiment, a TALE domain comprises more than thirty repeat modules. In another embodiment, a TALEN fusion protein comprises an N-terminal domain, one or more of TALE-repeat modules followed by a half-repeat module, a linker, and a nucleotide cleavage domain.

Chemical mutagenesis using an agent such as Ethyl Methyl Sulfonate (EMS) can be employed to obtain a population of point mutations and screen for mutants of the GAME25, or GAME31 genes, or a combination thereof that may become silent or down-regulated. In plants, methods relaying on introgression of genes from natural populations can be used. Cultured and wild types species are crossed repetitively such that a plant comprising a given segment of the wild genome is isolated. Certain plant species, for example Maize (corn) or snapdragon have natural transposons. These transposons are either autonomous, i.e. the transposase is located within the transposon sequence or non-autonomous, without a transposase. A skilled person can cause transposons to “jump” and create mutations. Alternatively, a nucleic acid sequence can be synthesized having random nucleotides at one or more predetermined positions to generate random amino acid substituting.

In some embodiments, the expression of endogenous GAME25 or GAME31 genes can be altered by the introduction of one or more point mutations into their regulatory sequences. In some embodiments, the expression of exogenous GAME25 or GAME31 genes can be altered by the introduction of one or more point mutations into their regulatory sequences. A skilled artisan would appreciate that “regulatory sequences” refers to nucleotide sequences located upstream (5′non-coding sequences), within, or downstream (3′ non-coding sequences) of a coding sequence, and which influence the transcription, RNA processing or stability, or translation of the associated coding sequence. In some embodiments, regulatory sequences comprise promoters. In some embodiments, regulatory sequences comprise translation leader sequences. In some embodiments, regulatory sequences comprise introns. In some embodiments, regulatory sequences comprise polyadenylation recognition sequences. In some embodiments, regulatory sequences comprise RNA processing sites. In some embodiments, regulatory sequences comprise effector binding sites. In some embodiments, regulatory sequences comprise stem-loop structures.

A skilled artisan would appreciate that “promoter” refers to a DNA sequence capable of controlling the expression of a coding sequence or functional RNA. In some embodiments, a coding sequence is located 3′ to a promoter sequence. It is understood by those skilled in the art that different promoters may direct the expression of a gene in different tissues or cell types, or at different stages of development, or in response to different environmental or physiological conditions. In some embodiments, the promoter comprises a constitutive promoter, i.e., a promoter that causes a gene to be expressed in most cell types at most times. In some embodiments, the promoter comprises a regulated promoter, i.e., a promoter that causes a gene to be expressed in response to sporadic specific stimuli. It is further recognized that in many cases the exact boundaries of regulatory sequences have not been completely defined yet.

A skilled artisan would appreciate that the term “3′ non-coding sequences” or “transcription terminator” refers to DNA sequences located downstream of a coding sequence. In some embodiments, 3′ non-coding sequences comprise polyadenylation recognition sequences. In some embodiments, 3′ non-coding sequences comprise sequences encoding regulatory signals capable of affecting mRNA processing. In some embodiments, 3′ non-coding sequences comprise sequences encoding regulatory signals capable of affecting gene expression. The polyadenylation signal is usually characterized by affecting the addition of polyadenylic acid tracts to the 3′ end of the mRNA precursor. In some embodiments, mutations in the 3′ non-coding sequences affect gene transcription. In some embodiments, mutations in the 3′ non-coding sequences affect RNA processing. In some embodiments, mutations in the 3′ non-coding sequences affect gene stability. In some embodiments, mutations in the 3′ non-coding sequences affect translation of the associated coding sequence.

### Biological Activity

In some embodiments, the biological activity of GAME25 or GAME31 is altered compared with a control GAME25 enzyme or a control GAME31 enzyme.

A skilled artisan would recognize that the term “biological activity” refers to any activity associated with a protein that can be measured by an assay. In some embodiments, the biological activity of GAME25 and/or GAME31 comprises biosynthesis of steroidal alkaloids and glycosylated derivatives thereof. In some embodiments, the biological activity of GAME25 and/or GAME31 affect the levels of steroidal alkaloids in at least apart of a plant. In some embodiments, an altered biological activity comprises increased enzyme activity. In some embodiments, an altered biological activity comprises decreased enzyme activity. In some embodiments, an altered biological activity comprises increased stability of the polypeptide. In some embodiments, an altered biological activity comprises decreased stability of the polypeptide.

In some embodiments, the altered biological activity comprises
(a) increased enzyme activity of said 3-β-hydroxysteroid dehydrogenase/isomerase (GAME25) or said 2-oxoglutarate-dependent dioxygenase (GAME31), or the combination thereof, or
(b) increased stability of said 3-β-hydroxysteroid dehydrogenase/isomerase (GAME25) or said 2-oxoglutarate-dependent dioxygenase (GAME31), or the combination thereof, or
(c) decreased enzyme activity of said 3-β-hydroxysteroid dehydrogenase/isomerase (GAME25) or said 2-oxoglutarate-dependent dioxygenase (GAME31), or the combination thereof, or
(d) decreased stability of said 3-β-hydroxysteroid dehydrogenase/isomerase (GAME25) or said 2-oxoglutarate-dependent dioxygenase (GAME31), or the combination thereof, compared to the biological activity in an unmodified plant.

In some embodiments, the biological activity of a GAME25 enzyme is increased compared with a control GAME25 enzyme. In some embodiments, the biological activity of a GAME 25 enzyme is decreased compared with a control GAME25 enzyme. In some embodiments, a GAME25 enzyme has increased stability compared with a control GAME25 enzyme. In some embodiments, a GAME25 enzyme has decreased stability compared with a control GAME25 enzyme.

In some embodiments, the biological activity of a GAME31 enzyme is increased compared with a control GAME31 enzyme. In some embodiments, the biological activity of a GAME 31 enzyme is decreased compared with a control GAME31 enzyme. In some embodiments, a GAME31 enzyme has increased stability compared with a control GAME31 enzyme. In some embodiments, a GAME31 enzyme has decreased stability compared with a control GAME31 enzyme.

In some embodiments, the biological activity of a GAME25 enzyme is increased compared with a control GAME25 enzyme, and the biological activity of GAME 31 is unchanged. In some embodiments, the biological activity of a GAME25 enzyme is increased compared with a control GAME25 enzyme, and the biological activity of GAME 31 is also increased compared with a control GAME31. In some embodiments, the biological activity of a GAME25 enzyme is increased compared with a control GAME25 enzyme, and the biological activity of GAME 31 is decreased compared with a control GAME31. In some embodiments, the biological activity of a GAME 25 enzyme is decreased compared with a control GAME25 enzyme and the biological activity of GAME31 is unchanged. In some embodiments, the biological activity of a GAME 25 enzyme is decreased compared with a control GAME25 enzyme and the biological activity of GAME31 is also decreased compared with a control GAME31. In some embodiments, the biological activity of a GAME 25 enzyme is decreased compared with a control GAME25 enzyme and the biological activity of GAME31 is increased compared with a control GAME31.

In some embodiments, the stability of a GAME25 enzyme is increased compared with a control GAME25 enzyme, and the stability of GAME 31 is unchanged. In some embodiments, the stability of a GAME25 enzyme is increased compared with a control GAME25 enzyme, and the stability of GAME 31 is also increased compared with a control GAME31. In some embodiments, the stability of a GAME25 enzyme is increased compared with a control GAME25 enzyme, and the stability of GAME 31 is decreased compared with a control GAME31. In some embodiments, the stability of a GAME 25 enzyme is decreased compared with a control GAME25 enzyme and the stability of GAME31 is unchanged. In some embodiments, the stability of a GAME 25 enzyme is decreased compared with a control GAME25 enzyme and the stability of GAME31 is also decreased compared with a control GAME31. In some embodiments, the stability of a GAME 25 enzyme is decreased compared with a control GAME25 enzyme and the stability of GAME31 is increased compared with a control GAME31.

In some embodiment, the biological activity comprises biosynthesis of steroidal alkaloids and glycosylated derivatives thereof. Thus, the biological activity of GAME25 and/or GAME31 affect the levels of steroidal alkaloids in at least a part of a plant.

In some embodiments, a genetically modified plant comprising an altered the biological activity of GAME25 alters the content of at least one cholesterol derived compound selected from the group comprising a steroidal alkaloid or a glycosylated derivative thereof and an unsaturated or saturated steroidal saponin or a glycoside derivative thereof compared to a corresponding unmodified plant. In some embodiments, a genetically modified plant comprising an altered the biological activity of GAME31 alters the content of at least one steroidal alkaloid or a glycosylated derivative thereof compared to a corresponding unmodified plant. In some embodiments, a genetically modified plant comprising an altered the biological activity of GAME25 and GAME31 alters the content of at least one cholesterol derived compound selected from the group comprising a steroidal alkaloid or a glycosylated derivative thereof and an unsaturated or saturated steroidal saponin or a glycoside derivative thereof compared to a corresponding unmodified plant.

In some embodiments, disclosed herein is a method of altering the content of at least one cholesterol derived compound selected from the group comprising a steroidal alkaloid or a glycosylated derivative thereof and an unsaturated or saturated steroidal saponin or a glycoside derivative thereof in a plant, comprising altering the biological activity of at least one protein selected from the group comprising a 3-β-hydroxysteroid dehydrogenase/isomerase (GAME25) and a 2-oxoglutarate-dependent dioxygenase (GAME31), or a combination thereof. In some embodiments, altering the biological activity of at least one protein selected from the group comprising a 3-β-hydroxysteroid dehydrogenase/isomerase (GAME25) and a 2-oxoglutarate-dependent dioxygenase (GAME31), or a combination thereof, comprises introducing one or more point mutations into a DNA sequence coding GAME25, or GAME31, or a combination thereof, wherein said mutated DNA sequence is expressed in at least one plant cell within said plant. In some embodiments, altering the biological activity of at least one protein selected from the group comprising a 3-β-hydroxysteroid dehydrogenase/isomerase (GAME25) and a 2-oxoglutarate-dependent dioxygenase (GAME31), or a combination thereof, comprises introducing one or more point mutations into a DNA regulatory sequence that is operably linked to the DNA sequence encoding GAME25, or GAME31, or a combination thereof, wherein said mutated regulatory DNA sequence alters the expression of the DNA sequence encoding GAME25, or GAME31, or a combination thereof in at least one plant cell within said plant.

In some embodiments, a method of producing a plant with an altered content of at least one cholesterol derived compound selected from the group comprising a steroidal alkaloid or a glycosylated derivative thereof and an unsaturated or saturated steroidal saponin or a glycoside derivative thereof comprises altering the biological activity of a 3-β-hydroxysteroid dehydrogenase/isomerase (GAME25) and a 2-oxoglutarate-dependent dioxygenase (GAME31), or a combination thereof, as described herein, or altering the expression level of a 3-β-hydroxysteroid dehydrogenase/isomerase (GAME25) and a 2-oxoglutarate-dependent dioxygenase (GAME31), or a combination thereof as described herein, or altering the activity and expression level of a 3-β-hydroxysteroid dehydrogenase/isomerase (GAME25) and a 2-oxoglutarate-dependent dioxygenase (GAME31), or a combination thereof as described herein, compared to a corresponding non-transformed plant. In some embodiments, altering the content of at least one cholesterol derived compound selected from the group comprising asteroidal alkaloid or a glycosylated derivative thereof and an unsaturated or saturated steroidal saponin or a glycoside derivative thereof in a plant comprises increasing said content. In some embodiments, altering the content of at least one cholesterol derived compound selected from the group comprising a steroidal alkaloid or a glycosylated derivative thereof and an unsaturated or saturated steroidal saponin or a glycoside derivative thereof in a plant comprises decreasing said content. In some embodiments, altering the content of at least one cholesterol derived compound selected from the group comprising a steroidal alkaloid or a glycosylated derivative thereof and an unsaturated or saturated steroidal saponin or a glycoside derivative thereof in a plant comprises increasing the content of at least one cholesterol derived compound selected from the group comprising a steroidal alkaloid or a glycosylated derivative thereof and an unsaturated or saturated steroidal saponin or a glycoside derivative thereof and decreasing the content of at least one other cholesterol derived compound selected from the group comprising a steroidal alkaloid or a glycosylated derivative thereof and an unsaturated or saturated steroidal saponin or a glycoside derivative thereof.

In some embodiments, the biological activity of GAME25 comprises a 3-β-hydroxysteroid dehydrogenase/isomerase enzyme activity. In some embodiments, the biological activity of GAME25 comprises a step in the multi-step conversion of dehydrotomatidine to tomatidine. In some embodiments, the biological activity of GAME25 comprises a step in the multi-step conversion of solanidine to demissidine. In some embodiments, the biological activity of GAME25 comprises a step in the multi-step conversion of solasoidine to dihydrosolasodine.

In some embodiments, the biological activity of GAME31 comprises a 2-oxoglutarate-dependent dioxygenase enzyme activity. In some embodiments, the biological activity of GAME31 comprises a step in the conversion of dehydrotomatine to hydroxy-dehydrotomatine. In some embodiments, the biological activity of GAME31 comprises a step in the conversion of α-tomatine to hydroxytomatine. In some embodiments, the biological activity of GAME31 comprises a step in the conversion of α-solanine to leptinine II. In some embodiments, the biological activity of GAME31 comprises a step in the conversion of α-chaconine to leptinine I. In some embodiments, the biological activity of GAME31 comprises a step in the conversion of solasonine to hydroxysolasonine. In some embodiments, the biological activity of GAME31 comprises a step in the conversion of α-solamargine to hydroxysolamargine.

In some embodiments, the biological activity of GAME 25, GAME31, or a combination thereof, comprises altering the content of a steroidal alkaloid or a glycosylated derivative thereof selected from the group comprising: tomatidine, α-tomatine, α-tomatine isomer (1 and 2), hydroxytomatine, acetoxytomatine, acetoxy-hydroxytomatine, tomatidine+4 hexose, esculeosides, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydroesculeosides, leptinine I, leptinine II, leptine I, leptine II, lycoperosides, and hydroxysolamargine, or any derivatives thereof, or any combination thereof.

In some embodiments, the biological activity of GAME 25, GAME31, or a combination thereof, comprises altering the content of a steroidal alkaloid or a glycosylated derivative thereof selected from the group comprising: dehydrotomatine or an isomer thereof, hydroxy-dehydrotomatine, acetoxy-dehydrotomatine, acetoxy-hydroxy-dehydrotomatine, dehydrotomatidine+4 hexose, dehydroesculeoside A, dehydroesculeoside A+hexose, solanidine, α-solanine, α-chaconine, leptinine I, leptinine II, leptine I, leptine II, solasoidine, α-solasonine, α-solamargine, hydroxysolasonine, and hydroxysolamargine, or any derivatives thereof, or any combination thereof.

In some embodiments, the biological activity of GAME 25, GAME31, or a combination thereof, comprises increasing plant resistance to at least one plant pathogen, pest, or predator, or any combination thereof. In some embodiments, the biological activity of GAME 25, GAME31, or a combination thereof, comprises generating precursor molecules for steroidal alkaloid molecules that provide resistance to at least one plant pathogen, pest, or predator, or any combination thereof.

In some embodiments, the biological activity of GAME 25, GAME31, or a combination thereof, comprises synthesizing anti-nutritional steroidal alkaloids or glycosylated derivatives thereof.

In some embodiments, the biological activity of GAME25 is enhanced by the introduction of one or more point mutations into its DNA coding sequences. In some embodiments, the biological activity of GAME25 is diminished by the introduction of one or more point mutations into its DNA coding sequences. In some embodiments, the biological activity of GAME31 is enhanced by the introduction of one or more point mutations into its DNA coding sequences. In some embodiments, the biological activity of GAME31 is diminished by the introduction of one or more point mutations into its DNA coding sequences. In some embodiments, the biological activity of GAME25, GAME31, or a combination thereof, is enhanced by increasing the stability of GAME25, GAME31, or a combination thereof, by the introduction of one or more point mutations into their DNA coding sequences. In some embodiments, the biological activity of GAME25, GAME31, or a combination thereof, is decreased by decreasing the stability of GAME25, GAME31, or a combination thereof, by the introduction of one or more point mutations into their DNA coding sequences.

### Overexpression

According to yet additional embodiments, provided herein is a genetically modified plant having enhanced expression of at least one gene selected from the group comprising a gene encoding 3-β-hydroxysteroid dehydrogenase/isomerase (GAME25), and a gene encoding 2-oxoglutarate-dependent dioxygenase (GAME31), or a combination thereof, wherein the genetically modified plant has an increased amount of at least one cholesterol derived compound selected from the group comprising a steroidal alkaloid or a glycosylated derivative thereof and an unsaturated or saturated steroidal saponin or a glycoside derivative thereof compared to a corresponding unmodified plant. In plants, steroidal alkaloids play a role in protecting the plant from various pathogens. Steroidal alkaloids are referred to as phytoanticipins, i.e. low molecular weight anti-microbial compounds that are present in the plant before challenge by microorganisms or produced after infection solely from preexisting constituents. Over-expression of GAME25, or GAME31, or any combination thereof in non-edible parts of the plant can thus enhance the plant resistance to steroidal-alkaloid-sensitive pathogens.

### Transgenic Plants

Cloning of a polynucleotide encoding a protein of the present invention selected from the group comprising of 3-β-hydroxysteroid dehydrogenase/isomerase (GAME25, 2-oxoglutarate-dependent dioxygenase (GAME31), and a combination thereof or a molecule that silences a gene encoding same may be performed by any method as is known to a person skilled in the art. Various DNA constructs may be used to express the desired gene or silencing molecule targeted to the gene in a desired organism.

In certain embodiments, the gene or a silencing molecule targeted thereto form part of an expression vector comprising all necessary elements for expression of the gene or its silencing molecule. In certain embodiments, the expression is controlled by a constitutive promoter. In certain embodiments, the constitutive promoter is specific to a plant tissue. According to these embodiments, the tissue specific promoter is selected from the group consisting of root, tuber, leaves and fruit specific promoter. Root specific promoters are described, e.g. in Martinez, E. et al. 2003. Curr. Biol. 13:1435-1441. Fruit specific promoters are described among others in Estornell L. H et al. 2009. Plant Biotechnol. J. 7:298-309 and Fernandez A. I. Et al. 2009 Plant Physiol. 151:1729-1740. Tuber specific promoters are described, e.g. in Rocha-Sosa M, et al., 1989. EMBO J. 8:23-29; McKibbin R. S. et al., 2006. Plant Biotechnol J. 4(4):409-18. Leaf specific promoters are described, e.g. in Yutao Yang, Guodong Yang, Shijuan Liu, Xingqi Guo and Chengchao Zheng. Science in China Series C: Life Sciences. 46: 651-660.

In certain embodiments, the expression vector further comprises regulatory elements at the 3′ non-coding sequence. A skilled artisan would appreciate that the term “3′ non-coding sequences” encompasses DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht I L et al. (1989. Plant Cell 1:671-680).

Those skilled in the art would appreciate that the various components of the nucleic acid sequences and the transformation vectors described in the present invention are operatively linked, so as to result in expression of said nucleic acid or nucleic acid fragment. Techniques for operatively linking the components of the constructs and vectors of the present invention are well known to those skilled in the art. Such techniques include the use of linkers, such as synthetic linkers, for example including one or more restriction enzyme sites.

One skilled in the art would appreciate that the term “operably linked” may encompass the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

Methods for transforming a plant, described herein, are known to those skilled in the art. One skilled in the art would appreciate that the term “transformation” or “transforming” describes a process by which a foreign DNA, such as a DNA construct, including expression vector, enters and changes a recipient cell into a transformed, genetically altered or transgenic cell. Transformation may be stable, wherein the nucleic acid sequence is integrated into the organism genome and as such represents a stable and inherited trait, or transient, wherein the nucleic acid sequence is expressed by the cell transformed but is not integrated into the genome, and as such represents a transient trait. According to preferred embodiments the nucleic acid sequence of the present invention is stably transformed into the plant cell.

The genetically altered plants having altered content of the desired steroidal alkaloid(s) or steroidal glycoalkaloid(s), disclosed herein, are typically first selected based on the expression of the gene or protein. Plants having enhanced or aberrant expression of the gene or protein, are then analyzed for the content of steroidal alkaloids and steroidal glycoalkaloids.

Detection of mutated GAME25, or GAME31 genes, or a combination thereof and/or the presence of silencing molecule targeted to the gene is performed employing standard methods of molecular genetics, known to a person of ordinary skill in the art.

For measuring the gene(s) or silencing molecule(s) expression, cDNA or mRNA should be obtained from an organ in which the nucleic acid is expressed. The sample may be further processed before the detecting step. For example, the polynucleotides in the cell or tissue sample may be separated from other components of the sample, may be amplified, etc. All samples obtained from an organism, including those subjected to any sort of further processing are considered to be obtained from the organism.

Detection of the gene(s) or the silencing molecule(s) typically requires amplification of the polynucleotides taken from the candidate altered organism. Methods for DNA amplification are known to a person skilled in the art. Most commonly used method for DNA amplification is PCR (polymerase chain reaction; see, for example, PCR Basics: from background to Bench, Springer Verlag, 2000; Eckert et al., 1991. PCR Methods and Applications 1:17). Additional suitable amplification methods include the ligase chain reaction (LCR), transcription amplification and self-sustained sequence replication, and nucleic acid based sequence amplification (NASBA).

In certain embodiments, the nucleic acid sequence comprising the GAME25, or GAME31 genes or its silencing molecule further comprises a nucleic acid sequence encoding a selectable marker. In certain embodiments, the selectable marker confers resistance to antibiotic or to an herbicide; in these embodiments the transgenic plants are selected according to their resistance to the antibiotic or herbicide.

The content of steroidal alkaloids and/or steroidal glycoalkaloids is measured as exemplified hereinbelow and as is known to a person skilled in the art.

In one embodiment, the genetically modified plant disclosed herein comprising at least one cell having an altered expression, comprises at least one transcribable polynucleotide. In another embodiment, the genetically modified plant disclosed herein comprising at least one cell having an altered expression, comprises at least one transcribable polynucleotide encoding at least one protein, said at least one protein selected from the group comprising a GAME25 3-β-hydroxysteroid dehydrogenase/isomerase and a GAME31 2-oxoglutarate-dependent dioxygenase, or any combination thereof. In another embodiment, the genetically modified plant disclosed herein comprising at least one cell having an altered expression, comprises at least one transcribable polynucleotide complementary of anti-sense to a GAME25 gene or portion thereof. In another embodiment, the genetically modified plant disclosed herein comprising at least one cell having an altered expression, comprises at least one transcribable polynucleotide complementary or anti-sense to a GAME31 gene or portion thereof. In another embodiment, the genetically modified plant disclosed herein comprising at least one cell having an altered expression, comprises at least one transcribable polynucleotide complementary of anti-sense to a GAME25 gene or portion thereof, or complementary or anti-sense to a GAME31 gene or portion thereof, or any combination thereof.

In some embodiments, the at least one transcribable polynucleotide comprises the nucleic acid sequence set forth in SEQ ID NO: 8. In some embodiments, the at least one transcribable polynucleotide comprises the nucleic acid sequence set forth in SEQ ID NO: 58. In some embodiments, the at least one transcribable polynucleotide comprises the nucleic acid sequence set forth in SEQ ID NO: 59. In some embodiments, the at least one transcribable polynucleotide comprises the nucleic acid sequence set forth in any one of SEQ ID NO: 8, SEQ ID NO: 58, and SEQ ID NO: 59, or any combination thereof.

One skilled in the art would appreciate that edible components of plants may go through ripening stages. For example, for a tomato, six ripening stages may be identified: Green, Breakers, Turning, Pink, Light Red, and Red. In one embodiment, Green-stage one-means that the surface of the tomato is completely green in color, wherein the shade of green may vary from light to dark. In one embodiment, Breakers-stage two-means there is a definite “break” in color from green to tannish-yellow, pink or red on not more than 10% of the surface. In one embodiment, Turning-stage 3-means that more than 10%, but not more than 30%, of the surface, in the aggregate, shows a definite change in color from green to tannish-yellow, pink, red, or a combination thereof. In one embodiment, Pink-stage four-means that more than 30%, but not more than 60%, of the surface, in the aggregate, shows pink or red in color. Light Red-stage 5-means that more than 60% of the surface, in the aggregate, shows pinkish-red or red, provided that not more than 90% of the surface is red. Red-stage 6—means that more than 90% of the surface, in the aggregate, is red.

In one embodiment, the at least one cell having altered expression is selected from the group consisting of an immature green tissue cell, a mature green tissue cell, an or ange tissue cell, a breaker tissue cell, and a ripe tissue cell.

In another embodiment, the at least one cell having altered expression is selected from the group consisting of leaf cell, a bud cell, a petal cell, a root cell, a peal cell, a flower cell, a stem cell, a shoot cell, and a fruit cell. One skilled in the art would appreciate that it may be advantageous for a plant to have increased expressions of at least one SA/SGA in order to provide the plant with resistance to pathogens and pests during development and growth, wherein at the same time it would be advantageous for an edible part of the plant to have reduced SA/SGA, wherein the SA/SGA comprising anti-nutritional compounds. In some embodiments, a leaf cell comprises a young leaf cell. In some embodiments, a leaf cell comprises a mature leaf cell.

In one embodiment, an edible part of a plant is a fruit. In another embodiment, an edible part of a plant is a tuber. In another embodiment, an edible part of a plant is a leaf, a young leaf, or a mature leaf. In another embodiment, an edible part of a plant is a bud. In one embodiment, a fruit comprises a green fruit, a breaker fruit, a turning fruit, a pink fruit, a light red fruit, or a red ripe fruit. In another embodiment, a fruit comprises a green fruit, a breaker fruit, or a red ripe fruit.

In one embodiment, disclosed herein is a method of reducing the content of at least one cholesterol derived compound selected from the group comprising a steroidal alkaloid or a glycosylated derivative thereof and an unsaturated or saturated steroidal saponin or a glycoside derivative thereof in a plant, comprising transforming at least one plant cell within said plant with at least one silencing molecule targeted to a nucleic acid sequence encoding at least one protein selected from the group comprising a3-β-hydroxysteroid dehydrogenase/isomerase (GAME25) and a 2-oxoglutarate-dependent dioxygenase (GAME31), or a combination thereof, thereby producing a plant with a reduced content of said at least one cholesterol derived compound selected from the group comprising a steroidal alkaloid or a glycosylated derivative thereof and an unsaturated or saturated steroidal saponin or a glycoside derivative thereof compared to a corresponding non-transformed plant. In another embodiment, a method of reducing the content of at least one cholesterol derived compound selected from the group comprising a steroidal alkaloid or a glycosylated derivative thereof and an unsaturated or saturated steroidal saponin or a glycoside derivative thereof comprises reducing the content of at least one cholesterol derived compound selected from the group comprising a steroidal alkaloid or a glycosylated derivative thereof and an unsaturated or saturated steroidal saponin or a glycoside derivative thereof comprising an anti-nutritional compound.

In some embodiments, a method of reducing the content of at least one cholesterol derived compound selected from the group comprising a steroidal alkaloid or a glycosylated derivative thereof and an unsaturated or saturated steroidal saponin or a glycoside derivative thereof in a plant, said method comprising a step
transforming at least one plant cell within said plant with at least one silencing molecule targeted to a nucleic acid sequence encoding at least one protein selected from the group comprising 3-β-hydroxysteroid dehydrogenase/isomerase (GAME25), 2-oxoglutarate-dependent dioxygenase (GAME31), or any combination thereof, or
transforming at least one plant cell within said plant with at least one polynucleotide sequence encoding at least one protein selected from the group comprising 3-β-hydroxysteroid dehydrogenase/isomerase (GAME25), or a 2-oxoglutarate-dependent dioxygenase (GAME31), or any combination thereof, wherein said at least one polynucleotide sequence comprises a mutation in a coding region or a regulatory region; or
a combination of (a) and (b);
   thereby producing a plant with a reduced content of said at least one cholesterol derived compound selected from the group comprising a steroidal alkaloid or a glycosylated derivative thereof and an unsaturated or saturated steroidal saponin or a glycoside derivative thereof compared to a corresponding non-transformed plant.

In another embodiment, the method of reducing the content of at least one steroidal alkaloid or a glycosylated derivative thereof comprising reducing the content of at least one anti-nutritional compound, maintains or essentially maintains the resistance to at least one pathogen or predator in the plant, compared to a corresponding non-transformed plant. In an alternate embodiment, the plant resistance to at least one pathogen or predator is increased compared to a corresponding non-transformed plant.

In some embodiments, the reduced at least one steroidal alkaloid or a glycosylated derivative thereof comprises any of the SA or SGA disclosed herein. In some embodiments, the reduced at least one steroidal alkaloid or a glycosylated derivative thereof comprises a sub-set of the SA or SGA disclosed herein. In some embodiments, the sub-set of reduced at least one steroidal alkaloid or a glycosylated derivative thereof comprises α-tomatine, α-tomatine isomer (1 and 2), hydroxytomatine, acetoxytomatine, acetoxy-hydroxytomatine, esculeosides, lycoperosides, or derivatives thereof, or any combination thereof. In some embodiments, while a sub-set of SA or SGA are reduced, additionally at least one steroidal alkaloid or a glycosylated derivative thereof is increased, wherein the at least one steroidal alkaloid or a glycosylated derivative thereof comprising a dehydrotomatine, a dehydrotomatine isomer 1, a dehydrotomatidine+4-hexose, a hydroxy-dehydrotomatine, an acetoxy-dehydrotomatine, an acetoxy-hydroxy-dehydrotomatine, an dehydroesculeosides, an dehydrolycoperosides, or any derivatives thereof, or any combination thereof.

In some embodiments, the reduced at least one unsaturated or saturated steroidal saponin or a glycosylated derivative thereof comprises any unsaturated or saturated steroidal saponin or glycosylated derivative thereof. In some embodiments, the reduced at least one unsaturated or saturated steroidal saponin or a glycosylated derivative thereof comprises dioscin, diosgenin, parillin, or sarasapogenin. In some embodiments, the reduced at least one unsaturated or saturated steroidal saponin or a glycosylated derivative thereof comprises aescin, araloside A, astragaloside, bacopaside, bacoside, bacoside A, chaconine, charantin, daucosterol, esculeoside A, ginsenoside, glycyrrhizin, α-hederin, holothurin, momordicine, momordin, osladin, protodioscin, pseudoginsenoside F11, QS21, solanine, triterpenoid saponin, and ziziphin

### Breeding

In some embodiments, disclosed herein is a method for breeding a plant having altered content of at least one cholesterol derived compound selected from the group comprising a steroidal alkaloid or a glycosylated derivative thereof and an unsaturated or saturated steroidal saponin or a glycoside derivative thereof; said method comprising providing a first plant, wherein the expression level of GAME25, GAME31, or a combination thereof, in said first plant is in a pre-determined range of values; providing a second plant; crossing said first and second plants to generate an offspring plant; and selecting an offspring plant that has a significantly different content of at least one cholesterol derived compound selected from the group comprising a steroidal alkaloid or a glycosylated derivative thereof and an unsaturated or saturated steroidal saponin or a glycoside derivative thereof compared to said second plant. In some embodiments, a method for breeding a plant having altered content of at least one cholesterol derived compound selected from the group comprising a steroidal alkaloid or a glycosylated derivative thereof and an unsaturated or saturated steroidal saponin or a glycoside derivative thereof; comprises (a) providing a first plant, wherein the expression level of a polynucleotide encoding a 3-β-hydroxysteroid dehydrogenase/isomerase (GAME25), a 2-oxoglutarate-dependent dioxygenase (GAME31), or a combination thereof is in a pre-determined range of values, or a biological activity of a 3-β-hydroxysteroid dehydrogenase/isomerase (GAME25), a 2-oxoglutarate-dependent dioxygenase (GAME31), or a combination thereof, is in a pre-determined range of values; (b) providing a second plant; (c) crossing said first and second plants to generate an offspring plant; and (d) selecting an offspring plant that has a significantly different content of at least one cholesterol derived compound selected from the group comprising a steroidal alkaloid or a glycosylated derivative thereof and an unsaturated or saturated steroidal saponin or a glycoside derivative thereof compared to said second plant.

In some embodiments, a pre-determined value of expression comprises under-expression or over-expression compared to a non-genetically modified plant. In some embodiments, a pre-determined value of expression comprises under-expression or over-expression compared to a wild-type plant. In some embodiments, a pre-determined value of expression comprises under-expression or over-expression compared to a different genetically modified plant.

In some embodiments, a pre-determine value of biological activity comprises increase enzyme activity, or decreased enzyme activity, or increased stability, or decreased stability of said GAME25 or GAME31, or the combination thereof, compared to a non-genetically modified plant. In some embodiments, a pre-determine value of biological activity comprises increase enzyme activity, or decreased enzyme activity, or increased stability, or decreased stability of said GAME25 or GAME31, or the combination thereof, compared to a wild-type plant. In some embodiments, a pre-determine value of biological activity comprises increase enzyme activity, or decreased enzyme activity, or increased stability, or decreased stability of said GAME25 or GAME31, or the combination thereof, compared to a different genetically modified plant.

In some embodiments, disclosed herein is a method for breeding a plant with decreased content of at least one cholesterol derived compound selected from the group comprising a steroidal alkaloid or a glycosylated derivative thereof and an unsaturated or saturated steroidal saponin or a glycoside derivative thereof, said method comprising providing a first plant, wherein the expression level of GAME25, GAME31, or a combination thereof, in said first plant is below a pre-determined value; providing a second plant; crossing said first and second plants to generate an offspring plant; and selecting an offspring plant that has decreased content of at least one cholesterol derived compound selected from the group comprising a steroidal alkaloid or a glycosylated derivative thereof and an unsaturated or saturated steroidal saponin or a glycoside derivative thereof compared to said second plant.

In some embodiments, disclosed herein is a method for breeding a plant with increased content of at least one cholesterol derived compound selected from the group comprising a steroidal alkaloid or a glycosylated derivative thereof and an unsaturated or saturated steroidal saponin or a glycoside derivative thereof, said method comprising providing a first plant, wherein the expression level of GAME25, GAME31, or a combination thereof, in said first plant is above a pre-determined value; providing a second plant; crossing said first and second plants to generate an offspring plant; and selecting an offspring plant that has increased content of at least one cholesterol derived compound selected from the group comprising a steroidal alkaloid or a glycosylated derivative thereof and an unsaturated or saturated steroidal saponin or a glycoside derivative thereof compared to said second plant.

In some embodiments, alternative methods may be used to breed a plant having an altered expression of at least one gene. For example, in some embodiments, a method for breeding a plant having an altered expression of at least one gene selected from the group comprising a gene encoding 3-β-hydroxysteroid dehydrogenase/isomerase (GAME25), a gene encoding a 2-oxoglutarate-dependent dioxygenase (GAME31), or a combination thereof, said method comprising:
providing a first transformed plant, wherein said first transformed plant is transformed with an expression vector comprising a polynucleotide comprising at least one silencing molecule targeted to a nucleic acid sequence encoding at least one protein selected from the group comprising a 3-β-hydroxysteroid dehydrogenase/isomerase (GAME25) and a 2-oxoglutarate-dependent dioxygenase (GAME31), or a combination thereof, wherein said at least one silencing molecule is operably linked to a promoter; or
providing a first transformed plant, wherein said first transformed plant is transformed with an expression vector comprising at least one polynucleotide which overexpresses at least one protein selected from the group comprising a 3-β-hydroxysteroid dehydrogenase/isomerase (GAME25), a 2-oxoglutarate-dependent dioxygenase (GAME31), or a combination thereof, or
providing a first transformed plant, wherein said first transformed plant is transformed with an expression vector comprising at least one polynucleotide which comprises a mutation in a gene encoding at least one protein selected from the group comprising a 3-β-hydroxysteroid dehydrogenase/isomerase (GAME25), a 2-oxoglutarate-dependent dioxygenase (GAME31), or a combination thereof,
providing a second non-transformed plant;
crossing said first transformed plant of (a) or (b) or (c) with a second plant to generate a hybrid plant, wherein the hybrid plant comprises the expression vector; and
selecting a hybrid plant that has an altered expression of at least one of said genes compared with an unmodified plant. Further, said plant may, in certain embodiments comprise an altered content of at least one cholesterol derived compound selected from the group comprising a steroidal alkaloid or a glycosylated derivative thereof and an unsaturated or saturated steroidal saponin or a glycoside derivative thereof, compared to a corresponding unmodified plant.

In some embodiments, the at least one silencing molecule or said overexpressing polynucleotide is operably linked to a constitutive promoter, an inducible promoter, a tissue-specific promoter, or a developmental-stage specific promoter. In some embodiments, the at least one polynucleotide comprising a mutation is operably linked to a constitutive promoter, an inducible promoter, a tissue-specific promoter, or a developmental-stage specific promoter. In some embodiments, the expression level and/or biological activity of the 3-β-hydroxysteroid dehydrogenase/isomerase (GAME25), or the 2-oxoglutarate-dependent dioxygenase (GAME31), or a combination thereof, provide a biological marker for a plant comprising altered content of at least one cholesterol derived compound selected from the group comprising a steroidal alkaloid or a glycosylated derivative thereof and an unsaturated or saturated steroidal saponin or a glycoside derivative thereof. In some embodiments, the altered content comprises reduced content of an anti-nutritional or toxic steroidal alkaloid or a glycosylated derivative thereof. In some embodiments, the altered content comprises increased content of a steroidal alkaloid or a glycosylated derivative thereof that provides resistance to a plant pathogen, pest, or predator.

In some embodiments, increased content of at least one SA or SGA can be produced in a genetically modified plant. In some embodiments, a method of enhancing the content of at least one steroidal alkaloid or a glycosylated derivative thereof in a plant, comprising
transforming at least one plant cell within said plant with a nucleic acid sequence encoding 3-β-hydroxysteroid dehydrogenase/isomerase (GAME25), 2-oxoglutarate-dependent dioxygenase (GAME31), or a combination thereof, wherein said transforming results in overexpression of said GAME25, GAME31, or a combination thereof, or
transforming at least one plant cell with at least one polynucleotide sequence encoding at least one protein selected from the group comprising 3-β-hydroxysteroid dehydrogenase/isomerase (GAME25), or a 2-oxoglutarate-dependent dioxygenase (GAME31), or any combination thereof, wherein said at least one polynucleotide sequence comprises a mutation in a coding region or a regulatory region;
thereby producing a plant with an enhanced content of said at least one cholesterol derived compound selected from the group comprising a steroidal alkaloid or a glycosylated derivative thereof and an unsaturated or saturated steroidal saponin or a glycoside derivative thereof, compared to a corresponding non-transformed plant.

In some embodiments, increased content of at least one SA of SGA comprises any SA or SGA disclosed herein. In some embodiments, increased content of at least one SA of SGA comprises a subset of SA or SGA disclosed herein. In some embodiments, a sub-set of increased SA of SGA comprises at least one of a α-tomatine, α-tomatine isomer (1 and 2), hydroxytomatine, acetoxytomatine, soladulcidine, β-soladulcine, soladulcine A, an unsaturated or saturated steroidal saponin or a glycoside derivative thereof, a leptin, or a leptinine, or any combination thereof. In some embodiments, increased SA or SGA results in a plant, wherein said plant resistance to at least one plant pathogen, pests or predator is increased, compared to a corresponding non-transformed plant.

In some embodiments, provided herein is a method for selecting plant progenitors for plant breeding, said method comprising a step of determining the expression level of GAME25, GAME31, or a combination thereof, wherein expression levels of GAME25, GAME31, or a combination thereof, below a pre-determined value are predictive of low content of at least one cholesterol derived compound selected from the group comprising a steroidal alkaloid or a glycosylated derivative thereof and an unsaturated or saturated steroidal saponin or a glycoside derivative thereof in the offspring plants.

In some embodiments, provided herein is a method for selecting plant progenitors for plant breeding, said method comprising a step of determining the expression level of GAME25, GAME31, or a combination thereof, wherein expression levels of GAME25, GAME31, or a combination thereof, above a pre-determined value are predictive of high content of at least one cholesterol derived compound selected from the group comprising a steroidal alkaloid or a glycosylated derivative thereof and an unsaturated or saturated steroidal saponin or a glycoside derivative thereof in the offspring plants.

In some embodiments, a method for selecting plant progenitors, comprises a step of
(a) determining the expression level of a gene encoding a 3-β-hydroxysteroid dehydrogenase/isomerase (GAME25), or a 2-oxoglutarate-dependent dioxygenase (GAME31), or a combination thereof, wherein expression levels of said GAME25 gene, or said GAME31 gene, or the combination thereof, is predictive of altered content of at least one cholesterol derived compound selected from the group comprising a steroidal alkaloid or a glycosylated derivative thereof and an unsaturated or saturated steroidal saponin or a glycoside derivative thereof in an offspring plant; or
(b) determining the biological activity of a 3-β-hydroxysteroid dehydrogenase/isomerase (GAME25), or a 2-oxoglutarate-dependent dioxygenase (GAME31), or a combination thereof, wherein biological activity of said GAME25 enzyme, or said GAME31 enzyme, or the combination thereof, is predictive of altered content of at least one cholesterol derived compound selected from the group comprising a steroidal alkaloid or a glycosylated derivative thereof and an unsaturated or saturated steroidal saponin or a glycoside derivative thereof in an offspring plant.

In some embodiments, a method for determining the capacity of a plant to produce steroidal alkaloids or glycosylated derivatives thereof in at least a part of said plant, comprises a step of measuring the expression level of GAME25, or GAME31, or a combination thereof, in at least a part of said plant; or measuring the biological activity of a 3-β-hydroxysteroid dehydrogenase/isomerase (GAME25), or a 2-oxoglutarate-dependent dioxygenase (GAME31), or a combination thereof, in at least a part of said plant; or a combination thereof. In some embodiments, the steroidal alkaloids or glycosylated derivatives thereof are selected from the group comprising: tomatidine, α-tomatine, α-tomatine isomer (1 and 2), α-tomatine isomer 1, α-tomatine isomer 2, hydroxytomatine, acetoxytomatine, dehydrotomatidine, dehydrotomatine, dehydrotomatine isomer 1, dehydrotomatine+4-hexose, acetoxy-hydroxytomatine, acetoxy-hydroxy-dehydrotomatine, tomatidine+4 hexose, esculeosides, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydroesculeosides, dehydroesculeoside A, dehydroesculeoside A+hexose, lycoperosides, leptinine I, leptinine II, leptine I, leptine II, soladulcidine, B3-soladulcine, soladulcine A, an unsaturated or saturated steroidal saponin or a glycoside derivative thereof, solanidine, α-solanine, α-chaconine, solasoidine, α-solasonine, α-solamargine, hydroxysolasonine, and hydroxysolamargine, or any derivatives thereof, or any combination thereof.

In some embodiments, disclosed herein are methods for breeding a plant having an altered expression of at least one gene selected from the group comprising GAME25, GAME31, or a combination thereof, said method comprising providing a first transformed plant, wherein said first transformed plant is transformed with an expression vector comprising a polynucleotide comprising at least one silencing molecule targeted to a nucleic acid sequence encoding at least one protein selected from the group comprising GAME25, GAME31, or a combination thereof, wherein said at least one silencing molecule is operably linked to a promoter; providing a second non-transformed plant; crossing said first and second plants to generate an offspring hybrid plant, wherein the offspring hybrid plant comprises the expression vector; and selecting an offspring hybrid plant that has an altered content of at least one cholesterol derived compound selected from the group comprising a steroidal alkaloid or a glycosylated derivative thereof and an unsaturated or saturated steroidal saponin or a glycoside derivative thereof compared to a corresponding unmodified plant.

In some embodiments, an expression vector is operably linked to a different promoter so that the expression of the silencing molecule can be controlled under different conditions. In another embodiment, the silencing molecule is operably linked to a constitutive promoter. In another embodiment, the silencing molecule is operably linked to an inducible promoter. In another embodiment, the silencing molecule is operably linked to a tissue active or specific promoter. In another embodiment, the silencing molecule is operably linked to a developmental-stage active or specific promoter. When the silencing molecule is linked to a constitutive promoter, changes in expression of a gene will be observed in all tissues and at all times and a broad overview of the effects of the expression of the gene on a plant will be observed. When the silencing molecule is linked to a tissue specific promoter or an inducible promoter or developmental-stage promoter, the expression of the silencing molecule may be turned on or off in a particular tissue such as seed, roots, flowers, leaves, shoots, fruits or stems, during a particular period in development, such as early, middle or late stages in development, or under particular conditions, such as specific environmental or disease stresses. In some embodiments, a plant may be transformed with more than one expression vector. In one embodiment, the at least one silencing molecule is operably linked to a constitutive promoter, an inducible promoter, a tissue-specific promoter, or a developmental-stage specific promoter.

In some embodiments, a hybrid plant is then selected wherein said plant comprises the desired expression of GAME25, or GAME31, or a combination thereof. In some embodiment, the expression level of GAME25, GAME31, or a combination thereof, provide a marker for a plant comprising altered content of at least one cholesterol derived compound selected from the group comprising a steroidal alkaloid or a glycosylated derivative thereof and an unsaturated or saturated steroidal saponin or a glycoside derivative thereof. In some embodiments, use of GAME25 levels, or GAME31 levels, or a combination thereof as a marker provides the ability to select a plant breed to comprise a reduced content of an anti-nutritional or toxic cholesterol derived compound selected from the group comprising a steroidal alkaloid or a glycosylated derivative thereof and an unsaturated or saturated steroidal saponin or a glycoside derivative thereof, as compared to a control plant. In some embodiments, use of GAME25 levels, or GAME31 levels, or a combination thereof as a marker provides the ability to select a plant breed to comprise improved resistance to a plant pathogen, pest, or predator, as compared to a control plant.

In one embodiment, the method combines genomic and plant breeding techniques. In one embodiment, the method alters the expression level or levels of GAME25, GAME31, or a combination thereof. Expression levels may be altered constitutively, or altered selectively to monitor tissue specific expression, inducible expression, developmental-stage specific expression or the like in a high-throughput manner. In another embodiment, the expression of these known genes, or the enzymes they encode, act as markers for breeding plants having SA or SGA derivatives comprising beneficial properties, for example SA or SGA derivatives that provide increased resistance to pathogens, pests, or predators, plants having a decreased anti-nutritional content or decreased toxins, or any combination thereof.

In some embodiments, transformation techniques including breeding through transgene editing, use of transgenes, use of transient expression of a gene or genes, or use of molecular markers, or any combination thereof, may be used in the breeding of a plant having an altered expression. If transformation techniques require use of tissue culture, transformed cells may be regenerated into plants in accordance with techniques well known to those of skill in the art. The regenerated plants may then be grown and crossed with the same or different plant varieties using traditional breeding techniques to produce seed, which are then selected under the appropriate conditions.

In some embodiments, an offspring plant comprises decreased anti-nutritional contents or decreased toxins compared to at least one of the progenitor plants. In some embodiments, an offspring plant comprises improved resistance to a plant pathogen, pest, or predator compared to at least one of the progenitor plants.

In one embodiment, a plant as disclosed herein comprises a Solanaceae crop plant. In some embodiments, a Solanaceae crop plant is selected from the group comprising Solanum lycopersicum, Solanum pennellii, Solanum tuberosum, Solanum chacoense, Capiscum annuum, and Solanum melongena. In some embodiments, a Solanaceae plant is selected from the group comprising ground cherry, eggplant, potato, tomato, pepper, bell pepper, cayenne pepper, chili pepper, pimiento, tabasco pepper, tobacco, and bittersweet. In some embodiments, a Solanaceae plant comprises any Solanaceae plant that produces a steroidal alkaloid or a glycosylated derivative thereof, or an unsaturated or saturated steroidal saponin or a glycoside derivative thereof, or any combination thereof.

A skilled artisan would appreciate that plant breeding can be accomplished through many different techniques ranging from simply selecting plants with desirable characteristics for propagation, to methods that make use of knowledge of genetics and chromosomes, to more complex molecular techniques.

A skilled artisan would appreciate that the term “hybrid plant” may encompass a plant generated by crossing two plants of interest, propagating by seed or tissue and then growing the plants. When plants are crossed sexually, the step of pollination may include cross pollination or self-pollination or back crossing with an untransformed plant or another transformed plant. Hybrid plants include first generation and later generation plants. Disclosed herein is a method to manipulate and improve a plant trait, for a non-limiting example—increasing plant resistance, decreasing anti-nutritional properties in a plant, or decreasing toxins in a plant, or any combination thereof.

### Biomarkers

A skilled artisan would appreciate that the term “biomarker” comprises any measurable substance in an organism whose presence is indicative of a biological state or a condition of interest. In some embodiments, the presence of a biomarker is indicative of the presence of a compound or a group of compounds of interest. In some embodiments, the concentration of a biomarker is indicative of the concentration of a compound or a group of compounds of interest. In some embodiments, the concentration of a biomarker is indicative of an organism phenotype.

The enzymes GAME25 and GAME31, are hereby disclosed to have an essential role in the biosynthesis of steroidal alkaloids found in Solanaceae plants. Thus, in some embodiments, the expression levels of GAME25, GAME31, or a combination thereof, are indicative of the capacity of a plant to produce steroidal alkaloids or glycosylated derivatives thereof.

In some embodiments, disclosed herein is a method for determining the capacity of a plant to produce steroidal alkaloids or glycosylated derivatives thereof in at least a part of said plant, the method comprising a step of determining the expression level of GAME25, or GAME31, or a combination thereof, in at least a part of said plant. In some embodiments, said expression level is compared to a pre-determined value. In some embodiments, expression levels above a pre-determined value indicate a high capacity of a plant to produce steroidal alkaloids or glycosylated derivatives thereof. In some embodiments, expression levels below a pre-determined value indicate a low capacity of a plant to produce steroidal alkaloids or glycosylated derivatives thereof.

In some embodiments, the expression level of GAME25, or GAME31 are determined in at least a part of the plant, wherein said part of said plant is selected from the group comprising: a peel, a leaf, a bud, a petal, a root, an edible part of the plant, and any combination thereof. In some embodiments, the plant is a Solanaceae crop plant. In some embodiments, the steroidal alkaloids or glycosylated derivatives thereof are produced in at least a part of the plant, wherein said part of said plant is selected from the group comprising: a peel, a leaf, a bud, a petal, a root, an edible part of the plant, and any combination thereof. In some embodiments, a leaf comprises a young leaf. In some embodiments, a leaf comprises a mature leaf.

In some embodiments, said steroidal alkaloids or glycosylated derivatives thereof are selected from the group comprising: tomatidine, α-tomatine, α-tomatine isomer (1 and 2), hydroxytomatine, acetoxytomatine, acetoxy-hydroxytomatine, tomatidine+4 hexose, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydroesculeosides, leptinine I, leptinine II, leptine I, leptine II, and hydroxysolamargine, or any derivatives thereof, or any combination thereof.

In some embodiments, disclosed herein is a method for determining the capacity of a plant to produce tomatidine in at least a part of said plant, said method comprising a step of determining the expression level of GAME25, or GAME31, or a combination thereof, in at least a part of said plant. In some embodiments, disclosed herein is a method for determining the capacity of a plant to produce α-tomatine in at least a part of said plant, said method comprising a step of determining the expression level of GAME25, or GAME31, or a combination thereof, in at least a part of said plant. In some embodiments, disclosed herein is a method for determining the capacity of a plant to produce hydroxytomatine in at least a part of said plant, said method comprising a step of determining the expression level of GAME25, or GAME31, or a combination thereof, in at least a part of said plant. In some embodiments, disclosed herein is a method for determining the capacity of a plant to produce acetoxytomatine in at least a part of said plant, said method comprising a step of determining the expression level of GAME25, or GAME31, or a combination thereof, in at least a part of said plant.

In some embodiments, disclosed herein is a method for determining the capacity of a plant to produce acetoxy-hydroxytomatine in at least a part of said plant, said method comprising a step of determining the expression level of GAME25, or GAME31, or a combination thereof, in at least a part of said plant. In some embodiments, disclosed herein is a method for determining the capacity of a plant to produce tomatidine+4 hexose in at least a part of said plant, said method comprising a step of determining the expression level of GAME25, or GAME31, or a combination thereof, in at least a part of said plant. In some embodiments, disclosed herein is a method for determining the capacity of a plant to produce esculeoside A in at least a part of said plant, said method comprising a step of determining the expression level of GAME25, or GAME31, or a combination thereof, in at least a part of said plant. In some embodiments, disclosed herein is a method for determining the capacity of a plant to produce esculeoside A+hexose in at least a part of said plant, said method comprising a step of determining the expression level of GAME25, or GAME31, or a combination thereof, in at least a part of said plant.

In some embodiments, disclosed herein is a method for determining the capacity of a plant to produce esculeoside B in at least a part of said plant, said method comprising a step of determining the expression level of GAME25, or GAME31, or a combination thereof, in at least a part of said plant. In some embodiments, disclosed herein is a method for determining the capacity of a plant to produce acetoxyesculeoside B in at least a part of said plant, said method comprising a step of determining the expression level of GAME25, or GAME31, or a combination thereof, in at least a part of said plant. In some embodiments, disclosed herein is a method for determining the capacity of a plant to produce demissidine in at least a part of said plant, said method comprising a step of determining the expression level of GAME25, or GAME31, or a combination thereof, in at least a part of said plant. In some embodiments, disclosed herein is a method for determining the capacity of a plant to produce demissine in at least a part of said plant, said method comprising a step of determining the expression level of GAME25, or GAME31, or a combination thereof, in at least a part of said plant. In some embodiments, disclosed herein is a method for determining the capacity of a plant to produce dehydrosolasodine in at least a part of said plant, said method comprising a step of determining the expression level of GAME25, or GAME31, or a combination thereof, in at least a part of said plant.

In some embodiments, disclosed herein is a method for determining the capacity of a plant to produce hydroxy-dehydrotomatine in at least a part of said plant, said method comprising a step of determining the expression level of GAME25, or GAME31, or a combination thereof, in at least a part of said plant. In some embodiments, disclosed herein is a method for determining the capacity of a plant to produce acetoxy-hydroxy-dehydrotomatine in at least a part of said plant, said method comprising a step of determining the expression level of GAME25, or GAME31, or a combination thereof, in at least a part of said plant. In some embodiments, disclosed herein is a method for determining the capacity of a plant to produce dehydroesculeosides in at least a part of said plant, said method comprising a step of determining the expression level of GAME25, or GAME31, or a combination thereof, in at least apart of said plant. In some embodiments, disclosed herein is a method for determining the capacity of a plant to produce leptinine I in at least a part of said plant, said method comprising a step of determining the expression level of GAME25, or GAME31, or a combination thereof, in at least a part of said plant.

In some embodiments, disclosed herein is a method for determining the capacity of a plant to produce leptinine II in at least a part of said plant, said method comprising a step of determining the expression level of GAME25, or GAME31, or a combination thereof, in at least a part of said plant. In some embodiments, disclosed herein is a method for determining the capacity of a plant to produce leptine I in at least a part of said plant, said method comprising a step of determining the expression level of GAME25, or GAME31, or a combination thereof, in at least a part of said plant. In some embodiments, disclosed herein is a method for determining the capacity of a plant to produce leptine II in at least a part of said plant, said method comprising a step of determining the expression level of GAME25, or GAME31, or a combination thereof, in at least a part of said plant. In some embodiments, disclosed herein is a method for determining the capacity of a plant to produce hydroxysolamargine in at least a part of said plant, said method comprising a step of determining the expression level of GAME25, or GAME31, or a combination thereof, in at least a part of said plant.

In some embodiments, disclosed herein is a method for selecting a plant with reduced content of an anti-nutritional or toxic cholesterol derived compound selected from the group comprising a steroidal alkaloid or a glycosylated derivative thereof and an unsaturated or saturated steroidal saponin or a glycoside derivative thereof, as compared to a control plant, said method comprising a step of determining the expression level of GAME25, or GAME31, or a combination thereof, in said plant, wherein expression level below a pre-determined value indicates reduced content of an anti-nutritional or toxic cholesterol derived compound selected from the group comprising a steroidal alkaloid or a glycosylated derivative thereof and an unsaturated or saturated steroidal saponin or a glycoside derivative thereof.

In some embodiments, disclosed herein is a method for selecting a plant with improved resistance to a plant pathogen, pest, or predator, as compared to a control plant, said method comprising a step of determining the expression level of GAME25, or GAME31, or a combination thereof, in said plant, wherein expression level above a pre-determined value indicates improved resistance to a plant pathogen, pest, or predator.

In some embodiments, disclosed herein is a method for selecting a plant with altered content of steroidal alkaloids or glycosylated derivatives thereof, as compared to a control plant, said method comprising a step of determining mutations in GAME25, or GAME31, or a combination thereof, in said plant, wherein mutations indicate altered content of steroidal alkaloids or glycosylated derivatives thereof. In some embodiments, disclosed herein is a method for selecting a plant with decreased content of steroidal alkaloids or glycosylated derivatives thereof, as compared to a control plant, said method comprising a step of determining mutations in GAME25, or GAME31, or a combination thereof, in said plant, wherein mutations indicate decreased content of steroidal alkaloids or glycosylated derivatives thereof.

Further, one skilled in the art would appreciate that the term “comprising” used throughout is intended to mean that the genetically modified plants disclosed herein, and methods of altering expression of genes, and altering production of SA and/or SGA within these genetically modified plants includes the recited elements, but not excluding others which may be optional. “Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited.

The following examples are presented in order to more fully illustrate embodiments described herein above. They should in no way be construed, however, as limiting.

### EXAMPLES

### Example 1

Expression of GAME25, a Short-Chain Dehydrogenases/Reductases Family Member Accords with the Accumulation of the Typical Green Tissue Steroidal Glycoalkaloids

Objective: GAME9 AP2-type transcription factor is associated with the regulation of steroidal glycoalkaloids (SGAs) biosynthesis in tomato and potato. Transcriptome analysis of GAME9 overexpressing (GAME9-Ox) and GAME9 silenced (GAME9-RNAi) tomato lines revealed a concise set of 27 genes that were common among up- and down-regulated genes. Among these genes, a putative 3-β-hydroxysteroid dehydrogenase/isomerase was identified and termed GAME25 (SEQ ID NO: 1). To understand the role of this gene, the expression pattern of GAME25 was examined in 14 different tomato tissue types.

### Methods

### Plant Materials

Tomato (Solanum lycopersicum) cultivar (cv). MicroTom plants were grown in a climate-controlled greenhouse at 24° C. during the day and 18° C. during the night, with natural light.

The database SolGenomics (Femandez-Pozo N, Menda N, Edwards J D, Saha S, Tecle I Y, Strickler S R, Bombarely A, Fisher-York T, Pujar A, Foerster H, Yan A, Mueller L A. The Sol Genomics Network (SGN) from genotype to phenotype to breeding. (2015) Nucleic Acids Res. Volume 43 (Database issue):D1036-41) was used for searching and analyzing sequences.

Quantitative Real-Time PCR (qPCR) Analysis

Total RNA was isolated from tomato (leaf, green fruit, breaker and red fruits) tissues using the Trizol method (Sigma-Aldrich). DNase I (Sigma-Aldrich)-treated RNA was reverse transcribed using a high-capacity cDNA reverse transcription kit (Applied Biosystems). Gene-specific oligonucleotides were designed with Primer Express 3 software (Applied Biosystems). The TIP41 gene was used as a reference gene for tomato samples.
GAME25 qRT Forward primer: GAAGCAATTTACGGTAATGGACAC (SEQ ID NO: 4)
GAME25 qRT Reverse primer: GAACTTAGTCCACCATCAACAGC (SEQ ID NO: 5)

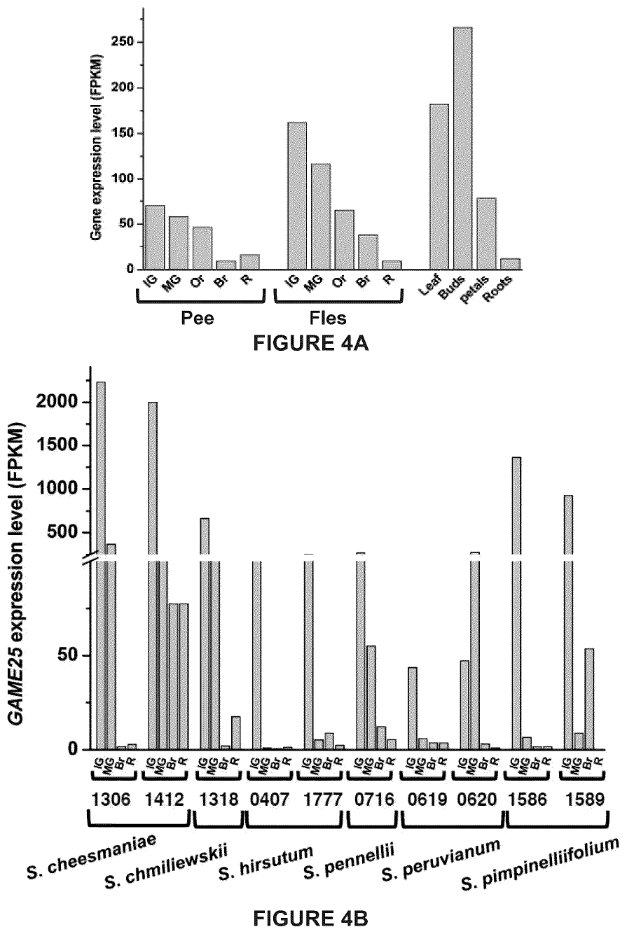

Results: GAME25 showed higher expression in flower buds and young leaves compared to fruit specific tissues. During fruit development highest expression of GAME25 was observed at the early stage of fruit development, i.e. immature green (IG) fruit (FIG. 4A).

The expression pattern of GAME25 was similar to the profile of steroidal glycoalkaloids (SGAs) α-tomatine and dehydrotomatine that accumulate predominantly in green tissues (leaves, buds, peel and flesh of immature green fruit). For example, the reduced transcript levels of the GAME25 gene observed at later time points of fruit development correlates with the typical pattern of reduction of α-tomatine and dehydrotomatine levels observed during fruit development and ripening. Moreover, GAME25 expression in pattern during tomato fruit developmental stages was similar to the one observed in wild tomato accessions (FIG. 4B).

Conclusion: The association between GAME25 transcript level and accumulation of α-tomatine and dehydrotomatine SGAs in the tissues examined suggests a possible role of GAME25 in steroidal glycoalkaloid metabolism.

### Example 2

### Characterization of GAME25 as a Short-Chain Dehydrogenases/Reductases Family Member

Objective: To characterize GAME25 structurally and functionally.

### Methods

### Phylogenetic Analysis

GAME25 and its homologous sequences from various plants were obtained using the BLASTP program. Additionally, literature search was performed for known SDR family proteins that partake in secondary metabolism among various plant species. Amino acid sequences were aligned using ClustalOmega. The Maximum Likelihood tree was inferred in MEGA6 using 1000 bootstrap replications. Evolutionary distances are in units of number of amino acid substitutions per site. All positions containing gaps and missing data were eliminated. The amino acid sequences used in the phylogenetic analysis are provided in below.

S. lycopersicum GAME25:
(SEQ ID NO: 3)
MANKLRLEGKVAIITGAASGIGEASARLFVEHGARVVVADIQDELGQKV
VDSIGSDKASYRHCDVTDEKQVEETVAYAVEKYGTLDIMFSNVGTLNFCSVLD
MDVLAFDETMAINVRGSALAVKHAAKVMVDKKIRGSIICNASLEGILAGAASL
AYIASKHAVVGIIKAAARELGPHGIRVNGVSPYGIATPLVTKAYGLDAALLEEAI
YGNGHLKGVKLSTMHVAQSALFLASDESAYTSGQNLAVDGGLSSILKLQ
S. pennellii GAME25:
(SEQ ID NO: 12)
MANKLRLEGKVAIITGAASGIGEASARLFVEHGARVVVADIQDELGQKV
VDSIGADKASYRHCDVTDEKQVEETVAYAVEKYGTLDIMFSNVGTLNFCSVLD
MDVMAFDETMAINVRGSALAVKHAAKVMVDKKIRGSIICNASLEGILAGAASL
AYIASKHAVVGIIKAAARELGPHGIRVNGVSPYGIATPLVCKAYGLDAALLEEAI
YGNGHLKGVKLSTMHVAQSALFLASDESAYTSGQNLAVDGGLSSILKLQ
S. tuberosum GAME25:
(SEQ ID NO: 15)
MANKLRLEGKVAIITGAASGIGEASARLFAEHGARIVVADIQDELGLKV
VESIGADKASYRHCDVTDEKQVEDTVAYTVEKYGTLDIMFSNVGTLNFCSVLD
MDVMVFDKTMAINARGSALAVKHAARFMVDKKIRGSIICNASLDGIVAGATSL
AYIASKHAVVGIVKAAARDLGPYGIRVNGVSPYGIATPLVCKAYGLDAGPLEA
AIYGNGNLKGVRLSTMHVAQSALFLASDESAYTSGQNLAVDGGLSSILKVQ
S. lycopersicum 3βHSD:
(SEQ ID NO: 66)
MASKLRLEGKVAIITGGASGIGEASARLFVQHGARVVVADIQDELGLQV
VQSIGIHKATYRHCDVTDEKQVEDTVAYAVQKYATLDIMFSNVGTLNFCSVLD
MDMTAFDETMTVNVRGSALAVKHAARVMVDKKIRGSIICNVSLEGILAGAASL
AYIASKHAVVGIVKAAARELGPYGIRVNGVSPYGIATPLVCKAYGLDAAPLEA
AINGNANLKGVTLSTMHVAQSALFLASDESAYTSGQNLAVDGGLSSILKLQ
S. pennellii 3βHSD:
(SEQ ID NO: 67)
MASKLRLEGKVAIITGGASGIGEASARLFVQHGARVVVADIQDELGLQV
VQSIGIHKATYRHCDVTDEKQVEDTVAYAVQKYATLDVMFSNVGTLNFCSVL
DMDMTAFDETMTVNVRGSALAVKHAARVMVDKKIRGSIICNVSLEGILAGAAS
LAYIASKHAVVGIVKAAARELGPYGIRVNGVSPYGIATPLVCKAYGLDAAPLEA
AINGNANLKGVTLSTMHVAQSALFLASDESAYTSGQNLAVDGGLSSILKLQ
C. annuum 3βHSD:
(SEQ ID NO: 68)
LEGKVAVITGAASGIGEASARLFVEHGARVVIADIQDELGLQIAASIGTD
KASYIHCDVTDEKQVEEAVAYAVENSVLDLDVKAFDETMVINARGSAVAVKH
AARVMVEKKIRGSIICTASLEGILAGAASLAYVSSKHAVVGLVKAAARELGVH
GIRVNGVSPYGIATPLVCKAYGLDAGPLETAIYGNAHLKGVTLSTMHVAQAAL
FLASDESAYISGQNLAVDGGLSSILKLE
N. benthamiana 3βHSD:
(SEQ ID NO: 69)
MANKLRLEGKVAVITGGASGIGEATARLFVEHGARVVIADIQDELGLQV
VASIGTDKASYRHCDVTDENKVEETVAYAVEKYGTLDIMFSNVGTLNFCSVLD
IDVTAFDKTMALNVRGTALAVKHAARVMVAKQVKGSIICNASIEAILAGAASL
AYVASKHAVVGIVKAAARELGLHGIRVNGVSPYGIATPLVCKAYGCEDAASLE
AGISVNAHLKGVTLSTEHIAQAALFLASDESAYISGHNLAVDGGLTSMLKLSY
S. melongena ADH1:
(SEQ ID NO: 70)
MANKLKLEGKVAVITGGASGIGEESARLFVEHGARVVIADIQDDLGLEV
VTSIGADKACYRHCDVSEEKQVKETVAYAVEKYGTLDIMFSNAGTLGTLGSVL
EMDMTAFDMTMAVNMRGSALAVKHAARVMVANKIRGSIICTASVEAILAGAA
PLAYVASKHAILGVMKAAARELGQYGIRVNCVSPYGIATPLVCKAYHSDAGSL
EASIYERAHLKGITLSTKHIANASLFLASDESAYVSGHNLAVDGALSSIMS
C. annuum ADH1:
(SEQ ID NO: 71)
MIFFFCGTRLEGKIAIITGAASGIGEASARLFVEHGAHVIIADIQDELGLQV
VSSIGTDKACYRHCDVTDEKQVEETVAYAVEKYGTLDIMFSNAGMLGTFGSLL
DMDVKEFDLTIAVNTRGAALAVKHAARVMVAKNIRGSIICTASVESILAGAAPL
AYIASKHGILGVVKAAARELGKNGIRVNCVSPFGIATPMVCKSYGAEASYIETS
VGGHANLKGVSLTTKHIAEAALFLASEESAYISGQNLAVDGGLSAIMRLD
S. melongena ADH2:
(SEQ ID NO: 72)
MANKLRLEGKVAVITGGASGIGEASARLFVEHGARVVIADIQDELSLQV
VSSIGGDKACYRRCDVSDEKQVEETVAYAVEKYGTLDIMFSNAGILGSFGSLLE
MDMTAFDRIMAVNTRGAALAVKHAARVMVANKIRGSIICTASVEAILAGEASL
AYIASKHAILGVVKAAARDLGQYGIRVNCVSPYGIATPMVCKSIGADAATIEAR
ICGNANLKGVSLNTKHIAEAALFLGSDESAYVSAHNLAVDGGLSSIMKLN
S. tuberosum ADH1:
(SEQ ID NO: 73)
MANKLRLEGKVAVITGGASGIGEATARLFVEHGAHVVIADIQDELALQV
VSSIGSDNVCYRRCDVTDEKQVDETVAFAVQKYGTLDIMFSNAGILGSSGSLLE
MDMAVFDRTMAVNTRGAALAVKHAAKVMVAKKIRGSIICTASVESILAGAAS
LAYIASKHAVLGVVKAAARELGQHGIRVNCVSPFGVATPMVCKSFGADAAAM
EATIRGNANLKGVSLTTMHIAEAALFLASDESAYISAHNLAIDGGLSSIMKINVN
S. lycopersicum ADH1:
(SEQ ID NO: 74)
MANKLRLEGKVAVITGGASGIGEAAARLFVEHGARVVIADIQDELALQV
ASSIGSDNVCYQRCDVSDEKQVNETVAFAVEKYGTLDIMFSNAGILNPFESILE
MDMTVFDRTIAVNARGAALAVKHAARVMVANKIRGSIICTASVESILAGAAPL
AYIASKHAVLGVVKAAARELGQHGIRVNCVSPFGIATPMVCKSFGADAAAIEA
KICGNANLKGVSLTTMHIAEAALFLASDESAYISAQNLAVDGGLSSMMKLM
S. pennellii ADH1:
(SEQ ID NO: 75)
MANKLRLEGKVAVITGGASGIGEAAARLFVEHGARVVIADIQDELALQV
ASSIGSVNVCCRRCDVSDEKQVNETVAFAVEKYGTLDIMFSNAGILNPFESILEM
DMTVFDRTIAVNARGAALAVKHAARVMVANKIRGSIICTASVESILAGAAPLA
YIASKHAVLGVVKAAARELGQHGIRVNCVSPFGIATPMVCKSFGADAAAIEAKI
CGNANLKGVSLTTMHIAEAALFLASDESAYISAQNLAVDGGLSSMMKLM
N. benthamiana ADH1:
(SEQ ID NO: 76)
MANKRRLEGKVAVITGAASGIGEATARLFVEHGARVVIADIQDELGHQV
VASIGTDKASYRHCDVTDEKQVEDTVVYTVEKYGTLDIMFSNAGTIGTLGSILD
MDMTVFDRTMAINARGSALAVKHAARVMVTKKIQGSIICTASLEATLAGAAPL
AYVTSKHAILGVVKAAARELGQHGIRVNCVSPYGIATPMVCKTFGGDAAPIEAS
ISGNANLKGITLSTKHIAEAALFLASDESAYVSAHNLAVDGGLSSIMKLD
N. benthamiana ADH2:
(SEQ ID NO: 77)
MANKLRLEGKVAVITGAASGIGEATARLFVEHGARVVIADIQDELGHQV
VASIGTDKASYRHCDVTDEKQVEDTVVYAVEKYGTLDIMFSNAGTIGTLSSILD
MDMTVFDRTMAINARGSALAVKHAARIMVTKKIQGSIICTASLEAILAGAAPLA
YVASKHAILGVVKAAARELGQHGIRVNCVSPYGIATPMVCKTFGGDAAPIEASI
SGNANLKGITLSTKHIAEAALFLASDESAY
A. thaliana ADH:
(SEQ ID NO: 78)
MSGKRLDGKIVIITGGASGIGAESVRLFTEHGARVVIVDVQDELGQNVA
VSIGEDKASYYHCDVTNETEVENAVKFTVEKYGKLDVLFSNAGVIEPFVSILDL
NLNELDRTIAINLRGTAAFIKHAARAMVEKGIRGSIVCTTSVAAEIAGTAPHGYT
TSKHGLLGLIKSASGGLGKYGIRVNGVAPFGVATPLVCNGFKMEPNVVEQNTS
ASANLKGIVLKARHVAEAALFLASDESAYVSGQNLAVDGGYSVVKP
M. truncatula ADH:
(SEQ ID NO: 79)
MSRKRLEGKVAIVTGGASGIGAETAKTFVENGAFVVIADINDELGHQVA
TSIGLDKVSYHHCDVRDEKQVEETVAFALEKYGTLDIMFSNAGIEGGMSSSILEF
DLNEFDNTMAINVRGSLAAIKHAARFMVERKIRGSIICTASVAASVAGNRGHDY
VTSKHGLLGLVRSTCGELGAYGIRVNSISPYGVATPLACRALNMEMSKVEANM
KDSANLKGITLKATHIAEAALFLASEESAYISGHNLVVDGGFSVINSCVPTTIKK
C. annuum ADH2:
(SEQ ID NO: 80)
MAVVMQKLKGKVAIVTGGASGIGEATVRLFAEHGARAVVIADIQDEKG
RAVAESIPLQVCSYVHCDVSDENQVKGLVDWTVKKYGQLDIMFSNAGTVGNS
GQKVLDLDLSEFDRVIRVNARGMAACVKHAARAMVEQGGRGSIICTGSVGAS
KGAAWRTDYTMSKHAVLGLVTSASRQLGKYGIRVNSISPSAVMTPLMSSAEAE
TSMKVLKMYGPLTSLKGITLTVKHLADAVLFLASDDSAFVNGHDLLVDGGLLH
LPDPMSSL
S. tuberosum ADH2:
(SEQ ID NO: 81)
MAEVTQKLKGKVAIVTGGASGIGEATARLFAQHGARAVVIADIQDGKG
RAVAVSIPSQICSYVQCDVSDENQVKAMVDWTVQKYGQLDIMFSNAGVVGNS
GQKVLDLDLSEFDRVMNVNARGMAACVKHAARAMVDKRVRGSIICTGSIGAS
RGGAWRTDYIMSKHAVLGLVRSACRQLGEYGIRVNSISPSAVMTPLMISAEPEV
SMKSLKRYGPQTSLKGITLTVKHLAEAALFLASDDSAFSSRSNEFIVKQREQPNL
SLFFF
S. melongena ADH3:
(SEQ ID NO: 82)
MSAITQKLNGKVAIVTGGASGIGEATVRLFAQHGARAVVIADIQDEKGR
AVAQSIPSQICIYVKCDVSDENQVKSMVDWTVQQYGQLDIMFSNAGTVGNSGQ
KILDLDLSEFDRVMNVNARGMAACVKHAARAMVEKRVRGSIICTGSIAASRAG
AWRTDYAMSKHAVLGLMRSASRQLGEYGIRVNSISPSAVMTPLMISAEAEASM
RVLKMYGSVTSLKGITLTVKHLADAVLFLASDDSVFVSGHDLAVDGGLISLPDPMSSL
O. sativa MIS2:
(SEQ ID NO: 83)
MFTAMHRILSRGRRTPAASSSSVTAFATASDSQRLAGKVAVITGGASGIG
RATAEEFVRNGAKVILADVQDDLGHAVAAELGADAASYARCDVTDEAQVAA
AVDLAVARHGRLDVVFNNAGIPGDLTPTPVGALDLADFDRVMAVNTRAVVAG
VKHAARVMVPRRRGSIICTASTAGVIGGVAVPHYSVSKAAVLGLVRAVAGEM
ARSGVRVNAISPNYIWTPMAAVAFARWYPSRSADDHRRIVENDINEMDGVTLE
AEDVARAAVFLASDEAKYVNGHNLVVDGGYTVGKVPNMPVPDGH
O. sativa MIS3:
(SEQ ID NO: 84)
MFRAAQLLLRETNRALGAATSPAGFVSGFSTASNSAQRLAGKVAVITGG
ASGIGKATAKEFIENGAKVIMADVQDDLGHSTAAELGPDASYTRCDVTDEAQV
AAAVDLAVKRHGHLDILYNNAGVMGAMPQDDMASVDLANFDRMMAINARA
ALVGIKHAARVMSPRRSGVILCTASDTGVMPMPNIALYAVSKATTIAIVRAAAE
PLSRHGLRVNAISPHGTRTPMAMHVLSQMYPGVSKDDLEKMADAAMDAGEV
MEPKYVARAALYLASDEAKYVNGHNLVVDGGFTSHKGSDTRLN
A. thaliana ABA2:
(SEQ ID NO: 85)
MSTNTESSSYSSLPSQRLLGKVALITGGATGIGESIVRLFHKHGAKVCIVD
LQDDLGGEVCKSLLRGESKETAFFIHGDVRVEDDISNAVDFAVKNFGTLDILIN
NAGLCGAPCPDIRNYSLSEFEMTFDVNVKGAFLSMKHAARVMIPEKKGSIVSLC
SVGGVVGGVGPHSYVGSKHAVLGLTRSVAAELGQHGIRVNCVSPYAVATKLA
LAHLPEEERTEDAFVGFRNFAAANANLKGVELTVDDVANAVLFLASDDSRYIS
GDNLMIDGGFTCTNHSFKVFR
O. sativa MS3:
(SEQ ID NO: 86)
MAGSSYGDVHESARKLVGKVALITGGASGIGECTARLFVKHGAQVVVA
DIQDEAGARLCAELGSATASYVRCDVTSEDDVAAAVDHAVARYGKLDVMFN
NAGIGGAACHSILESTKADFDRVLAVNLTGPFLGTKHAARVMVAAGRGGCIIG
TASLASAVAGTASHAYTCAKRALVGLTENAAAELGRHGIRVNCVSPAAAATPL
ATGYVGLEGEAFEAAMEAVANLKGVRLRVEDIAAAVLFLASDDARYVSGHNL
LIDGGCSIVNPSFGIFKD
O. sativa MS1:
(SEQ ID NO: 87)
MAAGSSHVSADARKLVGKVAVITGGASGIGACTARLFVKHGARVVVA
DIQDELGASLVAELGPDASSYVHCDVTNEGDVAAAVDHAVARFGKLDVMFNN
AGVSGPPCFRMSECTKEDFERVLAVNLVGPFLGTKHAARVMAPARRGSIISTAS
LSSSVSGAASHAYTTSKHALVGFTENAAGELGRHGIRVNCVSPAGVATPLARA
AMGMDDEAIEAIMANSANLKGAGALKADDIAAAALFLASDDGRYVSGQNLRV
DGGLSVVNSSFGFFRD
O. sativa MS2:
(SEQ ID NO: 88)
MAGSSHVSADARKLVGKVAVITGGASGIGACTARLFVKHGARVVVADI
QDELGASLVAELGPDASSYVHCDVTNEGDVAAAVDHAVATFGKLDVMFNNA
GVTGPPCFRITESTKEDFERVLAVNLIGPFLGTKHAARVMAPARRGSIISTASLSS
SVSGTASHAYTTSKRALVGFTENAAGELGRHGIRVNCVSPAAVATPLARAAMG
MDMDDETIEAIMEKSANLKGVGLKVDDIAAAALFLASDDGRYVSGQNLRVDG
GVSVVNSSFGFFRD
A. thaliana 11/17-βHSD:
(SEQ ID NO: 89)
MELINDFLNLTAPFFTFFGLCFFLPPFYFFKFLQSIFSTIFSENLYGKVVLIT
GASSGIGEQLAYEYACRGACLALTARRKNRLEEVAEIARELGSPNVVTVHADV
SKPDDCRRIVDDTITHFGRLDHLVNNAGMTQISMFENIEDITRTKAVLDTNFWG
SVYTTRAALPYLRQSNGKIVAMSSSAAWLTAPRMSFYNASKAALLSFFETMRIE
LGGDVHITIVTPGYIESELTQGKYFSGEGELIVNQDMRDVQVGPFPVASASGCA
KSIVNGVCRKQRYVTEPSWFKVTYLWKVLCPELIEWGCRLLYMTGTGMSEDT
ALNKRIMDIPGVRSTLYPESIRTPEIKSD
A. thaliana TR:
(SEQ ID NO: 90)
METDKRWSLAGKTALVTGGTRGIGRAVVEELAKFGAKVHTCSRNQEEL
NACLNDWKANGLVVSGSVCDASVRDQREKLIQEASSAFSGKLNILINNVGTNV
RKPTVEYSSEEYAKIMSTNLESAFHLSQIAHPLLKASGVGSIVFISSVAGLVHLSS
GSIYGATKGALNQLTRNLACEWASDNIRTNCVAPWYIKTSLVETLLEKKEFVEA
VVSRTPLGRVGEPEEVSSLVAFLCLPASSYITGQVISVDGGFTVNGFSYAMKP
Z. mays SDR:
(SEQ ID NO: 91)
MDAAAAAASPTSKRIALVTGGNKGIGLETCRQLASRGVRVVLTARNEA
RGLEAVERVRCARGDAEVYFHQLDVTDPCSAARLADFVRDQFGRLDILINNAG
ISGVHRDPVLSAAVKDKVDGMDVNQRVEWNIKENSKETYEEAVQCMKTNYYG
AKLVTEALLPLLQLSSSGRIVNVSSGFGLLRNFNSEDLRKEFEDIDNLTESRLEEL
MDKFLEDFKANLVEEHGWPTGGSSAYKVVKAALNAYTRILAKKYPTLRINCLT
PGYVKTDISMHMGVLTLEEGARNPVKVALLPDDGPTGAYFDLNGEASFV
A. thaliana SR:
(SEQ ID NO: 92)
MAEETPRLFNGFCRYAVVTGANRGIGFEICRQLASEGIRVVLTSRDENRG
LEAVETLKKELEISDQSLLFHQLDVADPASITSLAEFVKTQFGKLDILVNNAGIG
GIITDAEALRAGAGKEGFKWDEIITETYELTEECIKINYYGPKRMCEAFIPLLKLS
DSPRIVNVSSSMGQLKNVLNEWAKGILSDAENLTEERIDQVINQLLNDFKEGTV
KEKNWAKFMSAYVVSKASLNGYTRVLAKKHPEFRVNAVCPGFVKTDMNFKT
GVLSVEEGASSPVRLALLPHQETPSGCFFSRKQVSEF
P. bracteatum SR:
(SEQ ID NO: 93)
MPETCPNTVTKMRCAVVTGGNKGIGFEICKQLSSSGIMVVLTCRDVTRG
LEAVEKLKNSNHENVVFHQLDVTDPITTMSSLADFIKARFGKLDILVNNAGVAG
FSVDADRFKAMISDIGEDSEEVVKIYEKPEAQELMSETYELAEECLKINYYGVK
SVTEVLLPLLQLSDSPRIVNVSSSTGSLKYVSNETALEILGDGDALTEERIDMVV
NMLLKDFKENLIETNGWPSFGAAYTTSKACLNAYTRVLAKKIPKFQVNCVCPG
LVKTEMNYGIGNYTADEGAKHVVRIALFPDDGPSGFFYDCSELSAF
Z. mays LCR:
(SEQ ID NO: 94)
MSTGGRKMRTACVTGGNGYIASALIKVLLEKGYAVKTTVRNPDDMEK
NSHLKDLQALGSLEVFRADLDEDGSFDDAVAGCDYAFLVAAPVNLHTKNPEEE
MIEPAVRGTLNVMRSCVKAGTVRRVVLTSSAAAVTTRPQLQGDGHVLDEESW
SDVEYLRAHKPAGPWGYPVSKVLLEKEASRFAEEHGIGLVTVCPGLTVGAAPA
PTARTSVPNCLSLLSGDEAAFAVLDAIESATGCLPLVHVDDVCRAELFAAEEGA
AARRYVCCGLNTTVAELARFLADKYPQYGVKTNLLSGERLEKPRVCLSSAKLV
KEGFEFRYRTLDDIYDDMVEYGKALGILPDL
A. thaliana ACR:
(SEQ ID NO: 95)
MDQTLTHTGSKKACVIGGTGNLASILIKHLLQSGYKVNTTVRDPENEKK
IAHLRKLQELGDLKIFKADLTDEDSFESSFSGCEYIFHVATPINFKSEDPEKDMIK
PAIQGVINVLKSCLKSKSVKRVIYTSSAAAVSINNLSGTGIVMNEENWTDVEFLT
EEKPFNWGYPISKVLAEKTAWEFAKENKINLVTVIPALIAGNSLLSDPPSSLSLS
MSFITGKEMHVTGLKEMQKLSGSISFVHVDDLARAHLFLAEKETASGRYICCAY
NTSVPEIADFLIQRYPKYNVLSEFEEGLSIPKLTLSSQKLINEGFRFEYGINEMYD
QMIEYFESKGLIKAK
P. somniferum NOS:
(SEQ ID NO: 96)
MHGQKNISERYQKFKEMEGTGKIVCVTGGAGYLASWLIMRLLERGYSV
RTTVRSDPKFREDVSHLKALPEATEKLQIFEADLENPESFDDAINGCVGVFLVA
QGMNFAEEYTLEKIIKTCVEGTLRILQSCLKSKTVKKVVYTSSADAAMMISNLK
AVKEIDETIWSEVDNFISKPEQVIPGLPSYVVSKVLTERACLKFSEEHGLDVVTIL
PPLVVGPFITPHPPPSVSIALSIISGDVSMMLGVRLENAVHIDDVALAHIFVFECE
KAKGRHICSSVDFPMHDLPKFISENYPEFNVPTDLLKDIEEQEPVHLSSDKLLSM
GFQFKYDFAEIFGDAIRCAKEKGFL
A. thaliana 4-DFR:
(SEQ ID NO: 97)
MVREEEEDDNNGGGGERKLPVADETVPSLLDGTGLVCVTGGTGFVAS
WLIMRLLQRGYSVRATVRTNPEGNKKDISYLTELPFASERLQIFTADLNEPESFK
PAIEGCKAVFHVAHPMDPNSNETEETVTKRTVQGLMGILKSCLDAKTVKRFFY
TSSAVTVFYSGKNGGGGGEVDESVWSDVEVFRNQKEKRVSSSYVVSKMAAET
AALEFGGKNGLEVVTLVIPLVVGPFISPSLPSSVFISLAMLFGNYKEKYLFDTYN
MVHIDDVARAMILLLEKPVAKGRYICSSVEMKIDEVFEFLSTKFPQFQLPSIDLK
NYKVEKRMSLSSKKLRSEGFEFKYGAEEIFGGAIRSCQARGFL
H. sapiens AKR:
(SEQ ID NO: 98)
MDPKYQRVELNDGHFMPVLGFGTYAPPEVPRNRAVEVTKLAIEAGFRHI
DSAYLYNNEEQVGLAIRSKIADGSVKREDIFYTSKLWCTFFQPQMVQPALESSL
KKLQLDYVDLYLLHFPMALKPGETPLPKDENGKVIFDTVDLSATWEVMEKCK
DAGLAKSIGVSNFNCRQLEMILNKPGLKYKPVCNQVECHPYLNQSKLLDFCKS
KDIVLVAHSALGTQRHKLWVDPNSPVLLEDPVLCALAKKHKRTPALIALRYQL
QRGVVVLAKSYNEQRIRENIQVFEFQLTSEDMKVLDGLNRNYRYVVMDFLMD
HPDYPFSDEY
M. musculus AKR:
(SEQ ID NO: 99)
MDSKQQTVRLSDGHFIPILGFGTYAPQEVPKSKATEATKIAIDAGFRHIDS
ASMYQNEKEVGLAIRSKIADGTVKREDIFYTSKVWCTFHRPELVRVCLEQSLKQ
LQLDYVDLYLIHFPMAMKPGENYLPKDENGKLIYDAVDICDTWEAMEKCKDA
GLAKSIGVSNFNRRQLEKILKKPGLKYKPVCNQVECHPYLNQGKLLDFCRSKDI
VLVAYSALGSHREKQWVDQSSPVLLDNPVLGSMAKKYNRTPALIALRYQLQR
GVVVLAKSFSEKRIKENMQVFEFQLTSEDMKVLDDLNKNIRYISGSSFKDHPDF
PFWDEY
S. lycopersicum AKR2:
(SEQ ID NO: 100)
MAEATEMPYIELNTGFSIPAVGLGTWQSDPGVVGKAVETAIKMGYRHID
CAQIYKNEKEIGEVLSRLFKDGVVKRRELFITSKLWNTNHAPEDVPVALDKTLQ
DLQLEYVDLYLIHWPVSMKPGSVDFKPENLMPTNIPRIWEAMEKVYDSGKARV
IGVSNFSTKKLEDLLQVARTPPAVNQVECHPSWQQAKLRELCKSNNVHLSAYS
PLGSPGTTWLKSDVLKQPAVISVAEKLGKTPAQVCLRWGIQMGQSVLPKSTHE
ARIKENLDVLNWSIPDDLLAKFSEIPQARLLKGASFAHETHGQYRTLEELWDGEI
S. lycopersicum AKR1:
(SEQ ID NO: 101)
MTMNLIKQMLVPNVNLNSGHKMPLIGMGTAPSLPEHDQLVSTLIDAIEI
GYRHFDTAAVYGSEEALGQAVVEAIQRGLIKSREQVFITSKLWCTETHRHLVLP
ALKRTLGRLKMDYLDLYLIHLPVTMKKKVNSKDDEMRVDKEDIIPFDMRGTW
EAMEECCRLGLAKSIGVSNFTCTKISQILHYATILPAVNQVEMHVAWRQEKML
EFCKEKGIHVSAWSPLGANGLTPWGIHSVMESPVLKDIAIHKRKSVAQVALRW
VYEQGASVIVKSFNKERMKENLQILDWELSNEEIAQIQEIPPCTGFNVDMVLVH
PNGPYKSANQFWDGEI
M. truncatula AKR:
(SEQ ID NO: 102)
MGSVEIPTKVLTNTSSQLKMPVVGMGSAPDFTCKKDTKDAIIEAIKQGY
RHFDTAAAYGSEQALGEALKEAIELGLVTRQDLFVTSKLWVTENHPHLVIPALQ
KSLKTLQLDYLDLYLIHWPLSSQPGKFTFPIDVADLLPFDVKGVWESMEEGLKL
GLTKAIGVSNFSVKKLENLLSVATILPAVNQVEMNLAWQQKKLREFCNANGIV
LTAFSPLRKGASRGPNEVMENDMLKEIADAHGKSVAQISLRWLYEQGVTFVPK
SYDKERMNQNLCIFDWSLTKEDHEKIDQIKQNRLIPGPTKPGLNDLYDD
P. somniferum COR:
(SEQ ID NO: 103)
MESNGVPMITLSSGIRMPALGMGTAETMVKGTEREKLAFLKAIEVGYRH
FDTAAAYQTEECLGEAIAEALQLGLIKSRDELFITSKLWCADAHADLVLPALQN
SLRNLKLDYLDLYLIHHPVSLKPGKFVNEIPKDHILPMDYKSVWAAMEECQTL
GFTRAIGVCNFSCKRLQELMETANSPPVVNQVEMSPTLHQKNLREYCKANNIMI
TAHSVLGAVGAAWGTNAVMHSKVLHQIAVARGKSVAQVSMRWVYQQGASL
VVKSFNEARMKENLKIFDWELTAEDMEKISEIPQSRTSSAAFLLSPTGPFKTEEE
FWDEKD
A. thaliana AKR:
(SEQ ID NO: 104)
MSALTFPIGSVHHLMPVLALGTAASPPPEPIVLKRTVLEAIKLGYRHFDTS
PRYQTEEPLGEALAEAVSLGLIQSRSELFVTSKLWCADAHGGLVVPAIQRSLETL
KLDYLDLYLIHWPVSSKPGKYKFPIEEDDFLPMDYETVWSEMEECQRLGVAKC
IGVSNFSCKKLQHILSIAKIPPSVNQVEMSPVWQQRKLRELCKSKGIVVTAYSVL
GSRGAFWGTHKIMESDVLKEIAEAKGKTVAQVSMRWAYEEGVSMVVKSFRK
DRLEENLKIFDWSLTEEEKQRISTEISQSRIVDGEVYISEKGPIKSVTEMWDGEI
D. lanata 3βHSD:
(SEQ ID NO: 105)
MSSKPRLEGKVAIITGAASGIGEETARLFVEHGASVVVADVQDELGRQV
VASVNSDDKISYYHCDVRDEKQVAATVRYAVEKYGRLDIMLSNAGVFGALMT
NVIDLDMVDFENVLATNVRGVANTIKHAARAMVEGKVKGSIICTASVSASLGG
MGPPAYTASKHAVLGLVKGACAELGVHGIRVNSVAPYGVATPMPCSAYGMTP
SQMEEANNSRANLKGVVLKAKHVAEAALFLASDESAYVSGQNLAVDGGFTVVR

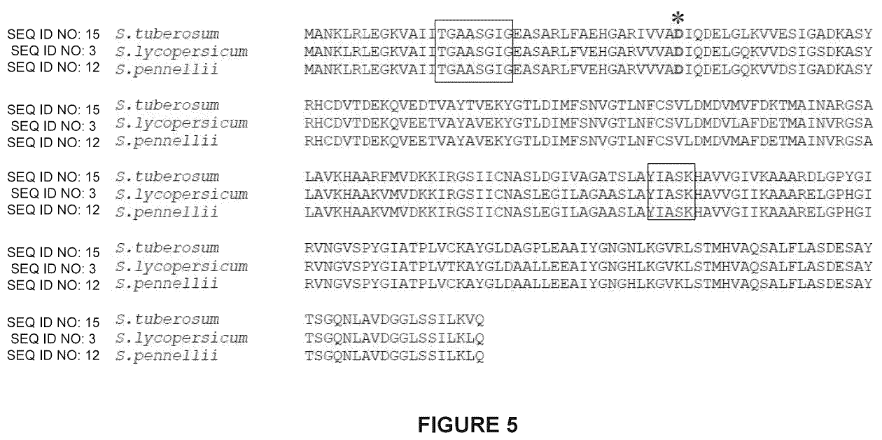

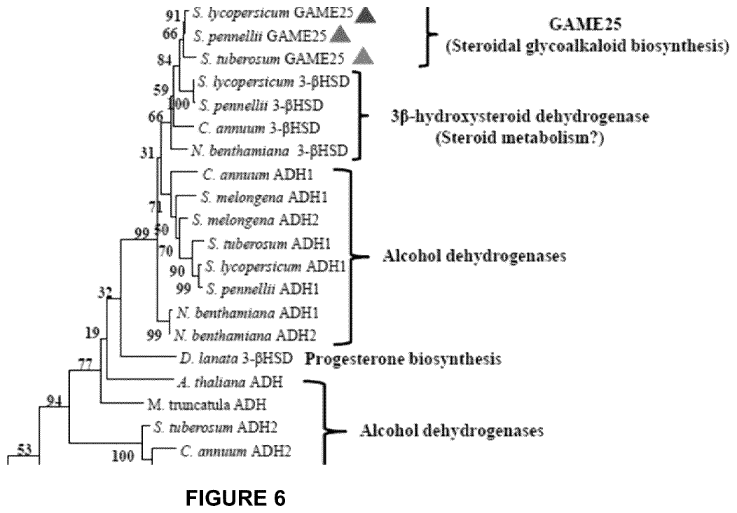

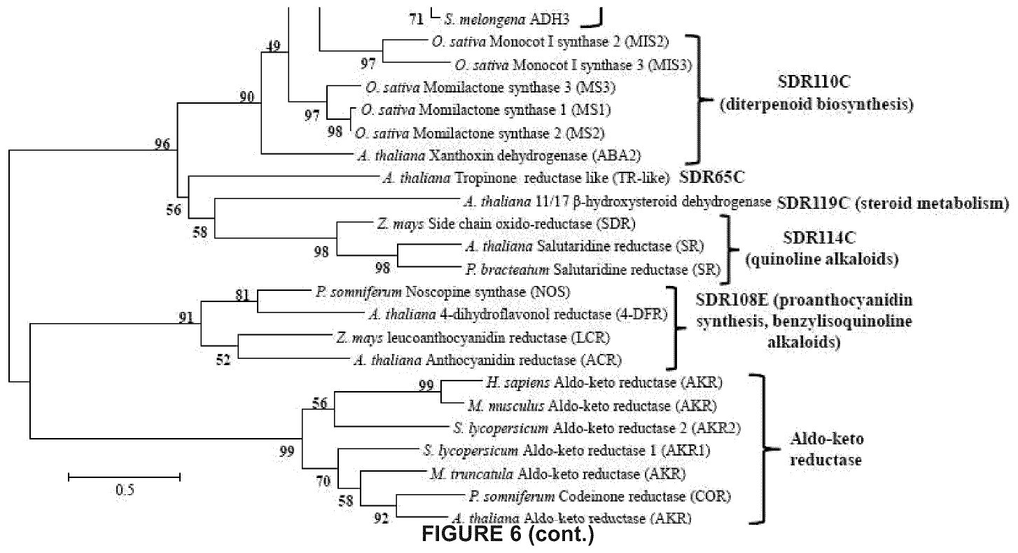

Results: Short-chain dehydrogenases/reductases (SDR) represents one of the largest and most diverse NAD(P)(H)-dependent enzyme superfamilies' that have evolved in plants and were recently categorized into 49 sub-families. SDR is over-represented in plants and was recently categorized into sets of 49 sub-families. The 259 amino acid GAME25 protein sequence shows the characteristics of a classical SDR family member containing the TGxxxGxG cofactor binding and the YxxxK catalytic motifs (FIG. 5). The presence of a specific Asp residue (40^{th} amino acid) indicated the likely preference of GAME25 for NAD over NADP⁺ cofactor (FIG. 5). Phylogenetic analysis showed that GAME25 homologs of certain Solanaceae species (i.e. tomato, potato, and Solanum pennellii) form a distinct sub-clade compared to other plant SDRs (FIG. 6). The most related sub-clade next to GAME25 proteins in the phylogenetic tree included 3β-hydroxysteroid dehydrogenase homologs from tomato and Solanum pennellii (3-βHSD, ˜90% amino acid identity with GAME25 sub-clade proteins) that may possibly be involved in plant steroid metabolism. Phylogenetic analysis also suggested that there is no homolog of GAME25 protein in eggplant and capsicum (FIG. 6). Moreover, GAME25 proteins are clearly separated from Digitalis lanata 3-βHSD protein (showing ˜75% amino acid identity with the GAME25 proteins) that is involved in removal of C-5,6 double bond from steroid derivatives during the progesterone and cardenolide biosynthesis (FIG. 6). The distinctiveness of the tomato GAME25 sub-clade suggested unique catalytic capabilities for this sub-clade that is most likely different even from the relatively similar 3-βHSD sub-clade members (FIG. 6).

### Methods for Examples 3-9

### Plant Materials

Tomato (Solanum lycopersicum) cv. Micro Tom, potato (Solanum tuberosum) cv. Desiree and eggplant (Solanum melongena) cv. Tudela plants were grown in a climate-controlled greenhouse at 24° C. during the day and 18° C. during the night, with natural light.

### Analytical Standards

Analytical standards Tomatidine (the commercial standard of tomatidine also contains dehydrotomatidine as impurity, Sigma-Aldrich), solanidine (ChemFaces), solasodine (ChemFaces), α-tomatine (the commercial standard of α-tomatine also contains dehydrotomatine as impurity, Carbosynth USA), α-solanine (Sigma Aldrich), α-chaconine (Sigma-Aldrich) and α-solamargine (ChemFaces), solanid-4-en-3-one (Sigma-Aldrich) and diosgenin (Sigma-Aldrich) were dissolved in methanol at concentration of 1 mg/ml.

### Generation of GAME25 Transgenic Tomato, Potato, Eggplant and S. Pennellii Plants

The GAME25 silencing construct (RNAi, GAME25i) for tomato was generated by introducing a 199 bp 3′ UTR fragment to pENTR/D-TOPO (Invitrogen) (by NotI and AscI, Forward primer: GCGGCCGCATTGTCACGCTATTTGTGTTGG (SEQ ID NO: 6), Reverse primer: GGCGCGCCGAAATTTATATCTTTTTAAGTCACAACG (SEQ ID NO: 7) and further cloning of the GAME25 fragment to the pK7GWIWG2 (II) binary vector using the Gateway LR Clonase II enzyme mix (Invitrogen). The GAME25 silencing construct for S. pennellii(Forward primer: GCGGCCGCATTGTCACTCTATTGTGTTGGCGTG (SEQ ID NO: 106), Reverse primer: GGCGCGCCTAAATTTATATCTTTTCAAGTCACAATG (SEQ ID NO: 107) was prepared using the same methods described as above. For the over-expression (GAME25-Ox), corresponding GAME25 coding sequence from tomato and potato was cloned into pDONR221 using the Gateway BP Clonase II enzyme mix (Invitrogen) and then transferred to the pK2GW7 binary vector using Gateway LR Clonase II enzyme mix. Constructs were transformed into tomato cv. Micro Tom, potato, eggplant cv. Tudela and S. pennellii as described previously. Positive transgenic lines were selected by quantitative Real Time-PCR (qPCR) and further used for metabolite analysis.

### Fragment Used for GAME25-RNAi Construction

The nucleic acid sequence of the GAME25-RNAi construct is set forth in SEQ ID NO: 8.

(SEQ ID NO: 8)
ATTGTCACGCTATTTGTGTTGGCGTGCTGTGGCGTGGGCCTTAATCCTC
ACTCTCTTGTGTCTGTACTTCTGTTTCATCTCGTTTCGTTTCAAATTTT
CAACTTAATAATACTCTCATATTTTATGCGATATTTTTCAGATTTATAC
TAAGTTTTTTATAGATATTTTAAACGTTGTGACTTAAAAAGATATAAAT
TTC

### Plants Extract Preparation and Targeted Profiling of Steroidal Metabolites

Preparation of extracts and the profiling of steroidal alkaloids (SAs) and steroidal glycoalkaloids (SGAs) in various tomato (leaves, green and red fruit), potato (leaves, tuber skin and flesh), eggplant (leaves), and S. pennellii (leaves) tissues were performed with same methods as described by Itkin et al. (2011) and Cardenas et al. (2016) Nat. Commun. 7:10654. Three biological replicates (n=3) from each genotype were used for metabolic analysis (e.g. #2, #3 and #4 are three independent GAME25-RNAi transgenic lines and each transgenic line represents three biological samples collected from three different plants). Briefly, 100 mg frozen powder of plant tissue was extracted with 80% methanol and 0.1% formic acid, and followed by brief vortex and sonication for 20 min at room temperature. Finally, the extracts were centrifuged for 15 min at 14,000×g and filtered through 0.22 μm filters.

Samples were analyzed using a high-resolution UPLC/qTOF system comprised of a UPLC (Waters Acquity) connected to a qTOF detector (tandem quadrupole/time-of-flight mass spectrometer, Waters). Separation of metabolites was performed on the 100×2.1-mm i.d., 1.7-um UPLC BEH C18 column (Waters Acquity). The mobile phase consisted of 0.1% formic acid in acetonitrile:water (5:95, v/v; phase A) and 0.1% formic acid in acetonitrile (phase B). The following linear gradient was used for steroidal alkaloids analysis and for the enzyme assays products: from 100 to 72% phase A over 22 min, from 72 to 0% phase A over 14 min, then held at 100% phase B for further 2 min; and then returned to the initial conditions (100% phase A) in 0.5 min and conditioning at 100% phase A for 1.5 min. For the separation of GAME25 enzyme assay product with dehydrotomatidine as a substrate, that is tomatid-4-en-3-one a different, shorter linear gradient was used: from 75 to 55% phase A over 10.5 min, from 55 to 0% phase A over 0.5 min, then held at 100% phase B for further 1.5 min; and then returned to the initial conditions (75% phase A) in 0.5 min and conditioning at 75% phase A for 1 min. The flow rate was 0.3 m/min, and the column temperature was kept at 35° C. Masses of the eluted compounds were detected with two different qTOF detectors, equipped with an electrospray ionization source (ESI): either with a XEVO MS or with Synapt HDMS. The following settings were used for XEVO MS: capillary −1 kV; cone −27 V; source temperature was set to 140° C., desolvation −400° C., desolvation gas flow −800 L/h. The following settings were used for Synapt HDMS: capillary −3.4 kV; cone −24 V; source temperature was set to 125° C., desolvation −275° C., desolvation gas flow −650 L/h. Argon was used as a collision gas. ESI was used in positive ionization mode at the m/z range from 50 to 1600 Da. The MS system was calibrated using sodium formate, and Leu enkephalin was used as the lock mass. MassLynx software version 4.1 (Waters) was used to control the instrument and calculate accurate masses and elemental compositions. In addition, a mixture of 15 standard metabolites, injected after each 10 samples, was used for quality control. For steroidal alkaloids analysis, data acquisition was performed in the MS^{E} mode with energy ramp that records an exact mass precursor and fragment ion information from every detectable component in a sample. MS^{E} mode rapidly alternates between two functions: the first acquiring low-energy exact mass precursor ion spectra and the second acquiring elevated-energy exact mass fragment ion spectra. The collision energy was set to 4 eV for low-energy function, and for the high-energy function the collision energy was set either to 10-30 eV ramp for Synapt HDMS, or 15-45 eV for XEVO MS. Metabolites were identified using standard compounds (see the Analytical Standards section above) by comparison of their retention times and mass fragments. When the corresponding standards were not available, compounds were putatively identified by comparing their retention times, elemental composition and fragmentation pattern with those described in literature (see Itkin et al. (2011); Wu et al. (2013); Schwahn et al. (2015)). Relative quantification of the compounds was carried out using TargetLynx™ (Waters), which is a targeted analysis application.

For enzyme assay experiments, an additional MS-MS analysis with a collision energy ramp from 20 to 50 eV was performed to identify the structures of the enzymatic reaction products. The solanid-4-en-3-one product was identified based on the comparison of its retention time and MS-MS spectrum to those measured for its corresponding standard. Another three products (tomatid-4-en-3-one, solasod-4-en-3-one and diosgen-4-en-3-one) were putatively assigned as follows: first, elemental composition based on accurate masses and the isotopic pattern was calculated using the MassLynx software. Then the MS-MS fragmentation pattern was analyzed, and the fragments were assigned applying the fragmentation rules for steroid-based compounds.

### Quantitative Real-Time PCR Analysis

Total RNA was isolated from wild type and transgenic tomato lines (leaf, green fruit, breaker fruit and red ripe fruit), transgenic potato (leaves, tuber skin and flesh), transgenic eggplant (leaves) and transgenic S. pennellii leaves with three biological replicates (n=3) using the Trizol method (Sigma-Aldrich). Three biological replicates (for each genotype) denote three separate tissue samples obtained from three different plants. DNase I (Sigma-Aldrich)-treated RNA was reverse transcribed using a high-capacity cDNA reverse transcription kit (Applied Biosystems). Gene-specific oligonucleotides were designed with the Primer Express 3 software (Applied Biosystems). The TIP41 gene was used as a reference gene for tomato and S. pennellii samples and the NAC gene was used as an endogenous control for potato. For eggplant, the cyclophilin gene was used as a reference for Real Time PCR analysis.

### Example 3

Tomato Steroidal Glycoalkaloid Metabolism is Re-Routed from the Native, Predominant Saturated α-Tomatine Branch to the Unsaturated Dehydrotomatine Branch in GAME25-Silenced Leaves

Objective: To determine the role of GAME25 in SGA metabolism.

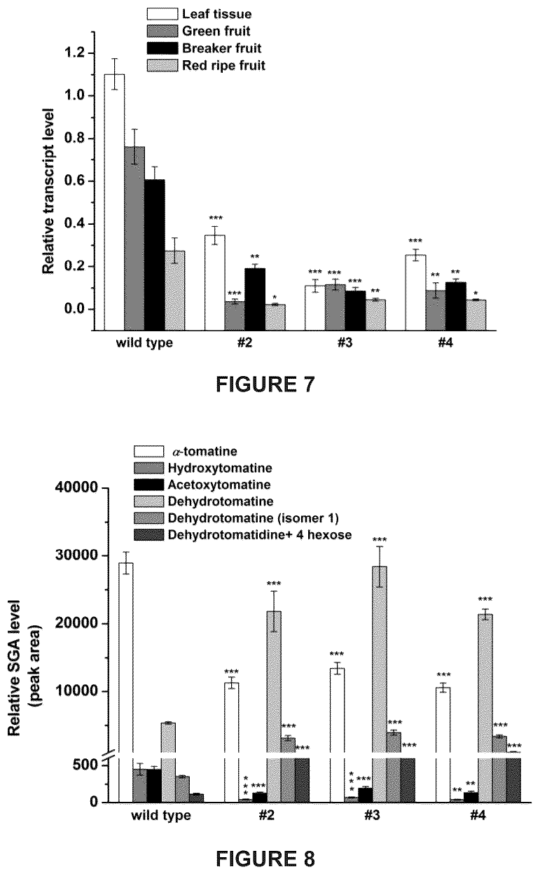

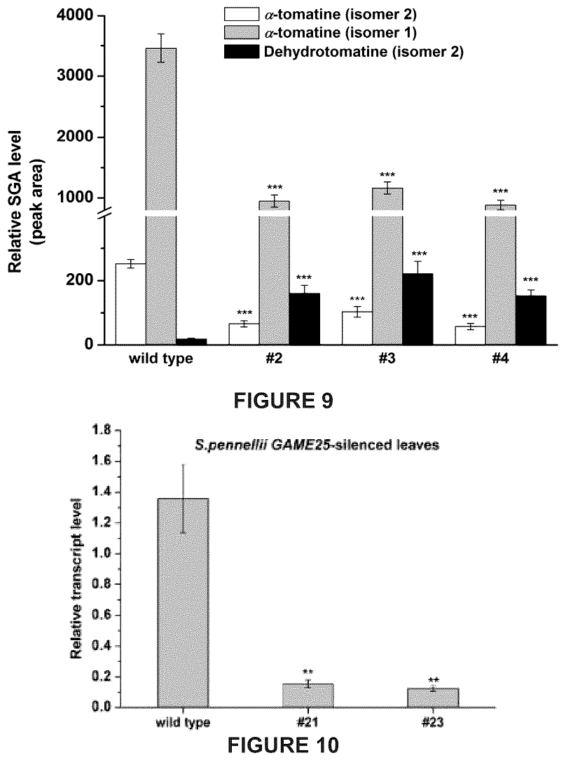

Results: 3 independent GAME25-RNAi transgenic tomato lines (#2, #3 and #4) were generated as described above. GAME25 transcript level was significantly reduced in GAME25i leaves and various fruit tissues of developmental stages (green, breaker and red ripe fruit) (FIG. 7). GAME25-RNAi leaves showed significant reduction in α-tomatine (˜2.5-3 fold), hydroxytomatine (˜6-10 fold), acetoxytomatine (˜2.5-3 fold), and α-tomatine isomers (1 and 2; ˜3-4 fold) compared to wild-type leaves (FIG. 8). On the other hand, dehydrotomatine (˜4-6 fold), dehydrotomatine isomer 1 (˜9-11 fold) and dehyrotomatidine+4 hexose (˜6-9 fold) were increased considerably compared to wild-type leaves (FIG. 8 and FIG. 9).

Conclusion: Reduction in α-tomatine and its downstream metabolite levels, yet, accumulation of dehydrotomatine and its isomers in GAME25i lines suggest that either (i) GAME25 is involved in α-tomatine biosynthesis directly from the dehydrotomatine glycoside or (ii) it mediates tomatidine biosynthesis from dehydrotomatidine (also called tomatidenol)

No significant accumulation of dehydrotomatidine (tomatidenol), which is a dehydrotomatine precursor, was observed in GAME25i lines. This SGA appeared to be converted to its glycosylated derivative, dehydrotomatine that actually did show a major accumulation (FIGS. 1A-1C, and FIG. 8). The accumulation of dehyrotomatidine+4 hexoses metabolite derived from dehydrotomatidine (FIGS. 1A-1C, and FIG. 8) after GAME25 silencing indicate a role of GAME25 upstream of the SA aglycone glycosylation steps in SGA biosynthesis. Hence, it is hereby proposed that GAME25 is involved in the conversion dehydrotomatidine to tomatidine, rather than acting on dehydrotomatine.

Subsequent in vitro enzyme assays confirmed that GAME25 could not act on glycosylated SAs (e.g. dehydrotomatine). Similar to the cultivated species, silencing of GAME25 in the wild tomato species, S. pennellii also resulted in reduced tomatine derived alkaloids levels, as α-tomatine and acetoxytomatine, while dehydro-alkaloids were enriched, i.e. dehydrotomatine and acetoxy-dehydrotomatine (FIG. 10).

### Example 4

### GAME25-Silencing Results in Gradual Loss of Saturated SGAs in Developing and Ripening Tomato Fruit and has a Major Impact on SGAs in Tomato Green Fruit

Objective: Observe the SGA profile in tomato green fruit when GAME25 expression is altered.

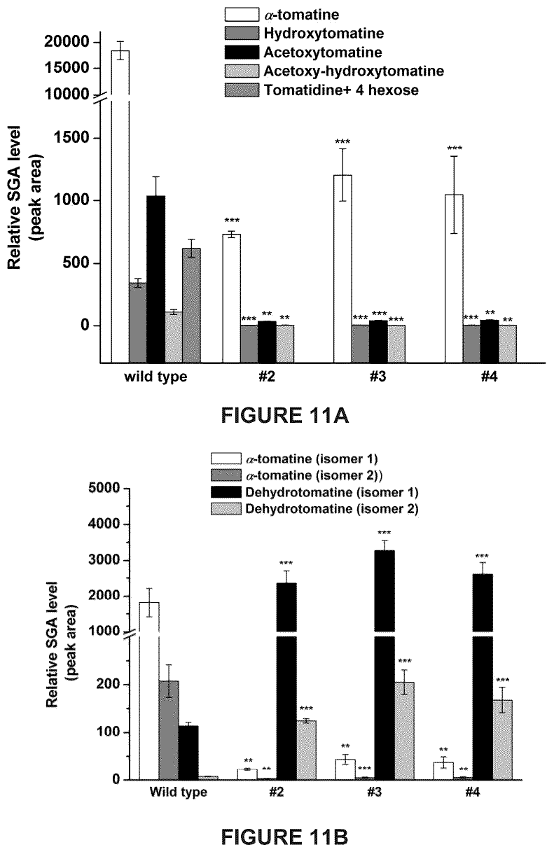

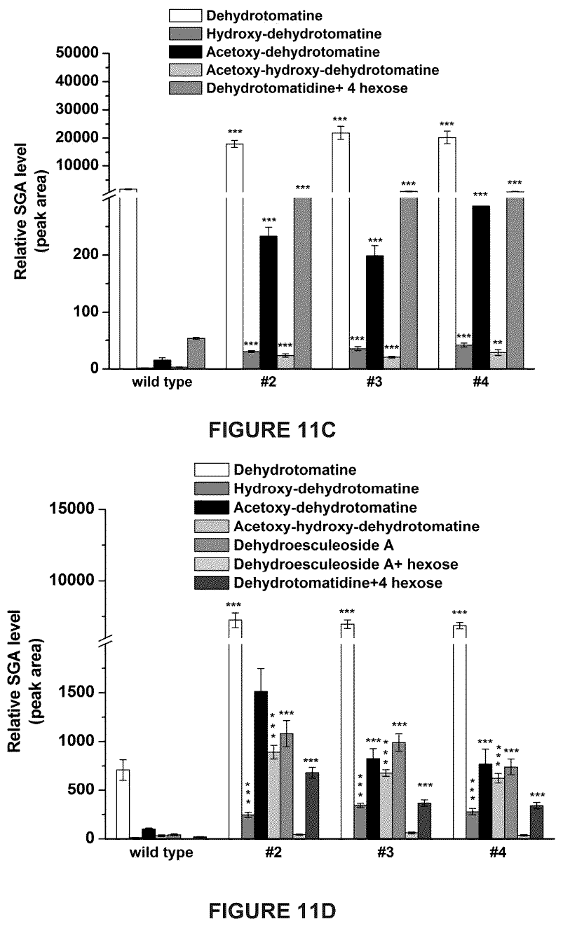

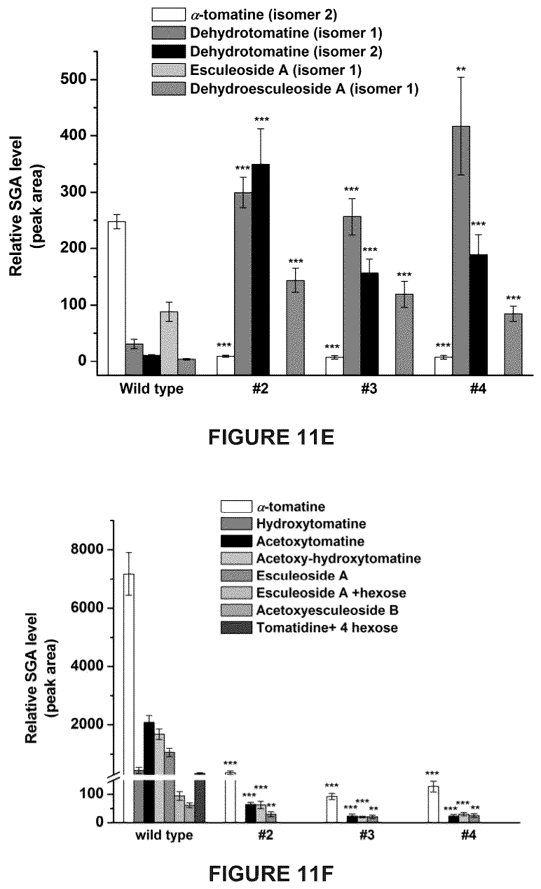

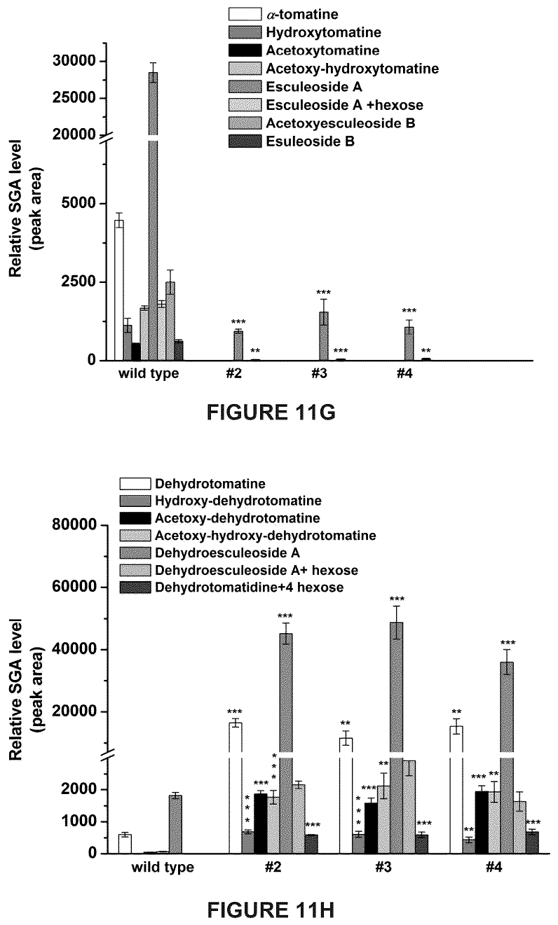

Results: The SGAs profile of green fruit of GAME25i lines was compared to wild-type green fruits. During the transition from green to red fruit in tomato, α-tomatine is converted to esculeosides and lycoperosides (saturated SGAs), while dehydrotomatine is converted to dehydroesculeosides and dehydrolycoperosides (unsaturated SGAs) (FIGS. 1A-1C). Esculeoside A and its derivatives are the major SGAs found in tomato red ripe fruit, while dehydroesculeosides and dehydrolycoperosides are minor SGAs found in this tissue. GAME25i green fruits displayed drastic reduction in levels of α-tomatine (˜15-25 fold), hydroxytomatine (˜100 fold), acetoxytomatine (˜30 fold), acetoxy-hydroxytomatine (˜50 fold), α-tomatine isomer (1 and 2) and complete absence of tomatidine+4 hexose as compared to wild-type (FIGS. 11A-11B). Thus, α-tomatine and its downstream saturated SGA intermediates were severely affected in green fruit tissue due to GAME25 silencing.

In contrast, unsaturated SGAs as dehydrotomatine (˜10-12 fold), hydroxy-dehydrotomatine (˜25 fold), dehydrotomatine isomer (1 and 2), acetoxy-dehydrotomatine (˜12-15 fold), acetoxy-hydroxy-dehydrotomatine (˜6-8 fold) and dehydrotomatidine+4 hexose (˜12-18 fold) were increased significantly compared to wild-type suggesting redirection of the flux towards unsaturated dehydro-SGAs due to GAME25 silencing (FIGS. 11B-11C).

Conclusion: Silencing of GAME25 diverted metabolic flux from the α-tomatine derived pathway to the dehydrotomatine dependent SGAs branch.

### Example 5

### GAME25i Breaker Fruit Accumulates Dehydrotomatine Derived SGAs

Objective: To determine the SGAs in GAME25i breaker fruit.

Results: SGAs analysis was performed in breaker fruits from GAME25i lines and compared to wild-type breaker fruits. The metabolic flux from α-tomatine to the dehydro-SGAs pathway was further extended to dehydrotomatine and additional downstream SGAs. SGAs metabolites accumulated following GAME25-silencing included hydroxy-dehydrotomatine (˜20-30 fold), acetoxy-dehydrotomatine (˜7-15 folds), acetoxy-hydroxy-dehydrotomatine (˜20-25 fold) as well as dehydroesculeoside A (˜20-25 fold) and its derivatives (FIGS. 11D-11E). On the other hand, tomatine derived SGAs including hydroxytomatine, acetoxytomatine, acetoxy-hydroxytomatine and esculeoside A, were almost non-existent in GAME25i lines (FIGS. 11E-11F).

### Example 6

### Dehydroesculeoside A, is the Predominant SGA in GAME25i Red Ripe Fruit

Objective: To determine the SGAs in GAME25i red ripe fruit.

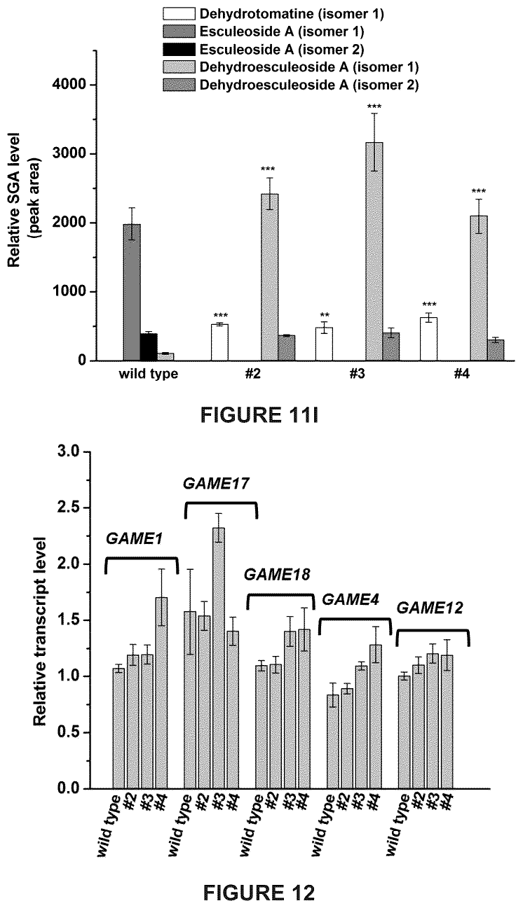

Results: Esculeoside A and its derivatives are the predominant SGAs found in wild-type tomato red ripe fruits. The drastic reduction in α-tomatine levels at the green stage fruit in GAME25i lines resulted in severe decline of esculeoside A and lycoperosides (˜20-25 folds) in red ripe fruit stage (FIG. 11G). On the other hand, the significant accumulation of dehydrotomatine in green fruit in GAME25i lines resulted in a massive accumulation of dehydroesculeoside A (˜20-25) and derivatives in red ripe fruit (FIGS. 11G-11I), providing additional evidence regarding the role of GAME25 in the early divergence of the saturated (α-tomatine) and unsaturated (dehydrotomatine) SGA biosynthesis branches. It appeared that GAME25 silencing did not affect expression of core SGA biosynthesis genes including GAME4 and GAME12 involved in the formation of the dehydrotomatidine aglycone and GAME1, GAME17 and GAME18 performing the glycosylation of the SA-aglycones (FIG. 12).

### Example 7

### GAME25 Over-Expression in Tomato Resulted in the Accumulation of Saturated α-Tomatine and its Downstream SGAs

Objective: To examine the role of GAME25 in SGA biosynthesis.

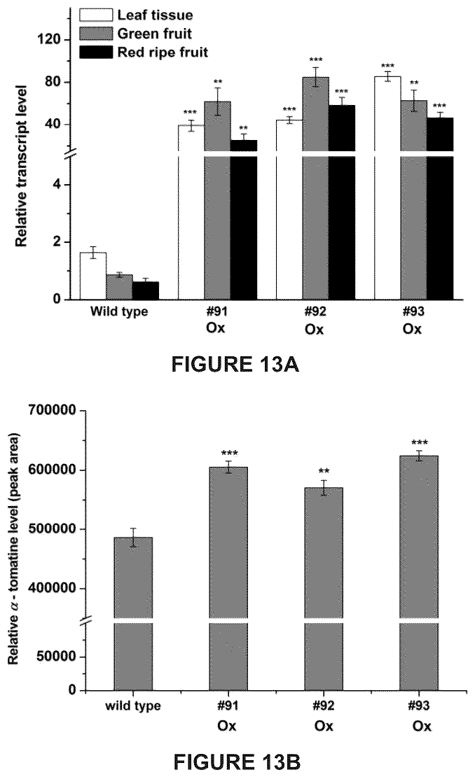

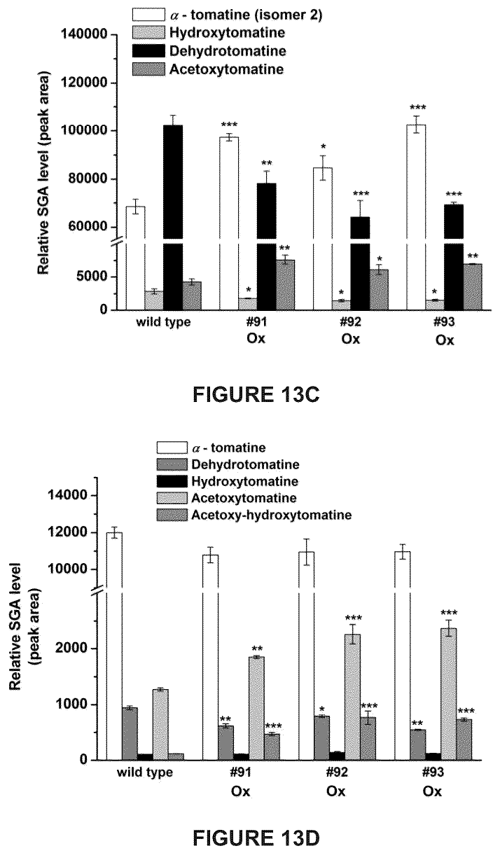

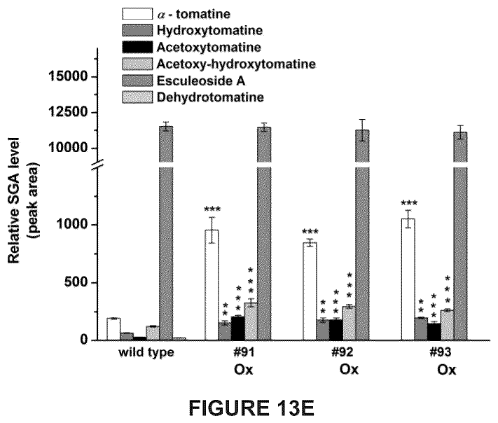

Results: 3 independent transgenic tomato lines over-expressing GAME25 (GAME25-Ox; lines #91, #92 and #93) were generated as described above. GAME25 expression in leaves and fruit tissues (green and red fruit) of transgenic tomato lines was significantly higher than in wild-type tomato plants as determined by qRT PCR (FIG. 13A). In leaves of GAME25-Ox lines, levels of α-tomatine, α-tomatine (isomer 2) and acetoxytomatine increased significantly with simultaneous reduction of dehydrotomatine compared to wild-type leaves (FIGS. 13B-13C). GAME25-Ox tomato green fruits displayed a significant reduction in dehydrotomatine levels, whereas no change in α-tomatine content was observed in the same tissues (FIG. 13D). However, significant increase in acetoxytomatine and acetoxy-hydroxytomatine (α-tomatine derived SGAs) was detected when compared to wild-type green fruits (FIG. 13D). Analysis of red fruit derived from GAME25-Ox lines clearly showed accumulation of α-tomatine (˜4-6 fold) and its downstream saturated derivatives (i.e. hydroxytomatine (˜2-3 fold), acetoxytomatine (˜5-7 fold), and acetoxy-hydroxytomatine (˜2-3 fold)) (FIG. 13E). Yet, red fruit of the GAME25-Ox plants did not show a change in esculeoside A content suggesting that the glycosylation step of the SGA steroidal backbone (likely of acetoxy-hydroxytomatine) is a limiting factor (FIGS. 1A-1C, and 13E).

### Example 8

### Tomato GAME25 Overexpression in Cultivated Eggplant Results in Newly Produced Saturated SGAs and Steroidal Saponins

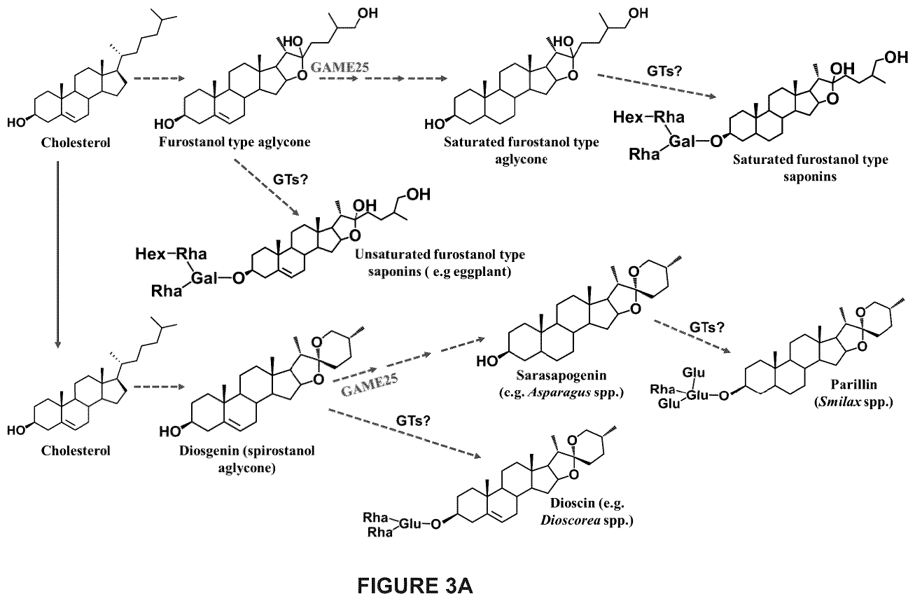

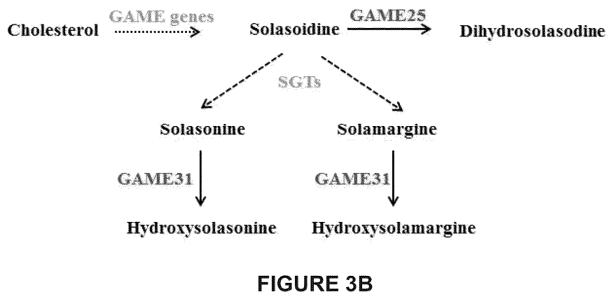

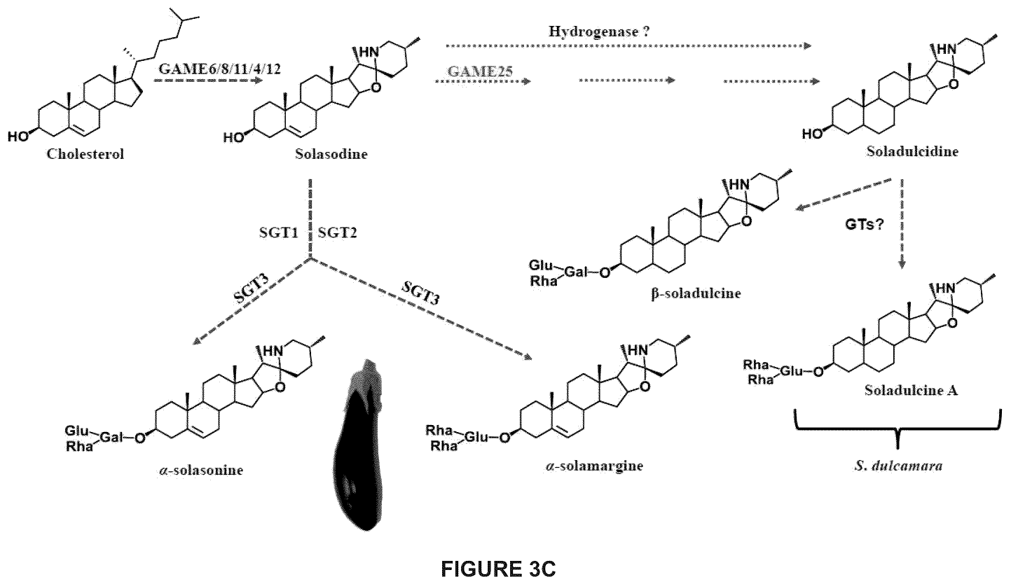

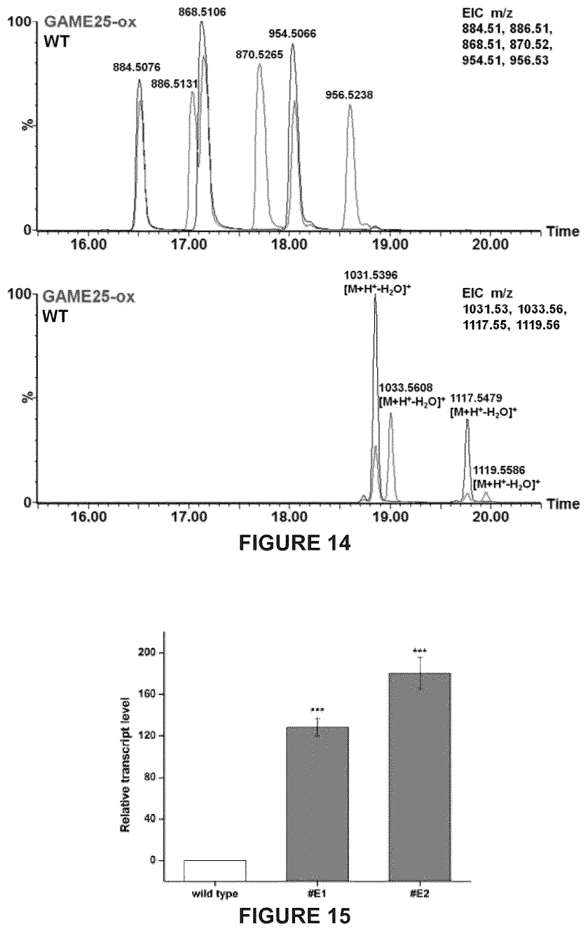

Unlike tomato, saturated SGAs (without C5,6-double bond) are normally not present in cultivated eggplant suggesting the absence of GAME25 activity in this species. This is further supported by the absence of a GAME25 homolog in eggplant as observed in phylogenetic analysis of GAME25 proteins (FIG. 6). In cultivated eggplant (wild type), α-solasonine [m/z 884.5, M+H⁺], α-solamargine [m/z 868.5, M+H⁺] and malonyl-solamargine [m/z 954.5, M+H⁺] are the major unsaturated SGAs (with C-5,6 double bond) derived from the solasodine aglycone (FIG. 14). Moreover, cultivated eggplant also produces unsaturated furostanol type steroidal saponin glycosides [m/z 1031.5, (M+H⁺—H₂O)⁺ and m/z 1117.5, (M+H—H₂O)⁺] from unsaturated furostanol type saponin aglycone (FIG. 14). Henceforth, the impact of tomato GAME25 activity in eggplant was examined in order to see whether GAME25 can shift SGA metabolism in eggplant from predominant unsaturated SGA branch to normally not occurring saturated SGA branch. Transgenic eggplant lines were generated overexpressing the tomato GAME25 gene (#E1 and #E2 are two independent transgenic eggplant lines; FIG. 15). Surprisingly, in eggplant leaves, GAME25 overexpression resulted in reduction of the unsaturated α-solasonine, α-solamargine, malonyl-solamargine SGAs as well as unsaturated furostanol saponin glycosides (FIG. 14). Conversely, major accumulation of 3-soladulcine [m/z 886.5, M+H⁺], soladulcine A [m/z 870.5, M+H⁺] and saturated form of malonyl-solamargine [m/z 956.5, M+H⁺](FIG. 14) was observed in GAME25-Ox eggplant leaves. Both β-soladulcine and soladulcine A (lacking the C-5,6 double bond) are SGAs derived from the saturated soladulcidine aglycone and are typically found in S. dulcamara (FIG. 3A). Thus, S. dulcamara must have an active GAME25 homolog that mediates the formation of above mentioned saturated SGAs. Surprisingly, saturated furostanol type steroidal saponin glycosides (m/z 1033.5, (M+H⁺—H₂O)⁺ and m/z 1119.5, (M+H⁺—H₂O)⁺ in FIG. 14) were also found in GAME25 overexpressing transgenic eggplant lines that were completely absent in wild type. Thus, tomato GAME25 drives the formation of saturated soladulcidine (SA aglycone) from solasodine and saturated saponin aglycone from unsaturated ones respectively in eggplant that further produce their respective glycosylated forms.

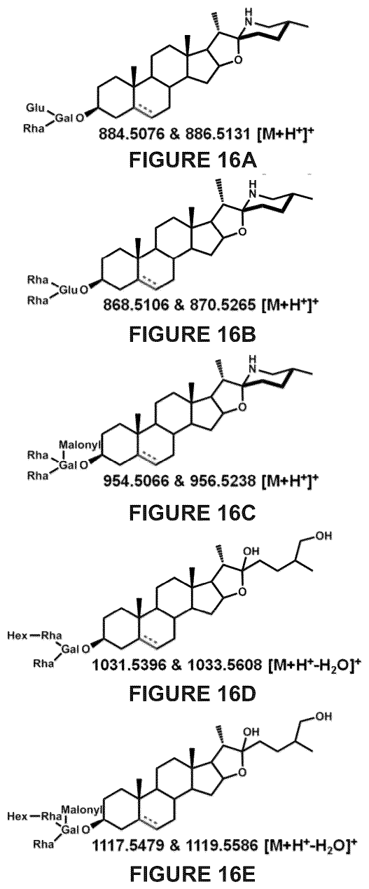

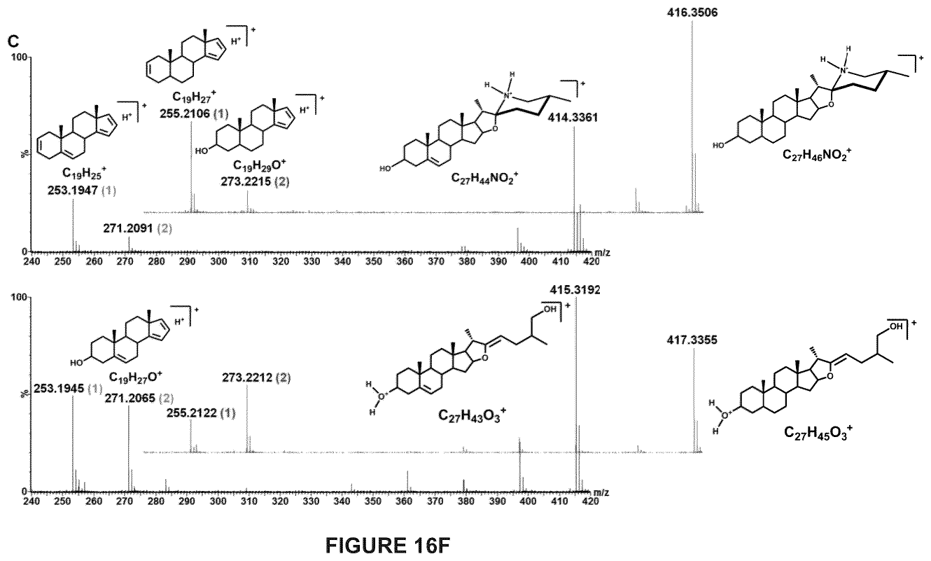

The chemical structures of unsaturated and saturated, SGAs as well unsaturated and saturated steroidal saponins metabolites identified here are provided in FIGS. 16A-16E. The identification of metabolites was based on Mass fragmentation pattern. Loss of C-3 sugar moieties in unsaturated SGAs led to the formation of the fragment ion with m/z 414.3 that corresponds to unsaturated steroidal aglycone backbone (FIG. 16F). Further loss of E and F ring in unsaturated steroidal backbone resulted in the fragment ion m/z 271.2 which further produced ion with m/z 253.19 after dehydration (FIGS. 16F, upper panel). On the other hand, due to absence of C-5,6 double bond, saturated SGAs formed the fragment ion with m/z 416.3 corresponding to saturated steroidal aglycone that further produced the fragment ions m/z 273.2 and 255.2 after loss of E/F rings and dehydration respectively (FIGS. 16F, upper panel). Similarly, unsaturated furostanol type steroidal saponins showed m/z 415.3, 271.2 and 253.19 fragment ions whereas saturated furostanol type steroidal saponins displayed m/z 417.3, 273.2 and 255.2 fragment ions after MS-fragmentation analysis (FIGS. 16F, lower panel).

In tomato, it is hereby predicted that GAME25 catalyzes conversion of unsaturated dehydrotomatidine aglycone to saturated tomatidine aglycone. As tomato GAME25 induces the formation of saturated SGAs (e.g. β-soladulcine and soladulcine A) from solasodine aglycone as well as saturated saponins from unsaturated furostanol saponin aglycone in eggplant, it is possible that GAME25 might have broad preference for unsaturated SA aglycone and saponin substrates in Solanum plants.

### Example 9

### GAME25 Overexpression Results in Reduced Levels of α-Solanine and α-Chaconine, the Major SGAs in Potato

Objective: To examine the impact of GAME25 activity on SGAs metabolism in potato.

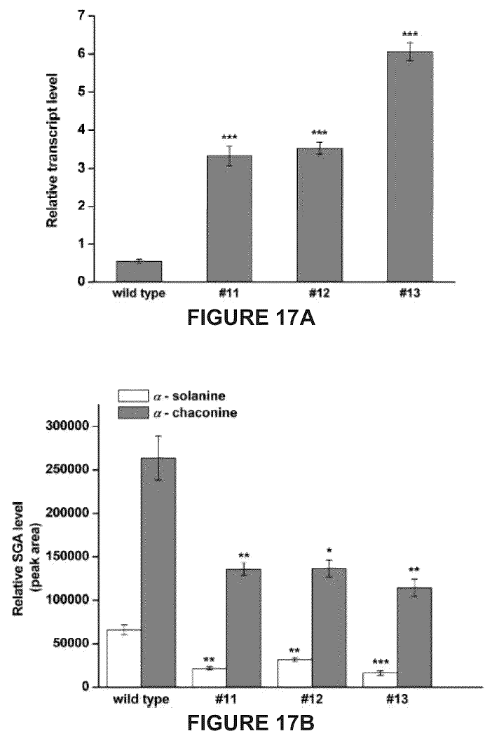

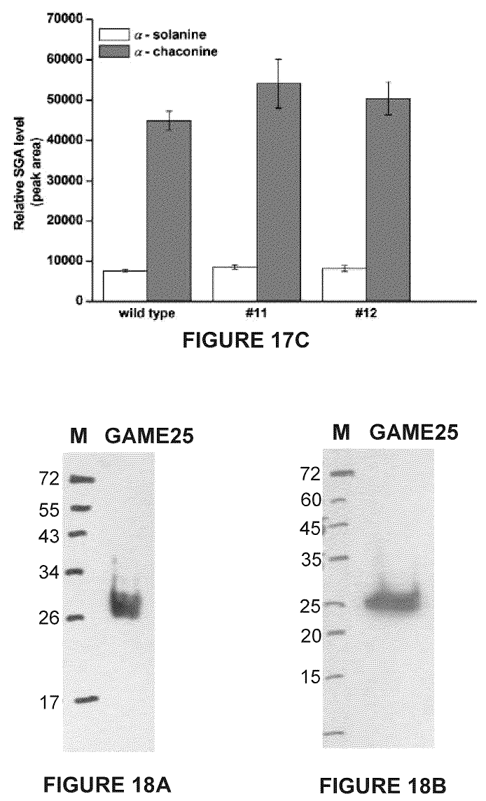

Results: Potato plants overexpressing GAME25 (GAME25-Ox) were generated as described above (FIG. 17A). The pathway from the aglycone solanidine to demissidine and its glycosylated form (i.e. demissine) in potato corresponds to the tomato pathway in which the C-5,6 double bond is eliminated from dehydrotomatidine towards tomatidine and the glycosylated α-tomatine (FIGS. 2A and 2B). While α-tomatine and its derivatives accumulate to high levels in tomato, the domesticated potato does not accumulate demissidine or demissine SGAs in any plant part (while wild potato species do). In contrast to tomato, the potato GAME25 showed very low expression in green and vegetative potato tissues. Yet, a GAME25 transcript is abundant in various potato tuber tissues (i.e. skin and cortex).

Over-expression of GAME25 in potato leaves resulted in a significant reduction in levels of α-solanine and α-chaconine, the major SGAs in potato (FIG. 17B). Yet, accumulation of demissidine or demissine was not detected in these leaves. Moreover, analysis of tuber skin tissue in GAME25-Ox potato plants showed no accumulation of demissidine or demissine along with no major change in α-solanine and α-chaconine SGAs (FIG. 17C).

### Example 10

Functional Characterization of the S. lycopersicum GAME25 Encoded Recombinant Enzyme Produced in Sf9 Insect Cells and E. coli

Objective: To functionally characterize the enzyme activity encoded by GAME25.

Methods: The recombinant tomato and potato GAME25 enzymes were produced in Sf9 insect cells and examined for activity with dehydrotomatidine, solanidine and solasodine as substrates (the major SGAs aglycones in tomato, potato and eggplant, respectively).

Heterologous Expression of the S. lycopersicum GAME25 Encoded Enzyme in Baculovirus Infected Sf9 Insect Cells

The GAME25 gene was cloned into the pVL1393 baculovirus expression vector using the following primers.

GAME25 baculo Forward primer:
(SEQ ID NO: 9)
CCCGGGATGGCAAATAAGCTCAGGTTGG.
GAME25 baculo Reverse primer:
(SEQ ID NO: 10)
TCTAGATTACAGATCTTCTTCAGAAATAAGTTTTTGTTCTTGTAGCTTC
AAAATAGAACTTAGTCC.

Each expression vector construct was co-transfected with the ProGreen GFP linearized baculovirus DNA (AB vector) to generate the recombinant viruses in Spodoptera frugiperda (Sf9) insect cells. Infection efficiency was monitored by fluorescence of recombinant GFP viruses infected cells. Sf9 cells were grown in ESF921, protein free culture medium (Expression Systems). Three days post-infection cells were collected and washed twice in PBS. Microsomal fractions were isolated by suspending the cell pellet in 20 mM potassium phosphate buffer pH 7.25, 20% (v/v) glycerol, 1 mM EDTA and 1 mM DTT. The resuspended cells were sonicated, and the cell debris was removed by centrifugation at 10,000×g for 15 min. The supernatant was further centrifuged at 100,000×g for 60 min. The pellet containing the microsomal fractions was homogenized in lysis buffer. GAME25 was expressed with a myc-tag at the C-terminus. The isolated microsomal fractions were analyzed by SDS-PAGE and immune-blot with c-myc antibodies (A2S Technologies Ltd.).

GAME25 and GAME35 Expression in E. coli BL21 (DE3) Cells and their Protein Purification

GAME25 and GAME35 genes were cloned into pET28 vector separately and expressed in E. coli BL21 (DE3). Bacterial cells were grown in LB medium at 37° C. When cultures reached A₆₀₀-0.6, protein expression was induced with 200 μM of isopropyl-1-thio-β-d-galactopyranoside (IPTG) at 15° C. for 24 h. Bacterial cells were lysed by sonication in 50 mM Tris-HCl pH 7.5, 500 mM NaCl, 1 mM PMSF and protease inhibitor cocktail (Calbiochem). An aliquot of whole cell extract was kept for further analysis. Soluble protein was purified using Ni-NTA agarose beads (Adar Biotech) and eluted with 500 mM imidazole in buffer containing 50 mM NaH₂PO₄ pH-7.5 and 300 mM NaCl. The whole cell extract and the eluted fractions were analyzed by SDS-PAGE staining with InstatBlue and by Western blot with HRP conjugated anti-His antibodies (Sigma).

### In Vitro Assays of the Recombinant GAME25 Enzyme(s) Expressed in Insect Cells

The steroidal alkaloid aglycones dehydrotomatidine, solanidine and solasodine as well as their glycosylated forms (i.e. SGAs) α-tomatine, α-solanine, α-chaconine and α-solamargine were used as substrates for GAME25 enzyme assays. In vitro assays for the tomato and potato recombinant GAME25 enzymes were performed as follows: Briefly, microsomal fractions of Sf9 cells expressing tomato or potato GAME25 proteins (0.2 mg total protein was used per reaction) were separately incubated in sodium phosphate buffer (pH 7.4) with 100 μM of each individual substrate (details are mentioned above), DTT (5 mM) and NAD (150 μM) at 37° C. for 4 hrs. The reactions were stopped by addition of 300 μl of 80% methanol and 0.1% formic acid, followed by brief vortex and sonication for 15 min. Finally, the extracts were centrifuged for 15 min at 14,000×g, filtered through 0.22 μm filters, and analyzed by LC-MS (see Plant extracts preparation and targeted profiling of steroidal metabolites section above). Sf9 cells (without the baculovirus vector) microsomes were used in control enzyme reactions.

In Vitro Assays of the Recombinant Tomato GAME25 Enzyme Expressed in E. coli

Assay for purified recombinant tomato GAME25 enzyme (5 μg) from E. coli cells using solanidine (steroidal alkaloid aglycone) and diosgenin (steroidal saponin aglycone) substrates was performed under standard assay conditions similarly as described above.

In Vitro Assays of the Recombinant Tomato GAME25 Enzyme Expressed in E. coli

Assay for purified recombinant tomato GAME25 enzyme (5 μg) from E. coli cells using solanidine (steroidal alkaloid aglycone) and diosgenin (steroidal saponin aglycone) substrates was performed under standard assay conditions similarly as described above.

### GAME35 Enzyme Assay

Purified GAME35 protein (5 μg) was incubated with solanid-4-en-3-one substrate (150 μM), DTT (1 mM), NADH or NADPH (500 μM) in sodium phosphate buffer (pH 7.4) at 30° C. for 3 hrs. The reactions were stopped by addition of 250 μl of 80% methanol and 0.10% formic acid, and followed by brief vortex and sonication for 15 min. Finally, the extracts were centrifuged for 10 min at 13,000×g and filtered through 0.22 μm filters, and analyzed by LC-MS, as described in the section above (plant extracts preparation and targeted profiling of steroidal metabolites). Fractions from pET28 vector (empty) transformed into BL21 (DE3) cells was used as a control in enzyme reaction.

### Results:

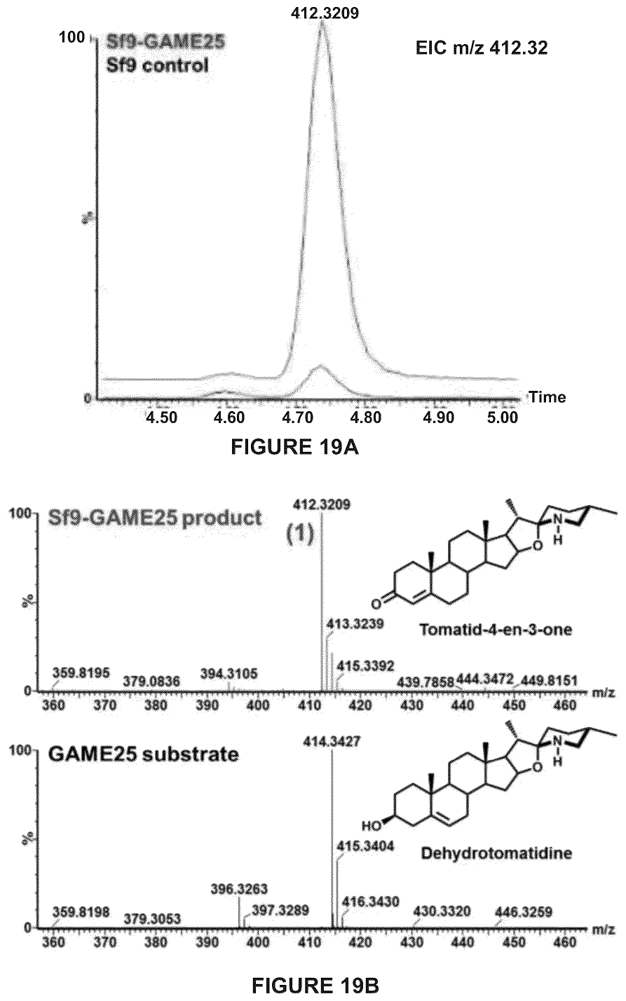

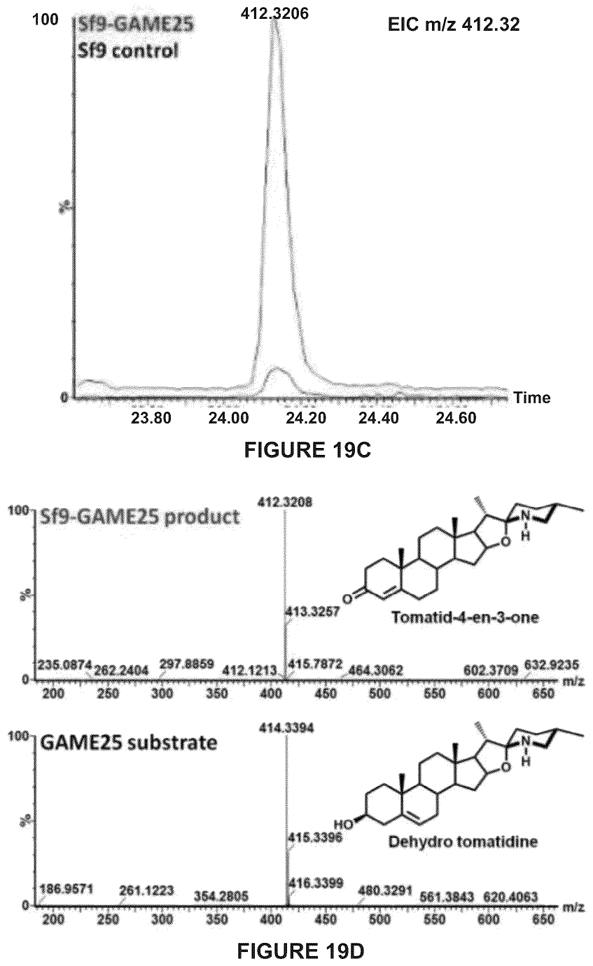

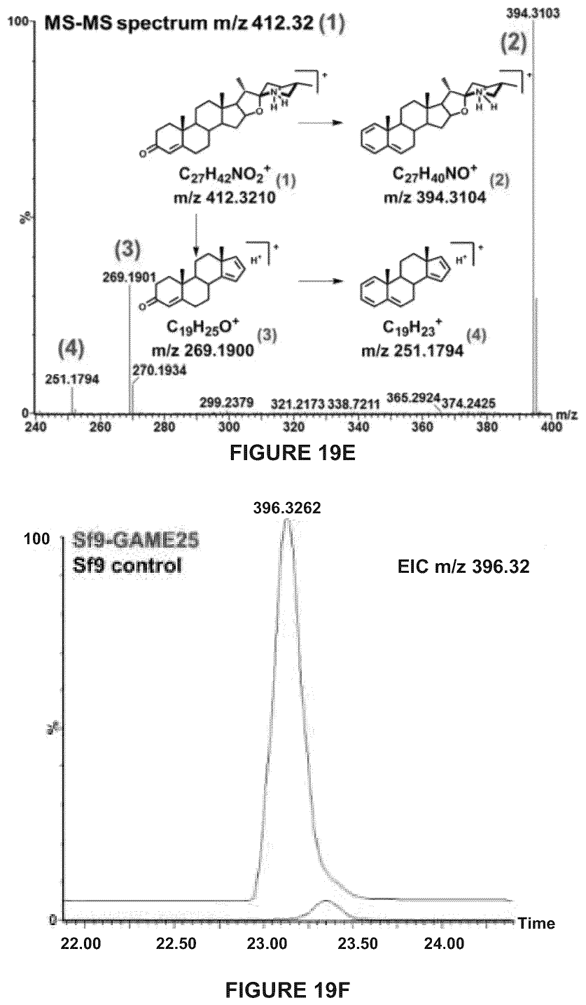

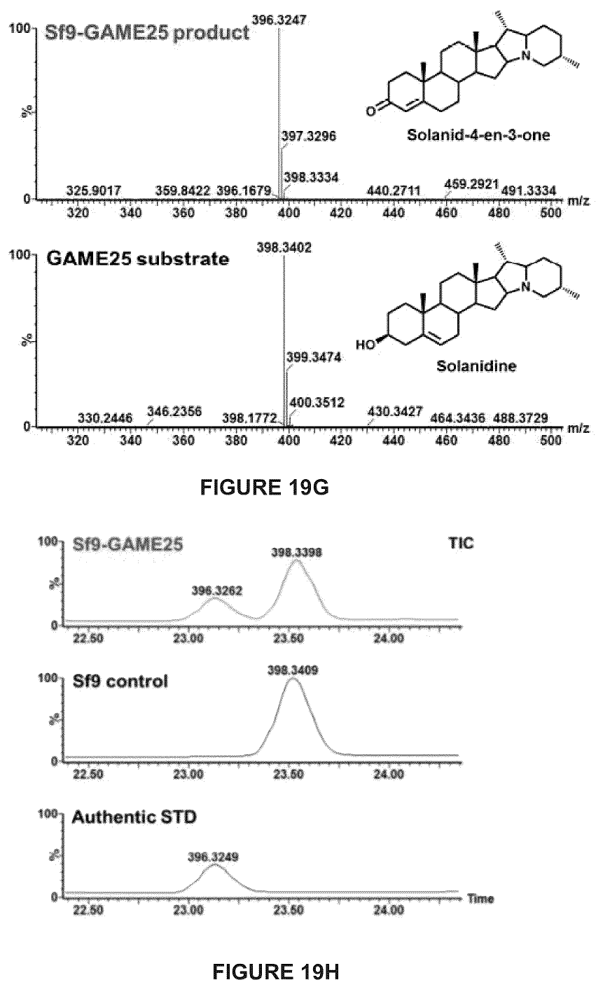

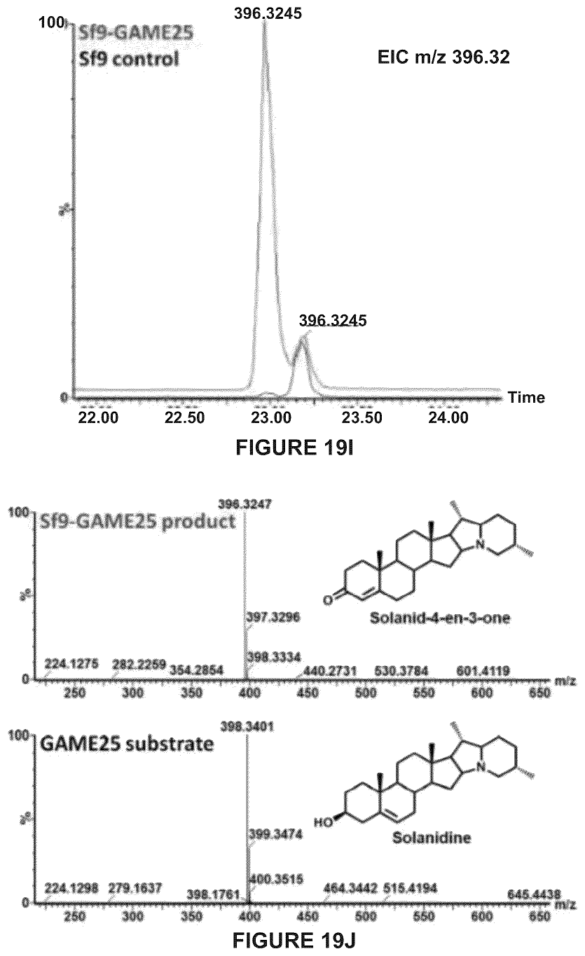

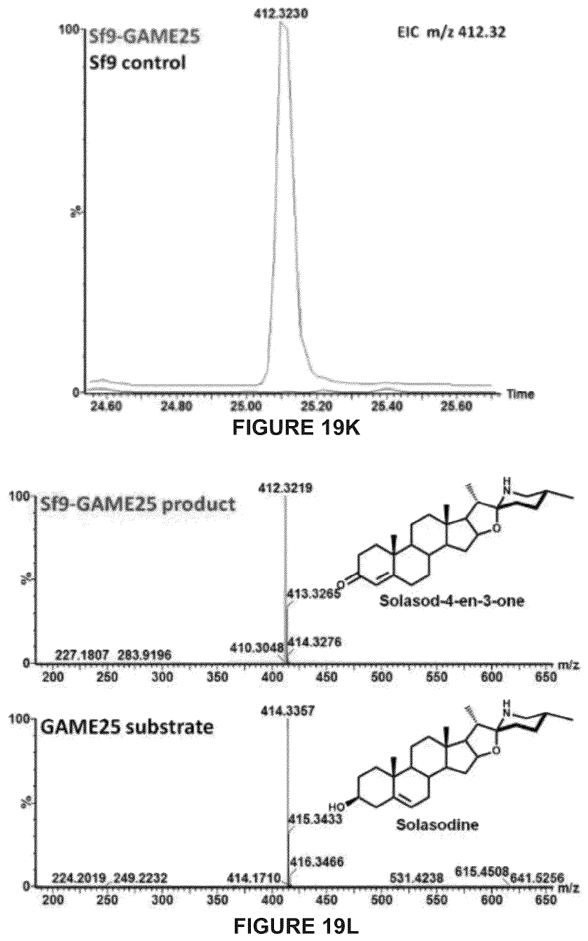

The suggested role of GAME25 in conversion of dehydrotomatidine to tomatidine was examined by expressing the recombinant tomato and potato enzymes in sf9 insect cells and testing microsomal fractions for activity with dehydrotomatidine, solanidine and solasodine (SA aglycones) as substrates respectively. The expression of potato and tomato GAME25 recombinant proteins in sf9 cell microsomes was confirmed by western blot using c-myc antibody (FIGS. 18A and 18B). Enzymatic assays were performed in the presence of NAD⁺ as a cofactor. Assays with either tomato or potato recombinant GAME25 did not result in the formation of tomatidine, demissidine and soladulcidine (saturated steroidal alkaloid aglycones), the expected reaction products. However, assay of both recombinant GAME25 enzymes with dehydrotomatidine (m/z 414.3, M+H⁺) resulted in the formation of a novel compound with the mass m/z 412.3 (M+H⁺) in tomato (FIGS. 19A and 19B) and potato (FIGS. 19C and 19D) GAME25 assay. To identify the newly formed compounds, MS-MS fragmentation pattern analysis was employed (FIGS. 19A, 19B and 19E). The observed fragmentation pattern contains three major fragment ions from 2 parallel fragmentation routes. Loss of the carbonyl-oxygen and formation of an additional double bond leads to the fragment ion with m/z 394.3. Loss of the E and F ring of the steroidal skeleton leads to the fragment ion m/z 269.2 which afterwards gets dehydrated to form fragment m/z 251.17 (FIG. 19E). In conclusion, the newly formed compound was putatively assigned as tomatid-4-en-3-one.

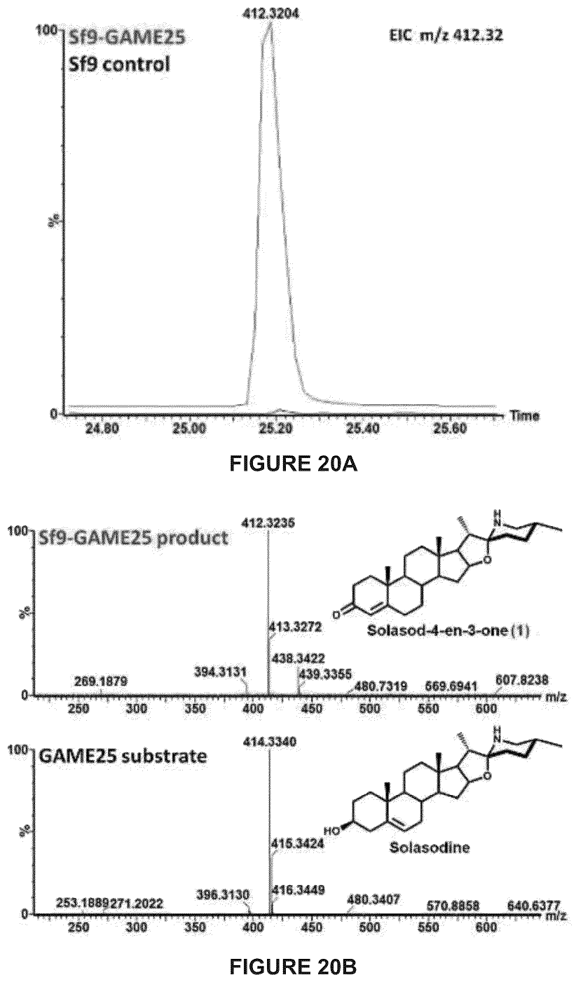

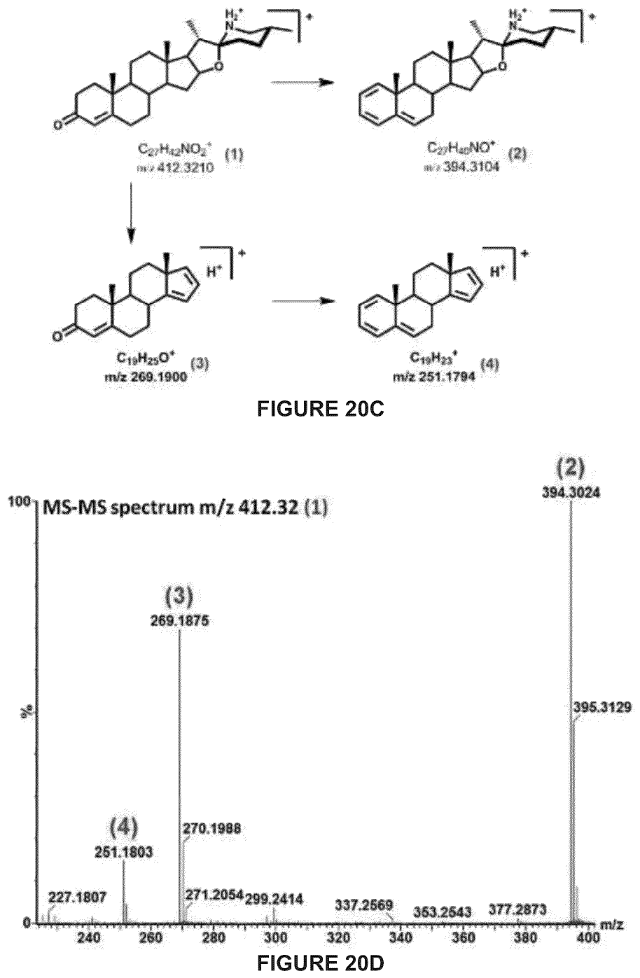

Using solanidine as a substrate (M+H⁺, m/z 398.3), GAME25 enzyme assay (with both tomato and potato GAME25 enzymes) led to formation of a new product with an apparent molecular ion of m/z 396.3 (M+H⁺). This product was identified as solanid-4-en-3-one by comparing retention time and mass spectrum to the authentic commercially available solanid-4-en-3-one standard both for tomato GAME25 (FIGS. 19F-19H) and for tomato GAME25 (FIGS. 17B, 19I, and 19J) assay. The recombinant GAME25 enzymes (both from tomato and potato) were also able to convert solasodine, the eggplant aglycone (M+H⁺, m/z 414.3) to the putative solasod-4-en-3-one compound (M+H⁺, m/z 412.3) identified based on MS-MS fragmentation pattern analysis similarly as described above for assignment of tomadid-4-en-3-one for potato GAME25 (FIGS. 19K and 19L) and for tomato GAME25 (FIG. 20A-20D) assay.

Tomato and potato recombinant GAME25 enzymes were also tested with glycosylated SAs; α-tomatine, dehydrotomatine, α-solanine, α-chaconine and α-solamargine, however, no activity was observed with these substrates. These results suggest that GAME25 catalyzes the oxidation of the 3β-hydroxyl group (3β-hydroxysteroid dehydrogenase activity) and the isomerization of double bond from the C-5,6 to the C-4,5 position (3-oxosteroid Δ^{5,4} isomerase activity) in SA aglycone substrates to form the 3-oxo-Δ^{5,4} SA aglycone intermediates that have been identified here (tomatid-4-en-3-one, solanid-4-en-3-one and solasod-4-en-3-one) (FIGS. 19B, 19D, 19E, 19G, 19J, 19L, and 20B-20D). Furthermore, these results show that GAME25 has activity on various SA aglycones and possibly possesses a novel 3β-hydroxysteroid dehydrogenase/Δ^{5,4} isomerase activity.

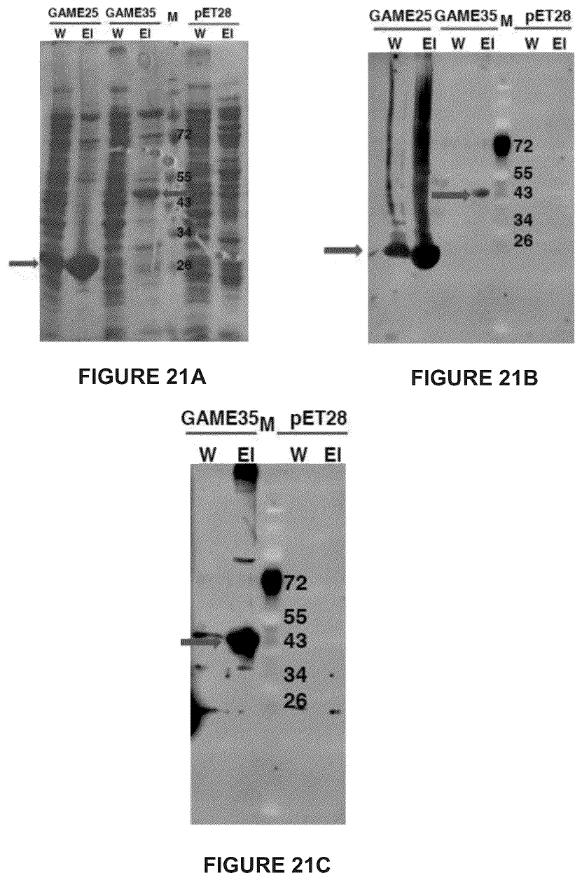

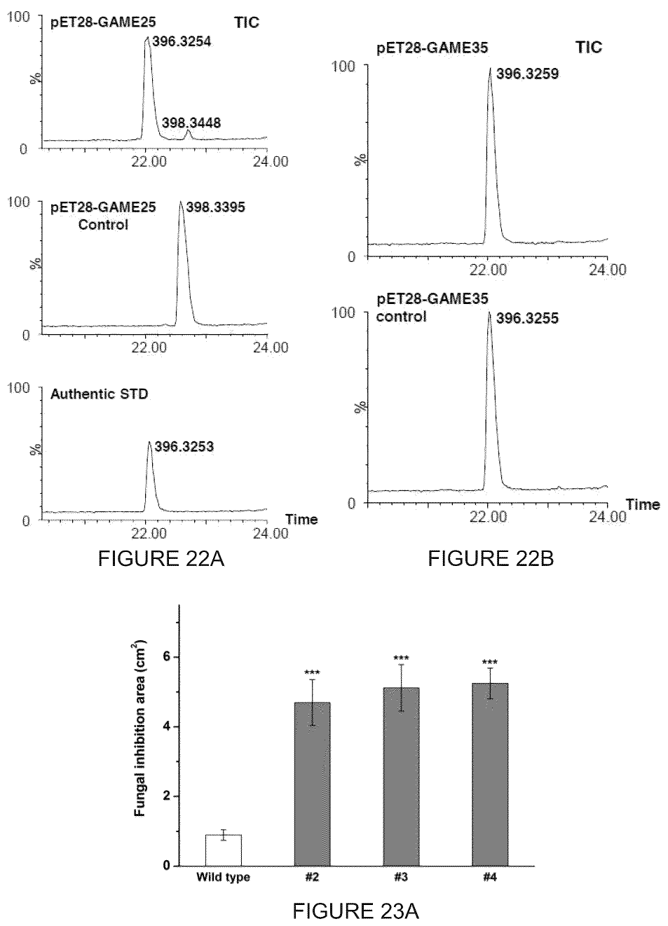

The observed 3β-hydroxysteroid dehydrogenase/Δ^{5,4} isomerase activity for tomato and potato recombinant GAME25 enzymes is rather uncommon since other SDR family enzymes partaking in secondary metabolism typically possess 3β-hydroxysteroid dehydrogenase activity only. For example, in Digitalis, the oxidation (3β-hydroxyl group) and isomerization (C-5,6 to the C-4,5 position) steps which are required during the conversion of pregnenolone to progesterone are carried out successively by two separate enzymes, the 3-βHSD (3β-hydroxysteroid dehydrogenase activity) and the 3-KSI (Δ⁵-3-ketosteroid isomerase activity). In order to confirm that the Δ^{5,4} isomerase activity observed previously for recombinant GAME25 enzymes produced in insect cells came originally from GAME25 enzymes and not from any endogenous enzymes of insect cells, tomato GAME25 protein was expressed in E. coli and purified by Ni-NTA chromatography (FIGS. 21A-21C). Enzyme assay with the purified GAME25 recombinant enzyme using solanidine substrate and NAD⁺ as a cofactor resulted in the formation of the same solanid-4-en-3-one product (FIG. 22A) as described before. The solanid-4-en-3-one product's identity was confirmed by comparison of retention time and mass spectrum with authentic solanid-4-en-3-one standard as described above (FIGS. 17B and 19F-19H). Thus, it is hereby shown doubtlessly that the recombinant GAME25 enzyme possesses both the 3β-hydroxysteroid dehydrogenase and the Δ^{5,4} isomerase activities. These results also indicate that the removal of C-5,6 double bond in the SA aglycones is not a single step reaction as suggested previously.

### A Putative 5β-Progesterone Reductase Homolog Doesn't Act as Second Enzyme Following GAME25 Reaction in Tomato

Recombinant enzyme assay of GAME25 with various SA aglycones clearly shows that GAME25 alone is not sufficient to catalyze the entire Δ⁵ reduction (removal of C-5,6 double bond) in SA aglycone substrates and thus additional enzymes are required to form saturated SA aglycone and further saturated SGAs. In Digitalis, following the reactions of 3-βHSD and 3-KSI, conversion of progesterone to 5β-pregnan-3,20-dione (removal of C-4,5 bond) is catalyzed by 5β-progesterone reductase (5β-POR). Thus, it was hypothesized that a 5B3-POR homolog in tomato might act as a second enzyme and catalyzing a similar reduction with the various 3-oxo-Δ^{5,4} steroidal alkaloid aglycone intermediates (e.g. solanid-4-en-3-one). Sequence homology search with Digitalis 5β-POR against the tomato genome resulted in two candidates; Solyc10g049600 (71% identity) and Solyc10g049620 (78% identity). Among these two genes, only Solyc10g049620 (termed here GAME35) was found to be expressed in different tomato tissues (Cirdenas et al. (2016), Tomato RNA seq data in NCBI Sequence Read Archive with BioProject ID PRJNA307656). In order to examine whether GAME35 catalyzes the enzymatic reaction following GAME25, GAME35 was expressed in E. coli and purified by Ni-NTA chromatography (FIGS. 21A-21C). For GAME35 activity assay, solanid-4-en-3-one was used as a substrate. GAME35 did not react with the solanid-4-en-3-one substrate, suggesting its minimal role in the reduction of the C-4,5 double bond in SA aglycone substrates (FIG. 22B). Thus, a different reductase enzyme might be required to carry out second reaction (removal of C-4,5 bond) during the biosynthesis of saturated SA aglycone from unsaturated one in Solanum plants.

### Example 11

### The Presence of the C-5,6 Double Bond in SGAs is Significant to Pathogenicity and Growth of Tomato Fungi

Objective: Given the finding that silencing of GAME25 in tomato redirects SGA metabolism towards the dehydro-SGAs branch instead of its typical tomatine derived SGAs, it was examined whether dehydro-SGAs levels affect pathogenicity or growth of disease causing fungi.

### Methods

### Fungal Inhibition Assay

Methanolic extracts from 2.5 g wild-type leaves and three independent GAME25i transgenic plant leaves (#2, #3 and #4 are three independent GAME25i transgenic lines, and for each transgenic line, three separate tissue samples (weighing 0.8, 0.8 and 0.9 g each) were collected from three different plants respectively) were evaporated to dryness and redissolved in 700 μl of methanol. Fungal inhibition activity of the GAME25i and wild-type methanolic extracts was determined by the disc diffusion method. Botrytis cinerea (B05.10) and Colletotrichum gloeosporioides (Cg14) were grown on PDA (potato dextrose agar) plates for 10 days at 28° C. After 10 days, a conidial suspension was prepared and 4×10⁴ conidia were spread in each petri dish containing PDA medium. Whatman filter paper (13 mm) was soaked with 100 μL of extract and allowed it to dry. Furthermore, the loaded Whatman filter paper was placed on the pre-inoculated petri dishes. Discs soaked with methanol were used as a control. The plates were then incubated at 28° C. for 2 days. Fungal inhibition activity was evaluated as the area of inhibition of mycelium growth subtracted by the area of the disc. Inhibition of conidia germination by GAME25i and wild-type extracts was tested as described earlier. Briefly, fungal conidia were collected from respective PDA plates and adjusted to the final conidia concentration of 1×10⁵ conidia/ml using sterile distilled water. A drop of conidia suspension was incubated with GAME25i and wild-type methanol extracts (diluted by 5 times) on glass slides with 5 replicates and incubated in a humid chamber for 19 hrs. After incubation, slides were examined under microscope (×40) and each slide was evaluated in three different fields for recording the percentage of conidia germination. This experiment was repeated three times.

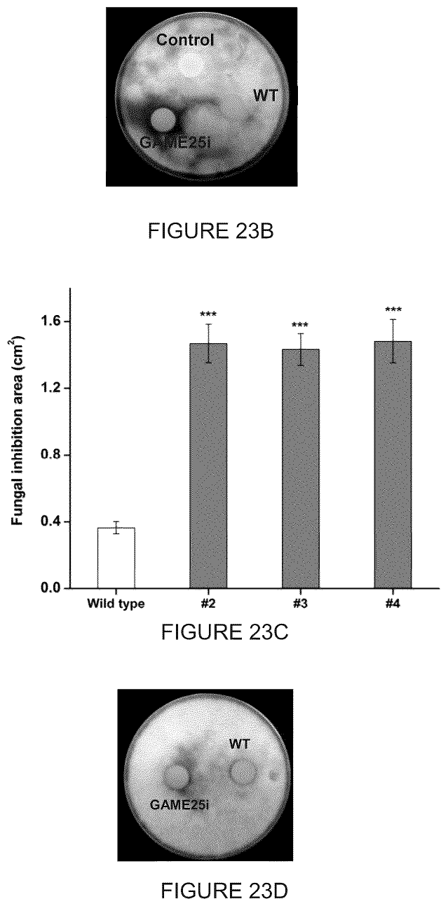

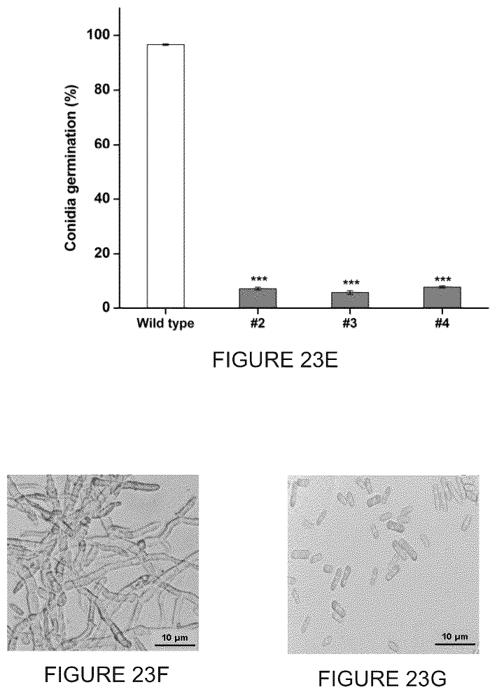

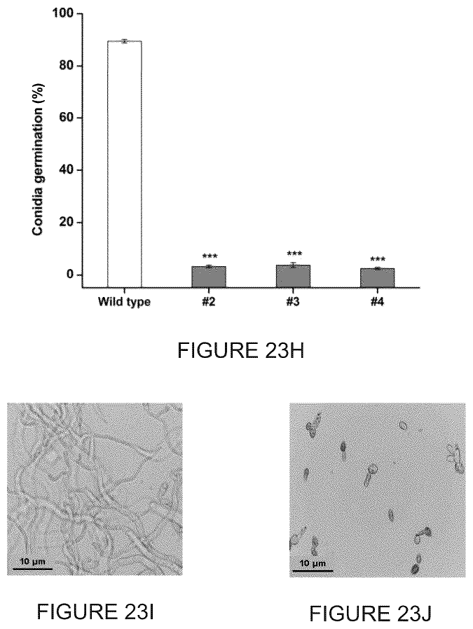

Results: The predominant SGA α-tomatine in tomato green tissues is renowned to affect the growth of many pathogenic fungi, including Botrytiscinerea, Fusariumoxysporum and Colletotrichumgloeosporioides. As it normally produced in small amounts in tomato tissues, the role of dehydrotomatine in phytopathogenicity was not examined. Analysis of GAME25i leaf extracts (producing predominantly dehydrotomatine and downstream SGAs) showed a clear and significant mycelial growth inhibition of the pathogenic fungi B. cinerea and C. gloeosporioides as compared to wild-type extracts (accumulating α-tomatine and derived SGAs) (FIGS. 23A-23D). In addition, fungal conidia germination was severely affected in treatments with GAME25i extracts as compared to wild-type ones (FIGS. 23E-23J).

### Example 12

### The Tomato Recombinant GAME25 Enzyme Also Converts Diosgenin, a Spirostanol Type Saponin Aglycone to Diosgen-4-En-3-One, 3-Oxo-Δ^{5,4} Steroidal Saponin Intermediate

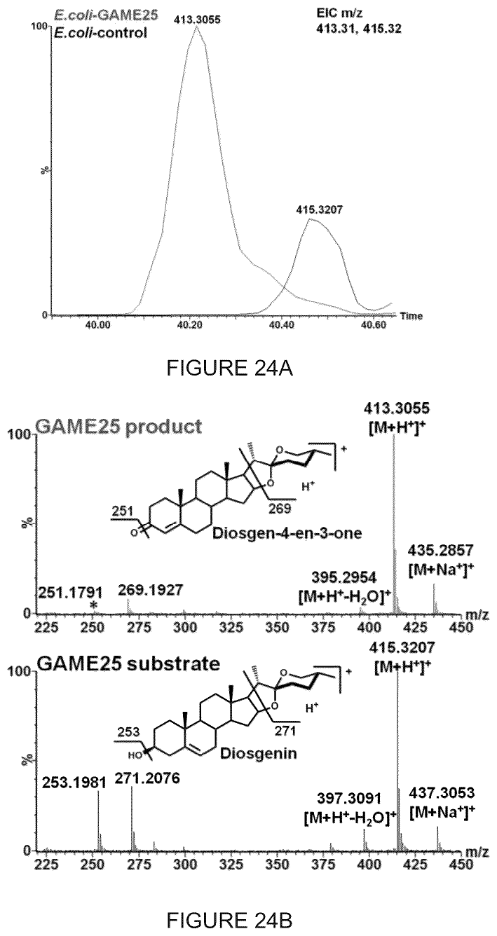

Similar to SGAs, steroidal saponins also display two structural forms, unsaturated or saturated based on C-5,6 double bond position in saponin aglycone (that is either furostanol or spirostanol type) (FIG. 3A). Formation of new saturated furostanol type saponins by GAME25 overexpression in eggplant suggests its possible role also in removal of C-5,6 double bond from steroidal saponin metabolites. Furthermore, recombinant GAME25 enzyme exhibits the 3β-hydroxysteroid dehydrogenase and Δ^{5,4} isomerase activities on various SA aglycone substrates to form the 3-oxo-Δ^{5,4} SA aglycone intermediates. To examine whether GAME25 can catalyze similar reactions but on steroidal saponin aglycone substrate, a recombinant enzyme assay was performed using diosgenin [(M+H⁺, m/z 415.3); a major spirostanol type steroidal saponin aglycone produced by Dioscorea species] as substrate. Interestingly, GAME25 assay resulted in the formation of novel compound with molecular ion m/z 413.3 (M+H⁺) representing oxidation of 3β-hydroxyl group and isomerization of double bond from C-5,6 position to C-4,5 position (FIGS. 24A-24B). While unsaturated diosgenin substrate produce three major fragment ions with m/z 415.3, 271.2 and 253.2, the newly formed compound (m/z 413.3) showed fragment ions with m/z 413.3, 269.2 and 251.2 respectively. Based on mass fragmentation analysis and assignment described as above for SA aglycones (e.g. dehydrotomatidine), this newly formed compound was putatively assigned as diosgen-4-en-3-one (FIGS. 24A-24B). Thus, similar to SA aglycones, GAME25 also catalyzes oxidation of the 3β-hydroxyl group and the isomerization of double bond from the C-5,6 to the C-4,5 position in steroidal saponin aglycone substrates to form the 3-oxo-Δ^{5,4} steroidal saponin aglycone intermediates. The results also suggest that formation of saturated steroidal saponin aglycone (removal of C-5,6 double bond) from unsaturated saponin aglycone is also not a single step reaction and GAME25 is catalyzing the first of them.

### Summary for Examples 1-12

GAME25 is a Key Branch Point Enzyme that Determines the Diversity of SGAs Produced in Solanum Species and Modulates their Level of Toxicity

The enormous diversity of chemical structures produced by plants are often due to what seems to be a minor change in one or more of the core scaffolds that are consequently metabolized by various downstream modifications. This occurs in steroidal glycoalkaloids; a renowned class of secondary metabolites that likely evolved separately in the Solanaceae and Liliaceae families. In Solanum species investigated here, the presence or absence of a double bond at the C-5,6 in the core scaffolds is a major source of structural diversity. More specifically, in tomato, two SA aglycones, namely, dehydrotomatidine and tomatidine, differing in the presence or absence of the C-5,6 double bond, are similarly glycosylated to their corresponding glycoalkaloids dehydrotomatine and α-tomatine, respectively. Both dehydrotomatidine and tomatidine aglycones are highly toxic to plant cells and likely undergo glycosylation to prevent self-toxicity. Studies in animal models demonstrated that SGAs deficient in the C-5,6 double bond, e.g. α-tomatine, are much less toxic to animals and humans as compared to those possessing it (e.g. α-chaconine and α-solanine from potato). This suggests that dehydrotomatine is likely a more toxic SGA when compared to α-tomatine.

Although less toxic to humans and animals, α-tomatine is a highly active molecule that is involved in a range of host-plant resistance mechanisms in tomato plants. Yet, the contribution of dehydrotomatine, typically produced to lower levels in tomato, to the plant resistance against pathogens was not clear. In the present disclosure, severe growth and conidia germination inhibition of the pathogenic fungi B. cinerea and C. gloeosporioides pointed towards enhanced toxicity of extracts enriched with dehydro-derivatives (due to GAME25 silencing) as compared to those (wild-type) predominantly containing α-tomatine and related metabolites (FIG. 23). Thus, in planta, α-tomatine and dehydrotomatine SGAs could be acting synergistically against pathogens and both of these SGAs might have co-evolved in order to exert synergistic effect against a broad range of diseases. GAME25 reported here, catalyzes the first step in the conversion of dehydrotomatidine to tomatidine in which the double bond at the C-5,6 position is reduced (FIG. 22). This reaction is therefore a key branch point that not only determines the diversity of SGAs produced in more than hundreds of Solanum species known to date but also modulates the toxic effects of these molecules in planta, animals and likely also in pathogens and herbivores.

The Formation of Tomatidine from Dehydrotomatidine in Tomato Involves the Reaction Catalyzed by GAME25 and Further Requires Additional Biochemical Reactions.

During the past decades the biosynthesis of dehydrotomatine and α-tomatine was hypothesized to occur through several different pathways. In one scenario, dehydrotomatidine was proposed to be derived from cholesterol which contains a C-5,6 double bond while tomatidine from cholestanol lacking the C-5,6 double bond. Thus, conversion of cholesterol to cholestanol might be responsible for formation of tomatidine. An alternative pathway suggested that teneimine having the double bond, an intermediate derived from cholesterol, is partitioned, leading to tomatidine through the action of a hypothetical hydrogenase reducing the double bond while a portion of it is converted to dehydrotomatidine. In another hypothesis, tomatidine was proposed to be partly dehydrogenated to form dehydrotomatidine by a hypothetical dehydrogenase. In another case, the formation of tomatidine from dehydrotomatidine was hypothesized as a single step reaction catalyzed by a hypothetical hydrogenase. The present disclosure functionally characterizes GAME25 through in planta and in vitro assays and rules out these previous hypotheses. Instead, it is suggested that the formation of tomatidine from dehydrotomatidine, i.e. the reduction of Δ⁵ (C-5,6 position) bond in the SA aglycones, is carried out in multiple steps and GAME25 catalyzes the first of them.

Furthermore, the examples above show that, unexpectedly, SA/SGA levels can be severely reduced in a tomato plant by modifying expression of an enzyme involved in the steroidal alkaloids biosynthetic pathway.

### GAME25 Exhibits a Novel Dual-Function Activity in Solanum Plants

In vitro enzyme assays with the recombinant tomato and potato GAME25 enzyme pointed towards its dual activity, i.e., (i) oxidation of the 3β-hydroxyl group (3β-hydroxysteroid dehydrogenase activity) and (ii) isomerization of the double bond from the C-5,6 position to the C-4,5 position (3-oxosteroid Δ⁵ isomerase activity) in SA and steroidal saponin aglycone substrates (FIGS. 19, 20, and 22A). The dual activity reported here for GAME25 is apparently novel as most if not all 3-βHSD enzymes participating in plant secondary metabolism (i.e. members of the 3-βHSD and SDR family) possess only 3β-hydroxysteroid dehydrogenase activity but not isomerase activity; for example, the 3-βHSD enzyme from Digitalis, Noscapine synthase (NOS) from poppy and momilactone synthase (MS) in rice. Interestingly, in mammals' steroid hormone metabolism, the conversion of pregnenolone to progesterone includes a 3βHSD enzyme that is similar to GAME25 and possesses a dual dehydrogenase and isomerase activity.

Additional results obtained in these assays showed that GAME25 is active with steroidal aglycones produced by tomato (dehydrotomatidine), potato (solanidine) and eggplant (solasodine) but not with the glycosylated steroidal alkaloids forms (e.g. α-tomatine, dehydrotomatine, α-solanine, α-chaconine and α-solamargine). The latter, more bulky structures that contain multi-glycoside residues might not be able to bind to the active site pocket in the GAME25 enzyme. The GAME25 enzyme was also not reactive with cholesterol and cholestenol that were predicted in the past to be precursor molecules for dehydrotomatidine and tomatidine, respectively. Furthermore, a similar three-step enzymatic conversion in wild potato species producing demissidine and further demissine SGAs as well as additional Solanum species like S. dulcamara that produce both the more toxic double bond containing SGAs and the less toxic, saturated ones (FIG. 22) is predicted.

Absence of GAME25 Activity in Cultivated Potato and Eggplants is Responsible for Lack of Saturated SGAs in them

The pathway from the unsaturated SA aglycone solanidine to saturated SA aglycone demissidine and its glycosylated form (i.e. demissine) in wild potato species corresponds to the tomato pathway in which the C-5,6 double bond is eliminated from dehydrotomatidine towards tomatidine and the glycosylated α-tomatine (FIG. 2B). While α-tomatine and its derivatives accumulate to high levels in tomato, the domesticated potato does not accumulate saturated demissidine or demissine SGAs in any plant part (while wild potato species do). The absence of the saturated SGAs demissidine or demissine in cultivated potato tubers albeit GAME25 gene in it suggests that these SGAs were selected against and lost during the domestication process (possibly through altered GAME25 activity). In addition, even if GAME25 is considered to be active in vivo, additional enzymes required for further conversion of solanid-4-en-3-one (3-oxo-Δ^{5,4} SA aglycone intermediate formed after GAME25 activity) might be absent or inactive in potato. Although recombinant GAME25 enzyme from potato showed in vitro activity with various SA aglycone substrates tested, the involvement of GAME25 gene in SGA metabolism in cultivated potato remains unclear (FIG. 19).

In the case of eggplant, merely GAME25 activity might be lacking as overexpression of the tomato GAME25 gene resulted in the production of saturated SGAs, suggesting the availability of the additional enzymes that are required to produce final saturated forms of SGAs after GAME25 action. In addition to saturated SGAs, novel saturated steroidal saponins were also formed in GAME25-Ox eggplant transgenic lines underscoring its crucial role in steroidal saponin biosynthesis in solanum plants. Based on these results, for other plant species that produce steroidal saponins, it is hereby predicted the existence of a GAME25-like enzyme and other additional enzymes in them that are required for eliminating the C-5,6 double bond from unsaturated steroidal saponin aglycone substrates and further to form saturated steroidal saponin aglycone and saponin glycosides (FIG. 3C).

### GAME25 is a Unique Classical SDR Family Member for Specialized Metabolism in Solanum Plants

In plants, more than 3000 known SDR enzymes catalyze NAD(P)(H)-dependent oxidation/reduction reactions on a wide range of substrates including alcohols, sugars, steroids, aromatic compounds and xenobiotics and thus contribute to a wide array of biochemical processes. As found in other eukaryotes, the majority of plant SDRs is either classical or extended types SDRs, yet, high number of SDRs in plants could not be classified to a particular functional category. With respect to distribution, the number of SDRs in land plants varies significantly, e.g. 126 in P. patens to around 315 in G. max. Members of SDR family have been reported to participate in the metabolism of various specialized metabolites including cardiac glycosides in Digitalis spp. (3βHSD), tropane-like alkaloids (SDR65C), terpenoids like xanthoxin, menthone and zerumbone (SDR110C, SDR114C), benzylisoquinoline alkaloids in poppy (NOS), oryzalexin diterpenoids in rice (MSI and MI1-3) and phenolic substrates, e.g. 4-dihydroflavanol, anthocyanidin, cinnamoyl-CoA and phenylacetaldehyde (SDR108E). The present disclosure shows that GAME25 is a key enzyme in the biosynthesis of saturated SGAs and unsaturated and saturated steroidal saponins, renowned classes of specialized metabolites in the Solanum genus. Phylogenetic analysis of SDR family proteins from plants partaking in specialized metabolism suggested that GAME25 proteins of the Solanaceae family have undergone significant diversification as compared to other family proteins that are known to be involved in plant secondary metabolism (FIG. 6). This can be supported by novel dual enzyme activities (dehydrogenase and isomerase) of GAME25 proteins in Solanaceae plants which could be acquired during the evolution over other SDR members that has evolved only with a single classic activity (e.g. only dehydrogenase).

Three Steps Enzymatic Conversion is Required for Formation of Saturated SA and Saponin Aglycones from Respective Unsaturated Forms

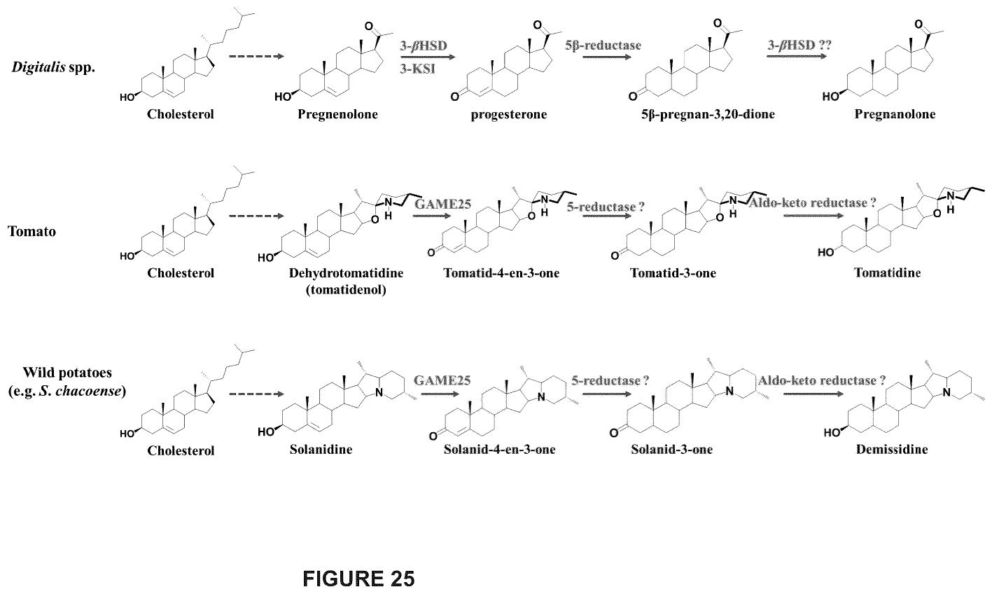

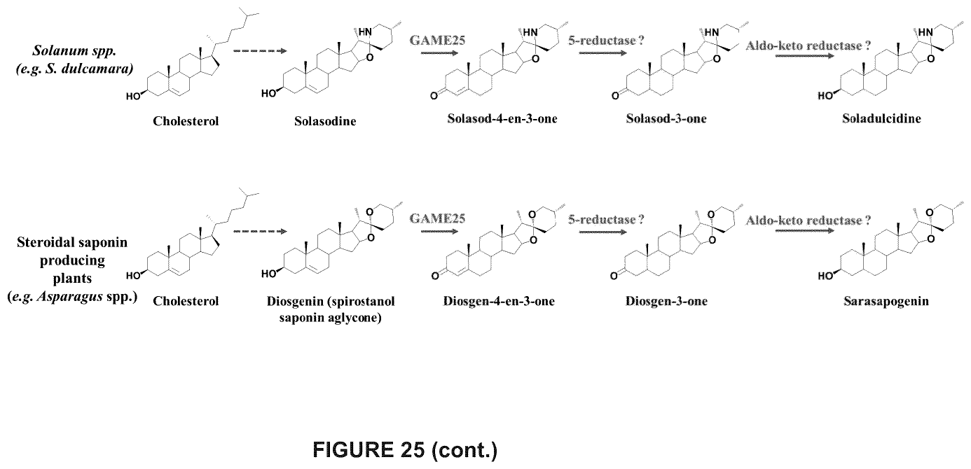

Digitalis species are known to produce an important class of specialized metabolites, the cardenolides or cardiac glycosides. During the biosynthesis of cardenolides, pregnenolone is converted to pregnanolone. Both pregnenolone and pregnanolone are steroid derivatives which differ only by the presence or absence of the double bond at the C-5,6 position (FIG. 25). The conversion of pregnenolone to pregnanolone (reduction of Δ⁵ bond) occurs in four steps: (i) oxidation of (3β-hydroxyl group) pregnenolone by 3βHSD enzyme followed by (ii) isomerization of double bond from the C-5,6 to the C-4,5 position by 3-KSI enzyme to form progesterone, (iii) In the third step, progesterone is converted to 5β-pregnan-3,20-dione (removal of C-4,5 bond) by 5β-progesterone reductase (5β-POR) which is subsequently converted to pregnanolone by 3βHSD enzyme. Thus, the conversion of pregnenolone to pregnanolone resembles to formation of tomatidine from dehydrotomatidine aglycone in tomato, or demissidine from solanidine aglycone in wild potato plants where C-5,6 double bond is also removed (FIG. 25). Interestingly, formation of saturated SA aglycone (e.g. tomatidine or demissidine) in Solanaceae plants was predicted to occur in single step by hypothetical hydrogenase enzyme. The present disclosure clearly demonstrates that the removal of C-5,6 bond of steroid derivatives in Digitalis spp. and SA aglycones in Solanaceae plants is strikingly different because of a novel dual activity of GAME25 enzyme that catalyzes first step in Solanum plants as compared to two separate enzyme activities required for same first step in Digitalis spp. (FIG. 25). In addition, 5β-progesterone reductase (5β-POR) homolog in tomato doesn't seem to be appropriate enzyme for catalyzing second step following GAME25 activity in saturated SGA biosynthesis. Based on further requirement of analogous enzymes after GAME25 reaction step that has been characterized extensively here in various Solanaceae plants, it is hereby proposed that the conversion of dehydrotomatidine to tomatidine in tomato is a three step reaction sequence with GAME25 catalyzing the first step, converting dehydrotomatidine to tomatid-4-en-3-one (3-oxo-Δ^{5,4} SA aglycone intermediate (FIG. 25). Tomatid-4-en-3-one is subsequently reduced to tomatidine by the successive action of a putative 5-reductase and an aldo-keto reductase (FIG. 25). The possible steroidal glycoalkaloids pathway reactions from the unsaturated aglycone solanidine to saturated demissidine and its glycosylated form (i.e. demissine) in wild potato species (e.g. S. chacoense) corresponds to the tomato SGA pathway reactions where the C-5,6 double bond is eliminated from dehydrotomatidine towards tomatidine in three steps (FIG. 25). A similar three-step conversion in wild potato species producing saturated demissidine from solanidine aglycone is predicted hereby. In addition, tomato GAME25 overexpression in cultivated eggplants generated saturated SGAs (soladulcidine and its glycosylated derivatives) from solasodine aglycone that are actually native to certain solanum species (e.g. S. dulcamara), thus indicating presence of active GAME25 in them. Based on this, it is hereby suggested that three step conversion reactions in those solanum species (e.g. S. dulcamara) produce saturated soladulcidine from solasodine aglycone (FIG. 25). Finally, based on in vivo (GAME25-Ox in eggplant) and in vitro (enzyme activity) results, it is hereby proposed three step reactions in the hundreds of steroidal saponin producing plant species that produce saturated saponin aglycones from their unsaturated forms and further saturated steroidal saponin glycosides (FIG. 25).

The characterization of GAME25 activity in the SGAs and biosynthesis pathway is a significant step towards resolving the complete core SGAs pathway in Solanaceae species. Likewise, it further contributes to the understanding of how a large portion of the structural diversity in the SGAs steroidal saponin producing Solanaceae species is formed. Nevertheless, the enzymes completing the elimination of the C-5,6 double bond from the core SGAs aglycones succeeding GAME25, are yet to be identified. The dramatic shift from saturated (without C-5,6 double bond) to unsaturated SGAs (with C-5,6 double bond) in GAME25 silenced tomato plants including the dominance of dehydro-esculeosides in ripening fruit make these genetic materials an excellent resource for future investigation. It will allow probing the significance of this structural variance to the potency of these molecules with respect to plant pathogens and herbivores. Ripe fruit accumulating α-tomatine on behalf of the typical esculeosides as a result of GAME25 overexpression will also be of value for carrying out similar interaction studies in tomato. The presence of the double bond at the C-5,6 position is not merely an issue of structural variation, but ample evidence suggests its relevance to the level of toxicity these molecules possess to humans. SGAs, largely the unsaturated ones, prevalent in potato tubers are renowned ‘anti-nutritionals’ and their levels in cultivated potato are tightly regulated. Together with the previously reported structural and regulatory genes, overexpression of GAME25 affords a valuable strategy to reduce the levels of these substances in commercial potato varieties.

### Methods for Examples 12-17

### Plant Material

The tomato Introgression Line (IL) and Backcross Inbred Line (BIL) populations derived from crosses of the M82 tomato cultivar and Solanum pennellii were obtained from publicly available collections of inbred lines as described in Ofner, I et al., (2016) Solanum pennellii backcross inbred lines (BILs) link small genomic bins with tomato traits. Plant J. 87(2):151-160; and Eshed and Zamir (1995) An introgression line population of Lycopersicon pennellii in the cultivated tomato enables the identification and fine mapping of yield-associated QTL. Genetics 141: 1147-1162. These populations (IL and BIL) consist of 671 lines, including the two parental lines M82 and S. pennellii, F1, 132 ILs and 536 BILs. The plants were grown in a climate-controlled greenhouse at 24° C. during the day and 18° C. during night, with natural light.

The GAME31-RNAi construct was created by introducing a GAME31 fragment to pENTR/D-TOPO (Invitrogen) (by NotI and AscI) and further transfer of the resulting cloned fragment to the pK7GWIWG2 (II) binary vector (Karimi et al., (2002) GATEWAY™ vectors for Agrobacterium-mediated plant transformation. Trends Plant Sci. 7: 193-195) using Gateway LR Clonase II enzyme mix (Invitrogen). The GAME31-Cosup (co-suppression) construct was generated by introducing the corresponding tomato GAME31 coding sequences into pDONR221 using the Gateway BP Clonase II enzyme mix (Invitrogen) and then transferred to the pK2GW7 binary vector using Gateway LR Clonase II enzyme mix. Constructs were transformed into tomato (cv. Microtom) as described previously (Itkin et al., 2011, ibid, 2013, ibid). Primers used in this work are listed in Table I, below:

TABLE 1
Primers used for Examples 12-17.

| Name | Sequence | SEQ ID NO: | Use |
|---|---|---|---|
| RF-Tomato- | tccgcgggtgaaaacctgtacttccaggg | 54 | RF cloning |
| GAME31-Fw | tgcatctatcaaatcagttaaagttc | | |
| RF-Tomato- | gtggtggtgctcgagtgcggccgcaagc | 55 | RF cloning |
| GAME31-Rv | tttcaaacaccacaataaatcttgaaaag | | |
| RF-Eggplant- | tccgcgggtgaaaacctgtacttccaggg | 56 | RF cloning |
| GAME31-Fw | tggatctaccaaatcaattaaagttc | | |
| RF-Eggplant- | gtggtggtgctcgagtgcggccgcaagc | 57 | RF cloning |
| GAME31-Rv | tttcaaacaccacaataagcctt | | |
| Diox460-Fw | agtacaagtaatcacgttggttcctatga | 60 | qRT-PCR |
| Diox460-Rv | cactagaaagttttttactttataggtgagg | 61 | qRT-PCR |
| | aaa | | |
| Dioxy-NotI- | aaaaagcggccgcgggatcgaggaaat | 62 | Cloning |
| RNAi-Fw | tcgtct | | SlGAME31 for |
| | | | RNAi |
| Dioxy-AscI- | aaaaaggcgcgccctaaaagattacggt | 63 | Cloning |
| RNAi-Rv | gaatcctctt | | SlGAME31 for |
| | | | RNAi |
| Dioxy-attB1- | ggggacaagtagtacaaaaaagcaggct | 64 | Cloning |
| Fw | atggcatctatcaaatcagttaaagt | | SlGAME31 for co- |
| | | | suppression |
| Dioxy-attB2- | ggggaccactttgtacaagaaagctgggt | 65 | Cloning |
| Rv | tcaaacaccacaataaatcttgaaa | | SlGAME31 for co- |
| | | | suppression |

### Screening of SGAs in Tomato BIL and IL Populations

For screening experiments for SGAs, a rapid extraction method and a short 10-min run in the UPLC-qTOF-MS (Xevo, Waters) was developed. For the extraction, the leaflet next to the youngest leaf was selected (about −1-month old plants). The leaflet was dipped in 1 ml of isopropanol: acetonitrile: water (3:3:2 v/v) containing 0.100 formic acid (in a 2 ml Eppendorfftube) and gently rocked for 1 min. Then the solvent was transferred to a LC vial for injection. The leaflets were dried in an oven to obtain their weight (mg).

Data from UPLC-qTOF-MS analyses were manually inspected in order to determine the major SGA metabolites present in the populations. Elemental composition and MS/MS fragmentation patterns were used for putative identification of SGAs previously reported (Itkin et al., 2011, ibid). A list of peaks was generated using retention time (RT) and m/z as identifiers for each compound. Peak area quantification was performed by the program TargetLynx (Waters). The resulting values were normalized to dry leaf weights and considered to be the amount of metabolite present in each sample. For ground-tissue, preparation of plant extracts and metabolite analysis by UPLC-qTOF-MS was carried out as described previously (Cirdenas et al., (2016) GAME9 regulates the biosynthesis of steroidal alkaloids and upstream isoprenoids in the plant mevalonate pathway. Nat. Commun. 7: 10654.).

Briefly, tomato plant tissues were frozen in liquid nitrogen and ground to a fine powder using an analytical mill or mortar and pestle. Then, frozen tissue (100 mg) was extracted with 80% methanol:water (v/v) containing 0.1% formic acid [the solid:liquid ratio was kept at 1:3 (w/v)]. The mixture was vortexed for 30 s, sonicated for 30 min at room temperature, vortexed again for 30 s, centrifuged (20,000×g, 10 min), and filtered through a 0.22-mm polytetrafluoroethylene membrane filter. Metabolite targeted analysis was performed using the targeted analysis program, TargetLynx program (Waters).

### Genotyping and Mapping of QTL

The BIL/IL populations were constructed and genotyped as previously described (Ofner et al., 2016, ibid).

### Generation of Recombinant GAME31 Enzyme

GAME31 was amplified from cDNA (SEQ ID NO: 17) and cloned into pET28 vector by restriction free (RF) cloning as described previously (Unger et al., 2010, J Struct Biol. 172:34-44). The primers used for the cloning of GAME31 are listed in Table 1 above.

The resulting plasmids were verified by sequencing and transformed to E. coli BL21 DE3. For isolation of the GAME31-His tagged enzymes, fresh overnight cultures were diluted 1:100 in 1000 ml of LB medium (Formedium) with 50 μg/ml kanamycin and incubated at 37° C. and 250 rpm until an A_{600 nm} of 0.5 was reached. Subsequently, for induction of expression of the recombinant proteins, IPTG was added to a final concentration of 0.2 μM and the incubation was continued overnight at 16° C. and 250 rpm. Then cells were harvested by centrifugation at 10,000×g and bacteria were lysed by sonication in a buffer (pH 7.4) containing 0.02 M NaH₂PO₄ 0.02M, 0.5 M NaCl, and SIGMAFAST protease inhibitor tablets (Sigma-Aldrich) and benzonase nuclease (Sigma-Aldrich) according to manufacturer's instructions. Lysed cultures were centrifuged, and the soluble fraction was purified by nickel affinity chromatography which was operated with an AKTA liquid chromatography system (AKTA avant, GE Healthcare) according to the manufacturer's instructions. Proteins were stored at −80° C. until further analysis.

### Enzymatic Activity Assays for GAME31 Enzymes

All the SA/SGAs substrates were prepared at a concentration of 1 mg/ml, dissolved in methanol, except dehydrotomatine that was prepared in DMSO. The enzymatic activity was performed according to Kawai et al. (2014) Evolution and diversity of the 2-oxoglutarate-dependent dioxygenase superfamily in plants. Plant J. 78: 328-43. Briefly, the standard full reaction (100 μL) consisted of 10 mM L-ascorbic acid, 10 mM α-ketoglutaric acid, 500 μM FeSO₄, 20 μM substrate, 50 mM potassium phosphate buffer (pH 7.5) and purified enzyme. All the components, except the enzyme, were pre-incubated for 10 min at 30° C., after which the reaction was started by addition of the enzyme. After incubation at 30° C. for 1 h, the reaction was stopped by freezing in liquid nitrogen. Finally, the reaction was mixed with 300 μL methanol, extracted and analyzed by UPLC-qTOF-MS as described above.

### Quantitative Real-Time PCR

Gene expression analysis was performed with three biological replicates (n=3) for each genotype. RNA isolation was performed by the Trizol method (Sigma-Aldrich). DNase I (Sigma-Aldrich)-treated RNA was reverse transcribed using a high-capacity cDNA reverse transcription kit (Applied Biosystems). Gene-specific oligonucleotides were designed with Primer Express 2 software (Applied Biosystems). The TIP41 gene (Expósito-Rodriguez et al., (2008) Selection of internal control genes for quantitative real-time RT-PCR studies during tomato development process. BMC Plant Biol. 8: 131-142) was used as an endogenous control for tomato samples. Oligonucleotides used are listed in Table 1, above.

### SGA Targeted Analysis

Preparation of plant extracts and metabolite analysis by UPLC-qTOF-MS was carried out as described previously (Cirdenas et al., 2016, Nat. Commun. 7:10654). Briefly, tomato plant tissues were frozen in liquid nitrogen and ground to a fine powder using an analytical mill or mortar and pestle. Then, frozen tissue (100 mg) was extracted with 80% methanol:water (v/v) containing 0.1% formic acid (the solid:liquid ratio was kept at 1:3 [w/v]). The mixture was vortexed for 30 s, sonicated for 30 min at room temperature, vortexed again for 30 s, centrifuged (20,000 g, 10 min), and filtered through a 0.22-mm polytetrafluoroethylene membrane filter. Metabolite targeted analysis was performed using the TargetLynx program (Waters).

### Constructs

The GAME31-RNAi construct was created by introducing a GAME31 fragment into pENTR/D-TOPO (Invitrogen) (by NotI and AscI) and further transferring the resulting plasmid to the pK7GWIWG2 (II) binary vector (Karimi et al., 2002, Trends Plant Sci. 7:193-195) using Gateway LR Clonase II enzyme mix (Invitrogen).

The GAME31-Cosup (co-suppression) construct was generated by introducing the corresponding tomato GAME31 coding sequences (SEQ ID NO: 59) into pDONR221 using the Gateway BP Clonase II enzyme mix (Invitrogen) and then transferred to the pK2GW7 binary vector using Gateway LR Clonase II enzyme mix.

Constructs were transformed into tomato (cv. Microtom) as described previously (Itkin et al., 2011, Plant Cell 23:4507-45-25; Itkin et al., 2013, Science 341:175-179.). Primers used in this work are listed in Table 1.

### Example 12

### Screening of SGAs in Backcross Inbred Line (BIL) and Introgression Line (IL) Populations

Objective: To find new candidate genes involved in the metabolism of steroidal glycoalkaloids (SGAs). FIGS. 1-3 provide the core genes (GLYCOALKALOID METABOLISM genes; GAMEs) required for synthesis of SA aglycones, starting from the precursor cholesterol, and its glycosylation in tomato have been elucidated (Itkin et al., 2011 ibid., 2013, ibid). In tomato, cholesterol is generated from the cytosolic mevalonic acid pathway and further modified by GLYCOALKALOID METABOLISM (GAME) enzymes (in blue) through hydroxylation, oxidation and transamination to generate the aglycone tomatidine. Then, tomatidine is glycosylated generating α-tomatine, the major SGA in green tissues along with dehydrotomatine. Subsequently, hydroxy- and acetoxy-derivatives accumulate at the fruit breaker stage. In the red ripe tomato fruit, the most abundant SGA correspond to esculeoside A. SGAs detected in the leaf-dip screening of the BIL/IL populations are shown in green. Dashed arrows represent multiple biosynthetic reactions whereas solid arrows represent a single step.

### Results:

In the search for new candidate genes involved in the metabolism of steroidal glycoalkaloids (SGAs), SGA levels were screened in tomato Introgression Line (IL) and Backcross Inbred Line (BIL) populations derived from crosses of the M82 tomato cultivar and Solanum pennellii LA0716. This whole set consisted of 671 lines, including the two parental lines M82 and S. pennellii, F1, 132 ILs and 536 BILs. In these lines, single (in the case of the ILs) or multiple (for the BILs) regions from the wild species S. pennellii, replaced the homologous counterpart of the cultivated M82 variety. In order to analyze alkaloid content in the large number of lines in a high-throughput manner, metabolite analysis was performed using a leaf-dip method (Schilmiller et al., (2010) Mass spectrometry screening reveals widespread diversity in trichome specialized metabolites of tomato chromosomal substitution lines. Plant J. 62: 391-403), instead of extracting the metabolites from ground tissues. Leaflets from ˜1-month old plants were harvested and immediately extracted by dipping in the extraction solvent and gentle agitation. This method offers information on a relatively low number of SGAs as compared to the number of SGAs detected in ground-tissue extracts; however, it provided a more efficient screening procedure.

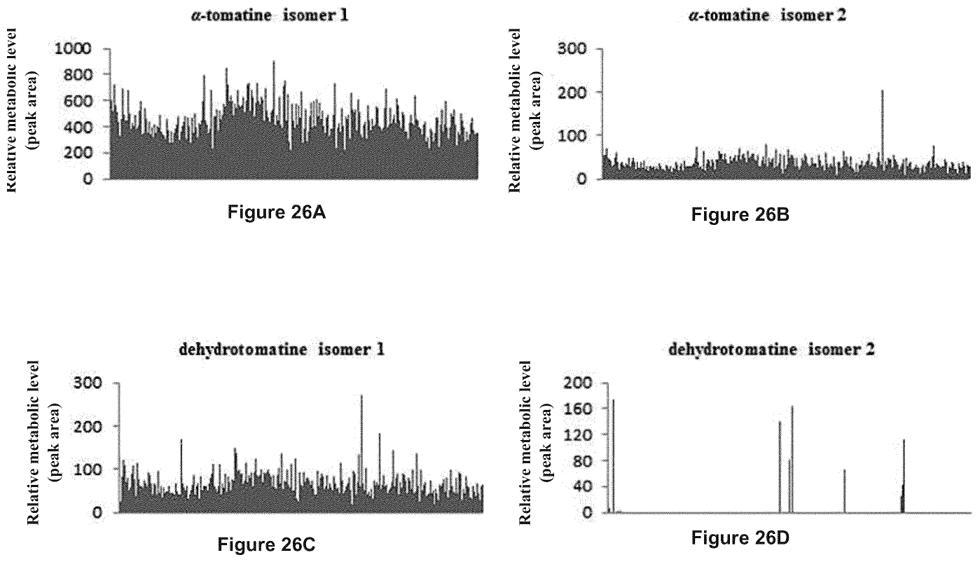

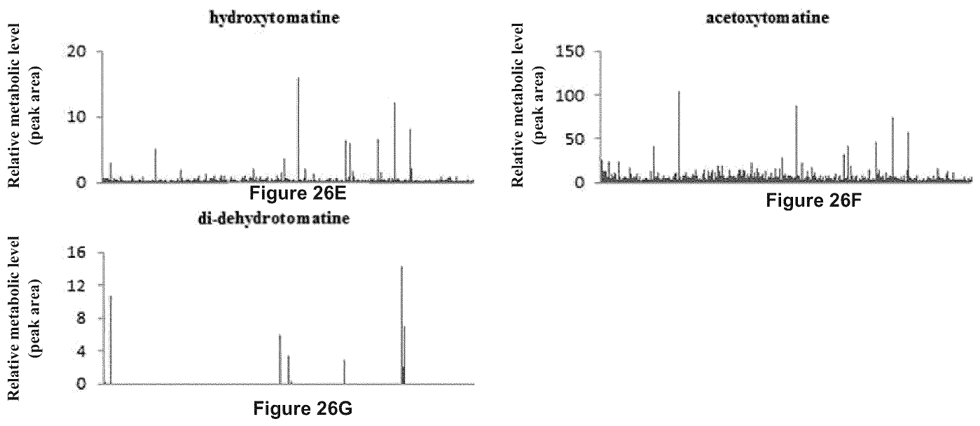

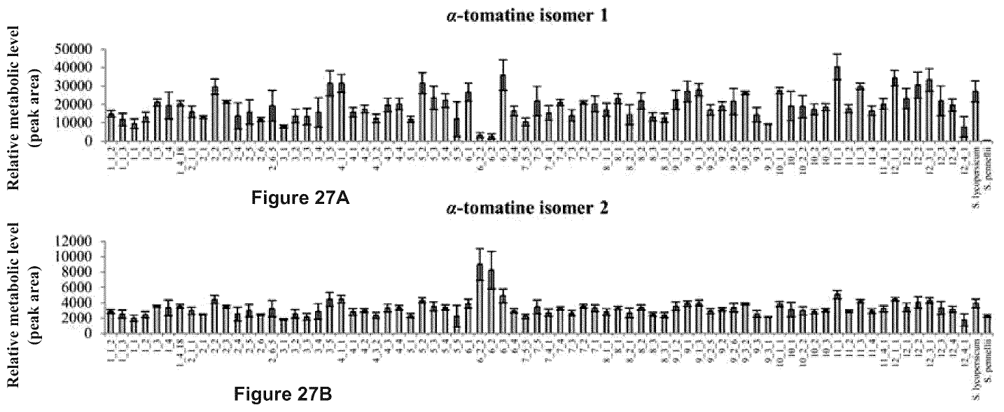

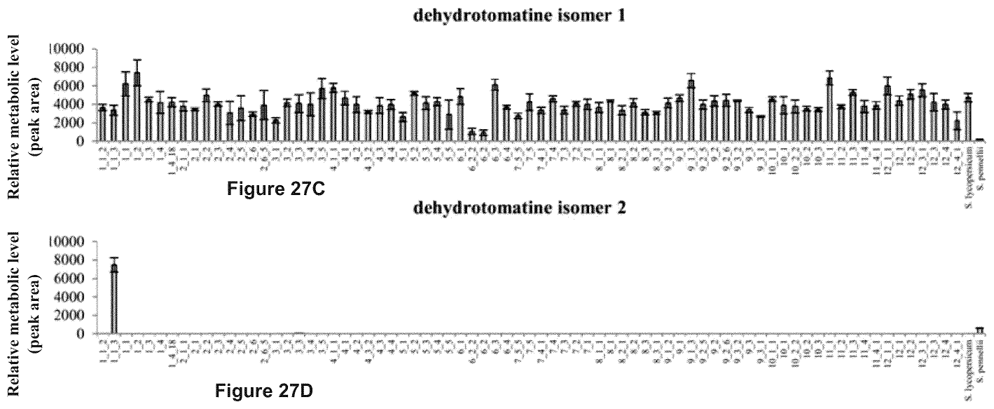

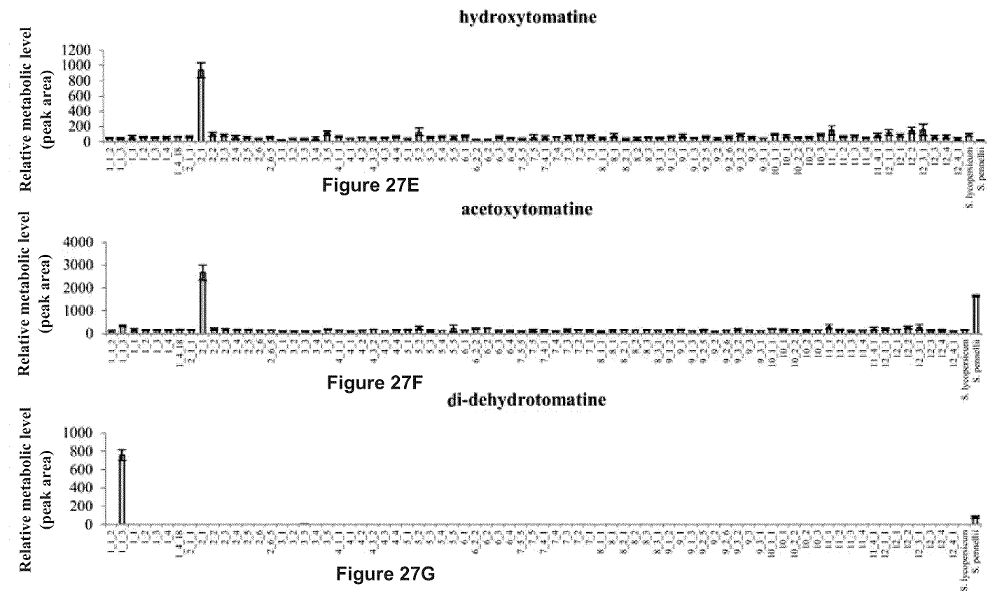

For profiling of SGAs, a rapid method using a short 10-minute run in the Ultra Performance Liquid Chromatography coupled to Quadrupole Time-Of-Flight Mass Spectrometry (UPLC-qTOF-MS) was developed. This allowed screening 7 different SGAs: 2 isomers of α-tomatine, 2 isomers of dehydrotomatine and 1 isomer of di-dehydrotomatine, acetoxytomatine and hydroxytomatine (FIGS. 1A-1C, 2A-2B, 3A-3C, and FIGS. 26A-26G). Under tested conditions, it was found that the α-tomatine isomers 1 and 2 and dehydrotomatine isomer 1 were the most abundant SGAs found in the population. Detailed inspection of the chromatograms revealed a second early-eluting isomer of dehydrotomatine present only in few samples. Similarly, hydroxytomatine, acetoxytomatine and di-dehydrotomatine were either absent or present at very low levels across the population and few lines accumulated them in larger quantities (FIGS. 26A-26G). These results were validated in ground-tissue extracts derived from the IL population and the relevant BILs (FIGS. 27A-27G).

### Example 13

### Mapping of GAME31 in Tomato and Identification in Other Solanum Species

Objective: To identify chromosomal areas linked to the variation of each SGA.

### Results:

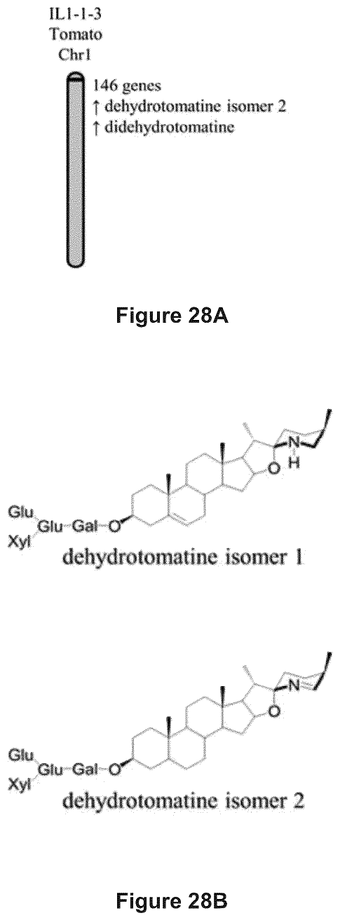

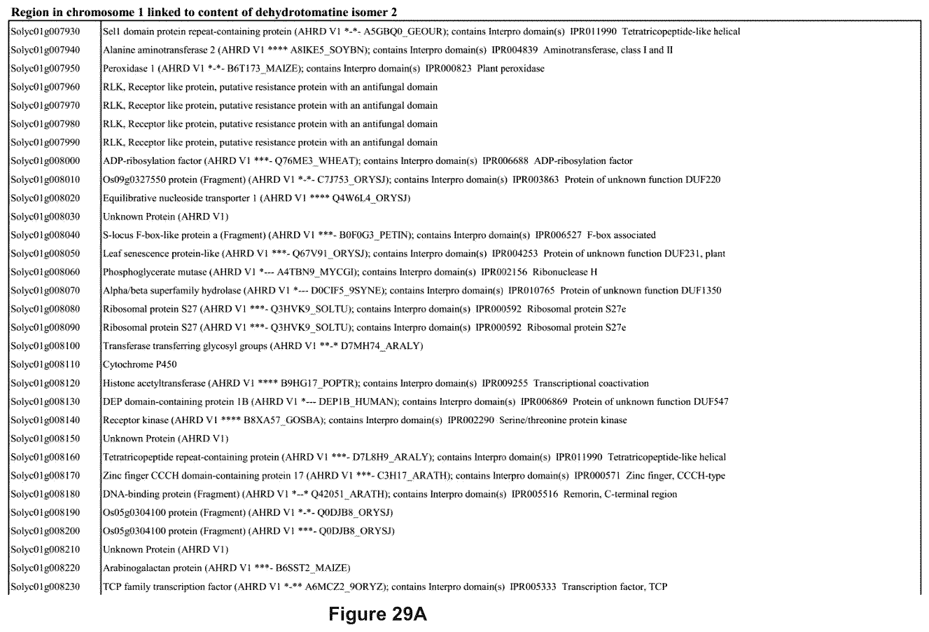

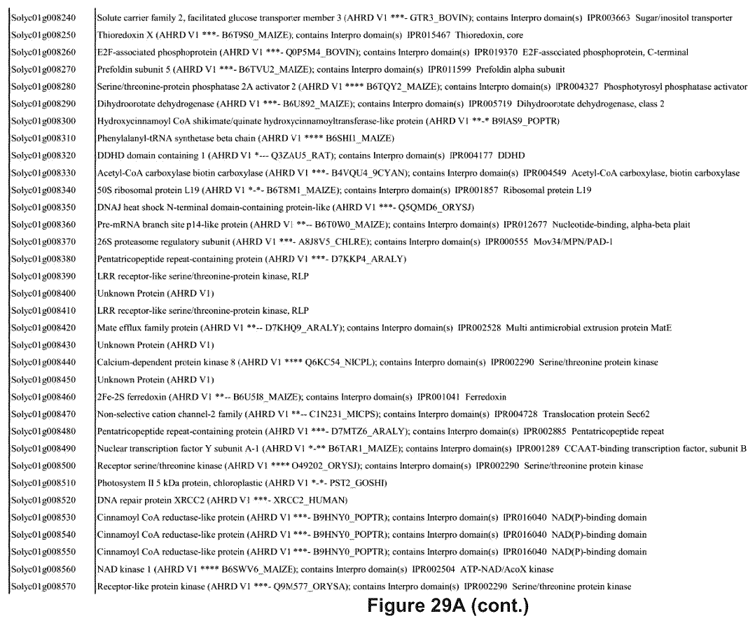

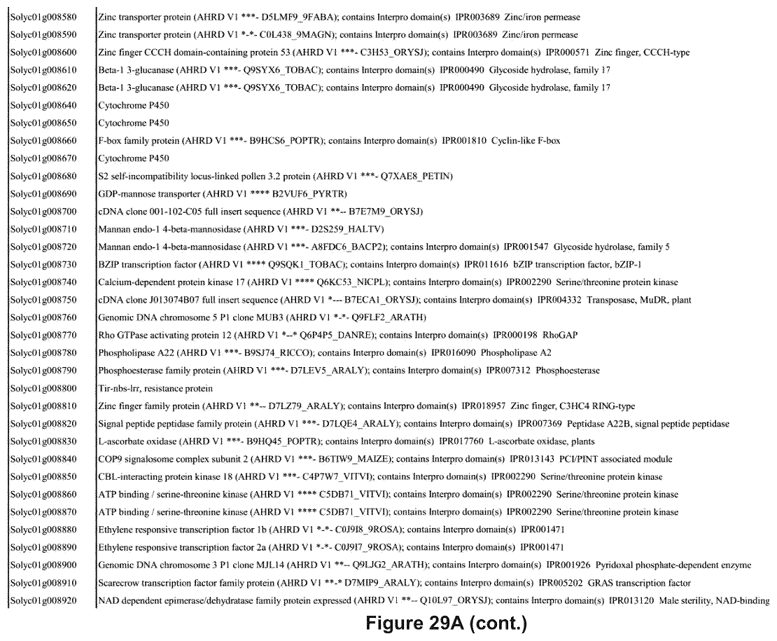

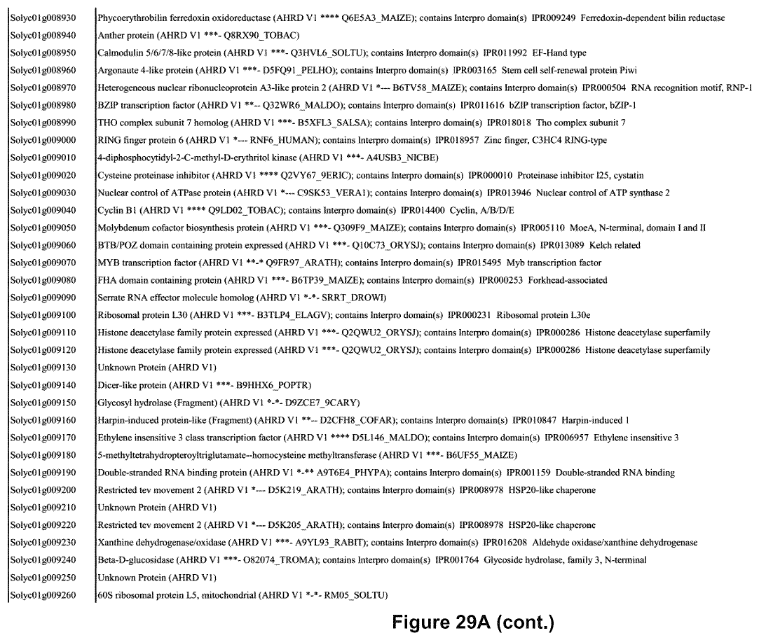

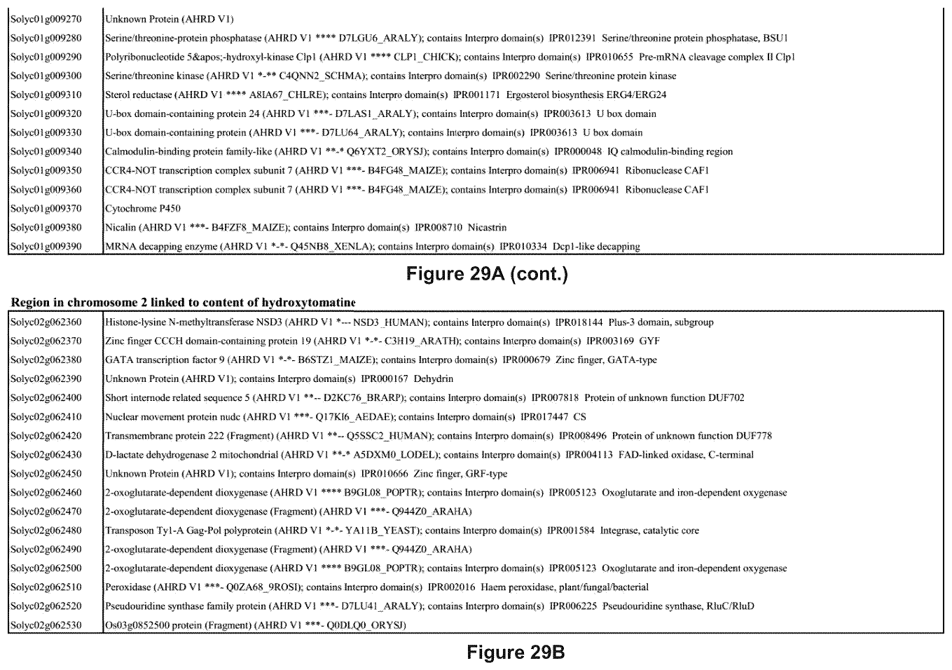

Using the SGA content information from the screened lines, chromosomal areas linked to the variation of each SGA were identified. For instance, a region in chromosome 1 (covering 146 genes) associated to accumulation of dehydrotomatine isomer 2 in IL1-1-3 was identified (FIGS. 28A and 28B). This region has been previously reported from the IL population, and it has been suggested that this accumulating metabolite could be converted to α-tomatine by the action of a reductase, and that this activity was deficient in the ILs accumulating dehydrotomatine isomer 2 (Schilmiller et al., (2010), ibid). A detailed examination of the 146 genes in the region (FIGS. 29A and 29B), allowed us to point a candidate reductase (Solyc01g009310) and several cytochrome P450 enzymes as the most likely responsible enzymes for the observed metabolic change (FIGS. 28A and 28B).

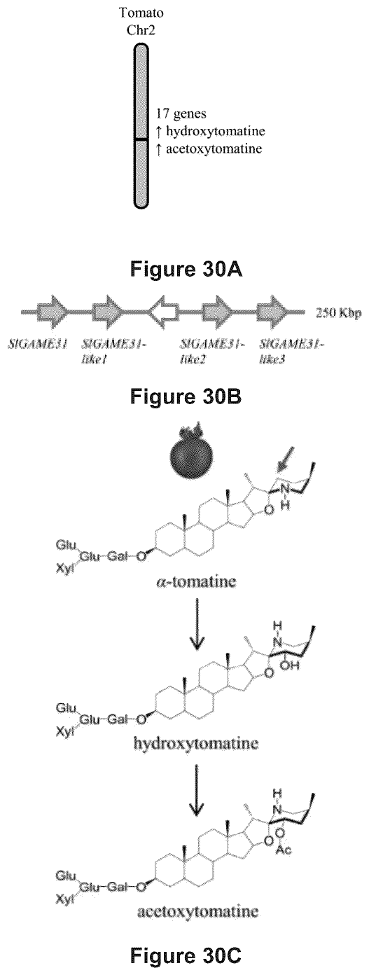

A region spanning ˜250 Kbp in chromosome 2 of tomato (Solanum lycopersicum) was also identified as associated with an increase in hydroxytomatine and acetoxytomatine content (FIGS. 30A-30C). The increased amounts of these compounds in leaf tissues suggested the presence of an active hydroxylating enzyme in these BIL/IL populations. An examination of the 17 genes found in the QTL region revealed the presence of four 2-oxoglutarate-dependent dioxygenases (FIGS. 29A and 29B). The gene annotation provided the first indication that these enzymes may be acting in the hydroxylation of α-tomatine into hydroxytomatine. The genes were named GLYCOALKALOID METABOLISM 31 (SlGAME31; Solyc02g062460; SEQ ID NO: 16), SlGAME31-like1 (Solyc02g062470), SlGAME31-like2 (Solyc02g062490) and SlGAME31-like3 (Solyc02g062500) (FIG. 30B). Two of them are full length 2-oxoglutarate-dependent dioxygenase coding proteins (i.e. SlGAME31 and SlGAME31-like3), while the two others are partial (i.e. SlGAME31-like1 and SlGAME31-like2).

The annotation as a dioxygenase provided the first indication of the GAME31 enzyme (SEQ ID NO: 18) as the putative enzyme responsible for hydroxylating α-tomatine to form hydroxytomatine.

Hydroxylated SGAs are also found in other Solanum species. In eggplant (S. melongena), α-solamargine is hydroxylated generating hydroxysolamargine. In cultivated potato (S. tuberosum), hydroxylated SGAs are found in minor amounts. However, in wild relatives, like S. chacoense, SGAs are accumulated in higher amounts. When hydroxylated, α-chaconine and α-solanine are converted into leptinine I and leptinine II, respectively. Furthermore, these two potato SGAs could be further acetylated into leptine I and leptine II, respectively.

In the tomato chromosomal region harboring GAME31, as described above and in FIGS. 30A-30C, three additional SlGAME31-like genes were found (Table 2).

TABLE 2
Sequence IDs for GAME31 and GAME31-like genes in Solanum spp.

| | | | SEQ |
|---|---|---|---|
| Solanum spp | | Sequence ID** | ID NO: |
| S. lycopersycum | SlGAME31 | Solyc02g062460 | 16 |
| | SlGAME31- | Solyc02g062470 | 17 |
| | like1 | | |
| | SlGAME31- | Solyc02g062490 | 22 |
| | like2 | | |
| | SlGAME31- | Solyc02g062500 | 25 |
| | like3 | | |
| S. melongena | SmGAME31 | Sme2.5_04260.1_g00001.1 | 28 |
| S. tuberosum | StGAME31 | Sotub01g007080 | 30 |
| | (CDS) | | |
| | StGAME31- | Sotub01g007070 | 33 |
| | like1 | | |
| | StGAME31- | Sotub01g007090 | 36 |
| | like2 | | |
| | StGAME31- | Sotub01g007100 | 39 |
| | like3 | | |
| | StGAME31- | Sotub01g007110 | 42 |
| | like4 | | |
| | StGAME31- | Sotub01g007120 | 45 |
| | like5 | | |
| | StGAME31- | Sotub01g007130 | 48 |
| | like6 | | |
| | StGAME31- | Sotub01g007150 | 53 |
| | like7 | | |

**Sol Genomics Network (for tomato and eggplant); Spud DB (for potato)- (Fernandez-Pozo N, Menda N, Edwards JD, Saha S, Tecle IY, Strickler SR, Bombarely A, Fisher-York T, Pujar A, Foerster H, Yan A, Mueller LA. The Sol Genomics Network (SGN)-from genotype to phenotype to breeding. (2015) Nucleic Acids Res. Volume 43 (Database issue): D1036-41.)

The knowledge of hydroxylated SGAs in eggplant and potato, led to an investigation of the presence of GAME31-like homologous genes in these species. In eggplant, one gene sitting on chromosome 1 (named SmGAME31) was found which has 88% similarity to SlGAME31 (78% identity), while in potato a region in chromosome 1 containing seven GAME31-like genes was found, with the closest gene having 86% similarity (77% identity) (Table 2).

### Example 14

Association of GAME31 with SGA Hydroxylation

Objective: To examine the function of GAME31 enzyme.

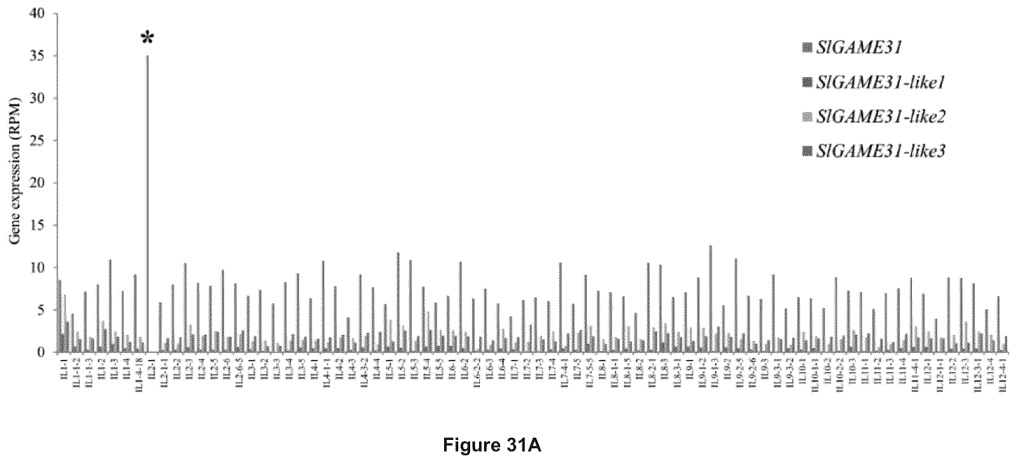

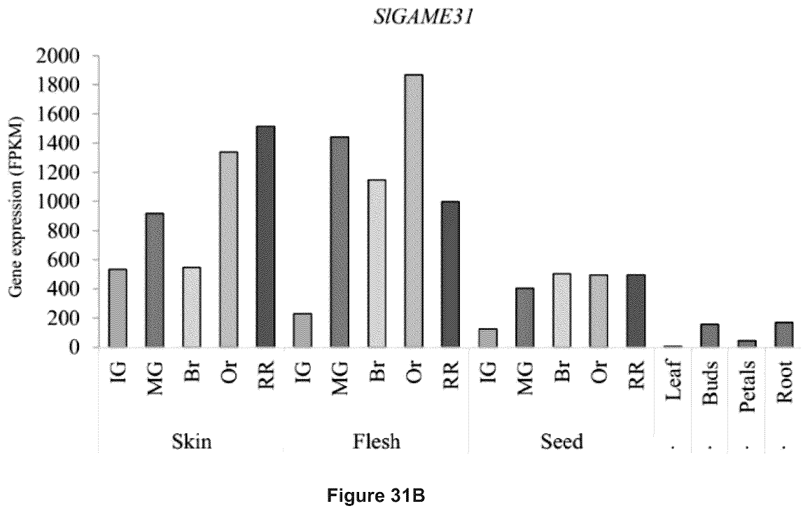

Results: The expression of genes found in the chromosome 2 region associated with higher hydroxytomatine and acetoxytomatine content in the BIL and IL lines was examined. RNA-Sequencing (RNA-Seq) gene expression data from vegetative apex, which had previously been reported for the IL population (Chitwood et al., (2013), A quantitative genetic basis for leaf morphology in a set of precisely defined tomato introgression lines. Plant Cell 25: 2465-2481), was used. By analyzing this dataset, expression of 12 out the 17 genes contained in the QTL region was detected. Excluding SlGAME31, all of the genes presented similar expression levels across the IL lines (<20 normalized Reads Per Million, RPM; data not shown). SlGAME31 showed 4.6-fold increased expression in IL2-1 (35 RPM) in comparison with the rest of the IL population (average 7.6 RPM) (FIG. 31A). SIGAME31-like1, SlGAME31-like2 and SlGAME31-like3 showed relatively low expression (<7 RPM) which appeared similar in all IL lines (FIG. 31A). The expression pattern of SlGAME31 in the ILs and increased accumulation of hydroxytomatine in IL2-1 (FIG. 27E) strongly suggested the role of this enzyme in hydroxylation of α-tomatine.

To provide additional evidence regarding the involvement of GAME31 in the hydroxylation of SGAs, the gene expression of GAME31 in RNA-Seq data covering various tissues and developmental stages of tomato (Cirdenas et al., (2016), ibid) was examined. It was found that SlGAME31 was highly expressed in fruit tissues among 19 different tomato tissues (FIG. 31B). In skin, flesh and seeds of tomato fruit the expression of GAME31 positively correlated with ripening, being less expressed in early immature stages of development. In other tissues, including leaf, flower buds, petals and roots SlGAME31 displayed lower expression levels. SlGAME31-like3 was found to be expressed at very low levels (<1.2 RPM), while SlGAME31-like1 and SlGAME31-like2 were not detected in this RNA-Seq dataset. The expression of SlGAME31 correlated with the previously reported accumulation of hydroxytomatine during tomato fruit ripening (Mintz-Oron et al., (2008) Gene expression and metabolism in tomato fruit surface tissues. Plant Physiol. 147: 823-851) supporting the hypothesis that SlGAME31 encodes an enzyme that potentially hydroxylates SGAs.

### Example 15

### Identification of SIGAME31 Homologs in Eggplant and Potato

Objective: To examine the genomes of other species of Solanaceae plants for homologs to SlGAME31 The knowledge of hydroxylated SGAs in other Solanaceae species like eggplant and potato led to the investigation of the presence of GAME31 homologous genes in these species.

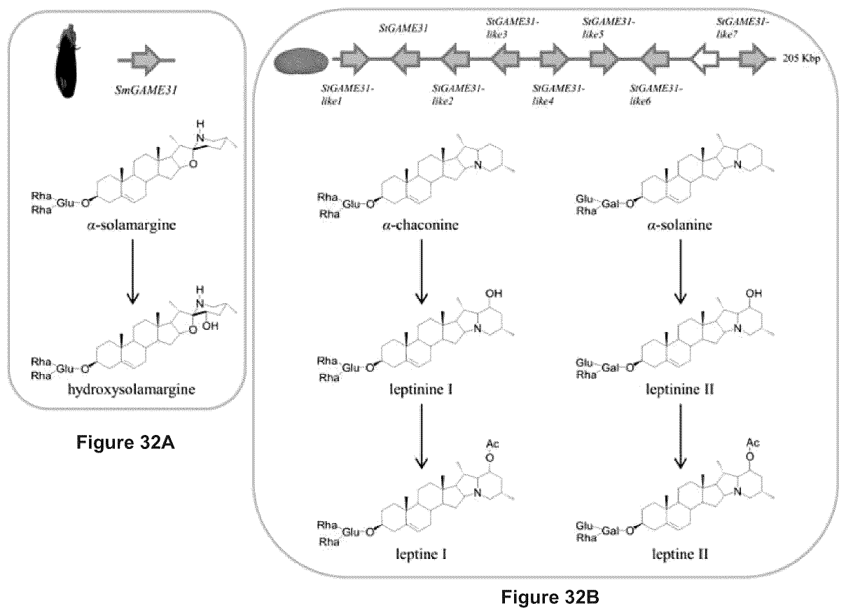

Results: Using sequence similarity searches in the Sol Genomics Network database, a single gene in eggplant (Solanum melongena) was identified located in chromosome 1 (SmGAME31; Sme2.5_04260.1_g00001.1) with 88% similarity (78% identity) at the protein level to SlGAME31 (FIGS. 32A-32B). In eggplant, SmGAME31 was predicted to catalyze the hydroxylation of α-solamargine to hydroxysolamargine.

In potato (Solanum tuberosum) a region was found in chromosome 1, spanning ˜205 Kbp and containing eight GAME31 homologs. One of these (StGAME31) displayed 86% similarity (77% identity) to SlGAME31 at the protein sequence level (FIGS. 32A-32B). Out of the eight homologs, four represented full length 2-oxoglutarate-dependent dioxygenase coding sequences [StGAME31 (Sotub01g007080), StGAME31-like1 (Sotub01g007070), StGAME31-like4 (Sotub01g007110) and StGAME31-like6 (Sotub01g007130)] and four possessed only partial coding sequences [StGAME3]-like2 (Sotub01g007090), StGAME31-like3 (Sotub01g007100), StGAME31-like5 (Sotub01g007120) and StGAME31-like7 (Sotub01g007150)]. In potato, StGAME31 is predicted to hydroxylate α-chaconine and α-solanine into leptinine I and II, respectively. These compounds are found in minor amounts in cultivated potatoes (Itkin et al., (2013), ibid); however, they are major relatively abundant in wild species like Solanum chacoense. Leptinine I and II can be further acetylated to produce leptine I and II, respectively (FIG. 32B).

### Example 16

### In Vitro Hydroxylation of SGAs by GAME31

Objective: To examine the enzyme activity of GAME31 in vitro.

Results: The in vitro function of the recombinant SIGAME31 produced in Escherichia coli was examined for hydroxylation activity with 8 SA/SGAs. From tomato α-tomatine and its aglycone tomatidine, dehydrotomatine were tested; from eggplant α-solamargine and its aglycone solasodine were tested; and in potato α-chaconine, α-solanine and its aglycone solanidine were tested.

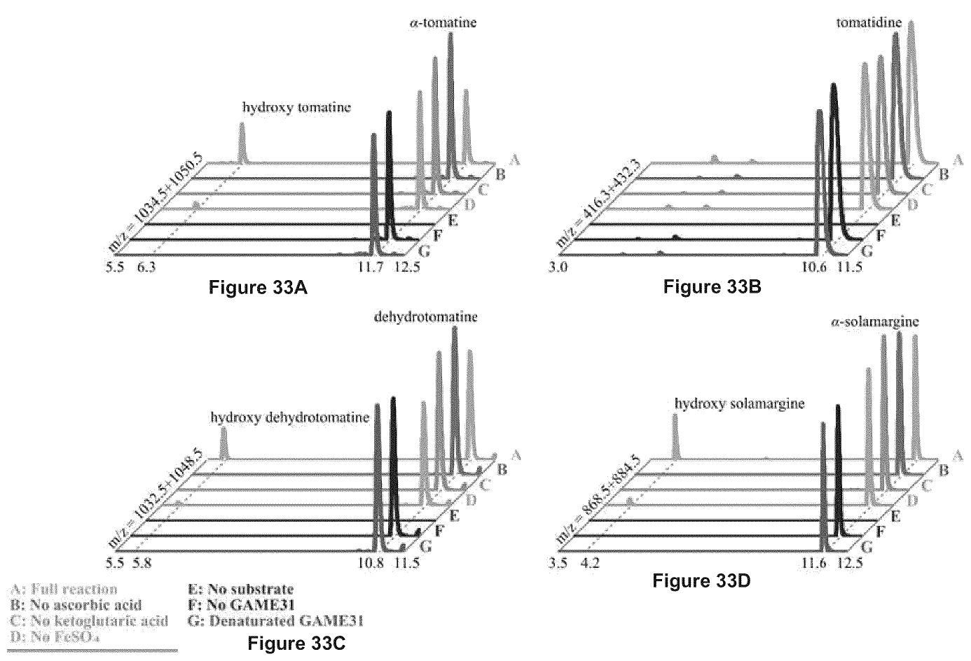

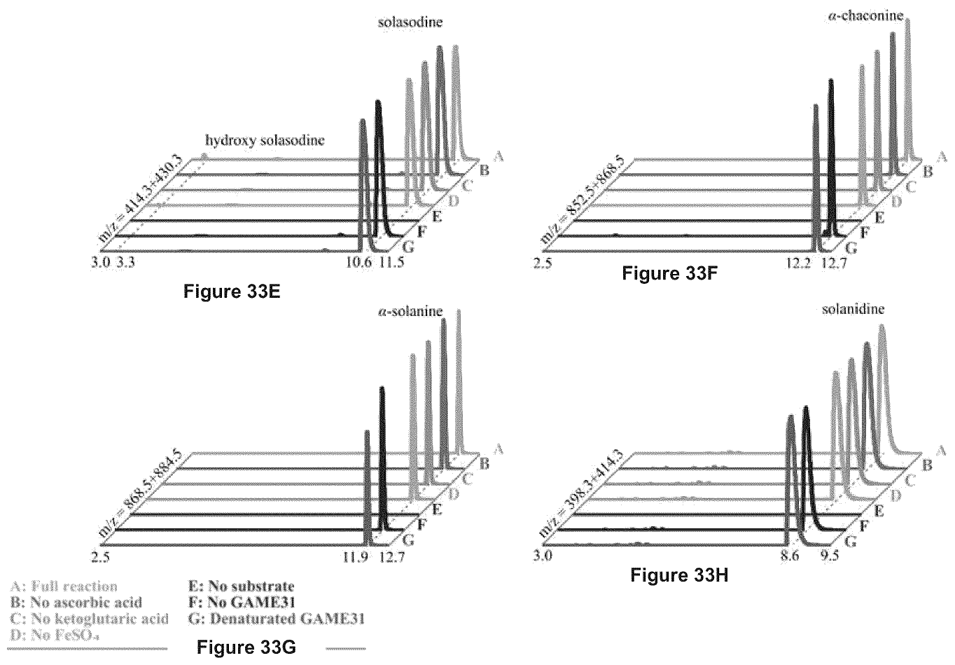

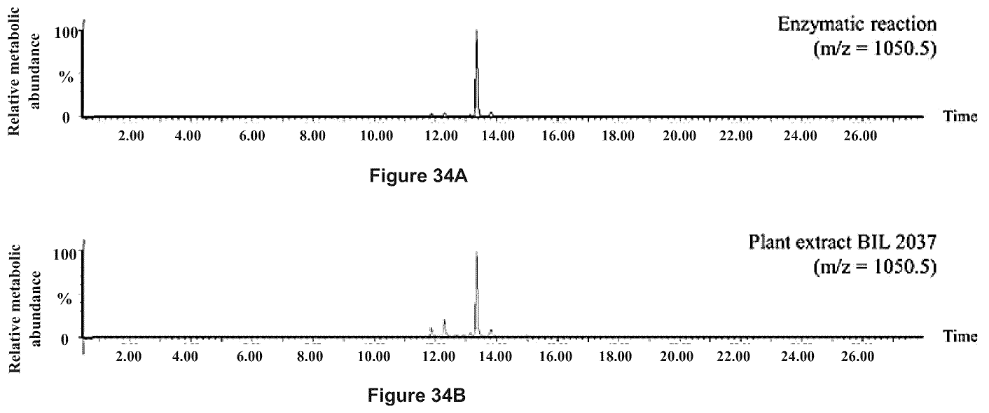

The recombinant SIGAME31 catalyzed the hydroxylation of α-tomatine forming hydroxytomatine (FIG. 33A) in the presence of the cofactors ketoglutaric and ascorbic acid. Removal of either cofactors resulted in lack of activity. Nevertheless, when the reaction was carried out in the absence of iron, minor amounts of hydroxytomatine were still generated. This is likely due to trace amounts of iron found in the reagents used; additionally previous reports have shown that the absence of iron had no effect in reactions catalyzed by a 2-oxoglutarate-dependent dioxygenase from Catharanthus roseus (De Carolis et al., (1990) Isolation and characterization of a 2-oxoglutarate dependent dioxygenase involved in the second-to-last step in vindoline biosynthesis. Plant Physiol. 94: 1323-1329). Interestingly, SIGAME31 generated the same hydroxytomatine isomer accumulating in the BIL lines containing the S. pennellii introgression on chromosome 2 (FIGS. 34A-34B). SIGAME31 did not show activity when the aglycone tomatidine was used as substrate, but it did perform hydroxylation of dehydrotomatine (FIGS. 33B-33C).

When SlGAME31 was tested with eggplant steroidal alkaloids, it hydroxylated α-solamargine generating hydroxysolamargine, and to a lesser extent the aglycone solasodine (FIGS. 33D and 33E). However, SlGAME31 did not hydroxylate any potato SA/SGA (FIGS. 33F-33H).

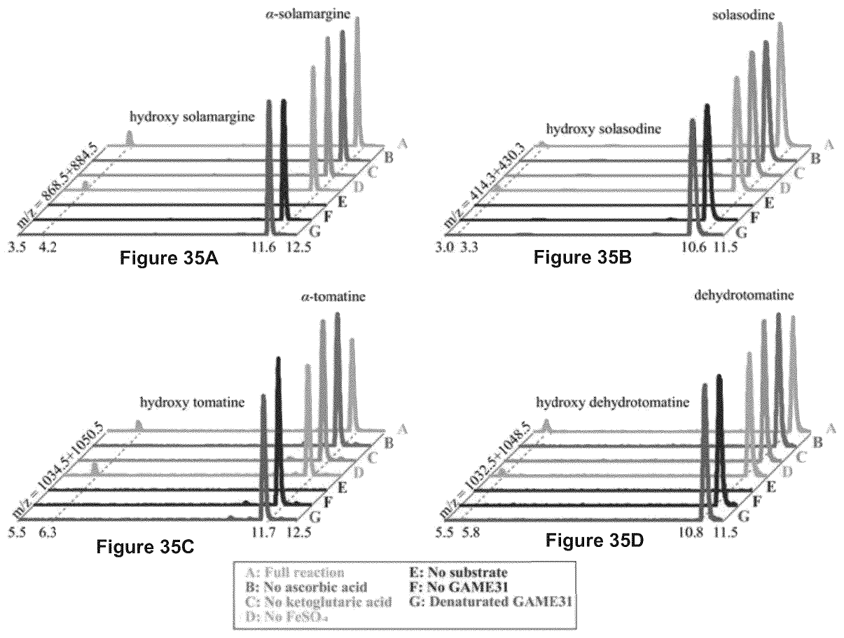

In the same way, enzymatic activity assays were performed using the recombinant enzyme cloned from eggplant, SmGAME31, and the same set of SA/SGAs used previously. Similar to the tomato enzyme, SmGAME31 performed hydroxylation of α-solamargine and to a lesser extent of solasodine, α-tomatine and dehydrotomatine (FIGS. 35A-35D). Conversely, SmGAME31 did not hydroxylate the aglycone tomatidine or potato SA/SGAs.

Conclusion: The preference of both tomato and eggplant GAME31 for glycosylated substrates (see FIGS. 33A-33H and 35A-35D) indicates that these enzymes act preferably after glucosyltransferases have added the sugar moiety to the 3-OH position on the A ring of the SA aglycone.

### Example 17

### Altering SlGAME31 Expression Impacts SGA Profile in Tomato Fruit During Ripening

Objective: To characterized in more detail the role of GAME31 enzyme and observe the SGA profile in tomato fruit when GAME31 expression is altered.

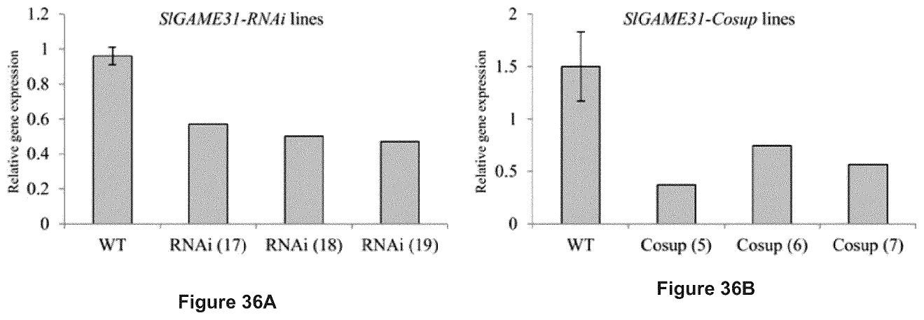

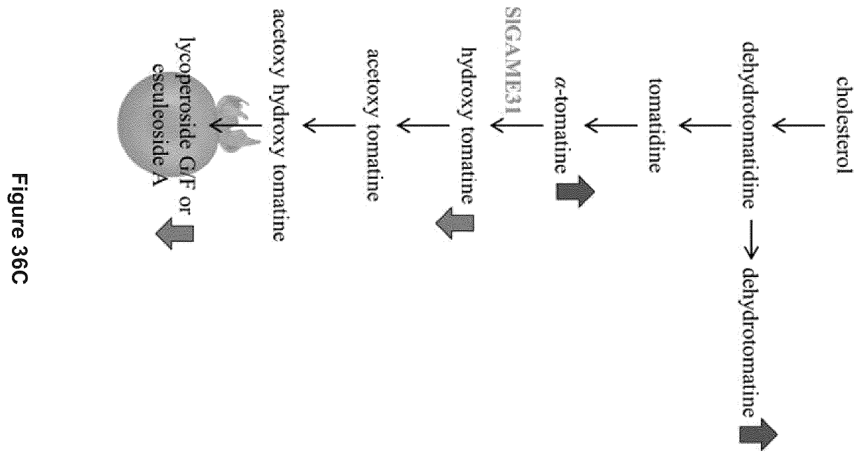

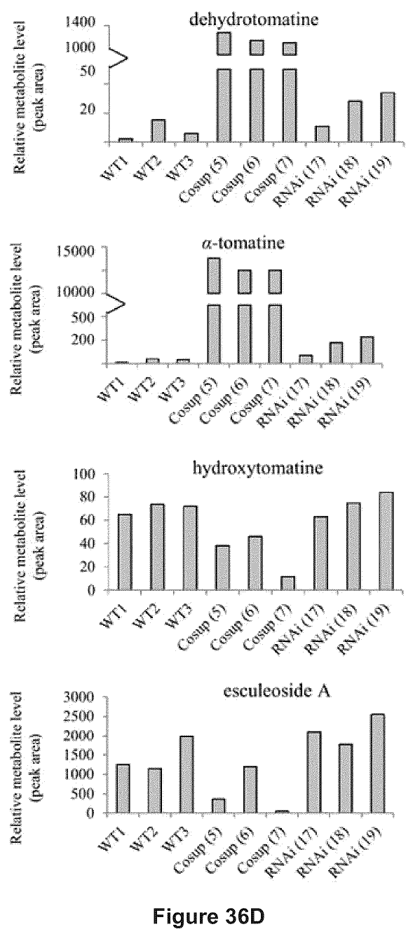

Results: To further understand the role of SlGAME31 in hydroxylation of SGAs, transgenic tomato lines in which SlGAME31 was silenced by RNA interference (SlGAME31-RNAi) were generated. Also, lines constitutively expressing the tomato GAME31 were generated but these turned to be co-suppressed (SlGAME31-Cosup). Thus, although these plants were generated as plants having overexpression of GAME31, co-suppression is a post-transcriptional mechanism where both the transgene and the endogenous gene are silenced. Quantitative Real-Time PCR (qRT-PCR) analyses of independent T₀ primary transgenic plants showed that expression of SlGAME31 was reduced in SlGAME31-RNAi and strongly decreased in SlGAME31-Cosup tomato leaves, allowing selection of those lines for further metabolic analyses (FIG. 36A).

To further understand the role of SIGAME31 in hydroxylation of SGAs, transgenic tomato lines in which SlGAME31 was silenced by RNA interference (SlGAME31-RNAi) were generated. Also, lines constitutively expressing the tomato GAME31 were generated but these turned to be co-suppressed (SlGAME31-Cosup). Quantitative Real-Time PCR (qRT-PCR) analyses of independent T₀ primary transgenic plants showed that expression of SlGAME31 was reduced in SlGAME31-RNAi and strongly decreased in SlGAME31-Cosup tomato leaves, allowing selection of those lines for further metabolic analyses (FIG. 36A).

To further understand the role of SIGAME31 in hydroxylation of SGAs, transgenic tomato lines in which SlGAME31 was silenced by RNA interference (SlGAME31-RNAi) were generated. Also, lines constitutively expressing the tomato GAME31 were generated but these turned to be co-suppressed (SlGAME31-Cosup). Quantitative Real-Time PCR (qRT-PCR) analyses of independent T₀ primary transgenic plants showed that expression of SlGAME31 was reduced in SlGAME31-RNAi and strongly decreased in SlGAME31-Cosup tomato leaves, allowing selection of those lines for further metabolic analyses (FIG. 36B).

FIG. 36C presents a schematic representation of the SGA biosynthetic pathway and GAME31 role in it. SGA profiling was carried out on extracts of skin and flesh of red ripe tomato fruit of these lines by UPLC-qTOF-MS (FIG. 33D). In fruit of SlGAME31-RNAi lines, the levels of α-tomatine increased 2.6-8.4 fold as compared to wild-type (WT) plants, while in the SlGAME31-Cosup lines, where the silencing was stronger, α-tomatine increased more than 340-520 fold relative to WT plants. Similarly, dehydrotomatine increased 1.4-4.3 fold in SlGAME31-RNAi and 105-177 fold in SlGAME31-Cosup as compared with WT plants. On the other hand, hydroxytomatine and esculeoside A remained in similar levels in SlGAME31-RNAi and WT tomato fruit. However, in the co-suppression lines, SlGAME31-Cosup, a strong decrease in hydroxytomatine (1.8-6 fold, lines #7, #5) and esculeoside A (4-29 fold, lines #7, #5) was observed when compared to WT tomato fruit (FIG. 36D).

Conclusion: Enzymatic activity assays using recombinant GAME31 and transgenic plants suppressing this gene, confirmed the role of SIGAME31 in hydroxylation of α-tomatine, the first step in the pathway leading to esculeoside A.

### Summary for Examples 12-17

Examples 12-17 above describe the identification of a gene (GAME31) that encodes a 2-oxoglutarate-dependent dioxygenase (GAME31), where the dioxygenase activity hydroxylates α-tomatine in the first step leading to fruit-related SGAs (esculeosides and derivatives) in tomato. Recombinant tomato GAME31 enzyme produced in E. coli could catalyze the formation of the same hydroxytomatine isomer accumulating in tomato fruit. Additionally, homologs of GAME31 were identified in potato and eggplant, which are the putative genes responsible for the production of hydroxylated alkaloids in these species. Further, reduction of GAME31 expression resulted in altered SGA content in tomato fruit.

While certain features disclosed herein have been illustrated and described herein, many modifications, substitutions, changes, and equivalents will now occur to those of ordinary skill in the art. It is, therefore, to be understood that the appended claims are intended to cover all such modifications and changes as fall within the true spirit of the genetically modified plants and methods disclosed herein.

What is claimed is:

1. A genetically modified plant comprising an altered content of at least one cholesterol derived compound, said cholesterol derived compound comprising a steroidal alkaloid or a glycosylated derivative thereof, or an unsaturated or saturated steroidal saponin or a glycoside derivative thereof, said plant comprising at least one cell having an altered expression of a gene encoding a 3-β-hydroxysteroid dehydrogenase/isomerase (GAME25) polypeptide as compared to GAME25 expression in a corresponding unmodified plant, wherein the amino acid sequence of said 3-β-hydroxysteroid dehydrogenase/isomerase (GAME25) polypeptide comprises the amino acid sequence set forth in any one of SEQ ID NO: 3, SEQ ID NO: 12, and SEQ ID NO: 15; and wherein said plant is a Solanaceae crop plant.

2. The genetically modified plant according to claim 1, wherein the said 3-β-hydroxysteroid dehydrogenase/isomerase (GAME25) polypeptide is encoded by a gene comprising the polynucleotide sequence set forth in any one of SEQ ID NO: 1, SEQ ID NO: 2, SEQ ID NO: 11, SEQ ID NO: 13, and SEQ ID NO: 14.

3. The genetically modified plant according to claim 1, wherein said altered expression comprises
(a) a reduced or inhibited expression of said GAME25 gene compared to its expression in a corresponding unmodified plant; or
(b) an increased expression of said GAME25 gene compared to its expression in a corresponding unmodified plant.

4. The genetically modified plant according to claim 1, wherein said Solanaceae crop plant comprises a cultivated tomato plant, a wild-tomato plant, a cultivated potato plant, or a wild-potato plant.

5. The genetically modified plant according to claim 1, wherein
(a) the altered content of said at least one cholesterol derived compound comprises a reduced content of at least one steroidal alkaloid or a glycosylated derivative thereof comprising a reduced content of at least one anti-nutritional steroidal alkaloid or a glycosylated derivative thereof, or a reduced content of at least one toxic steroidal alkaloid or a glycosylated derivative thereof, or a combination thereof; or
(b) the altered content of said at least one cholesterol derived compound comprises an increased content of at least one steroidal alkaloid or a glycosylated derivative thereof, said increased content optionally comprising precursor steroidal alkaloid molecules that provide resistance to at least one plant pathogen, pest or predator, or any combination thereof; or
(c) a combination thereof of (a) and (b).

6. The genetically modified plant according to claim 1, wherein
said at least one steroidal alkaloid or glycosylated derivative thereof comprises tomatidine, α-tomatine, α-tomatine isomer (1 and 2), α-tomatine isomer 1, α-tomatine isomer 2, hydroxytomatine, acetoxytomatine, dehydrotomatidine, dehydrotomatine, dehydrotomatine isomer 1, dehydrotomatine+4-hexose, acetoxy-hydroxytomatine, acetoxy-hydroxy-dehydrotomatine, tomatidine+4 hexose, esculeosides, esculeoside A, esculeoside A+hexose, esculeoside B, acetoxyesculeoside B, demissidine, demissine, dehydrosolasodine, hydroxy-dehydrotomatine, acetoxy-hydroxy-dehydrotomatine, dehydroesculeosides, dehydroesculeoside A, dehydroesculeoside A+hexose, leptinine I, leptinine II, leptine I, leptine II, lycoperosides, soladulcidine, 0-soladulcine, soladulcine A, solanidine, α-solanine, α-chaconine, solasodine, α-solasonine, α-solamargine, hydroxysolasonine, and hydroxysolamargine, or any derivatives thereof, or any combination thereof; and
said at least one unsaturated or saturated steroidal saponin or glycosylated derivative thereof comprises dioscin, diosgenin, parillin, and sarasapogenin.

7. The genetically modified plant according to claim 1, wherein
when said plant is a potato plant, said at least one steroidal alkaloid or glycosylated derivative thereof comprises α-solanine, α-chaconine, leptinine I, leptinine II, leptine I, or leptine II, or any combination thereof; and
when said plant is a tomato plant, said at least one steroidal alkaloid or glycosylated derivative thereof comprises α-tomatine, α-tomatine isomer (1 and 2), α-tomatine isomer 2, hydroxytomatine, acetoxytomatine, dehydrotomatidine, dehydrotomatine, dehydrotomatine isomer 1, dehydrotomatine+4-hexose, esculeosides, lycoperoside, or any derivatives thereof, or any combination thereof.

8. The genetically modified plant according to claim 1, wherein said genetically modified plant comprises a transgenic plant comprising said at least one cell comprising at least one silencing molecule targeted to a GAME25 gene, said silencing molecule comprising an RNA interference molecule and an antisense molecule.

9. The genetically modified plant according to claim 8, wherein said silencing molecule comprises the nucleic acid sequence set forth in SEQ ID NO: 8, or a fragment thereof.

10. The genetically modified plant according to claim 1, wherein said at least one cell is selected from the group consisting of a leaf cell, a young leaf cell, a mature leaf cell, a bud cell, a petal cell, a flower cell, a stem cell, a shoot cell, a peel cell, a root cell, a fruit cell, a tuber cell, and a vegetable cell.

11. A method of reducing the content of at least one cholesterol derived compound in a plant, said at least one cholesterol derived compound comprising a steroidal alkaloid or a glycosylated derivative thereof, or an unsaturated or saturated steroidal saponin or a glycoside derivative thereof, or a combination thereof, said method comprising:
(a) transforming at least one plant cell within said plant with at least one silencing molecule targeted to a nucleic acid sequence encoding 3-β-hydroxysteroid dehydrogenase/isomerase (GAME25) polypeptide; or
(b) transforming at least one plant cell within said plant with at least one polynucleotide sequence encoding 3-j-hydroxysteroid dehydrogenase/isomerase (GAME25) polypeptide, wherein said at least one polynucleotide sequence comprises a mutation in a coding region or a regulatory region; or
(c) a combination of (a) and (b);
thereby producing a plant with a reduced content of said at least one cholesterol derived compound compared to a corresponding non-transformed plant; and
wherein the at least one cholesterol derived compound comprises an anti-nutritional compound, or a toxic compound, or any combination thereof; or
wherein said reducing content of the at least one cholesterol derived compound maintains or increases plant resistance to at least one pathogen or predator in said plant compared to a corresponding non-transformed plant.

12. The method according to claim 11, wherein said silencing molecule comprises the nucleic acid sequence set forth in SEQ ID NO: 8, or a fragment thereof.

13. The method according to claim 11, wherein said plant comprises a Solanaceae crop plant.

14. The method according to claim 11, wherein said at least one steroidal alkaloid or a glycosylated derivative thereof comprises α-tomatine, α-tomatine isomer (1 and 2), hydroxytomatine, or acetoxytomatine, and said at least one unsaturated or saturated steroidal saponin or glycosylated derivative thereof comprises a sarasapogenin or any combination thereof.

15. The method according to claim 11, wherein said method further increases at least one cholesterol derived compound, said at least one cholesterol derived compound comprising
at least one steroidal alkaloid or glycosylated derivative thereof comprising a dehydrotomatine, a dehydrotomatine isomer 1, or a dehydrotomatidine+4-hexose, or any combination thereof; or
at least one unsaturated or saturated steroidal saponin or glycosylated derivative thereof comprising a diosgenin, or any combination thereof;
or any combination thereof.

16. The method according to claim 11, wherein said at least one plant cell is selected from the group consisting of a leaf cell, a young leaf cell, a mature leaf cell, a bud cell, a petal cell, a flower cell, a stem cell, a shoot cell, a peel cell, a root cell, a fruit cell, a tuber cell, and a vegetable cell.

17. A method of enhancing the content of at least one cholesterol derived compound in a plant, said cholesterol derived compound comprising a steroidal alkaloid or a glycosylated derivative thereof or an unsaturated or saturated steroidal saponin or a glycoside derivative thereof, or a combination thereof, said method comprising
(a) transforming at least one plant cell within said plant with a nucleic acid sequence encoding 3-β-hydroxysteroid dehydrogenase/isomerase (GAME25) polypeptide, wherein said transforming results in over-expression of said GAME25; or
(b) transforming at least one plant cell within said plant with at least one polynucleotide sequence encoding 3-β-hydroxysteroid dehydrogenase/isomerase (GAME25) polypeptide, wherein said at least one polynucleotide sequence comprises a mutation in a coding region or a regulatory region;
thereby producing a plant with an enhanced content of said at least cholesterol derived compound as compared to a corresponding non-transformed plant;
wherein said at least one steroidal alkaloid or a glycosylated derivative thereof comprises α-tomatine, α-tomatine isomer (1 and 2), hydroxytomatine, acetoxytomatine, soladulcidine, 0-soladulcine, soladulcine A, leptin, or a leptinine, or a combination thereof; or said unsaturated or saturated steroidal saponin or glycosylated derivative thereof comprises a sarasapogenin; or any combination thereof.

18. The method according to claim 17, wherein said method increases the plants resistance to at least one plant pathogen, pests or predator, compared to a corresponding non-transformed plant.

19. The method according to claim 17, wherein said plant comprises a Solanaceae crop plant.

20. The method according to claim 17, wherein said at least one plant cell is selected from the group consisting of a leaf cell, a young leaf cell, a mature leaf cell, a bud cell, a petal cell, a flower cell, a stem cell, a shoot cell, a peel cell, a root cell, a fruit cell, a tuber cell, and a vegetable cell.


SEQUENCE LISTING (107 sequences)

SEQ ID NO: 1 - DNA, 1468 bp, Solanum lycopersicum
  source 1..1468: mol_type = other DNA
gtgatatatttcaaaaaataattaaaatacatatatattgcatacataattcacttttaatacatattgcagatttaacttaaacattgttataaatggtgataaataaaaaaatcgttaaaattagtaattattcattaaactgcatctatttatgtaatttttccaattaaaaatctattattttttttcaatccaatccaaacaggctctaaagcatcaatgtttttgaaattaccaaaatagcctcggttgttaagcgcttccttctatatattagtgaattcaaactacagtcggtacaaaggaagttatttactcttataatggcaaataagctcaggtatagcatagttagtatttgtttaaattaatggtgctaatcagtacattaatttattttctcaaaattgtgtaattacatataattaaatgtgtttaatcaaatgtttttcttttttatatgcatcgatcctgtaggttggagggcaaagtagctataattaccggtgctgctagtggcattggagaagcaagtgctagattgttcgttgaacatggtgctcgtgtcgtcgtcgccgatattcaagatgaacttggtcaaaaagtagttgattctatcggatctgacaaagccagctaccggcactgcgacgttacagacgagaagcaagttgaggaaaccgtagcttacgcggtagagaaatacggtactcttgacattatgtttagtaatgtcgggacgctgaatttctgcagcgtcctcgacatggacgtgctggccttcgatgagaccatggccatcaacgtacgcggatccgcgttagcggttaagcacgcggctaaagttatggttgataagaaaattcggggatctattatatgtaacgcgagtttagaagggattttagctggggccgcttcgctcgcctacattgcgtcaaagcacgcagtggtaggcattataaaagcggccgcacgtgaactgggtccacatgggataagggtgaatggggtgtcgccctatggaatagcgacgccccttgtgactaaggcgtatggactggatgcggctctattggaagaagcaatttacggtaatggacacttgaaaggagttaagttgagcacgatgcatgtagcacaatcagcactttttttggcgtctgatgaatctgcttatacaagtggtcaaaatttagctgttgatggtggactaagttctattttgaagctacaataaattgtcacgctatttgtgttggcgtgctgtggcgtgggccttaatcctcactctcttgtgtctgtacttctgtttcatctcgtttcgtttcaaattttcaacttaataatactctcatattttatgcgatatttttcagatttatactaagttttttatagatattttaaacgttgtgacttaaaaagatataaatttcatttttttaaaattaaaaattttatg

SEQ ID NO: 2 - DNA, 780 bp, synthetic construct
  misc_feature 1..780: note = GAME25 CDS of Solanum Lycopersicum
  source 1..780: mol_type = other DNA
atggcaaataagctcaggttggagggcaaagtagctataattaccggtgctgctagtggcattggagaagcaagtgctagattgttcgttgaacatggtgctcgtgtcgtcgtcgccgatattcaagatgaacttggtcaaaaagtagttgattctatcggatctgacaaagccagctaccggcactgcgacgttacagacgagaagcaagttgaggaaaccgtagcttacgcggtagagaaatacggtactcttgacattatgtttagtaatgtcgggacgctgaatttctgcagcgtcctcgacatggacgtgctggccttcgatgagaccatggccatcaacgtacgcggatccgcgttagcggttaagcacgcggctaaagttatggttgataagaaaattcggggatctattatatgtaacgcgagtttagaagggattttagctggggccgcttcgctcgcctacattgcgtcaaagcacgcagtggtaggcattataaaagcggccgcacgtgaactgggtccacatgggataagggtgaatggggtgtcgccctatggaatagcgacgccccttgtgactaaggcgtatggactggatgcggctctattggaagaagcaatttacggtaatggacacttgaaaggagttaagttgagcacgatgcatgtagcacaatcagcactttttttggcgtctgatgaatctgcttatacaagtggtcaaaatttagctgttgatggtggactaagttctattttgaagctacaataa

SEQ ID NO: 3 - protein, 259 aa, Solanum lycopersicum
MANKLRLEGKVAIITGAASGIGEASARLFVEHGARVVVADIQDELGQKVVDSIGSDKASYRHCDVTDEKQVEETVAYAVEKYGTLDIMFSNVGTLNFCSVLDMDVLAFDETMAINVRGSALAVKHAAKVMVDKKIRGSIICNASLEGILAGAASLAYIASKHAVVGIIKAAARELGPHGIRVNGVSPYGIATPLVTKAYGLDAALLEEAIYGNGHLKGVKLSTMHVAQSALFLASDESAYTSGQNLAVDGGLSSILKLQ

SEQ ID NO: 4 - DNA, 24 bp, synthetic construct
  misc_feature 1..24: note = GAME25 qRT Forward primer
  source 1..24: mol_type = other DNA
gaagcaatttacggtaatggacac

SEQ ID NO: 5 - DNA, 23 bp, synthetic construct
  misc_feature 1..23: note = GAME25 qRT Reverse primer
  source 1..23: mol_type = other DNA
gaacttagtccaccatcaacagc

SEQ ID NO: 6 - DNA, 30 bp, synthetic construct
  misc_feature 1..30: note = GAME25 RNAi Forward primer
  source 1..30: mol_type = other DNA
gcggccgcattgtcacgctatttgtgttgg

SEQ ID NO: 7 - DNA, 36 bp, synthetic construct
  misc_feature 1..36: note = GAME25 RNAi Reverse primer
  source 1..36: mol_type = other DNA
ggcgcgccgaaatttatatctttttaagtcacaacg

SEQ ID NO: 8 - DNA, 199 bp, synthetic construct
  misc_feature 1..199: note = GAME25-RNAi construct
  source 1..199: mol_type = other DNA
attgtcacgctatttgtgttggcgtgctgtggcgtgggccttaatcctcactctcttgtgtctgtacttctgtttcatctcgtttcgtttcaaattttcaacttaataatactctcatattttatgcgatatttttcagatttatactaagttttttatagatattttaaacgttgtgacttaaaaagatataaatttc

SEQ ID NO: 9 - DNA, 28 bp, synthetic construct
  misc_feature 1..28: note = GAME25 baculo Forward primer
  source 1..28: mol_type = other DNA
cccgggatggcaaataagctcaggttgg

SEQ ID NO: 10 - DNA, 66 bp, synthetic construct
  misc_feature 1..66: note = GAME25 baculo Reverese primer
  source 1..66: mol_type = other DNA
tctagattacagatcttcttcagaaataagtttttgttcttgtagcttcaaaatagaacttagtcc

SEQ ID NO: 11 - DNA, 780 bp, synthetic construct
  misc_feature 1..780: note = GAME25 CDS of Solanum pennellii
  source 1..780: mol_type = other DNA
atggcaaataagctcaggttggagggcaaagtagctataattactggtgctgctagtggcattggagaggcaagtgctagattgttcgttgaacatggtgctcgtgtcgtcgtcgccgatattcaagatgaacttggtcaaaaagtagttgattctatcggagctgacaaagccagctaccggcactgcgacgttacagacgagaagcaagttgaggaaaccgtagcctacgcggtagagaaatacggtactcttgacattatgtttagtaatgtcgggacgctgaatttctgcagcgtcctcgacatggacgtgatggccttcgatgagacgatggccatcaacgtacgtggatccgcgctagcggttaagcacgcggctaaagttatggttgataagaaaattcggggatctattatatgtaacgcgagtttagaggggattttagctggggccgcttcgcttgcctacattgcgtcaaagcacgcagtcgtaggcataataaaagcggccgcacgtgaactgggtccacatgggataagggtgaatggggtgtcgccatatggaatagcgacgcccctggtgtgtaaggcgtatggactggatgcggctctattggaagaagcaatttatggtaatggacacttgaaaggtgttaagttgagcacgatgcatgtagcacaatcagcactttttttggcgtctgatgaatctgcttacacaagtggtcaaaatttagctgttgatggtggactaagttctattttgaagctacaataa

SEQ ID NO: 12 - protein, 259 aa, Solanum pennellii
MANKLRLEGKVAIITGAASGIGEASARLFVEHGARVVVADIQDELGQKVVDSIGADKASYRHCDVTDEKQVEETVAYAVEKYGTLDIMFSNVGTLNFCSVLDMDVMAFDETMAINVRGSALAVKHAAKVMVDKKIRGSIICNASLEGILAGAASLAYIASKHAVVGIIKAAARELGPHGIRVNGVSPYGIATPLVCKAYGLDAALLEEAIYGNGHLKGVKLSTMHVAQSALFLASDESAYTSGQNLAVDGGLSSILKLQ

SEQ ID NO: 13 - DNA, 1080 bp, Solanum tuberosum
  source 1..1080: mol_type = other DNA
aaaaaatttaacatacagttgctgcaaaggaagctacctactcgtataatggcaaataagctcaggtacttaattagtacattaatttctttctttcttttctcaaattgtatatgagaattaaatgtgtatttttagctttaatcaaatgtttttgtggtatattatatgcatcgtgtaggttggagggcaaagtggctataattacaggtgctgcaagtggcattggagaagcaagtgctagattgttcgccgaacatggtgctcgtattgtcgtagccgatattcaagatgaacttggtctgaaagtagttgaatctatcggagctgacaaagccagctaccgacactgcgacgttacagacgagaagcaagttgaggataccgtagcttacacggtagagaaatacggtactcttgacatcatgtttagtaatgttgggacgctgaatttttgcagcgtcctggacatggacgtgatggtcttcgataagacgatggccatcaacgcacgaggatccgcgttagcggtcaagcacgcggctagatttatggttgataagaaaattcggggatccattatatgcaacgcgagtttagatggtattgtagctggggccacttcgcttgcctacattgcgtcaaagcacgcagttgtaggcattgtgaaagcggccgcacgtgacctaggtccatacgggataagggtgaatggggtgtcgccatatggaatagcgacgcccctggtgtgcaaagcgtatgggttggatgcgggtccattggaagcagcaatatatggaaatggaaacttgaaaggtgttaggttgagcacgatgcatgtagcacaatcagcacttttcttggcgtctgatgaatctgcttacacaagtggtcaaaatttagctgttgatggtggacttagttctattttgaaggtacaatagattgtcactctattgtgctggtgtgctgtgatgtgtgcattagttctattttgaagctacaataattcctttgtcatgtagtactgtttatcttgtttcatttcgaattttcaacttaaataatattctctcacag

SEQ ID NO: 14 - DNA, 780 bp, synthetic construct
  misc_feature 1..780: note = CDS of GAME25 of Solanum tubersoum
  source 1..780: mol_type = other DNA
atggcaaataagctcaggttggagggcaaagtggctataattacaggtgctgcaagtggcattggagaagcaagtgctagattgttcgccgaacatggtgctcgtattgtcgtagccgatattcaagatgaacttggtctgaaagtagttgaatctatcggagctgacaaagccagctaccgacactgcgacgttacagacgagaagcaagttgaggataccgtagcttacacggtagagaaatacggtactcttgacatcatgtttagtaatgttgggacgctgaatttttgcagcgtcctggacatggacgtgatggtcttcgataagacgatggccatcaacgcacgaggatccgcgttagcggtcaagcacgcggctagatttatggttgataagaaaattcggggatccattatatgcaacgcgagtttagatggtattgtagctggggccacttcgcttgcctacattgcgtcaaagcacgcagttgtaggcattgtgaaagcggccgcacgtgacctaggtccatacgggataagggtgaatggggtgtcgccatatggaatagcgacgcccctggtgtgcaaagcgtatgggttggatgcgggtccattggaagcagcaatatatggaaatggaaacttgaaaggtgttaggttgagcacgatgcatgtagcacaatcagcacttttcttggcgtctgatgaatctgcttacacaagtggtcaaaatttagctgttgatggtggacttagttctattttgaaggtacaatag

SEQ ID NO: 15 - protein, 259 aa, Solanum tuberosum
MANKLRLEGKVAIITGAASGIGEASARLFAEHGARIVVADIQDELGLKVVESIGADKASYRHCDVTDEKQVEDTVAYTVEKYGTLDIMFSNVGTLNFCSVLDMDVMVFDKTMAINARGSALAVKHAARFMVDKKIRGSIICNASLDGIVAGATSLAYIASKHAVVGIVKAAARDLGPYGIRVNGVSPYGIATPLVCKAYGLDAGPLEAAIYGNGNLKGVRLSTMHVAQSALFLASDESAYTSGQNLAVDGGLSSILKVQ

SEQ ID NO: 16 - DNA, 2552 bp, Solanum lycopersicum
  source 1..2552: mol_type = other DNA
tgactataattgatactcaatctgttgaaatataatgaaccaattcttatcaaaacacagtggagtacaagtaatcacgttggttcctatgaaatggttcatctatttcccattatatataggctacttatttcctcacctataaagtaaaaaactttctagtgttttcttctttcttttgtttttttctctttgctcatattctaaaaatatttcatcaatggcatctatcaaatcagttaaagttcctactatagatttttccaattatcaagagctaaaaccaaacactccactatgggaatccacaaaaattcaagtttttgaagctttacaagaatatggttgttttgaagcaatatatgataaagtttcaaaggaaattagagaggaaacatttgatatgtcaaaagaaatatttgaatttcctttagagactaaagtgaaaaatatctcagaaaaaccaatgcatggctatatggggatgattccacaattgccattgtatgagagtttgtgtattcctgatttgcttaatcctcaaagtcttgaaaaattttctaatatcttttggcctcagggtaatcaacatttctggtatgtttacttttatttctttttcatttttgttttcttattatctttaaattttgttctagtggaactgttcaaaagctactatctttagaaataataatttttattagcttagttgattgattatgcgatattattaatagcttaaaaaaataatttttattagcttagaaataataatttttattaacttagttgattgactatgcgatattattaatagcttaaaagagcttagttgatcagactacgaaagtaaaataaaaagagacggaagtctgtgtctcgcatctattttttattgcaccgtttaaactaaataaaatatagacaacaacatcaaaatatttggtaggaagacacgatttattcaacagaaatatagacaacaacattaaagtatttggtacatgaaatcactatccaataagtgacagttcgttggccttctcattttttataaaataaatagaaacacaagagttgtctcaagtgaaaaaattgaattatgttcaaccttcttcatatgtttatactaatattacatgagcgttaatttttgcagcaatttgataaaatcttattctaatccacttgtggaattggatgggatgttgaaaaggatgatttcggagaatttgggattgaaaaatcacattgatgaattattgaatgccaattacttcctatttagatttacacattataagggatcatcaattgctagtggagatgaaaataataaagctgctggattgggtggccacacggatggtaacttcttgacttttatatcgcaaaatcaagttaatggattgcaaatcaacaaaaatggagaatggattgatgtgattatttcaccaaattcttacgttgttttggccggtgattccttcaaagtaagtattttaagttttgaactagtgttacttatcttgttgggaactgttttgtttgatttttaaaagaaaaaatattaaatgatcaaaaaaattataatatcttttttgttttaaggttaaataaattgatttaaaaatttcatttttaattaaaagagggtagtaaaatgcttaaaaagctaaaataatttagtgtgaaatatattattttattatcattctaatcaaaatttctggtcacaccttatagcataggggtttcagagggccccgagatattttgttttgatcttatatttctcgaatctatgaaaatgttattccactagtgtttatattattttctgaaatgcatatttttgaatgatttgatatatgctcaatattttcatgcaaaactgaaaatgaattttggtattattgaccgtatttgtattgttttactctccaaaaatattatcgatcgcatctatctttgtatttatacaggcttggacaaatggtcgattgcattcacctctccacagagtaacaatgtccggacaaaatgatagactctccattcaattgttttcattatcaaagccaggtcacttcatccaggcaccaaaagaactagtagatgaagaacacccattactcttcaagccatttgaaattcttgaattattcaagtatggtaccacagaagctggctatacagctcctccaagtgatcttttcaagatttattgtggtgtttgatatgctaattgttgaatttccgcttcaacaagcaacttttctaatgagtttcatcttgtttttttaagtagtatgcattttatgtttgaattgttgcagttggcaattcatgtttaatttgtttttgtttttttgagaaaatatttccaatgggtttcgttggaaattcgtcttgtttttttttttcaagtagtgtacatcttatttttggattgttgatgttgagcgctaatgtttaatttgtttgtgttttgaagaggatgattatactctttaagaggattcaccgtaatcttttagtattatttg

SEQ ID NO: 17 - DNA, 1495 bp, synthetic construct
  misc_feature 1..1495: note = cDNA of GAME31 of Solanum lycopersycum
  source 1..1495: mol_type = other DNA
tgactataattgatactcaatctgttgaaatataatgaaccaattcttatcaaaacacagtggagtacaagtaatcacgttggttcctatgaaatggttcatctatttcccattatatataggctacttatttcctcacctataaagtaaaaaactttctagtgttttcttctttcttttgtttttttctctttgctcatattctaaaaatatttcatcaatggcatctatcaaatcagttaaagttcctactatagatttttccaattatcaagagctaaaaccaaacactccactatgggaatccacaaaaattcaagtttttgaagctttacaagaatatggttgttttgaagcaatatatgataaagtttcaaaggaaattagagaggaaacatttgatatgtcaaaagaaatatttgaatttcctttagagactaaagtgaaaaatatctcagaaaaaccaatgcatggctatatggggatgattccacaattgccattgtatgagagtttgtgtattcctgatttgcttaatcctcaaagtcttgaaaaattttctaatatcttttggcctcagggtaatcaacatttctgcaatttgataaaatcttattctaatccacttgtggaattggatgggatgttgaaaaggatgatttcggagaatttgggattgaaaaatcacattgatgaattattgaatgccaattacttcctatttagatttacacattataagggatcatcaattgctagtggagatgaaaataataaagctgctggattgggtggccacacggatggtaacttcttgacttttatatcgcaaaatcaagttaatggattgcaaatcaacaaaaatggagaatggattgatgtgattatttcaccaaattcttacgttgttttggccggtgattccttcaaagcttggacaaatggtcgattgcattcacctctccacagagtaacaatgtccggacaaaatgatagactctccattcaattgttttcattatcaaagccaggtcacttcatccaggcaccaaaagaactagtagatgaagaacacccattactcttcaagccatttgaaattcttgaattattcaagtatggtaccacagaagctggctatacagctcctccaagtgatcttttcaagatttattgtggtgtttgatatgctaattgttgaatttccgcttcaacaagcaacttttctaatgagtttcatcttgtttttttaagtagtatgcattttatgtttgaattgttgcagttggcaattcatgtttaatttgtttttgtttttttgagaaaatatttccaatgggtttcgttggaaattcgtcttgtttttttttttcaagtagtgtacatcttatttttggattgttgatgttgagcgctaatgtttaatttgtttgtgttttgaagaggatgattatactctttaagaggattcaccgtaatcttttagtattatttg

SEQ ID NO: 18 - protein, 321 aa, Solanum lycopersicum
MASIKSVKVPTIDFSNYQELKPNTPLWESTKIQVFEALQEYGCFEAIYDKVSKEIREETFDMSKEIFEFPLETKVKNISEKPMHGYMGMIPQLPLYESLCIPDLLNPQSLEKFSNIFWPQGNQHFCNLIKSYSNPLVELDGMLKRMISENLGLKNHIDELLNANYFLFRFTHYKGSSIASGDENNKAAGLGGHTDGNFLTFISQNQVNGLQINKNGEWIDVIISPNSYVVLAGDSFKAWTNGRLHSPLHRVTMSGQNDRLSIQLFSLSKPGHFIQAPKELVDEEHPLLFKPFEILELFKYGTTEAGYTAPPSDLFKIYCGV

SEQ ID NO: 19 - DNA, 462 bp, Solanum lycopersicum
  source 1..462: mol_type = other DNA
atggcatctaccaaattagttaaagttcccacaatagatttttcaaatcatcaagatctaaaaccaaacactccactatgggaatccaaaaaaattcaagtttttgaagctttgcaagaatatggttgttttgaagcaatttatgataaagttccaaaagatattagagaggaaacatttagtatttcaaaagaaatatttgaatttcctttagagactaaattgaaaaatatttcagaaaaaccaacgcatggatatatgggaatgattccacaattgccattgtatgagagtttgtgtattcctgatttgcttaatcctaaaagtcttcaaagttttgctaatatcttttggcctcagggtaaccaacatttctggtatgtttacttatgtttttatttcgccctagcagaagtgttcaaaagtacgacattagaaattcttagtgacttaattgattga

SEQ ID NO: 20 - DNA, 462 bp, synthetic construct; identical to SEQ ID NO: 19
  misc_feature 1..462: note = cDNA of GAME31-like 1 of Solanum lycopersycum
  source 1..462: mol_type = other DNA

SEQ ID NO: 21 - protein, 153 aa, Solanum lycopersicum
MASTKLVKVPTIDFSNHQDLKPNTPLWESKKIQVFEALQEYGCFEAIYDKVPKDIREETFSISKEIFEFPLETKLKNISEKPTHGYMGMIPQLPLYESLCIPDLLNPKSLQSFANIFWPQGNQHFWYVYLCFYFALAEVFKSTTLEILSDLID

SEQ ID NO: 22 - DNA, 1113 bp, Solanum lycopersicum
  source 1..1113: mol_type = other DNA
tgagatgttgaaaaggatgatttcggagaatttgggattaaaaaatcacattgatgaattattgaatgccaattacatcctatttagatttacacagtataagggatcatcaattgctagtggagatgaaaataataaagcagctggattgggtggccacacagatggtaacttcttgtctattatatcacaaaatgaagttaatggattgcaaatcaacaaaaatggagagtggattgatgtcaacatttcgccaaattcttatgttgttttatccggtgattccttcacagtaagtgttaagttttgagctagtgttattatcttgttgggaactgtgttgtttgattttctaaagggataatgctaaatgacaagaaactcaaaaaatcaataagatatttgttgaatcttacgtctctaaatatattatcatgctagtgttaattatttcccgaaatgcatatttttgaagaatctgacatactgagtgatattctggaagagtccaaccaagacactttgttgaaactacatgctcaatattttcatgcaaaactgaaaatgaatcttgatatttgttgaccctatgttgctctattctccaaaaatactactgcgactatctttgtatttatgcaggcatggacaaatggccgattgcattctcctgttcatagagttgaaatgcccagaggaagtgatagatattccattcaattattttcattatcaaaaccaggtcacttcatcgaggcaccaaaagaaatggtggatgaagaacaccctttgcttttcaagccatttgaaattcttggattacttgggtatggtgccacagaagctggctatacaactcctcccagtgatcttttcaaggcatattgcggtgtctgatatgctaattgcgaatttccatttctattagaataaagttagtatttatgagatttttgttggtaattcatgtttaattggtttgtgtttttttggaaaatatttctaatgtgttccgttggaaattcgtgtgcatcttatgtttggattgttggtattgggaattcatgtttaatttgtttgtgttcttgggcaaataataaatttgaagcggat

SEQ ID NO: 23 - DNA, 547 bp, synthetic construct
  misc_feature 1..547: note = cDNA of GAME31-like 2 of Solanum lycopersycum
  source 1..547: mol_type = other DNA
tgagatgttgaaaaggatgatttcggagaatttgggattaaaaaatcacattgatgaattattgaatgccaattacatcctatttagatttacacagtataagggatcatcaattgctagtggagatgaaaataataaagcagctggattgggtggccacacagatggtaacttcttgtctattatatcacaaaatgaagttaatggattgcaaatcaacaaaaatggagagtggattgatgtcaacatttcgccaaattcttatgttgttttatccggtgattccttcacagcatggacaaatggccgattgcattctcctgttcatagagttgaaatgcccagaggaagtgatagatattccattcaattattttcattatcaaaaccaggtcacttcatcgaggcaccaaaagaaatggtggatgaagaacaccctttgcttttcaagccatttgaaattcttggattacttgggtatggtgccacagaagctggctatacaactcctcccagtgatcttttcaaggcatattgcggtgtctga

SEQ ID NO: 24 - protein, 181 aa, Solanum lycopersicum
EMLKRMISENLGLKNHIDELLNANYILFRFTQYKGSSIASGDENNKAAGLGGHTDGNFLSIISQNEVNGLQINKNGEWIDVNISPNSYVVLSGDSFTAWTNGRLHSPVHRVEMPRGSDRYSIQLFSLSKPGHFIEAPKEMVDEEHPLLFKPFEILGLLGYGATEAGYTTPPSDLFKAYCGV

SEQ ID NO: 25 - DNA, 4148 bp, Solanum lycopersicum
  source 1..4148: mol_type = other DNA
tgactagaaatagacataaaaccctgtactttttgacacacatgtaatagcactttctctatctaatacgcaactctttattaattttgcgtaaattttgagctatttcccattatatataggctacttatttcctcacctcttaagtaaaaaactttcaagtgtttcttctttcttttatttctctttgttcacatattctaaaaatatttcatcaatggcatctatcaaatcagttaaagttcctactatagatttttccaattatcaagagctaaaaccaaacactccactatgggaatccacaaaaattcaagtttttgaagcttttcaagaatatggttgttttgaagcaatatatgataaagttccaaatgaaattagagaggaaacatttgatatgtcaaaagaaatatttgaatttcctttagatactaaagtgaaaaatatttcagaaaaaccaatgcatggatatatgggaatgattccacaattgccattgtatgagagtttgtgtattcctgatttgcttaatcctcaaagtcttcaaaattttgctaatatcttttggcctcagggtaatcaacatttctggtatgtctatttcactgttttcatcttttttattttcttactatcattatctttaaatttaagaaaaaacgataaatatatccttaaatataaatggtatgcagatattctccatcatatttttgggacatatatttttaccgttcaaaaattaaagcatatataccattttatactaatggatatagacgtgtcataatcttatctaccgccccaacattggatcgatggataagattgtgccaagtttcctaatttaaccattcgttagagtgaagggcagaaattttcgactttttaaatgtcagggacatcaatgtcccaaaagtatgacggaggaaaaatataacgaaaaatatgtgcatactatttacgatcgtttgaaaaaatatttgtcttttttccttttaaattttggcctaatgaaagtgttcaaaaagtataacattagagattcccagtaaattgattaactatttgaccattcaatagttttatggaataagttaattttttctttgagaaaaacaaaataggagattataaatggaagtttccttctcgaatctattcattagcacacccctaaacaaacaaaagtgccgataacgttaaaatatttggtatgaagtctcgatctactcaataaaaacaaataaattaataggagacaatagacagtatctttctcgaatcaatttcgaatttatttcttttatcacattgttaataaaatgaaaattatcaacaacatcagaatatttggtatgtgtcacgatctaataattgtagaaatcgagatataaatacgatttgagaaatcaaagattgtattgatgaaaaataatgttaagttacaaggtttttatatggagagaattgtagagttctaagttaactataataaaatactattacaatacatattactattataataataataataatgcaaatcctagtcgtaatataattctaatcgacttcaactagtacaataagtaaataagtagcactgcgtctaatcctagtatgaatctaactcgtcagtcagttccgctttcccatttttgttttcactatcctagttttaaaaataaaataaaatataagacttagttaacgtaaatatggcctcatttgtttgtatttaattgggggtctaaatcttaatcaatcagattcgcctcattcagtatgtttgtttttttatgactgaatcttaattatttagatttaattcattaagtttgtttgttttattttcttagaagtctcttaacgagtctgaatacatctgagttaatcagatctgtaatacactcttaagaccattcagactcaaaagtaattcctatcttaattcaactacatcacaaaaactcataaaagtttttttcttattaaattaatgttaattacatgcttacccgttataatttcctttattttactaacttaatatacgtcctttactttcataaattaatcaatatatttgaattgataaacacttcatgatataatatttagcacgattctagaaaacaagaaagtattgattagttgatcgataacaataacaaatctgcattataaaataaaatgctttcataaacattatattactactctatataaactattttacattgcattatattagattatcaaagtttttgagtgcaaaaaagaatagtcatatattagtgatgtaacttaatgttaaattcttaatagataaatcatatgacctattcatgataagaatgtccaaaaatttattttccatataaaaaattattttactaaaatgaggtttttataattttttgttgatacatcgtttaatttcatatgtacattcaaatattaaaaacgaattatctcaataatccagttttcatattcagagaaaataccttaatattaaaatgtttattcagatttacatatctagatcttaatgcatattttaatatttagatgtatattcagattcagacgttttgatcttaatagaaacaaataaggcctaagtgaaagaatggtatcaacttgaaatgtttctaaatctgttcaaccttctttatatgtttataaacattatatgtgtattttttttttgcagcaatttggtaaagtcttattctaatccacttgtggaattggatgagattttgaaaaggatgatttcggagaatttgagattaaaaattcacattgatgaattgttgaatgccaattatttcctatttagatttacacattacaagggatcatcaattactggtggagatgagaataacaaagttgctggattgggtggccacacagatggtaacttcttgacttttatatcgcaaaatcaagtcaatggattgcaaatcaacaaaaatggagaatggattgatgtgaatatttcaccaaattcttatgttgttttggctggtgattccttcaaagtaagtgttaagttttgaattattgttattatcttgttgggaactgttttgtttgatttttaaaagaaaaatgctaaatggtcacaaatttttaaagtcaataatattttttttgttttaaggtaaataaattgataaaaaaagaattcatttttaattaaaagatattgaaattaaaagggtaaaaatactttaaacatagtgtgaattatgttattttatcattctaatcaaaatttgtggccaatattgttacaccttataggatttatcaaaaaaacatagttttcagaggctcaagatatttgttggatcttatgtttctcgaatctctgaaaatgttgttccgcttgtgttgaatgtattattttctgaaatgtatatttttgaagaatttgatatattaatgatatgctcaatattttcatgcaaaacggaaaatgaattttggtattattgaccctatttgtattgttctactctccaaaaatattatcgatcacgtctatctttgtatttatacaggcttggacaaatggtcgattgcattctcctcttcacagagtaacaatgtccggagaaaatgatagactctccattcaattattttcattatcaaaaccaggtcacttcatcgaggcaccaaaagaactagtggatgaagaacaccctttactcttcaagccatttgaaattattggattatttgagtatggtaccacagaagctggctatacagctcctccaagtgatcttctcaagagttattgcggtgtttgatatgctaattgcgaatttccgcttcagcaaccaacttttctaataagtttcgtctgaaattcgtgttgttttaattattatgcattttatgtttgaattgttgtagttggcaattcatgtttaatttgtttgtgtttttttttttgagaaaatattgcattgggtttcattggaaatttgtgttttttaaaaagtagtgtgcatcttatgtttggattgttggtgttgagaattcatttttaatttgtttttttttttgggcaaataatgaatttaaaattgttgattttactctttagtggaaatgat

SEQ ID NO: 26 - DNA, 1345 bp, synthetic construct
  misc_feature 1..1345: note = cDNA of GAME31-like 3 of Solanum lycopersycum
  source 1..1345: mol_type = other DNA
tgactagaaatagacataaaaccctgtactttttgacacacatgtaatagcactttctctatctaatacgcaactctttattaattttgcgtaaattttgagctatttcccattatatataggctacttatttcctcacctcttaagtaaaaaactttcaagtgtttcttctttcttttatttctctttgttcacatattctaaaaatatttcatcaatggcatctatcaaatcagttaaagttcctactatagatttttccaattatcaagagctaaaaccaaacactccactatgggaatccacaaaaattcaagtttttgaagcttttcaagaatatggttgttttgaagcaatatatgataaagttccaaatgaaattagagaggaaacatttgatatgtcaaaagaaatatttgaatttcctttagatactaaagtgaaaaatatttcagaaaaaccaatgcatggatatatgggaatgattccacaattgccattgtatgagagtttgtgtattcctgatttgcttaatcctcaaagtcttcaaaattttgctaatatcttttggcctcagggtaatcaacatttctgcaatttggtaaagtcttattctaatccacttgtggaattggatgagattttgaaaaggatgatttcggagaatttgagattaaaaattcacattgatgaattgttgaatgccaattatttcctatttagatttacacattacaagggatcatcaattactggtggagatgagaataacaaagttgctggattgggtggccacacagatggtaacttcttgacttttatatcgcaaaatcaagtcaatggattgcaaatcaacaaaaatggagaatggattgatgtgaatatttcaccaaattcttatgttgttttggctggtgattccttcaaagcttggacaaatggtcgattgcattctcctcttcacagagtaacaatgtccggagaaaatgatagactctccattcaattattttcattatcaaaaccaggtcacttcatcgaggcaccaaaagaactagtggatgaagaacaccctttactcttcaagccatttgaaattattggattatttgagtatggtaccacagaagctggctatacagctcctccaagtgatcttctcaagagttattgcggtgtttgatatgctaattgcgaatttccgcttcagcaaccaacttttctaataagtttcgtctgaaattcgtgttgttttaattattatgcattttatgtttgaattgttgtagttggcaattcatgtttaatttgtttgtgttttttttttttctttagtggaaatgat

SEQ ID NO: 27 - protein, 321 aa, Solanum lycopersicum
MASIKSVKVPTIDFSNYQELKPNTPLWESTKIQVFEAFQEYGCFEAIYDKVPNEIREETFDMSKEIFEFPLDTKVKNISEKPMHGYMGMIPQLPLYESLCIPDLLNPQSLQNFANIFWPQGNQHFCNLVKSYSNPLVELDEILKRMISENLRLKIHIDELLNANYFLFRFTHYKGSSITGGDENNKVAGLGGHTDGNFLTFISQNQVNGLQINKNGEWIDVNISPNSYVVLAGDSFKAWTNGRLHSPLHRVTMSGENDRLSIQLFSLSKPGHFIEAPKELVDEEHPLLFKPFEIIGLFEYGTTEAGYTAPPSDLLKSYCGV

SEQ ID NO: 28 - DNA, 963 bp, synthetic construct
  misc_feature 1..963: note = CDS of GAME31
  source 1..963: mol_type = other DNA
atgggatctaccaaatcaattaaagttcccactatcgatttttccaaccatcaagatctaaaaccaaacactccacaatgggaatccacaaaagatcaagtttttgaagcttttcaagaatttggttgttttgaagcaatatatgataaagtgccaaatgaaattagaaagggcatgtttgatgtttcaaaagaaatatttgaatttcccctagagaccaaattgaaaaacttatcagacaaaccattacatggctacatggggatgattccaaacttgcctttgtatgagagtttgtgtattcctgatttgcttaatcctcaaagtcttcaaaattttgaaaatatcttttggccacatggaaatcctgatttttgcaatttggtaaaatgttactcaaatccacttgtggaattggatgaaatgttgaagaggatgattttggagaaattgggagtagaaaatcagattgatgagttattggatcccaaatatgtcctatttagatttacacactacaaggggtcatcaccaactaatggagataaaaatactaaaagtgagggactaggtggccacactgatggtaacttcttgacttttatagcacaaaatcaagtaagtggattgcaaattaataaaaatggagagtggattgatgtcaacatctcaccaaattcttttgctgttttgtctgctgattccttcaaagcatggacaaatggtcgattgcattctccaattcacagagtaacaatggctggagaaaatgatagattctccattcaattattttcactatccaaaccaggtcacttcatagaggccccaaaagaacttgtggatgaacaacaccctttactcttcaaaccatatgaaatgcttggattatttaagtatgttacttcacaaagtggatatggagctcctggtgatgctttcaaggcttattgtggtgtttga

SEQ ID NO: 29 - protein, 320 aa, Solanum melongena
MGSTKSIKVPTIDFSNHQDLKPNTPQWESTKDQVFEAFQEFGCFEAIYDKVPNEIRKGMFDVSKEIFEFPLETKLKNLSDKPLHGYMGMIPNLPLYESLCIPDLLNPQSLQNFENIFWPHGNPDFCNLVKCYSNPLVELDEMLKRMILEKLGVENQIDELLDPKYVLFRFTHYKGSSPTNGDKNTKSEGLGGHTDGNFLTFIAQNQVSGLQINKNGEWIDVNISPNSFAVLSADSFKAWTNGRLHSPIHRVTMAGENDRFSIQLFSLSKPGHFIEAPKELVDEQHPLLFKPYEMLGLFKYVTSQSGYGAPGDAFKAYCGV

SEQ ID NO: 30 - DNA, 2345 bp, Solanum tuberosum
  source 1..2345: mol_type = other DNA
gaaactttgaagtctttcttgtttcctaaatattcctcaaatggcatctaccaaagttacgattcccaccatagatttttgcgattctgagcttaaaccaaacactccacaatgggaatcaacaaaagttcaagtttttgaagccttacaagaatttggttgttttgaagcaatatataacaaagttccaaatgaaattagagagggcatgtttgatactttaaaagaagtatttgattttccactgcccaaattgatagaatatagagagaaaccctttcatatatatgatgggcaaattccaagtgtaccactctttggtagtgtgtactctgctgatttggtcctcccaaatagtgttgaaacatttgccaataccttttggtctcatggaaaccctaattttaggtatgcattacttctttttcattaattatagggagtcacgatagtgtaagatgcattaaaaggagaaacgtttcctagtagaattgtttctattcctagggctagaatcaggaacctttagttaaattaatagagataatattcattccatcactaaaggtgaaaattaagtatcttatattgtccataaattttatatagaagagatgtgagaattaataaaataaaaattaaaaactcacgagtaaacaaaataattatatctaatttatattaataaagaagagtatttgattattatattaagtcaaatgataagctaataaatcaatattaacaatctaatcacatgatttatataaaattggttatgggtatgggaagggagggagggaagtacatttcattgaggaacaatgcaatagttagacaggatttaacatacttgaacaagatatcataatctaaaatgattaaaaataattttttaatattatctacacatcgcgcgaatatatatatatatatatattaagtgtatttcttaaataatattgtattactatttatataaattttgtatgttttaattttgcagcaatgtggcaaagtcctacttcaagcaacttatggaattaaatgacatggttaaaaagatggttttggagagtcttgggctaaaaaattacattgatgaattcttgaattccaatgtttatatgtcaagatttactaattacaaggtaattaaaggtgaaaatgagaataaatcaggattaccttcccacacagatagttcctacttgaccataattaaacaaaatcaaaatggattgcaagttctctacaaaaatggagagtggattgagctcaatcgtcaaaatggactgcaagttctctacaaaaatggagagtggattgagctcaatcatacttcaccaaattcctatattgttttatcagaagatgtttttatggtaagttattatttattttttattacagaagtcaaaaatacacctaaactttttatttatatgtatttttgacgcttaactctttatttttttgtgtgtaggggtggtttgttgctatagtagaggagaataaaagaaatagattttttttgtatgattgattattcaagcccaactagaagctaagattagaggagttttgaagcaacgaaaaaaaatgttgtgtgtgatttatagatattgatgcaggctcgatccgtgaaagaaatcactaatatttatattagattagatcgtttacctaactaaacatcccttgaagtactgccctttctccaaaccatatgtgaacgtcaaatattttatgcatcaacctgtctttttttatttggccccaactaacttcaatccacataaattattaaatcttgatattagttggaataacatatctcttttctgagaaattgaaaataatgccagaactatcataatctttttttaaaaaaattgtcttgttattatcttattaatttaaaattttctttcttcagaggaaatttaagtcaatctttttgttccttaattattaattaaacaaataaattcttatacatactttttatgtgttgatgctatgaattaattatacaggcatggacaaatgatagattgacatctgctcaacacagggttgtaacaacaggagacaaagaaagattctctattcaagttttttcctttccaaatccagattacactgtgaaggtcccacaagaattagtggatgaagaacaccctttaatgtacaagccttttaagatgtctgaatataataaatatattatgttaggtgctaaaaatggattgggtgtcaagaattattgtggtctttaaaaatttagtagctatgaaaatttatttatgtattgttttgatgaataaaatgtatcagatggc

SEQ ID NO: 31 - DNA, 963 bp, synthetic construct
  misc_feature 1..963: note = cDNA of GAME31of Solanum tuberosum
  source 1..963: mol_type = other DNA
atggcatctaccaaagttaagattcccaccatagatttttctaatctagaactaaaaccaaacactccactatgggaatccacaaaagttcaagtttttgaagctttaaaagaatatggttgttttgaagcaacatatgataaaattccaaatgaaattagagagggtatttttggtattacaaaagaaatatttcaatttcctttagagaccaaagtgaaaaattattcagatataacattacatggctatgtaggaatgattccacacttgccattttatgagagtttgtgtattcctgatttgcttaatcctcaaaatgttgaaacttttgctaatatcttttggcctcatggtaatcctgatttctgcaatttggtaaaagcttactcaaatccacttatggaattggatgaaatgttgaaaaagatgattttggagaatttgggattagaaaatcatattgatgaattgctggatattaattatatgagatttagatttacacattacaagggatcatcaattattagtggagatcatgaaaataatattaaacaagatggattgaatggccacacagatggtaacttcttgacttttatatcacaaaatcaagtcaatggtttgcaaatcaacaaaaatggagagtggattgatgtcaatatttcaccaaattcttatgttgttttgtctggtgattcattcaaagcatggacaaatggtcgattgcattctcccatccacaaggtaaaaatatttggtgaaagtgatagattctcaattcaattattttcattctcaaaaccaggtcactttataaaggccccaaaagaacttgtggatgaagaacaccctttactcttcaagccatttgaaatggttggattatctgagtatgttacttcccaagctggctatgcagctcccagtgatgctttcaaggcttattgtggtctttga

SEQ ID NO: 32 - protein, 320 aa, Solanum tuberosum
MASTKVKIPTIDFSNLELKPNTPLWESTKVQVFEALKEYGCFEATYDKIPNEIREGIFGITKEIFQFPLETKVKNYSDITLHGYVGMIPHLPFYESLCIPDLLNPQNVETFANIFWPHGNPDFCNLVKAYSNPLMELDEMLKKMILENLGLENHIDELLDINYMRFRFTHYKGSSIISGDHENNIKQDGLNGHTDGNFLTFISQNQVNGLQINKNGEWIDVNISPNSYVVLSGDSFKAWTNGRLHSPIHKVKIFGESDRFSIQLFSFSKPGHFIKAPKELVDEEHPLLFKPFEMVGLSEYVTSQAGYAAPSDAFKAYCGL

SEQ ID NO: 33 - DNA, 2345 bp, Solanum tuberosum; identical to SEQ ID NO: 30
  source 1..2345: mol_type = other DNA

SEQ ID NO: 34 - DNA, 990 bp, synthetic construct
  misc_feature 1..990: note = cDNA of GAME31-like1of Solanum tuberosum
  source 1..990: mol_type = other DNA
atggcatctaccaaagttacgattcccaccatagatttttgcgattctgagcttaaaccaaacactccacaatgggaatcaacaaaagttcaagtttttgaagccttacaagaatttggttgttttgaagcaatatataacaaagttccaaatgaaattagagagggcatgtttgatactttaaaagaagtatttgattttccactgcccaaattgatagaatatagagagaaaccctttcatatatatgatgggcaaattccaagtgtaccactctttggtagtgtgtactctgctgatttggtcctcccaaatagtgttgaaacatttgccaataccttttggtctcatggaaaccctaattttagcaatgtggcaaagtcctacttcaagcaacttatggaattaaatgacatggttaaaaagatggttttggagagtcttgggctaaaaaattacattgatgaattcttgaattccaatgtttatatgtcaagatttactaattacaaggtaattaaaggtgaaaatgagaataaatcaggattaccttcccacacagatagttcctacttgaccataattaaacaaaatcaaaatggattgcaagttctctacaaaaatggagagtggattgagctcaatcgtcaaaatggactgcaagttctctacaaaaatggagagtggattgagctcaatcatacttcaccaaattcctatattgttttatcagaagatgtttttatggcatggacaaatgatagattgacatctgctcaacacagggttgtaacaacaggagacaaagaaagattctctattcaagttttttcctttccaaatccagattacactgtgaaggtcccacaagaattagtggatgaagaacaccctttaatgtacaagccttttaagatgtctgaatataataaatatattatgttaggtgctaaaaatggattgggtgtcaagaattattgtggtctttaa

SEQ ID NO: 35 - protein, 329 aa, Solanum tuberosum
MASTKVTIPTIDFCDSELKPNTPQWESTKVQVFEALQEFGCFEAIYNKVPNEIREGMFDTLKEVFDFPLPKLIEYREKPFHIYDGQIPSVPLFGSVYSADLVLPNSVETFANTFWSHGNPNFSNVAKSYFKQLMELNDMVKKMVLESLGLKNYIDEFLNSNVYMSRFTNYKVIKGENENKSGLPSHTDSSYLTIIKQNQNGLQVLYKNGEWIELNRQNGLQVLYKNGEWIELNHTSPNSYIVLSEDVFMAWTNDRLTSAQHRVVTTGDKERFSIQVFSFPNPDYTVKVPQELVDEEHPLMYKPFKMSEYNKYIMLGAKNGLGVKNYCGL

SEQ ID NO: 36 - DNA, 1897 bp, Solanum tuberosum
  source 1..1897: mol_type = other DNA
ttattatttgcacaaaaaatacaaacaaagcatgaattcccatcgaaattactcactgttacaacaattcaaacataagatacacactactaaagaaacacgaatttcgacaagcattctgttagaaatcccatatatacttacagtattctaacaagaacgttcataaaaaaatcacgtttttttattctagcatatataaaacaaagaactctaaagctaaagatacacactattataaaaacacgaattccaaatgaaatccattggaaatgatgaagacattttcgatgaatattctgttagaaatcccaaatatactaacagtactctgacaaaaatgttcattagaaattcacgtttatcatatcaaagaccacaataagccttgaaagcatcactgggagctgcatagccagcttgggaagtaacatactcagataatccaaccatttcaaatggcttgaagagtaaagggtgttcttcatccacaagttcttttggggcctttataaagtgacctggttttgagaatgaaaataattgaattgagaatctatcactttcaccaaatatttttaccttgtggatgggagaatgcaatcgaccatttgtccatgcctgtataaattcacgtcgtcagtgcataaaactcaaacacatttatttgaaggatccaacatttttagagattcaaaaagcatagactccaacaaatatcaaaattcatttttcagttttgcatgaaaatattaaatatgtaggtagttccatttaatattcgagaaacataggttaattccaacaaatatgaagattcattttcaattttgcatcaagatattaaacctaaattttgtttggtatattccaaccttggaaatattcttcacaaatatcgttaggtatgcgtcagatcctctaaaatctatattttgtttttgaaggtgcaataataatatttttgaagagttcgagtaacatggatttcaacaaagtaatatgactagctaaaaaaataaaatgagtaacacttactttgaatgaatcaccagacaaaacaacataagaatttggtgaaatattgacatcaatccactctccatttttgttgatttgcaaaccattgacttgattttgtgatataaaagtcaagaagttaccatctgtgtggccattcaatccatcttgtttaatattattttcatgatctccactaataattgatgatcccttgtaatgtgtaaatctaaatctcatataattaatatccagcaattcatcaatatgattttctaatcccaaattctccaaaatcatctttttcaacatttcatccaattccataagtggatttgagtaagcttttaccaaattgctgcaaaaatttatcactatctcaaaaataactttctcgctactccacgactctaatcaatgcacaaaataattttattttaaaaaataaaataaagtagacataccagaaatcaggattaccatgaggccaaaagatattagcaaaagtttcaacattttgaggattaagcaaatcaggaatacacaaactctcataaaatggcaagtgtggaatcattcctacatagccatgtaatgttatatctgaataatttttcactttggtctctaaaggaaattgaaatatttcttttgtaataccaaaaataccctctctaatttcatttggaattttatcatatgttgcttcaaaacaaccatattcttttaaagcttcaaaaacttgaacttttgtggattcccatagtggagtgtttggttttagttctagattagaaaaatctatggtgggaatcttaactttggtagatgccatttgaaagaaacaaagaaggaattaaagacttcacaatgtgaa

SEQ ID NO: 37 - DNA, 339 bp, synthetic construct
  misc_feature 1..339: note = cDNA of GAME31-like2of Solanum tuberosum
  source 1..339: mol_type = other DNA
atgattccacacttgccattttatgggagtttgtgtattcctgatttgcttaatcctcaaaatgttgaaacttttgctaatatcttttggcctcatggtaatcctgatttctgcaatttggtaaaagcttactcaaatccacttatggaattggatgaattgttgaaaaggatgattttggagaatttgggattagaaaatcatattgatgaattgttggatcctaattatatgagatttagatttacacattacaagggatcatcaattattagtggagatcatgaaaataatattaaacatgatggattgaatgccacacagatggtagcttcttga

SEQ ID NO: 38 - protein, 112 aa, Solanum tuberosum
MIPHLPFYGSLCIPDLLNPQNVETFANIFWPHGNPDFCNLVKAYSNPLMELDELLKRMILENLGLENHIDELLDPNYMRFRFTHYKGSSIISGDHENNIKHDGLNATQMVAS

SEQ ID NO: 39 - DNA, 3623 bp, Solanum tuberosum
  variation 677..1284
  variation 1884..1901
  source 1..3623: mol_type = other DNA
attttttaataaaaataatacagtacaatacattacaatacaacgcaacacaatacaatacgttatgtaatcatatgcaataaccattcaaatataaattttcaaccacccatacatacgatacattttattcataaagaaaatacacatatataaattttcataccctacaaaaattttaaagaccacaataattcttgagattaattccatttttatcacctgacatagtttatttatgaaattcaagcaagttaaaaggcttgaagagtaaagggtggtcttcatccactaattcttttggggtccttcacagtataatctggatgtggtatggaaaataattgaatagataatctatctttgtctcctgttggtactactctgtgttcagcagatgtcaaactattatttgtccatgcctgtataaatccacaacatcgacacataaaaagcattagtgatttctttcacagagaaagggcagcacctcaaggggatgttaggaaaacaatctaatctaatagaaaatattagtgattttttctcgatcaagcccgcatcgtaatctatatatcacatacaaacaatcttatcgttacttcaaaactgctctgatcttagcttctagttgggcttaaataatcaaccatacaaaaaaaatctctatttctttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtgtgtttggtacgaaggaaaatattttctggaaaatgttttttaatttcccatgtttggttgactttaattatttggaaaatgttttccaaatcaacttattttcctcaaatttaaggaaaatgatttcccttaaaaattaaggaaaacattttcgaaacttctacttcatccttaaattataatatttttttacccctactaccaaaccagcccgccacccctgtcaaattttattttatttaaaaaattacttttgaaaaatatttcagggtcggaagtttggctggggtcctaggtcagatctctaggtcggattttgaacggtcatcaaggtcatgtcctagttcgggtgttagggtcgggtcctagattgattattgggattgattttcgagttaaaagttattttcctaaagagtattttctagtcttaagcgaaaaataaaagatattttctggaaaaaaaattcattcaccaaccaaacattaaaaaatagtttctactcatcaactaaatatgagaaaataagttagaaatccacttgttttccaagaaaacattttccttcataccaaacacacccttaatatatatnnnnnnnnnnnnnnnnnnttatatatgtatagatagctagtatacatactcgagcgatgtgcggaaaatattaatatgttatttttaatcttacccctacccatgcccctacccgaccttccccattcaaaaaaataaattaaaatttttaaaatttcaaatatatttttatcactaccaccaaactagctcccccgcccccctcccctcaaacataaaaaaaaaaaattaaaattatttttgaaaaatttttaaatcttaaattttttttacctcaccaacccctaccacccctactcccttccccctcatttttaatttcccatgtttggttgactttaatgatttggaaaatgttttccaaatcaacttattttcctcaaatttaaggaaaatgattatcctaaaagttattttcctaaagagtattttctagtctcaagagaaaataaaagatattttttcagaaaataattttcattcaccaaccaaacatgagaaaataagttagaaatccacttgttttccaagaaaacattttccttcataccaaacacacccttaatatataatgtatatatacctagtatacatactcgcgtgatgtgtgaaaaatatttaaatgttattttaaatcattttagattgtgatatcttgttcgagcatgttaaatcacgtccaactattgcattgttactcaatgaaatgtactcccctccatcccttcacatacccgtaaccaattctatatgaatagcgacaatgaatcatgtgattagattgttagtattgattttattagcttatcacttggatataattattaaatactcttctaattaatataaattagatatatttattttgtttactcgtgagttcttaatttttattttattaatgatcacatctcttttatataaaatgtatggacaataagatacttaattttcacctttagtgatggaatgaatattatctctataatttaactaaagattcatgattctagccttaggaataaaaacaattctagtaggaaacgtttctccttttaatgtgtcttacactatcgtgactccatattattaatctgaatcccaaaataaataccgaatacataatgaaaaaaaaaaaatctcctacaaatatatgagcaagaataattggtgtactaacatgactaaaactaaatgatatcacctacataaatcttattctttcaaatatcattaatgaaaaatgactttcataataccgagcacattatatgtaattaaaagaagtaatacatacctaaagttagggtttccatcagaccaaaaggtattggcaaatgtttcaatactatttgggaggaccaaatcagcagagctcacactaccatagagtggtatacttggaattttcccatcatatatatgaaagggtttttctctatattctatcaatttggacaatggaaaatcaaatacttcttttaaattatcaaacatgccctctctaatttcatttggaactttgtcatatattgcttcaaaacaaccaaattcttttaaggcttcaaaaacttgaacttttgttgattcccattgtggagtgtttggttttagctcaagattgcaaaaatctatggtgggaatcttacctttagtagatgccattgatgaataaattaaaaagagaggaaatagaaagaaataaagaaggagaaatactttacaatatgaaaattaaag

SEQ ID NO: 40 - DNA, 432 bp, synthetic construct
  misc_feature 1..432: note = cDNA of GAME31-like3of Solanum tuberosum
  source 1..432: mol_type = other DNA
atggcatctactaaaggtaagattcccaccatagatttttgcaatcttgagctaaaaccaaacactccacaatgggaatcaacaaaagttcaagtttttgaagccttaaaagaatttggttgttttgaagcaatatatgacaaagttccaaatgaaattagagagggcatgtttgataatttaaaagaagtatttgattttccattgtccaaattgatagaatatagagaaaaaccctttcatatatatgatgggaaaattccaagtataccactctatggtagtgtgagctctgctgatttggtcctcccaaatagtgttgaaacatttgccaataccttttggtctgatggaaaccctaactttaggtatgtattacttcttttaattatatataatgtgcttggtattatgaaagtcatttttcattaa

SEQ ID NO: 41 - protein, 143 aa, Solanum tuberosum
MASTKGKIPTIDFCNLELKPNTPQWESTKVQVFEALKEFGCFEAIYDKVPNEIREGMFDNLKEVFDFPLSKLIEYREKPFHIYDGKIPSIPLYGSVSSADLVLPNSVETFANTFWSDGNPNFRYVLLLLIIYNVLGIMKVIFH

SEQ ID NO: 42 - DNA, 2345 bp, Solanum tuberosum; identical to SEQ ID NO: 30
  source 1..2345: mol_type = other DNA

SEQ ID NO: 43 - DNA, 852 bp, synthetic construct
  misc_feature 1..852: note = cDNA of GAME31-like4of Solanum tuberosum
  source 1..852: mol_type = other DNA
atggcatctaccaaagttacgattcccaccatagatttttgcgattctgagcttaaaccaaacactccacaatgggaatcaacaaaagttcaagtttttgaagccttacaagaatttggttgttttgaagcaatatataacaaagttccaaatgaaattagagagggcatgtttgatactttaaaagaagtatttgattttccactgcccaaattgatagaatatagagagaaaccctttcatatatatgatgggcaaattccaagtgtaccactctttggtagtgtgtactctgctgatttggtcctcccaaatagtgttgaaacatttgccaataccttttggtctcatggaaaccctaattttagcaatgtggcaaagtcctacttcaagcaacttatggaattgaatgacatggtggaaaagatggttttggagagtcttgggctaaaaaattacactgatgaattcttgaattccaatgtttatatgtcaagatttactaattacaaggtaattaaaggtgaaaatgagaataaatcagcattaccttcacacacagatagttcctacttgaccataattaaacaaaatcaaaatggattgcaagcatggacaaatgatagattgacatctgctcaacacagggttgtaacaacaggagacaaagatagattctctgttcaattattttccctcctaaatccagattatactgtgaaggtcccaaaagaattagtggatgaagaacaccctttaatgtacaagccttttaagatgcctgaatataataaatatcttatgttaggtgctaaaaatggattgggtgtcaagaattattgtggtctttaa

SEQ ID NO: 44 - protein, 168 aa, Solanum tuberosum
MISCSGLKNYIDEFLNSNVFMSRFTNYRVIKGENENKSALPSHTDSSYLTIIKQNQNGLQVLYKNGEWIELNHTSPNSYIVLSEDVFMAWTNDRLTSAQHRVVTTGDKDRFSIQVFSFPNPDYTVKVPQELVDEEHPLMFKPFKLPEFNKYIKLGAKNGPGLKNYCGF

SEQ ID NO: 45 - DNA, 2345 bp, Solanum tuberosum; identical to SEQ ID NO: 30
  source 1..2345: mol_type = other DNA

SEQ ID NO: 46 - DNA, 507 bp, synthetic construct
  misc_feature 1..507: note = cDNA of GAME31-like5of Solanum tuberosum
  source 1..507: mol_type = other DNA
atgatatcttgttctgggctaaaaaattacattgatgaattcttgaattccaatgtttttatgtcaagatttactaattacagggtaattaaaggtgaaaatgagaataaatcagcactaccttcccacacagatagttcctacttgaccataattaaacaaaatcaaaatggattgcaagttctctacaaaaatggagagtggattgagctcaatcatacttcaccaaattcctatattgttttatcagaagatgtttttatggcatggacaaatgatagattgacatctgctcaacacagggttgtaacaacaggagacaaagatagattctctattcaagttttttcctttccaaatccagattacactgtgaaggtcccacaagaattagtggatgaagaacaccctttaatgttcaagccttttaagttgcctgaatttaataaatatattaagttaggtgctaaaaatggaccgggtctcaagaattattgtggtttttaa

SEQ ID NO: 47 - protein, 168 aa, Solanum tuberosum; identical to SEQ ID NO: 44

SEQ ID NO: 48 - DNA, 2762 bp, Solanum tuberosum
  source 1..2762: mol_type = other DNA
cactaatatatttttaataaaaacaatacagtacaatacattacaatacaacgcaacacaacacaatacaatacgttatgaaatcatatgcaataaccacacaaatataaattttcaaccacccatgcatacgatacattgtattcataaagaaaatacacataaataaataccctactaaatttttaaagaccacaataattcttgagattaattcaatttttattacctgacatagtttatttatgaaattcaagcaagttaaaaggcttgaagagtaagggtggtcttcatccactaattattttggggccttcacagcaaaatcttgatttgggaaaggaaaataattgaatagatagtctatctttgtctcctgttgttactactctgtgttcagcagatgtcaaactatcatttgtccatgcctgtataaatccacaacatcgacacataaaaagtattagtgatttcttttcacagagaaagggcaacacctcaagggatgttaggaaaacaatctaatctaatacaaaatattagtgattttttctcgatcaagcccgcatcgtaatctataaatcacatacaaacaatcttatcgttacttcaaaactgctctgatcttagcttctagttgggcttgaataatcaaccatacaaaaaaatctctatttcttttattctcctcttctatagcatcaacccaccccctcacacacaaaataataaagagttaagcgtcaaaaatacacataaataaataatttaggtgtattttttaccttgatactcaaaaataaataataacttaccctgaaagcatctgctgataaaacaatataggaatttggtgttgtattattgagctctatccactctccatttttgtagagaacttgcaatccattttgattttgtttaattatagtcaagtagccactatctgtgtggggaggtaattctgatctattctcatcttcacctttaattaccttgtaattagtaaatcttgacacaaaaacattggaattcaagatttcatcaatgtaatttgttttcccaagactctccaaaaccattttttccaccatgtcattcaattccataagttgcttgaagtaggactttgccacattgctgccaaattaaaacatacaaaacttatataatggtaatgcaatattgtgatattactcaaaattatgtaagaaatacacttagggtgtgtttggtacgaaggaaaatattttctagaaaatgttttttaatttcccatgtttggttgactttaatgatttggaaaatgttttccaaatcaacttattttcctcaaatttaaggaaaatgattatcctaaaagttattttcctaaagagtattttctagtcttaagtgaaaaataagttagaaatccacttgttttccaagaaaacattttccttcataccaaacacacccttaatatataatgtatatatacctagtatacatactcgcgcgatgtgtgaaaaatatttaaatgttattttaatcattttagattgtgatatcttgttcgagcatgttaaatcatgtccaactattgcattgttactcaatgaaatgtactcccctccatcccttcacatacccgtaaccaattctatatgaatagcgatgtgtgaaaaatatttaaatgttatttttaatcattttagattgtgatatcttgttcgagcatgttaaatcatgtccaactattgcattgttactcaatgaaatgtactcccctccatcccttcacatacccgtaaccaattctatatgaatagcgacaatgaatcatgtgattagattgttaatattgattttattagcttatcacttggatataattattaaatactcttctaattaatataaattagatatatttattttgtttactcgtgagttcttaatttttattttattaatgatcacatctcttttatataaaatgtatggacaataagatacttaattttcacctttagtgatggaatgaatattatctctataatttaactaaagattcctgattctagccttaggaataaaaacaattctagtaggaaacgtttctccttttaatgtgtcttacactatcgtgactccatattattaaatctgaaatcccaaaaataaataccgaatacataatgaaaaaaaaaaaatctcctacaaaatatatgagcaagaataattggtgtactaacatgactaaactaaatgatatcacctacataaatcttattctttcaaatatcattaatgaaaaataactttcataataccgaacacattatatataattaaaagaagtaatacatacctaaagttagggtttccatcagaccaaaaggtattggcaaatgtttcaacactatttgggaggaccaaatcagcagagctcacactaccatagagtggtatacttggaatttgcccatcatataaatggggtttttctctatattctatcaatttggacactggaaaatcaaatacttcttttaaagtatcaaacatgccctctctaatttcatttggaactttgtcatatattgcttcaaaacaaccaaattcttttaaggcttcaaaaacttgaacttttgttgattcccattgtggagtgtttggttttagctcaagattgcaaaaatctatggtgggaatcttaactttggtagatgccattgatgaataaattaaatagagaggaaatagaaagaaataaa

SEQ ID NO: 49 - DNA, 828 bp, synthetic construct
  misc_feature 1..828: note = cDNA of GAME31-like6 of Solanum tuberosum
  source 1..828: mol_type = other DNA
atggcatctaccaaagttaagattcccaccatagatttttgcaatcttgagctaaaaccaaacactccacaatgggaatcaacaaaagttcaagtttttgaagccttaaaagaatttggttgttttgaagcaatatatgacaaagttccaaatgaaattagagagggcatgtttgatactttaaaagaagtatttgattttccagtgtccaaattgatagaatatagagaaaaaccccatttatatgatgggcaaattccaagtataccactctatggtagtgtgagctctgctgatttggtcctcccaaatagtgttgaaacatttgccaataccttttggtctgatggaaaccctaactttagcaatgtggcaaagtcctacttcaagcaacttatggaattgaatgacatggtggaaaaaatggttttggagagtcttgggaaaacaaattacattgatgaaatcttgaattccaatgtttttgtgtcaagatttactaattacaaggtaattaaaggtgaagatgagaatagatcagaattacctccccacacagatagtggctacttgactataattaaacaaaatcaaaatggattgcaagttctctacaaaaatggagagtggatagagctcaataatacaacaccaaattcctatattgttttatcagcagatgctttcagggcatggacaaatgatagtttgacatctgctgaacacagagtagtaacaacaggagacaaagatagactatctattcaattattttcctttcccaaatcaagattttgctgtgaaggccccaaaataattagtggatga

SEQ ID NO: 50 - protein, 275 aa, Solanum tuberosum
MASTKVKIPTIDFCNLELKPNTPQWESTKVQVFEALKEFGCFEAIYDKVPNEIREGMFDTLKEVFDFPVSKLIEYREKPHLYDGQIPSIPLYGSVSSADLVLPNSVETFANTFWSDGNPNFSNVAKSYFKQLMELNDMVEKMVLESLGKTNYIDEILNSNVFVSRFTNYKVIKGEDENRSELPPHTDSGYLTIIKQNQNGLQVLYKNGEWIELNNTTPNSYIVLSADAFRAWTNDSLTSAEHRVVTTGDKDRLSIQLFSFPKSRFCCEGPKIISG

SEQ ID NO: 51 - DNA, 1429 bp, Solanum tuberosum
  source 1..1429: mol_type = other DNA
gcagcaatgtggcaaagtcctacttcaagcaacttatggaattgaatggcatggtggaaaagatggttttggagagtcttgggctaaaaaattacattgatgaattcttgaattccaatgtttatatgtcaagatttactaattacaaggtaattaaaggtgaaaatgagaataaatcaggattaccttcccacacagatagttcctacttgaccataattaaacaaaatcaaaatggattgcaagttcttctacaaaaatggagagtggattgagctcaatcatacctcaccaaattcctatattgttttatcagcagatgctcttatggtaagttattatttatttttgattacagcagtcaaaaatacgcctaaacttctaatttatatgtatttttgacgctttaactcattattattttttgtgtgtggggtggtttgttgctatagtagaggagaataaaagaaatagagattttttttgtatgattgattattcaagcccaactagaagctaagattagaggagttttcaaccaacgaaaaaaatgtttgtgtgtgatttatatatcatgatgcaggctcaatccgtaaaagaaatcactaatatttgtattagattagattgtttacctaactaacatcccttgaagtgttgccctttctccaaaccctatgtgaacgtcaaatattttatgcatcaacctgtcttattttatttgacctcaactaacttcaatccacaaaaaatattaaatcttgatattatttggaataacgtatctctttttctggaaaattgaaaatgataccagaactatcataataattttttaaattgtcttgttattatcttattaatttaaaattttcattctccataggaaatttaagtcaatctttttgttccttaattattaattaaacaaataaattcttatacatactttttatgtgttgatgatatgaattaattatacaggcatggacaaatgatagattgacatctgctcaacatagggttgtaacaacaggagacaaagatagattctctgttcaattattttccctcgtaaatccagattatactttgaaggtcccaaaagaattagtggatgaagaacaccctttaatgtacaagccttttaagatgcctgaatataataaatatcttatgttaggtgctaaaaatggattgggtgtcaagaattattgtggtctttaaaaatttagtagctatgaaaatttatttatgtattgttttgatgaataaaatgtatcagatggctggttgaatactttgaatttatatttggatggttattacgtatgattgcgtaaagtattgtattgtattttattttgttgtgttgttttgtattgtgttgcgttgtatatattgttttgatgaataaaatatatgagtg

SEQ ID NO: 52 - DNA, 339 bp, synthetic construct
  misc_feature 1..339: note = cDNA of GAME31-like7 of Solanum tuberosum
  source 1..339: mol_type = other DNA
atggattgcaagttcttctacaaaaatggagagtggattgagctcaatcatacctcaccaaattcctatattgttttatcagcagatgctcttatggcatggacaaatgatagattgacatctgctcaacatagggttgtaacaacaggagacaaagatagattctctgttcaattattttccctcgtaaatccagattatactttgaaggtcccaaaagaattagtggatgaagaacaccctttaatgtacaagccttttaagatgcctgaatataataaatatcttatgttaggtgctaaaaatggattgggtgtcaagaattattgtggtctttaa

SEQ ID NO: 53 - protein, 112 aa, Solanum tuberosum
MDCKFFYKNGEWIELNHTSPNSYIVLSADALMAWTNDRLTSAQHRVVTTGDKDRFSVQLFSLVNPDYTLKVPKELVDEEHPLMYKPFKMPEYNKYLMLGAKNGLGVKNYCGL

SEQ ID NO: 54 - DNA, 55 bp, synthetic construct
  misc_feature 1..55: note = RF-Tomato-GAME31-Forward Primer
  source 1..55: mol_type = other DNA
tccgcgggtgaaaacctgtacttccagggtgcatctatcaaatcagttaaagttc

SEQ ID NO: 55 - DNA, 57 bp, synthetic construct
  misc_feature 1..57: note = RF-Tomato-GAME31-Reverse Primer
  source 1..57: mol_type = other DNA
gtggtggtgctcgagtgcggccgcaagctttcaaacaccacaataaatcttgaaaag

SEQ ID NO: 56 - DNA, 55 bp, synthetic construct
  misc_feature 1..55: note = RF-Eggplant-GAME31-Forward Primer
  source 1..55: mol_type = other DNA
tccgcgggtgaaaacctgtacttccagggtggatctaccaaatcaattaaagttc

SEQ ID NO: 57 - DNA, 51 bp, synthetic construct
  misc_feature 1..51: note = RF-Eggplant-GAME31-Reverse Primer
  source 1..51: mol_type = other DNA
gtggtggtgctcgagtgcggccgcaagctttcaaacaccacaataagcctt

SEQ ID NO: 58 - DNA, 149 bp, synthetic construct
  misc_feature 1..149: note = SlGAME31-RNAi
  source 1..149: mol_type = other DNA
gggtttcgttggaaattcgtcttgtttttttttttcaagtagtgtacatcttatttttggattgttgatgttgagcgctaatgtttaatttgtttgtgttttgaagaggatgattatactctttaagaggattcaccgtaatcttttag

SEQ ID NO: 59 - DNA, 966 bp, synthetic construct
  misc_feature 1..966: note = SlGAME31-Cosup
  source 1..966: mol_type = other DNA
atggcatctatcaaatcagttaaagttcctactatagatttttccaattatcaagagctaaaaccaaacactccactatgggaatccacaaaaattcaagtttttgaagctttacaagaatatggttgttttgaagcaatatatgataaagtttcaaaggaaattagagaggaaacatttgatatgtcaaaagaaatatttgaatttcctttagagactaaagtgaaaaatatctcagaaaaaccaatgcatggctatatggggatgattccacaattgccattgtatgagagtttgtgtattcctgatttgcttaatcctcaaagtcttgaaaaattttctaatatcttttggcctcagggtaatcaacatttctgcaatttgataaaatcttattctaatccacttgtggaattggatgggatgttgaaaaggatgatttcggagaatttgggattgaaaaatcacattgatgaattattgaatgccaattacttcctatttagatttacacattataagggatcatcaattgctagtggagatgaaaataataaagctgctggattgggtggccacacggatggtaacttcttgacttttatatcgcaaaatcaagttaatggattgcaaatcaacaaaaatggagaatggattgatgtgattatttcaccaaattcttacgttgttttggccggtgattccttcaaagcttggacaaatggtcgattgcattcacctctccacagagtaacaatgtccggacaaaatgatagactctccattcaattgttttcattatcaaagccaggtcacttcatccaggcaccaaaagaactagtagatgaagaacacccattactcttcaagccatttgaaattcttgaattattcaagtatggtaccacagaagctggctatacagctcctccaagtgatcttttcaagatttattgtggtgtttga

SEQ ID NO: 60 - DNA, 29 bp, synthetic construct
  misc_feature 1..29: note = qRT-PCR forward primer
  source 1..29: mol_type = other DNA
agtacaagtaatcacgttggttcctatga

SEQ ID NO: 61 - DNA, 34 bp, synthetic construct
  misc_feature 1..34: note = qRT-PCR reverse primer
  source 1..34: mol_type = other DNA
cactagaaagttttttactttataggtgaggaaa

SEQ ID NO: 62 - DNA, 35 bp, synthetic construct
  misc_feature 1..35: note = Cloning SlGAME31 for RNAi forward primer
  source 1..35: mol_type = other DNA
aaaaagcggccgcgggtttcgttggaaattcgtct

SEQ ID NO: 63 - DNA, 38 bp, synthetic construct
  misc_feature 1..38: note = Cloning SlGAME31 for RNAi reverse primer
  source 1..38: mol_type = other DNA
aaaaaggcgcgccctaaaagattacggtgaatcctctt

SEQ ID NO: 64 - DNA, 55 bp, synthetic construct
  misc_feature 1..55: note = Cloning SlGAME31 for Cosupression forward primer
  source 1..55: mol_type = other DNA
ggggacaagtttgtacaaaaaagcaggctatggcatctatcaaatcagttaaagt

SEQ ID NO: 65 - DNA, 54 bp, synthetic construct
  misc_feature 1..54: note = Cloning SlGAME31 for Cosupression reverse primer
  source 1..54: mol_type = other DNA
ggggaccactttgtacaagaaagctgggttcaaacaccacaataaatcttgaaa

SEQ ID NO: 66 - protein, 259 aa, Solanum lycopersicum
MASKLRLEGKVAIITGGASGIGEASARLFVQHGARVVVADIQDELGLQVVQSIGIHKATYRHCDVTDEKQVEDTVAYAVQKYATLDIMFSNVGTLNFCSVLDMDMTAFDETMTVNVRGSALAVKHAARVMVDKKIRGSIICNVSLEGILAGAASLAYIASKHAVVGIVKAAARELGPYGIRVNGVSPYGIATPLVCKAYGLDAAPLEAAINGNANLKGVTLSTMHVAQSALFLASDESAYTSGQNLAVDGGLSSILKLQ

SEQ ID NO: 67 - protein, 259 aa, Solanum pennellii
MASKLRLEGKVAIITGGASGIGEASARLFVQHGARVVVADIQDELGLQVVQSIGIHKATYRHCDVTDEKQVEDTVAYAVQKYATLDVMFSNVGTLNFCSVLDMDMTAFDETMTVNVRGSALAVKHAARVMVDKKIRGSIICNVSLEGILAGAASLAYIASKHAVVGIVKAAARELGPYGIRVNGVSPYGIATPLVCKAYGLDAAPLEAAINGNANLKGVTLSTMHVAQSALFLASDESAYTSGQNLAVDGGLSSILKLQ

SEQ ID NO: 68 - protein, 236 aa, Capsicum annuum
LEGKVAVITGAASGIGEASARLFVEHGARVVIADIQDELGLQIAASIGTDKASYIHCDVTDEKQVEEAVAYAVENSVLDLDVKAFDETMVINARGSAVAVKHAARVMVEKKIRGSIICTASLEGILAGAASLAYVSSKHAVVGLVKAAARELGVHGIRVNGVSPYGIATPLVCKAYGLDAGPLETAIYGNAHLKGVTLSTMHVAQAALFLASDESAYISGQNLAVDGGLSSILKLE

SEQ ID NO: 69 - protein, 261 aa, Nicotiana benthamiana
MANKLRLEGKVAVITGGASGIGEATARLFVEHGARVVIADIQDELGLQVVASIGTDKASYRHCDVTDENKVEETVAYAVEKYGTLDIMFSNVGTLNFCSVLDIDVTAFDKTMALNVRGTALAVKHAARVMVAKQVKGSIICNASIEAILAGAASLAYVASKHAVVGIVKAAARELGLHGIRVNGVSPYGIATPLVCKAYGCEDAASLEAGISVNAHLKGVTLSTEHIAQAALFLASDESAYISGHNLAVDGGLTSMLKLSY

SEQ ID NO: 70 - protein, 258 aa, Solanum melongena
MANKLKLEGKVAVITGGASGIGEESARLFVEHGARVVIADIQDDLGLEVVTSIGADKACYRHCDVSEEKQVKETVAYAVEKYGTLDIMFSNAGTLGTLGSVLEMDMTAFDMTMAVNMRGSALAVKHAARVMVANKIRGSIICTASVEAILAGAAPLAYVASKHAILGVMKAAARELGQYGIRVNCVSPYGIATPLVCKAYHSDAGSLEASIYERAHLKGITLSTKHIANASLFLASDESAYVSGHNLAVDGALSSIMS

SEQ ID NO: 71 - protein, 263 aa, Capsicum annuum
MIFFFCGTRLEGKIAIITGAASGIGEASARLFVEHGAHVIIADIQDELGLQVVSSIGTDKACYRHCDVTDEKQVEETVAYAVEKYGTLDIMFSNAGMLGTFGSLLDMDVKEFDLTIAVNTRGAALAVKHAARVMVAKNIRGSIICTASVESILAGAAPLAYIASKHGILGVVKAAARELGKNGIRVNCVSPFGIATPMVCKSYGAEASYIETSVGGHANLKGVSLTTKHIAEAALFLASEESAYISGQNLAVDGGLSAIMRLD

SEQ ID NO: 72 - protein, 260 aa, Solanum melongena
MANKLRLEGKVAVITGGASGIGEASARLFVEHGARVVIADIQDELSLQVVSSIGGDKACYRRCDVSDEKQVEETVAYAVEKYGTLDIMFSNAGILGSFGSLLEMDMTAFDRIMAVNTRGAALAVKHAARVMVANKIRGSIICTASVEAILAGEASLAYIASKHAILGVVKAAARDLGQYGIRVNCVSPYGIATPMVCKSIGADAATIEARICGNANLKGVSLNTKHIAEAALFLGSDESAYVSAHNLAVDGGLSSIMKLN

SEQ ID NO: 73 - protein, 262 aa, Solanum tuberosum
MANKLRLEGKVAVITGGASGIGEATARLFVEHGAHVVIADIQDELALQVVSSIGSDNVCYRRCDVTDEKQVDETVAFAVQKYGTLDIMFSNAGILGSSGSLLEMDMAVFDRTMAVNTRGAALAVKHAAKVMVAKKIRGSIICTASVESILAGAASLAYIASKHAVLGVVKAAARELGQHGIRVNCVSPFGVATPMVCKSFGADAAAMEATIRGNANLKGVSLTTMHIAEAALFLASDESAYISAHNLAIDGGLSSIMKINVN

SEQ ID NO: 74 - protein, 260 aa, Solanum lycopersicum
MANKLRLEGKVAVITGGASGIGEAAARLFVEHGARVVIADIQDELALQVASSIGSDNVCYQRCDVSDEKQVNETVAFAVEKYGTLDIMFSNAGILNPFESILEMDMTVFDRTIAVNARGAALAVKHAARVMVANKIRGSIICTASVESILAGAAPLAYIASKHAVLGVVKAAARELGQHGIRVNCVSPFGIATPMVCKSFGADAAAIEAKICGNANLKGVSLTTMHIAEAALFLASDESAYISAQNLAVDGGLSSMMKLM

SEQ ID NO: 75 - protein, 260 aa, Solanum pennellii
MANKLRLEGKVAVITGGASGIGEAAARLFVEHGARVVIADIQDELALQVASSIGSVNVCCRRCDVSDEKQVNETVAFAVEKYGTLDIMFSNAGILNPFESILEMDMTVFDRTIAVNARGAALAVKHAARVMVANKIRGSIICTASVESILAGAAPLAYIASKHAVLGVVKAAARELGQHGIRVNCVSPFGIATPMVCKSFGADAAAIEAKICGNANLKGVSLTTMHIAEAALFLASDESAYISAQNLAVDGGLSSMMKLM

SEQ ID NO: 76 - protein, 260 aa, Nicotiana benthamiana
MANKRRLEGKVAVITGAASGIGEATARLFVEHGARVVIADIQDELGHQVVASIGTDKASYRHCDVTDEKQVEDTVVYTVEKYGTLDIMFSNAGTIGTLGSILDMDMTVFDRTMAINARGSALAVKHAARVMVTKKIQGSIICTASLEATLAGAAPLAYVTSKHAILGVVKAAARELGQHGIRVNCVSPYGIATPMVCKTFGGDAAPIEASISGNANLKGITLSTKHIAEAALFLASDESAYVSAHNLAVDGGLSSIMKLD

SEQ ID NO: 77 - protein, 241 aa, Nicotiana benthamiana
MANKLRLEGKVAVITGAASGIGEATARLFVEHGARVVIADIQDELGHQVVASIGTDKASYRHCDVTDEKQVEDTVVYAVEKYGTLDIMFSNAGTIGTLSSILDMDMTVFDRTMAINARGSALAVKHAARIMVTKKIQGSIICTASLEAILAGAAPLAYVASKHAILGVVKAAARELGQHGIRVNCVSPYGIATPMVCKTFGGDAAPIEASISGNANLKGITLSTKHIAEAALFLASDESAY

SEQ ID NO: 78 - protein, 257 aa, Arabidopsis thaliana
MSGKRLDGKIVIITGGASGIGAESVRLFTEHGARVVIVDVQDELGQNVAVSIGEDKASYYHCDVTNETEVENAVKFTVEKYGKLDVLFSNAGVIEPFVSILDLNLNELDRTIAINLRGTAAFIKHAARAMVEKGIRGSIVCTTSVAAEIAGTAPHGYTTSKHGLLGLIKSASGGLGKYGIRVNGVAPFGVATPLVCNGFKMEPNVVEQNTSASANLKGIVLKARHVAEAALFLASDESAYVSGQNLAVDGGYSVVKP

SEQ ID NO: 79 - protein, 266 aa, Medicago truncatula
MSRKRLEGKVAIVTGGASGIGAETAKTFVENGAFVVIADINDELGHQVATSIGLDKVSYHHCDVRDEKQVEETVAFALEKYGTLDIMFSNAGIEGGMSSSILEFDLNEFDNTMAINVRGSLAAIKHAARFMVERKIRGSIICTASVAASVAGNRGHDYVTSKHGLLGLVRSTCGELGAYGIRVNSISPYGVATPLACRALNMEMSKVEANMKDSANLKGITLKATHIAEAALFLASEESAYISGHNLVVDGGFSVINSCVPTTIKK

SEQ ID NO: 80 - protein, 266 aa, Capsicum annuum
MAVVMQKLKGKVAIVTGGASGIGEATVRLFAEHGARAVVIADIQDEKGRAVAESIPLQVCSYVHCDVSDENQVKGLVDWTVKKYGQLDIMFSNAGTVGNSGQKVLDLDLSEFDRVIRVNARGMAACVKHAARAMVEQGGRGSIICTGSVGASKGAAWRTDYTMSKHAVLGLVTSASRQLGKYGIRVNSISPSAVMTPLMSSAEAETSMKVLKMYGPLTSLKGITLTVKHLADAVLFLASDDSAFVNGHDLLVDGGLLHLPDPMSSL

SEQ ID NO: 81 - protein, 266 aa, Solanum tuberosum
MAEVTQKLKGKVAIVTGGASGIGEATARLFAQHGARAVVIADIQDGKGRAVAVSIPSQICSYVQCDVSDENQVKAMVDWTVQKYGQLDIMFSNAGVVGNSGQKVLDLDLSEFDRVMNVNARGMAACVKHAARAMVDKRVRGSIICTGSIGASRGGAWRTDYIMSKHAVLGLVRSACRQLGEYGIRVNSISPSAVMTPLMISAEPEVSMKSLKRYGPQTSLKGITLTVKHLAEAALFLASDDSAFSSRSNEFIVKQREQPNLSLFFF

SEQ ID NO: 82 - protein, 266 aa, Solanum melongena
MSAITQKLNGKVAIVTGGASGIGEATVRLFAQHGARAVVIADIQDEKGRAVAQSIPSQICIYVKCDVSDENQVKSMVDWTVQQYGQLDIMFSNAGTVGNSGQKILDLDLSEFDRVMNVNARGMAACVKHAARAMVEKRVRGSIICTGSIAASRAGAWRTDYAMSKHAVLGLMRSASRQLGEYGIRVNSISPSAVMTPLMISAEAEASMRVLKMYGSVTSLKGITLTVKHLADAVLFLASDDSVFVSGHDLAVDGGLISLPDPMSSL

SEQ ID NO: 83 - protein, 302 aa, Oryza sativa
MFTAMHRILSRGRRTPAASSSSVTAFATASDSQRLAGKVAVITGGASGIGRATAEEFVRNGAKVILADVQDDLGHAVAAELGADAASYARCDVTDEAQVAAAVDLAVARHGRLDVVFNNAGIPGDLTPTPVGALDLADFDRVMAVNTRAVVAGVKHAARVMVPRRRGSIICTASTAGVIGGVAVPHYSVSKAAVLGLVRAVAGEMARSGVRVNAISPNYIWTPMAAVAFARWYPSRSADDHRRIVENDINEMDGVTLEAEDVARAAVFLASDEAKYVNGHNLVVDGGYTVGKVPNMPVPDGH

SEQ ID NO: 84 - protein, 300 aa, Oryza sativa
MFRAAQLLLRETNRALGAATSPAGFVSGFSTASNSAQRLAGKVAVITGGASGIGKATAKEFIENGAKVIMADVQDDLGHSTAAELGPDASYTRCDVTDEAQVAAAVDLAVKRHGHLDILYNNAGVMGAMPQDDMASVDLANFDRMMAINARAALVGIKHAARVMSPRRSGVILCTASDTGVMPMPNIALYAVSKATTIAIVRAAAEPLSRHGLRVNAISPHGTRTPMAMHVLSQMYPGVSKDDLEKMADAAMDAGEVMEPKYVARAALYLASDEAKYVNGHNLVVDGGFTSHKGSDTRLN

SEQ ID NO: 85 - protein, 285 aa, Arabidopsis thaliana
MSTNTESSSYSSLPSQRLLGKVALITGGATGIGESIVRLFHKHGAKVCIVDLQDDLGGEVCKSLLRGESKETAFFIHGDVRVEDDISNAVDFAVKNFGTLDILINNAGLCGAPCPDIRNYSLSEFEMTFDVNVKGAFLSMKHAARVMIPEKKGSIVSLCSVGGVVGGVGPHSYVGSKHAVLGLTRSVAAELGQHGIRVNCVSPYAVATKLALAHLPEEERTEDAFVGFRNFAAANANLKGVELTVDDVANAVLFLASDDSRYISGDNLMIDGGFTCTNHSFKVFR

SEQ ID NO: 86 - protein, 275 aa, Oryza sativa
MAGSSYGDVHESARKLVGKVALITGGASGIGECTARLFVKHGAQVVVADIQDEAGARLCAELGSATASYVRCDVTSEDDVAAAVDHAVARYGKLDVMFNNAGIGGAACHSILESTKADFDRVLAVNLTGPFLGTKHAARVMVAAGRGGCIIGTASLASAVAGTASHAYTCAKRALVGLTENAAAELGRHGIRVNCVSPAAAATPLATGYVGLEGEAFEAAMEAVANLKGVRLRVEDIAAAVLFLASDDARYVSGHNLLIDGGCSIVNPSFGIFKD

SEQ ID NO: 87 - protein, 274 aa, Oryza sativa
MAAGSSHVSADARKLVGKVAVITGGASGIGACTARLFVKHGARVVVADIQDELGASLVAELGPDASSYVHCDVTNEGDVAAAVDHAVARFGKLDVMFNNAGVSGPPCFRMSECTKEDFERVLAVNLVGPFLGTKHAARVMAPARRGSIISTASLSSSVSGAASHAYTTSKHALVGFTENAAGELGRHGIRVNCVSPAGVATPLARAAMGMDDEAIEAIMANSANLKGAGALKADDIAAAALFLASDDGRYVSGQNLRVDGGLSVVNSSFGFFRD

SEQ ID NO: 88 - protein, 274 aa, Oryza sativa
MAGSSHVSADARKLVGKVAVITGGASGIGACTARLFVKHGARVVVADIQDELGASLVAELGPDASSYVHCDVTNEGDVAAAVDHAVATFGKLDVMFNNAGVTGPPCFRITESTKEDFERVLAVNLIGPFLGTKHAARVMAPARRGSIISTASLSSSVSGTASHAYTTSKRALVGFTENAAGELGRHGIRVNCVSPAAVATPLARAAMGMDMDDETIEAIMEKSANLKGVGLKVDDIAAAALFLASDDGRYVSGQNLRVDGGVSVVNSSFGFFRD

SEQ ID NO: 89 - protein, 349 aa, Arabidopsis thaliana
MELINDFLNLTAPFFTFFGLCFFLPPFYFFKFLQSIFSTIFSENLYGKVVLITGASSGIGEQLAYEYACRGACLALTARRKNRLEEVAEIARELGSPNVVTVHADVSKPDDCRRIVDDTITHFGRLDHLVNNAGMTQISMFENIEDITRTKAVLDTNFWGSVYTTRAALPYLRQSNGKIVAMSSSAAWLTAPRMSFYNASKAALLSFFETMRIELGGDVHITIVTPGYIESELTQGKYFSGEGELIVNQDMRDVQVGPFPVASASGCAKSIVNGVCRKQRYVTEPSWFKVTYLWKVLCPELIEWGCRLLYMTGTGMSEDTALNKRIMDIPGVRSTLYPESIRTPEIKSD

SEQ ID NO: 90 - protein, 264 aa, Arabidopsis thaliana
METDKRWSLAGKTALVTGGTRGIGRAVVEELAKFGAKVHTCSRNQEELNACLNDWKANGLVVSGSVCDASVRDQREKLIQEASSAFSGKLNILINNVGTNVRKPTVEYSSEEYAKIMSTNLESAFHLSQIAHPLLKASGVGSIVFISSVAGLVHLSSGSIYGATKGALNQLTRNLACEWASDNIRTNCVAPWYIKTSLVETLLEKKEFVEAVVSRTPLGRVGEPEEVSSLVAFLCLPASSYITGQVISVDGGFTVNGFSYAMKP

SEQ ID NO: 91 - protein, 310 aa, Zea mays
MDAAAAAASPTSKRIALVTGGNKGIGLETCRQLASRGVRVVLTARNEARGLEAVERVRCARGDAEVYFHQLDVTDPCSAARLADFVRDQFGRLDILINNAGISGVHRDPVLSAAVKDKVDGMDVNQRVEWMKENSKETYEEAVQCMKTNYYGAKLVTEALLPLLQLSSSGRIVNVSSGFGLLRNFNSEDLRKEFEDIDNLTESRLEELMDKFLEDFKANLVEEHGWPTGGSSAYKVVKAALNAYTRILAKKYPTLRINCLTPGYVKTDISMHMGVLTLEEGARNPVKVALLPDDGPTGAYFDLNGEASFV

SEQ ID NO: 92 - protein, 303 aa, Arabidopsis thaliana
MAEETPRLFNGFCRYAVVTGANRGIGFEICRQLASEGIRVVLTSRDENRGLEAVETLKKELEISDQSLLFHQLDVADPASITSLAEFVKTQFGKLDILVNNAGIGGIITDAEALRAGAGKEGFKWDEIITETYELTEECIKINYYGPKRMCEAFIPLLKLSDSPRIVNVSSSMGQLKNVLNEWAKGILSDAENLTEERIDQVINQLLNDFKEGTVKEKNWAKFMSAYVVSKASLNGYTRVLAKKHPEFRVNAVCPGFVKTDMNFKTGVLSVEEGASSPVRLALLPHQETPSGCFFSRKQVSEF

SEQ ID NO: 93 - protein, 311 aa, Papaver bracteatum
MPETCPNTVTKMRCAVVTGGNKGIGFEICKQLSSSGIMVVLTCRDVTRGLEAVEKLKNSNHENVVFHQLDVTDPITTMSSLADFIKARFGKLDILVNNAGVAGFSVDADRFKAMISDIGEDSEEVVKIYEKPEAQELMSETYELAEECLKINYYGVKSVTEVLLPLLQLSDSPRIVNVSSSTGSLKYVSNETALEILGDGDALTEERIDMVVNMLLKDFKENLIETNGWPSFGAAYTTSKACLNAYTRVLAKKIPKFQVNCVCPGLVKTEMNYGIGNYTADEGAKHVVRIALFPDDGPSGFFYDCSELSAF

SEQ ID NO: 94 - protein, 343 aa, Zea mays
MSTGGRKMRTACVTGGNGYIASALIKVLLEKGYAVKTTVRNPDDMEKNSHLKDLQALGSLEVFRADLDEDGSFDDAVAGCDYAFLVAAPVNLHTKNPEEEMIEPAVRGTLNVMRSCVKAGTVRRVVLTSSAAAVTTRPQLQGDGHVLDEESWSDVEYLRAHKPAGPWGYPVSKVLLEKEASRFAEEHGIGLVTVCPGLTVGAAPAPTARTSVPNCLSLLSGDEAAFAVLDAIESATGCLPLVHVDDVCRAELFAAEEGAAARRYVCCGLNTTVAELARFLADKYPQYGVKTNLLSGERLEKPRVCLSSAKLVKEGFEFRYRTLDDIYDDMVEYGKALGILPDL

SEQ ID NO: 95 - protein, 340 aa, Arabidopsis thaliana
MDQTLTHTGSKKACVIGGTGNLASILIKHLLQSGYKVNTTVRDPENEKKIAHLRKLQELGDLKIFKADLTDEDSFESSFSGCEYIFHVATPINFKSEDPEKDMIKPAIQGVINVLKSCLKSKSVKRVIYTSSAAAVSINNLSGTGIVMNEENWTDVEFLTEEKPFNWGYPISKVLAEKTAWEFAKENKINLVTVIPALIAGNSLLSDPPSSLSLSMSFITGKEMHVTGLKEMQKLSGSISFVHVDDLARAHLFLAEKETASGRYICCAYNTSVPEIADFLIQRYPKYNVLSEFEEGLSIPKLTLSSQKLINEGFRFEYGINEMYDQMIEYFESKGLIKAK

SEQ ID NO: 96 - protein, 348 aa, Papaver somniferum
MHGQKNISERYQKFKEMEGTGKIVCVTGGAGYLASWLIMRLLERGYSVRTTVRSDPKFREDVSHLKALPEATEKLQIFEADLENPESFDDAINGCVGVFLVAQGMNFAEEYTLEKIIKTCVEGTLRILQSCLKSKTVKKVVYTSSADAAMMISNLKAVKEIDETIWSEVDNFISKPEQVIPGLPSYVVSKVLTERACLKFSEEHGLDVVTILPPLVVGPFITPHPPPSVSIALSIISGDVSMMLGVRLENAVHIDDVALAHIFVFECEKAKGRHICSSVDFPMHDLPKFISENYPEFNVPTDLLKDIEEQEPVHLSSDKLLSMGFQFKYDFAEIFGDAIRCAKEKGFL

SEQ ID NO: 97 - protein, 360 aa, Arabidopsis thaliana
MVREEEEDDNNGGGGERKLPVADETVPSLLDGTGLVCVTGGTGFVASWLIMRLLQRGYSVRATVRTNPEGNKKDISYLTELPFASERLQIFTADLNEPESFKPAIEGCKAVFHVAHPMDPNSNETEETVTKRTVQGLMGILKSCLDAKTVKRFFYTSSAVTVFYSGKNGGGGGEVDESVWSDVEVFRNQKEKRVSSSYVVSKMAAETAALEFGGKNGLEVVTLVIPLVVGPFISPSLPSSVFISLAMLFGNYKEKYLFDTYNMVHIDDVARAMILLLEKPVAKGRYICSSVEMKIDEVFEFLSTKFPQFQLPSIDLKNYKVEKRMSLSSKKLRSEGFEFKYGAEEIFGGAIRSCQARGFL

SEQ ID NO: 98 - protein, 323 aa, Homo sapiens
MDPKYQRVELNDGHFMPVLGFGTYAPPEVPRNRAVEVTKLAIEAGFRHIDSAYLYNNEEQVGLAIRSKIADGSVKREDIFYTSKLWCTFFQPQMVQPALESSLKKLQLDYVDLYLLHFPMALKPGETPLPKDENGKVIFDTVDLSATWEVMEKCKDAGLAKSIGVSNFNCRQLEMILNKPGLKYKPVCNQVECHPYLNQSKLLDFCKSKDIVLVAHSALGTQRHKLWVDPNSPVLLEDPVLCALAKKHKRTPALIALRYQLQRGVVVLAKSYNEQRIRENIQVFEFQLTSEDMKVLDGLNRNYRYVVMDFLMDHPDYPFSDEY

SEQ ID NO: 99 - protein, 323 aa, Mus musculus
MDSKQQTVRLSDGHFIPILGFGTYAPQEVPKSKATEATKIAIDAGFRHIDSASMYQNEKEVGLAIRSKIADGTVKREDIFYTSKVWCTFHRPELVRVCLEQSLKQLQLDYVDLYLIHFPMAMKPGENYLPKDENGKLIYDAVDICDTWEAMEKCKDAGLAKSIGVSNFNRRQLEKILKKPGLKYKPVCNQVECHPYLNQGKLLDFCRSKDIVLVAYSALGSHREKQWVDQSSPVLLDNPVLGSMAKKYNRTPALIALRYQLQRGVVVLAKSFSEKRIKENMQVFEFQLTSEDMKVLDDLNKNIRYISGSSFKDHPDFPFWDEY

SEQ ID NO: 100 - protein, 317 aa, Solanum lycopersicum
MAEATEMPYIELNTGFSIPAVGLGTWQSDPGVVGKAVETAIKMGYRHIDCAQIYKNEKEIGEVLSRLFKDGVVKRRELFITSKLWNTNHAPEDVPVALDKTLQDLQLEYVDLYLIHWPVSMKPGSVDFKPENLMPTNIPRIWEAMEKVYDSGKARVIGVSNFSTKKLEDLLQVARTPPAVNQVECHPSWQQAKLRELCKSNNVHLSAYSPLGSPGTTWLKSDVLKQPAVISVAEKLGKTPAQVCLRWGIQMGQSVLPKSTHEARIKENLDVLNWSIPDDLLAKFSEIPQARLLKGASFAHETHGQYRTLEELWDGEI

SEQ ID NO: 101 - protein, 331 aa, Solanum lycopersicum
MTMNLIKQMLVPNVNLNSGHKMPLIGMGTAPSLPEHDQLVSTLIDAIEIGYRHFDTAAVYGSEEALGQAVVEAIQRGLIKSREQVFITSKLWCTETHRHLVLPALKRTLGRLKMDYLDLYLIHLPVTMKKKVNSKDDEMRVDKEDIIPFDMRGTWEAMEECCRLGLAKSIGVSNFTCTKISQILHYATILPAVNQVEMHVAWRQEKMLEFCKEKGIHVSAWSPLGANGLTPWGIHSVMESPVLKDIAIHKRKSVAQVALRWVYEQGASVIVKSFNKERMKENLQILDWELSNEEIAQIQEIPPCTGFNVDMVLVHPNGPYKSANQFWDGEI

SEQ ID NO: 102 - protein, 312 aa, Medicago truncatula
MGSVEIPTKVLTNTSSQLKMPVVGMGSAPDFTCKKDTKDAIIEAIKQGYRHFDTAAAYGSEQALGEALKEAIELGLVTRQDLFVTSKLWVTENHPHLVIPALQKSLKTLQLDYLDLYLIHWPLSSQPGKFTFPIDVADLLPFDVKGVWESMEEGLKLGLTKAIGVSNFSVKKLENLLSVATILPAVNQVEMNLAWQQKKLREFCNANGIVLTAFSPLRKGASRGPNEVMENDMLKEIADAHGKSVAQISLRWLYEQGVTFVPKSYDKERMNQNLCIFDWSLTKEDHEKIDQIKQNRLIPGPTKPGLNDLYDD

SEQ ID NO: 103 - protein, 321 aa, Papaver somniferum
MESNGVPMITLSSGIRMPALGMGTAETMVKGTEREKLAFLKAIEVGYRHFDTAAAYQTEECLGEAIAEALQLGLIKSRDELFITSKLWCADAHADLVLPALQNSLRNLKLDYLDLYLIHHPVSLKPGKFVNEIPKDHILPMDYKSVWAAMEECQTLGFTRAIGVCNFSCKRLQELMETANSPPVVNQVEMSPTLHQKNLREYCKANNIMITAHSVLGAVGAAWGTNAVMHSKVLHQIAVARGKSVAQVSMRWVYQQGASLVVKSFNEARMKENLKIFDWELTAEDMEKISEIPQSRTSSAAFLLSPTGPFKTEEEFWDEKD

SEQ ID NO: 104 - protein, 320 aa, Arabidopsis thaliana
MSALTFPIGSVHHLMPVLALGTAASPPPEPIVLKRTVLEAIKLGYRHFDTSPRYQTEEPLGEALAEAVSLGLIQSRSELFVTSKLWCADAHGGLVVPAIQRSLETLKLDYLDLYLIHWPVSSKPGKYKFPIEEDDFLPMDYETVWSEMEECQRLGVAKCIGVSNFSCKKLQHILSIAKIPPSVNQVEMSPVWQQRKLRELCKSKGIVVTAYSVLGSRGAFWGTHKIMESDVLKEIAEAKGKTVAQVSMRWAYEEGVSMVVKSFRKDRLEENLKIFDWSLTEEEKQRISTEISQSRIVDGEVYISEKGPIKSVTEMWDGEI

SEQ ID NO: 105 - protein, 259 aa, Digitalis lanata
MSSKPRLEGKVAIITGAASGIGEETARLFVEHGASVVVADVQDELGRQVVASVNSDDKISYYHCDVRDEKQVAATVRYAVEKYGRLDIMLSNAGVFGALMTNVIDLDMVDFENVLATNVRGVANTIKHAARAMVEGKVKGSIICTASVSASLGGMGPPAYTASKHAVLGLVKGACAELGVHGIRVNSVAPYGVATPMPCSAYGMTPSQMEEANNSRANLKGVVLKAKHVAEAALFLASDESAYVSGQNLAVDGGFTVVR

SEQ ID NO: 106 - DNA, 33 bp, synthetic construct
  misc_feature 1..33: note = GAME25 silencing construct for S. pennellii forward primer
  source 1..33: mol_type = other DNA
gcggccgcattgtcactctattgtgttggcgtg

SEQ ID NO: 107 - DNA, 36 bp, synthetic construct
  misc_feature 1..36: note = GAME25 silencing construct for S. pennellii reverse primer
  source 1..36: mol_type = other DNA
ggcgcgcctaaatttatatcttttcaagtcacaatg